(19) United States
(12) Patent Application Publication
(10) Pub. No.: US 2026/0041100 A1
(43) Pub. Date: Feb. 12, 2026

(54) STABLE LIQUID FORMULATIONS FOR NITROGEN-FIXING MICROORGANISMS

(71) Applicant: Pivot Bio, Inc., Minnetonka, MN (US)

(72) Inventors: Farzaneh REZAEI, Berkeley, CA (US); Mahsa MOHITI-ASLI, Berkeley, CA (US); Naomi KREAMER, Berkeley, CA (US); Allison Nicole JOHNSON, Berkeley, CA (US); Tabitha AMENDOLARA, Berkeley, CA (US)

(21) Appl. No.: 19/367,462

(22) Filed: Oct. 23, 2025

Related U.S. Application Data
(63) Continuation of application No. 18/050,698, filed on Oct. 28, 2022, now Pat. No. 12,478,068.
Application No. 18/050,698 is a continuation of application No. PCT/US2021/029993, filed on Apr. 29, 2021.
(60) Provisional application No. 63/019,096, filed on May 1, 2020.

(51) Int. Cl. A01N 63/20; C05F 11/08; C12N 1/04; C12N 1/20

(52) U.S. Cl. CPC A01N 63/20; C05F 11/08; C12N 1/04; C12N 1/20

(57) ABSTRACT

The present disclosure provides agronomically stable liquid agricultural compositions, methods of formulation thereof, and methods of application thereof. The agricultural compositions comprise nitrogen-fixing microorganisms and one or more of a buffering agent, a microbial stabilizer, and a physical stabilizer. The disclosed liquid agricultural compositions have a longer shelf life and greater ease of application than other existing dry and liquid formulations. The disclosed liquid agricultural compositions are stable for a period of thirty days or longer with low toxin accumulation and high microbial stability. The compositions are suitable for use on agricultural plant tissues or the environs thereof for providing a source of fixed atmospheric nitrogen to the agricultural plant. The compositions are used to increase crop yield and decrease yield variance.

### CROSS-REFERENCE TO RELATED APPLICATIONS

This application is a continuation of U.S. application Ser. No. 18/050,698 entitled “STABLE LIQUID FORMULATIONS FOR NITROGEN-FIXING MICROORGANISMS” filed Oct. 28, 2022, which is a continuation of International Application No. PCT/US2021/029993 entitled “STABLE LIQUID FORMULATIONS FOR NITROGEN-FIXING MICROORGANISMS” filed Apr. 29, 2021, which claims the benefit of U.S. Provisional Application No. 63/019,096, entitled “STABLE LIQUID FORMULATIONS FOR NITROGEN-FIXING MICROORGANISMS” filed on May 1, 2020. The contents of each of the aforementioned documents are incorporated by reference herein in their entirety.

### REFERENCE TO AN ELECTRONIC SEQUENCE LISTING

The contents of the electronic sequence listing (PIVO_020_01US_SeqList_ST26.xml; Size: 638,663 bytes; and Date of Creation: Oct. 27, 2022) is herein incorporated by reference in its entirety.

### FIELD OF THE DISCLOSURE

The present disclosure relates to agronomically stable liquid agricultural compositions comprising nitrogen-fixing microorganisms, methods of formulation thereof, and methods of use thereof. The agricultural compositions may have improved stability at room temperature, allowing for greater ease of storage and use for agricultural crops. Also provided are methods of formulating agricultural compositions with improved stability comprising varying one or more parameters of the formulation process and/or ingredients. The agricultural compositions may be used to improve one or more aspects of the agricultural crop to which they are exposed, including yield and productivity.

### BACKGROUND

Plant beneficial microbes, such as gram-negative bacteria, have the potential to increase growth and increase yields of crops under a variety of environmental stresses. These beneficial microbes, specifically those used for nitrogen fixation, must be cultured and transplanted to the soil near the root structure of the plant. Existing agricultural formulations for administering such microbes to agricultural plant tissues or their environment face ongoing problems with microbial stability, shelf life, and ease of use.

Most existing agricultural formulations comprising nitrogen-fixing microorganisms come in either dry or liquid forms. Dry powders have the benefit of improved stability and ease of storage, but often require the steps of mixing with a liquid carrier and activation by the user-steps that can compromise the viability of the formulation if incorrectly performed. After activation, many of these formulations also suffer from a limited shelf life in liquid form, which can lead to further difficulties in their use. Liquid formulations of nitrogen-fixing microorganisms allow for more complete control over the formulation process and greater ease of application by the user, but suffer from poor shelf life, complicating their storage and delivery.

There is an unmet need for improved liquid agricultural formulations comprising nitrogen-fixing bacteria for improving one or more traits of agricultural crops.

### BRIEF SUMMARY

In one aspect, the present disclosure provides an agronomically stable liquid agricultural composition, comprising: a) a diazotrophic bacterium; b) a buffering agent; c) a microbial stabilizer; and d) a physical stabilizer, wherein the composition has a room temperature shelf life of at least 30 days.

In some embodiments, the microbial stability of the composition is greater than the microbial stability of the composition absent one or more of the buffering agent, microbial stabilizer, and physical stabilizer.

In some embodiments, the shelf life is at least two months, at least three months, at least four months, or at least five months.

In some embodiments, the shelf life is at least three months.

In some embodiments, the log loss of CFU/mL over the shelf life of the composition is less than 0.2.

In some embodiments, the bacterium is present at a cellular density that provides an acceptable rate of decay over the shelf life of the composition.

In some embodiments, the bacterium is present at a cellular density that minimizes the rate of decay over the shelf life of the composition.

In some embodiments, the bacterium is present at a cellular density that provides a reduced, but not minimized rate of decay.

In some embodiments, the bacterium is present at a cellular density that provides a rate of decay of less than 1.0 log loss of CFU/mL over 30 days in the agricultural composition absent the buffering agent, microbial stabilizer, and physical stabilizer.

In some embodiments, the bacterium is present at a cellular density of about 3E9-6E9 CFU/mL.

In some embodiments, the buffering agent maintains the pH of the composition over the shelf life of the composition.

In some embodiments, the buffering agent maintains the pH of the composition at about pH 6-8 over the shelf life of the composition.

In some embodiments, the buffering agent maintains the pH of the composition at about pH 6.5 over the shelf life of the composition.

In some embodiments, the buffering agent is selected from the list consisting of phosphate buffered saline (PBS); modified, high buffering capacity PBS; 3-Morpholinopropane-1-sulfonic acid (MOPS); and 2-[4-(2-hydroxyethyl)piperazin-1-yl]ethanesulfonic acid (HEPES).

In some embodiments, the buffering agent is modified, high buffering capacity PBS.

In some embodiments, the microbial stabilizer slows the doubling rate of the diazotrophic bacterium.

In some embodiments, the microbial stabilizer slows the toxin accumulation rate within the composition.

In some embodiments, the microbial stabilizer is a monosaccharide, disaccharide, polysaccharide, pentose, hexose, oligosaccharide, oligofructose, sugar alcohol, amino acid, protein or protein hydrolysate, or polymer.

In some embodiments, the microbial stabilizer is a monosaccharide or a disaccharide selected from the list consisting of glucose, fructose, trehalose, sucrose, lactose, melibiose, and lactulose.

In some embodiments, the microbial stabilizer is fructose or trehalose.

In some embodiments, the microbial stabilizer is fructose.

In some embodiments, the microbial stabilizer is fructose and is present in the composition at a concentration of about 0.5-2.5% w/v.

In some embodiments, the microbial stabilizer is fructose and is present in the composition at a concentration of about 1.3% w/v.

In some embodiments, the physical stabilizer decreases the local density of the diazotrophic bacterium within the composition.

In some embodiments, the physical stabilizer is a polysaccharide, protein or protein hydrolysate, polymer, or a natural gum or its derivative.

In some embodiments, the physical stabilizer is a polysaccharide.

In some embodiments, the physical stabilizer is a polysaccharide selected from the list consisting of maltodextrin, polyethylene glycol (PEG), xanthan gum, pectin, alginates, microcrystalline cellulose, and dextran.

In some embodiments, the physical stabilizer is xanthan gum.

In some embodiments, the physical stabilizer is xanthan gum and is present in the composition at a concentration of about 0.001-0.2% w/v.

In some embodiments, the physical stabilizer is xanthan gum and is present in the composition at a concentration of about 0.1% w/v.

In some embodiments, the bacterium is a gram-negative bacterium.

In some embodiments, the bacterium is of a genus selected from the group consisting of: Acetobacter, Achromobacter, Aerobacter, Anabaena, Azoarcus, Azomonas, Azorhizobium, Azospirillum, Azotobacter, Beijernickia, Bradyrhizobium, Burkholderia, Citrobacter, Derxia, Enterobacter, Herbaspirillum, Klebsiella, Kluyvera, Kosakonia, Mesorhizobium, Metakosakonia, Paraburkholderia, Nostoc, Rahnella, Rhizobium, Rhodobacter, Rhodopseudomonas, Rhodospirillum, Serratia Sinorhizobium, Spirillum, Trichodesmium, and Xanthomonas.

In some embodiments, the bacterium is of a species selected from the group consisting of: Achromobacter marplatensis, Achromobacter spiritinus, Azospirillum lipoferum, Enterobacter sacchari, Herbaspirillum aquaticum, Klebsiella variicola, Kluyvera intermedia, Kosakonia pseudosacchari, Kosakonia sacchari, Metakosakonia intestini, Paraburkholderia tropica, Rahnella aquatilis, and combinations thereof.

In some embodiments, the bacterium is a gram-positive bacterium.

In some embodiments, the bacterium is of a genus selected from the group consisting of: Arthrobacter, Agromyces, Bacillus, Clostridium, Corynebacterium, Frankia, Heliobacillus, Heliobacterium, Heliophilum, Heliorestis Methanobacterium, Microbacterium, Micrococcus, Micromonospora, Mycobacterium, Paenibacillus, Propionibacterium, and Streptomyces.

In some embodiments, the bacterium is of a species selected from the group consisting of: Bacillus amyloliquefaciens, Bacillus macerans, Bacillus pumilus, Bacillus thuringiensis, Clostridium acetobutylicum, Corynebacterium autitrophicum Methanobacterium formicicum, Methanobacterium omelionski, Microbacterium murale, Mycobacterium flavum, Paenibacillus polymyxa, Paenibacillus riograndensis, Propionibacterium acidipropio, Propionibacterium freudenreichii, Streptococcus lactis, Streptomyces griseus, and combinations thereof.

In some embodiments, the bacterium is of the genus Klebsiella.

In some embodiments, the bacterium is of the species Klebsiella variicola.

In some embodiments, the bacterium is of the strain Klebsiella variicola NCMA 201712002.

In some embodiments, the bacterium is of the genus Kosakonia.

In some embodiments, the bacterium is of the species Kosakonia sacchari.

In some embodiments, the bacterium is of the strain Kosakonia sacchari ATCC PTA-126743.

In some embodiments, the bacterium is endophytic, epiphytic, or rhizospheric.

In some embodiments, the bacterium is a wild type bacterium.

In some embodiments, the bacterium is an engineered bacterium.

In some embodiments, the bacterium is a transgenic bacterium.

In some embodiments, the bacterium is an intragenic bacterium.

In some embodiments, the bacterium is a remodeled bacterium.

In some embodiments, the bacterium comprises a non-intergeneric genomic modification.

In some embodiments, the bacterium is an engineered bacterium capable of fixing atmospheric nitrogen in the presence of exogenous nitrogen.

In some embodiments, the bacterium is an engineered bacterium comprising at least one genetic variation introduced into at least one gene, or non-coding polynucleotide, of the nitrogen fixation or assimilation genetic regulatory network.

In some embodiments, the bacterium is an engineered bacterium comprising an introduced control sequence operably linked to at least one gene of the nitrogen fixation or assimilation genetic regulatory network.

In some embodiments, the bacterium is an engineered bacterium comprising a heterologous promoter operably linked to at least one gene of the nitrogen fixation or assimilation genetic regulatory network.

In some embodiments, the bacterium is an engineered bacterium comprising at least one genetic variation selected from the group consisting of: nifA, nifL, ntrB, ntrC, polynucleotide encoding glutamine synthetase, glnA, glnB, glnK, drat, amtB, polynucleotide encoding glutaminase, glnD, glnE, nifJ, nifH, nifD, nifK, nifY, nifE, nifN, nifU, nifS, nifV, nifW, nifZ, nifM, nifF, nifB, nifQ, a gene associated with biosynthesis of a nitrogenase enzyme, and combinations thereof.

In some embodiments, the bacterium is an engineered bacterium comprising at least one genetic variation introduced into at least one gene, or non-coding polynucleotide, of the nitrogen fixation or assimilation genetic regulatory network that results in one or more of: increased expression or activity of NifA or glutaminase; decreased expression or activity of NifL, NtrB, glutamine synthetase, GlnB, GlnK, DraT, AmtB; decreased adenylyl-removing activity of GlnE; or decreased expression or uridylyl-removing activity of GlnD.

In some embodiments, the bacterium is an engineered bacterium comprising a mutated nifL gene that has been altered to comprise a heterologous promoter inserted into said nifl gene.

In some embodiments, the bacterium is an engineered bacterium comprising a mutated glnE gene that results in a truncated GlnE protein lacking an adenylyl-removing (AR) domain.

In some embodiments, the bacterium is an engineered bacterium comprising a mutated glnD gene that results in the lack of expression of said glnD gene.

In some embodiments, the bacterium is an engineered bacterium comprising a mutated amtB gene that results in the lack of expression of said amtB gene.

In some embodiments, the bacterium is an engineered bacterium comprising at least one of: a mutated nifL gene that has been altered to comprise a heterologous promoter inserted into said nifL gene; a mutated glnE gene that results in a truncated GlnE protein lacking an adenylyl-removing (AR) domain; a mutated amtB gene that results in the lack of expression of said amtB gene; a mutated glnD gene that results in the lack of expression of said glnD gene; and combinations thereof.

In some embodiments, the bacterium is an engineered bacterium comprising at least one genetic variation introduced into genes involved in a pathway selected from the group consisting of: exopolysaccharide production, endo-polygalaturonase production, trehalose production, and glutamine conversion.

In some embodiments, the bacterium is an engineered bacterium comprising at least one genetic variation introduced into genes selected from the group consisting of: bcsii, bcsiii, yjbE, fhaB, pehA, otsB, treZ, glsA2, and combinations thereof.

In some embodiments, the bacterium is selected from Table 1, or a variant, mutant, or derivative thereof.

In some embodiments, the bacterium comprises a nucleic acid sequence that shares at least about 90%, 95%, or 99% sequence identity to a nucleic acid sequence selected from SEQ ID NOs: 1-223.

In some embodiments, the bacterium comprises a nucleic acid sequence selected from SEQ ID NOs: 1-223.

In one aspect, the present disclosure provides an agronomically stable liquid agricultural composition with a room temperature shelf life of at least 3 months, comprising: a) a diazotrophic bacterium at a cellular density that provides a decay rate of less than 0.2 log loss CFU/mL over the shelf life of the composition; b) a buffering agent that maintains the pH of the composition around pH 6.7 over the shelf life of the composition; c) a microbial stabilizer that slows the doubling rate of the diazotrophic bacterium; and d) a physical stabilizer that decreases the local density of the diazotrophic bacterium within the composition, wherein the stability of the composition is greater than, and the presence of toxic byproducts is less than, the composition absent one or more of the buffering agent, microbial stabilizer, and physical stabilizer.

In one aspect, the present disclosure provides an agronomically stable liquid agricultural composition with a room temperature shelf life of at least 3 months, comprising: a) Klebsiella variicola at a concentration of at least about 4.5E9 CFU/mL; b) modified, high buffering capacity PBS; c) fructose at a concentration of at least about 1% w/v; and d) xanthan gum at a concentration of at least about 0.05% w/v.

In one aspect, the present disclosure provides an agronomically stable liquid agricultural composition with a room temperature shelf life of at least 3 months, comprising: a) Kosakonia sacchari at a concentration of about 3E9 CFU/mL; b) modified, high buffering capacity PBS; and c) fructose at a concentration of at least about 1% w/v.

In one aspect, the present disclosure provides agricultural plant tissue comprising the agronomically stable liquid agricultural composition of any of the foregoing embodiments.

In some embodiments, the agricultural plant is a legume or cereal grain.

In some embodiments, the agricultural plant is alfalfa, clover, bean, pea, chickpea, lentil, lupin, mesquite, carob, soybean, peanut, rooibos, or tamarind.

In some embodiments, the agricultural plant is corn, rice, wheat, barley, sorghum, millet, oats, or rye.

In some embodiments, the agricultural plant is corn.

In one aspect, the present disclosure provides a method for applying a diazotrophic bacterium to agricultural plant tissues comprising applying the composition of any one of the foregoing embodiments to agricultural plant tissues or the environs thereof.

In one aspect, the present disclosure provides a method for maintaining a population of a diazotrophic bacterium on an agricultural plant tissue comprising applying the composition of any one of the foregoing embodiments to said plant tissue or the environs thereof.

In one aspect, the present disclosure provides a method of increasing agricultural plant crop yield comprising applying the composition of any one of the foregoing embodiments to the agricultural plant tissues or the environs thereof prior to, during, or immediately following planting, thereby increasing the crop yield of the agricultural plant once planted.

In one aspect, the present disclosure provides a method of providing fixed atmospheric nitrogen to a cereal plant, comprising applying the composition of any one of the foregoing embodiments to the cereal plant tissues or the environs thereof.

In one aspect, the present disclosure provides a method of providing fixed atmospheric nitrogen to a corn plant that eliminates the need for the addition of in-season exogenous nitrogen application, comprising applying the composition of any one of the foregoing embodiments to the corn plant tissues or the environs thereof.

In one aspect, the present disclosure provides a method for increasing corn yield per acre, comprising applying the composition of any one of the foregoing embodiments to the corn plant tissues or the environs thereof.

In one aspect, the present disclosure provides a method for reducing infield variability for corn yield per acre, comprising applying the composition of any one of the foregoing embodiments to the corn plant tissues or the environs thereof, wherein the standard deviation of corn mean yield measured in bushels per acre is lower than for control plants to which the composition has not been applied.

In some embodiments, the method comprises: a) applying the composition to a locus; and b) providing to the locus a plurality of the plants.

In some embodiments, the composition comprises a plurality of said diazotrophic bacteria, wherein the diazotrophic bacteria comprise engineered bacteria, and wherein the engineered bacteria colonize the root surface of said plurality of plants and supply the plants with fixed nitrogen.

In some embodiments, the composition comprises a plurality of said diazotrophic bacteria, wherein the diazotrophic bacteria comprise engineered bacteria, and wherein the engineered bacteria colonize the root surface of said plurality of plants and supply the plants with fixed nitrogen, and wherein the plurality of engineered bacteria produce in the aggregate at least about 15 pounds of fixed nitrogen per acre over the course of at least about 10 days to about 60 days.

In some embodiments, exogenous nitrogen is not applied to the plant tissues or the environs thereof after the composition is applied.

In some embodiments, the agricultural plant is a legume or cereal grain.

In some embodiments, the agricultural plant is alfalfa, clover, bean, pea, chickpea, lentil, lupin, mesquite, carob, soybean, peanut, rooibos, or tamarind.

In some embodiments, the agricultural plant is corn, rice, wheat, barley, sorghum, millet, oats, or rye.

In some embodiments, the agricultural plant is corn.

In some embodiments, the method increases the crop yield of the agricultural plant.

In some embodiments, the method increases the crop yield of the agricultural plant with a win rate of greater than 65%.

In some embodiments, the method increases the crop yield of the agricultural plant with a win rate of about 75%.

In some embodiments, the method increases the crop yield of the agricultural plant by more than 3 bushels/acre.

In some embodiments, the method increases the crop yield of the agricultural plant by about 3 bushels/acre.

In some embodiments, the method reduces infield variability for the agricultural plant crop yield per acre.

In some embodiments, the method reduces infield variability for the agricultural plant crop yield per acre with a win rate of greater than 65%.

In some embodiments, the method reduces infield variability for the agricultural plant crop yield per acre with a win rate of about 75%.

In some embodiments, the method reduces infield variability for the agricultural plant crop yield per acre with a variance improvement of greater than 2 bushels/acre.

In one aspect, the present disclosure provides a method of preparing an agronomically stable liquid agricultural composition comprising a diazotrophic bacterium, the method comprising the steps of: a) providing a diazotrophic bacterium; b) selecting for inclusion in the composition a cellular density of the diazotrophic bacterium that provides an acceptable rate of decay of the bacterium; c) selecting a buffering agent for inclusion in the composition; d) selecting a microbial stabilizer for inclusion in the composition; and e) selecting a physical stabilizer for inclusion in the composition, wherein the composition is stable at room temperature for a period of more than 30 days, and wherein the stability of the composition is greater than the composition absent one or more of the buffering agent, microbial stabilizer, an physical stabilizer.

In one aspect, the present disclosure provides a method for improving the stability of a liquid agricultural composition comprising a diazotrophic bacterium, the method comprising the steps of a) providing a diazotrophic bacterium; b) selecting for inclusion in the composition a cellular density of the diazotrophic bacterium that provides an acceptable rate of decay of the bacterium; c) selecting a buffering agent for inclusion in the composition; d) selecting a microbial stabilizer for inclusion in the composition; and e) selecting a physical stabilizer for inclusion in the composition, wherein the composition has a room temperature shelf life of at least 30 days.

In some embodiments, step (b) comprises generating a titration curve to determine cellular density versus decay rate of the diazotrophic bacterium and selecting a cellular density that provides an acceptable decay rate.

In some embodiments, steps (c)-(e) can be performed in any order.

In some embodiments, any subset of steps (c)-(e) can be performed serially or in parallel.

In some embodiments, step (c) comprises selecting a buffering agent that improves microbial stability when included in the agricultural composition, either in the presence or absence of the microbial and/or physical stabilizer.

In some embodiments, step (c) comprises comparing two or more buffering agents and selecting the buffering agent that provides greater improvement to the microbial stability when included in the agricultural composition, either in the presence or absence of the microbial and/or physical stabilizer.

In some embodiments, step (d) comprises selecting a microbial stabilizer that improves microbial stability when included in the agricultural composition, either in the presence or absence of the buffering agent and/or physical stabilizer

In some embodiments, step (d) comprises comparing two or more microbial stabilizers and selecting the microbial stabilizer that provides greater improvement to the microbial stability when included in the agricultural composition, either in the presence or absence of the buffering agent and/or physical stabilizer.

In some embodiments, step (e) comprises selecting a physical stabilizer that improves microbial stability when included in the agricultural composition, either in the presence or absence of the buffering agent and/or microbial stabilizer.

In some embodiments, step (e) comprises comparing two or more physical stabilizers and selecting the physical stabilizer that provides greater improvement to the microbial stability when included in the agricultural composition, either in the presence or absence of the buffering agent and/or microbial stabilizer.

In some embodiments, the selections of the microbial stabilizer and the physical stabilizer in steps (d) and (e) are performed simultaneously.

In some embodiments, the method increases the shelf life of the liquid agricultural composition by a factor of at least 2, at least 3, or at least 4.

In some embodiments, any one of steps (b)-(e) alone provides less improvement to the microbial stability of the composition than all of the steps together.

In some embodiments, the composition is for application to agricultural plant tissues or the environs thereof.

In some embodiments, the method decreases the accumulation of toxic byproducts in the composition over the course of its shelf life.

In some embodiments, the method decreases the accumulation of ammonia in the composition over the course of its shelf life.

In some embodiments, the method decreases the accumulation of ammonia in the composition by at least two-fold over the course of its shelf life as compared to an agricultural composition absent the microbial stabilizer and buffering agent.

In some embodiments, the composition at the end of its' shelf life has a colonization potential approximately equal to the colonization potential of the composition when freshly formulated.

In some embodiments, the microbial stability of the composition is greater than the microbial stability of the composition absent one or more of the buffering agent, microbial stabilizer, and physical stabilizer.

In some embodiments, the composition has a shelf life of at least two months, at least three months, at least four months, or at least five months.

In some embodiments, the composition has a shelf life of at least three months.

In some embodiments, the log loss of CFU/mL over the shelf life of the composition is less than 0.2.

In some embodiments, the cellular density of the bacterium minimizes the rate of decay over the shelf life of the composition.

In some embodiments, the cellular density of the bacterium provides a reduced, but not minimized rate of decay.

In some embodiments, the cellular density of the bacterium provides a rate of decay of less than 1.0 log loss of CFU/mL over 30 days in the agricultural composition absent the buffering agent, microbial stabilizer, and physical stabilizer.

In some embodiments, the bacterium is present at a cellular density of about 3E9-6E9 CFU/mL.

In some embodiments, the buffering agent maintains the pH of the composition over the shelf life of the composition.

In some embodiments, the buffering agent maintains the pH of the composition at about pH 6-8 over the shelf life of the composition.

In some embodiments, the buffering agent maintains the pH of the composition at about pH 6.5 over the shelf life of the composition.

In some embodiments, the buffering agent is selected from the list consisting of phosphate buffered saline (PBS); modified, high buffering capacity PBS; 3-Morpholinopropane-1-sulfonic acid (MOPS); and 2-[4-(2-hydroxyethyl)piperazin-1-yl]ethanesulfonic acid (HEPES).

In some embodiments, the buffering agent is modified, high buffering capacity PBS.

In some embodiments, the microbial stabilizer slows the doubling rate of the diazotrophic bacterium.

In some embodiments, the microbial stabilizer slows the toxin accumulation rate within the composition.

In some embodiments, the microbial stabilizer is a monosaccharide, disaccharide, polysaccharide, pentose, hexose, oligosaccharide, oligofructose, sugar alcohol, amino acid, protein or protein hydrolysate, or polymer.

In some embodiments, the microbial stabilizer is a monosaccharide or a disaccharide selected from the list consisting of glucose, fructose, trehalose, sucrose, lactose, melibiose, and lactulose.

In some embodiments, the microbial stabilizer is fructose or trehalose.

In some embodiments, the microbial stabilizer is fructose.

In some embodiments, the microbial stabilizer is fructose and is selected for inclusion in the composition at a concentration of about 0.5-2.5% w/v.

In some embodiments, the microbial stabilizer is fructose and is selected for inclusion in the composition at a concentration of about 1.3% w/v.

In some embodiments, the physical stabilizer decreases the local density of the diazotrophic bacterium within the composition.

In some embodiments, the physical stabilizer is a polysaccharide, protein or protein hydrolysate, polymer, or a natural gum or its derivative.

In some embodiments, the physical stabilizer is a polysaccharide.

In some embodiments, the physical stabilizer is a polysaccharide selected from the list consisting of maltodextrin, polyethylene glycol (PEG), xanthan gum, pectin, alginates, microcrystalline cellulose, and dextran.

In some embodiments, the physical stabilizer is xanthan gum.

In some embodiments, the physical stabilizer is xanthan gum and is selected for inclusion in the composition at a concentration of about 0.001-0.2% w/v.

In some embodiments, the physical stabilizer is xanthan gum and is selected for inclusion in the composition at a concentration of about 0.1% w/v.

In some embodiments, the bacterium is a gram-negative bacterium.

In some embodiments, the bacterium is of a genus selected from the group consisting of: Acetobacter, Achromobacter, Aerobacter, Anabaena, Azoarcus, Azomonas, Azorhizobium, Azospirillum, Azotobacter, Beijernickia, Bradyrhizobium, Burkholderia, Citrobacter, Derxia, Enterobacter, Herbaspirillum, Klebsiella, Kluyvera, Kosakonia, Mesorhizobium, Metakosakonia, Paraburkholderia, Nostoc, Rahnella, Rhizobium, Rhodobacter, Rhodopseudomonas, Rhodospirillum, Serratia Sinorhizobium, Spirillum, Trichodesmium, and Xanthomonas.

In some embodiments, the bacterium is of a species selected from the group consisting of: Achromobacter marplatensis, Achromobacter spiritinus, Azospirillum lipoferum, Enterobacter sacchari, Herbaspirillum aquaticum, Klebsiella variicola, Kluyvera intermedia, Kosakonia pseudosacchari, Kosakonia sacchari, Metakosakonia intestini, Paraburkholderia tropica, Rahnella aquatilis, and combinations thereof.

In some embodiments, the bacterium is a gram-positive bacterium.

In some embodiments, the bacterium is of a genus selected from the group consisting of: Arthrobacter, Agromyces, Bacillus, Clostridium, Corynebacterium, Frankia, Heliobacillus, Heliobacterium, Heliophilum, Heliorestis Methanobacterium, Microbacterium, Micrococcus, Micromonospora, Mycobacterium, Paenibacillus, Propionibacterium, and Streptomyces.

In some embodiments, the bacterium is of a species selected from the group consisting of: Bacillus amyloliquefaciens, Bacillus macerans, Bacillus pumilus, Bacillus thuringiensis, Clostridium acetobutylicum, Corynebacterium autitrophicum Methanobacterium formicicum, Methanobacterium omelionski, Microbacterium murale, Mycobacterium flavum, Paenibacillus polymyxa, Paenibacillus riograndensis, Propionibacterium acidipropio, Propionibacterium freudenreichii, Streptococcus lactis, Streptomyces griseus, and combinations thereof.

In some embodiments, the bacterium is of the genus Klebsiella.

In some embodiments, the bacterium is of the species Klebsiella variicola.

In some embodiments, the bacterium is of the strain Klebsiella variicola NCMA 201712002.

In some embodiments, the bacterium is of the genus Kosakonia.

In some embodiments, the bacterium is of the species Kosakonia sacchari.

In some embodiments, the bacterium is of the strain Kosakonia sacchari ATCC PTA-126743.

In some embodiments, the bacterium is endophytic, epiphytic, or rhizospheric.

In some embodiments, the bacterium is a wild type bacterium.

In some embodiments, the bacterium is an engineered bacterium.

In some embodiments, the bacterium is a transgenic bacterium.

In some embodiments, the bacterium is an intragenic bacterium.

In some embodiments, the bacterium is a remodeled bacterium.

In some embodiments, the bacterium comprises a non-intergeneric genomic modification.

In some embodiments, the bacterium is an engineered bacterium capable of fixing atmospheric nitrogen in the presence of exogenous nitrogen.

In some embodiments, the bacterium is an engineered bacterium comprising at least one genetic variation introduced into at least one gene, or non-coding polynucleotide, of the nitrogen fixation or assimilation genetic regulatory network.

In some embodiments, the bacterium is an engineered bacterium comprising an introduced control sequence operably linked to at least one gene of the nitrogen fixation or assimilation genetic regulatory network.

In some embodiments, the bacterium is an engineered bacterium comprising a heterologous promoter operably linked to at least one gene of the nitrogen fixation or assimilation genetic regulatory network.

In some embodiments, the bacterium is an engineered bacterium comprising at least one genetic variation selected from the group consisting of: nifA, nifL, ntrB, ntrC, polynucleotide encoding glutamine synthetase, glnA, glnB, glnK, drat, amtB, polynucleotide encoding glutaminase, glnD, glnE, nifJ, nifH, nifD, nifK, nifY, nifE, nifN, nifU, nifS, nifV, nifW, nifZ, nifM, nifF, nifB, nifQ, a gene associated with biosynthesis of a nitrogenase enzyme, and combinations thereof.

In some embodiments, the bacterium is an engineered bacterium comprising at least one genetic variation introduced into at least one gene, or non-coding polynucleotide, of the nitrogen fixation or assimilation genetic regulatory network that results in one or more of: increased expression or activity of NifA or glutaminase; decreased expression or activity of NifL, NtrB, glutamine synthetase, GlnB, GlnK, DraT, AmtB; decreased adenylyl-removing activity of GlnE; or decreased expression or uridylyl-removing activity of GlnD.

In some embodiments, the bacterium is an engineered bacterium comprising a mutated nifL gene that has been altered to comprise a heterologous promoter inserted into said nifL gene.

In some embodiments, the bacterium is an engineered bacterium comprising a mutated glnE gene that results in a truncated GlnE protein lacking an adenylyl-removing (AR) domain.

In some embodiments, the bacterium is an engineered bacterium comprising a mutated glnD gene that results in the lack of expression of said glnD gene.

In some embodiments, the bacterium is an engineered bacterium comprising a mutated amtB gene that results in the lack of expression of said amtB gene.

In some embodiments, the bacterium is an engineered bacterium comprising at least one of: a mutated nif gene that has been altered to comprise a heterologous promoter inserted into said nifL gene; a mutated glnE gene that results in a truncated GlnE protein lacking an adenylyl-removing (AR) domain; a mutated amtB gene that results in the lack of expression of said amtB gene; a mutated glnD gene that results in the lack of expression of said glnD gene; and combinations thereof.

In some embodiments, the bacterium is an engineered bacterium comprising at least one genetic variation introduced into genes involved in a pathway selected from the group consisting of: exopolysaccharide production, endo-polygalaturonase production, trehalose production, and glutamine conversion.

In some embodiments, the bacterium is an engineered bacterium comprising at least one genetic variation introduced into genes selected from the group consisting of: bcsii, bcsiii, yjbE, fhaB, pehA, otsB, treZ, glsA2, and combinations thereof.

In some embodiments, the bacterium is selected from Table 1, or a variant, mutant, or derivative thereof.

In some embodiments, the bacterium comprises a nucleic acid sequence that shares at least about 90%, 95%, or 99% sequence identity to a nucleic acid sequence selected from SEQ ID NOs: 1-223.

In some embodiments, the bacterium comprises a nucleic acid sequence selected from SEQ ID NOs: 1-223.

### BRIEF DESCRIPTION OF THE SEVERAL VIEWS OF THE DRAWINGS

FIG. 1 shows a graph of cell density (log CFU/mL) at the time of formulation of an illustrative agricultural composition versus the decay rate in log loss of CFU/mL per day.

FIG. 2 shows the results of an ammonia toxicity assay. Sample identifiers are indicated in the graph. Toxicity ranges of ammonia for Klebsiella variicola NCMA 201708001 are shown in dotted lines: non-toxic from 0-50 mM; intermediate toxicity, 50-100 mM; and toxic, 100 mM and above.

FIG. 3 shows the results of an in planta microbial colonization assay. From left to right, the six tested conditions corresponded to: negative control (no microbe added), positive control (fresh culture), sample D, sample C, sample A, and sample B. The horizontal line shows the median colonization observed among the replicates for each condition.

FIG. 4 shows the results of a soluble ammonia accumulation assay in 3-month old samples comprising various combinations of physical and microbial stabilizers. The control condition comprises no stabilizers. The horizontal lines indicate the median ammonia accumulation among the replicates for each condition.

FIG. 5 shows a comparison between the bacterial stability of an exemplary liquid agricultural composition according to the present disclosure and the bacterial propagation of a suspended and activated dry formulation comprising the same bacterial strain.

FIG. 6 shows the data collection and quality control for harvest combine monitor data for an exemplary field trial comparing an exemplary liquid agricultural composition of the present disclosure to a suspended dry formulation and an untreated control. White areas correspond to data that were removed because they corresponded to header rows, or because they corresponded to areas where the harvest combine did not have a steady velocity or direction. The area outlined in a dotted line was treated with the exemplary liquid agricultural composition; the area outline in a solid line was treated with the suspended, activated dry formulation; and the remaining areas were the untreated control areas.

FIG. 7 shows the yield improvement across the eight field trials for a commercially available dry formulation, Pivot Bio PROVEN™ 2019 (solid bars) and an exemplary liquid agricultural composition (bars with diagonal lines).

FIG. 8 shows the variance improvement across the eight field trials for a commercially available dry formulation, Pivot Bio PROVEN™ 2019 (solid bars) and an exemplary liquid agricultural composition (bars with diagonal lines).

FIG. 9 shows a bar graph of wheat yield. K. variicola deposited strain NCMA 201708001 (“KV137-RTU”) was tested at ten trial locations where nitrogen was reduced by 25 pounds and compared to (1) a control which received 100% of the recommended nitrogen rate and (2) a reduced nitrogen control containing nitrogen reduced by 25 pounds.

FIG. 10 shows a bar graph of sorghum yield. K. variicola deposited strain NCMA 201708001 (“KV137-RTU”) was tested at ten trial locations where nitrogen was reduced by 25 pounds and compared to (1) a control which received 100% of the recommended nitrogen rate and (2) a reduced nitrogen control containing nitrogen reduced by 25 pounds.

FIG. 11 shows a bar graph of the cell viability of strain ATCC PTA126743 over the course of several months with different stabilizers.

### DETAILED DESCRIPTION

The present disclosure provides agronomically stable liquid agricultural compositions comprising nitrogen-fixing microorganisms and one or more of buffering agents, microbial stabilizers, and physical stabilizers. Also provided are methods of formulating these agronomically stable liquid agricultural compositions and methods of applying the same. The present disclosure also provides agricultural plant tissues comprising the agronomically stable liquid agricultural compositions.

### Definitions

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

As used herein, the term “about” is used synonymously with the term “approximately.” Illustratively, the use of the term “about” with regard to an amount indicates that values slightly outside the cited values, e.g., plus or minus 0.1% to 10%.

“Plant tissues,” as used herein, refers to any part of the plant during any aspect of the growing cycle, including seeds, seedlings, plants, or plant parts. Plant parts include leaves, roots, root hairs, rhizomes, stems, seed, ovules, pollen, flowers, fruit, cuttings, tubers, bulbs, etc. An agricultural plant tissue “comprising” an agronomically stable liquid agricultural composition of the disclosure includes agricultural plant tissues to which the agricultural composition has been applied by any of the means set forth herein, e.g., spraying, in-furrow application, seed treatment, etc.

“Plant productivity” refers generally to any aspect of growth or development of a plant that is a reason for which the plant is grown. For food crops, such as grains or vegetables, “plant productivity” can refer to the yield of grain or fruit harvested from a particular crop. As used herein, improved plant productivity refers broadly to improvements in yield of grain, fruit, flowers, or other plant parts harvested for various purposes, improvements in growth of plant parts, including stems, leaves and roots, promotion of plant growth, maintenance of high chlorophyll content in leaves, increasing fruit or seed numbers, increasing fruit or seed unit weight, and similar improvements of the growth and development of plants. Plant productivity, in the context of agricultural compositions with nitrogen fixing bacteria, is determined by comparing the productivity (e.g., yield) of a treated plant (e.g., via in furrow application), vs. a plant with no composition applied, and no additional fertilizer beyond what is provided to the treated plant. Thus, in some embodiments, the agricultural compositions of the present disclosure result in reductions in NO₂ emission due to reduced nitrogen fertilizer usage.

Microbes in and around food crops can influence the traits of those crops. Plant traits that may be influenced by microbes include: yield (e.g., grain production, biomass generation, fruit development, flower set); nutrition (e.g., nitrogen, phosphorus, potassium, iron, micronutrient acquisition); abiotic stress management (e.g., drought tolerance, salt tolerance, heat tolerance); and biotic stress management (e.g., pest, weeds, insects, fungi, and bacteria). Strategies for altering crop traits include: increasing key metabolite concentrations; changing temporal dynamics of microbe influence on key metabolites; linking microbial metabolite production/degradation to new environmental cues; reducing negative metabolites; and improving the balance of metabolites or underlying proteins.

As used herein, “in planta” may refer to in the plant, on the plant, or intimately associated with the plant, depending upon context of usage (e.g. endophytic, epiphytic, or rhizospheric associations). The plant may comprise plant parts, tissue, leaves, roots, root hairs, rhizomes, stems, seed, ovules, pollen, flowers, fruit, etc.

As used herein, “introduced” refers to the introduction by means of modern biotechnology, and not a naturally occurring introduction.

In some embodiments, the bacteria of the present disclosure have been modified such that they are not naturally occurring bacteria.

Fertilizers and exogenous nitrogen of the present disclosure may comprise the following nitrogen-containing molecules: ammonium, nitrate, nitrite, ammonia, glutamine, etc. Nitrogen sources of the present disclosure may include anhydrous ammonia, ammonia sulfate, urea, diammonium phosphate, urea-form, monoammonium phosphate, ammonium nitrate, nitrogen solutions, calcium nitrate, potassium nitrate, sodium nitrate, etc.

As used herein, “exogenous nitrogen” refers to non-atmospheric nitrogen readily available in the soil, field, or growth medium that is present under non-nitrogen limiting conditions, including ammonia, ammonium, nitrate, nitrite, urea, uric acid, ammonium acids, etc.

As used herein, “non-nitrogen limiting conditions” refers to non-atmospheric nitrogen available in the soil, field, media at concentrations greater than about 4 mM nitrogen, as disclosed by Kant et al. (2010. J. Exp. Biol. 62 (4): 1499-1509), which is incorporated herein by reference.

As used herein, an “intergeneric microorganism” is a microorganism that is formed by the deliberate combination of genetic material originally isolated from organisms of different taxonomic genera. An “intergeneric mutant” can be used interchangeably with “intergeneric microorganism”. An exemplary “intergeneric microorganism” includes a microorganism containing a mobile genetic element which was first identified in a microorganism in a genus different from the recipient microorganism. Further explanation can be found, inter alia, in 40 C.F.R. § 725.3.

In aspects, microbes taught herein are “non-intergeneric,” which means that the microbes are not intergeneric.

As used herein, an “intrageneric microorganism” is a microorganism that is formed by the deliberate combination of genetic material originally isolated from organisms of the same taxonomic genera. An “intrageneric mutant” can be used interchangeably with “intrageneric microorganism.”

As used herein, an “intragenic” microorganism, is a microorganism that is engineered to comprise a genetic edit, or genetic modification, or genetic element, or genetic material (e.g. a nucleic acid sequence), that has been sourced from within the organism's own genome.

As used herein, a “transgenic” microorganism, is a microorganism that is engineered to comprise a genetic edit, or genetic modification, or genetic element, or genetic material (e.g. a nucleic acid sequence), that has been sourced from outside the organism's own species.

As used herein, in the context of non-intergeneric microorganisms, the term “remodeled” is used synonymously with the term “engineered”. Consequently, a “non-intergeneric remodeled microorganism” has a synonymous meaning to “non-intergeneric engineered microorganism,” and will be utilized interchangeably.

A “wild type microbe,” e.g., a “wild type bacterium,” as used herein refers to a microbe that has not been genetically modified. Wild type microbes may be isolated and cultivated from a natural source. Wild type microbes may be selected for specific naturally occurring traits.

A “diazotroph” is a microbe that fixes atmospheric nitrogen gas into a more usable form, such as ammonia. A diazotroph is a microorganism that is able to grow without external sources of fixed nitrogen. All diazotrophs contain iron-molybdenum orvanadium nitrogenase systems.

In some embodiments, the increase of nitrogen fixation and/or the production of 1% or more of the nitrogen in the plant are measured relative to control plants, which have not been exposed to the bacteria of the present disclosure. All increases or decreases in bacteria are measured relative to control bacteria. All increases or decreases in plants are measured relative to control plants.

In aspects, “applying,” “coating,” and “treating” agricultural plant tissues or the environs thereof with the present agricultural compositions includes any means by which the plant tissues or the environs thereof are made to come into contact (i.e. exposed) to said agricultural compositions. Consequently, “applying” includes any of the following means of exposure to said agricultural compositions: spraying, dripping, submerging, applying as a seed coat, applying to a field that will then be planted with seed, applying to a field already planted with seed, etc. For the purposes of this disclosure, “the environs” of agricultural plant tissues include the elements of the vicinity around the agricultural plant tissues that come into contact with the agricultural plant tissues. For example, application to the environs of agricultural plant tissues would include soil application and in-furrow application means.

As used herein the term “plant” can include plant parts, tissue, leaves, roots, root hairs, rhizomes, stems, seeds, ovules, pollen, flowers, fruit, etc. Thus, when the disclosure discusses providing a plurality of corn plants to a particular locus, it is understood that this may entail planting a corn seed at a particular locus.

As used herein the terms “microorganism” or “microbe” should be taken broadly. These terms, used interchangeably, include but are not limited to, the two prokaryotic domains, Bacteria and Archaea. The term may also encompass eukaryotic fungi and protists.

As used herein, when the disclosure discuses a particular microbial deposit by accession number, it is understood that the disclosure also contemplates a microbial strain having all of the identifying characteristics of said deposited microbe, and/or a mutant thereof.

In certain aspects of the disclosure, the isolated microbes exist as “isolated and biologically pure cultures.” It will be appreciated by one of skill in the art, that an isolated and biologically pure culture of a particular microbe, denotes that said culture is substantially free of other living organisms and contains only the individual microbe in question. The culture can contain varying concentrations of said microbe. The present disclosure notes that isolated and biologically pure microbes often “necessarily differ from less pure or impure materials.” See, e.g. In re Bergstrom, 427 F.2d 1394, (CCPA 1970) (discussing purified prostaglandins), see also, In re Bergy, 596 F.2d 952 (CCPA 1979) (discussing purified microbes), see also, Parke-Davis & Co. v. H. K. Mulford & Co., 189 F. 95 (S.D.N.Y. 1911) (Learned Hand discussing purified adrenaline), aff'd in part, rev'd in part, 196 F. 496 (2d Cir. 1912), each of which are incorporated herein by reference. Furthermore, in some aspects, the disclosure provides for certain quantitative measures of the concentration, or purity limitations, that must be found within an isolated and biologically pure microbial culture. The presence of these purity values, in certain embodiments, is a further attribute that distinguishes the presently disclosed microbes from those microbes existing in a natural state. See, e.g., Merck & Co. v. Olin Mathieson Chemical Corp., 253 F.2d 156 (4th Cir. 1958) (discussing purity limitations for vitamin B12 produced by microbes), incorporated herein by reference.

Microbes of the present disclosure may include spores and/or vegetative cells. In some embodiments, microbes of the present disclosure include microbes in a viable but non-culturable (VBNC) state. As used herein, “spore” or “spores” refer to structures produced by bacteria and fungi that are adapted for survival and dispersal. Spores are generally characterized as dormant structures; however, spores are capable of differentiation through the process of germination. Germination is the differentiation of spores into vegetative cells that are capable of metabolic activity, growth, and reproduction. The germination of a single spore results in a single fungal or bacterial vegetative cell. Fungal spores are units of asexual reproduction, and In some embodiments are necessary structures in fungal life cycles. Bacterial spores are structures for surviving conditions that may ordinarily be nonconductive to the survival or growth of vegetative cells.

As used herein, the term “agronomically stable” refers to an agricultural composition comprising microorganisms that maintains the viability of the microorganisms over time. An agronomically stable agricultural composition may exhibit a decline in the bacterial concentration over time, but at a reduced rate compared to traditional liquid formulations. Loss of bacterial concentration may be measured in log loss of CFU per unit over time.

As used herein, the “shelf life” of an agricultural composition refers to the period of time over which the composition can be stored and still retain a desired level of efficacy for its intended purpose, e.g., application to agricultural plant tissues or the environs thereof for delivery of fixed nitrogen to the agricultural plant. In some embodiments, the shelf life is the period of time over which an agricultural composition can be stored at room temperature and experience less than log loss CFU/mL of 1. In some embodiments, the shelf life is the period of time over which an agricultural composition can be stored at room temperature and experience less than log loss CFU/mL of 0.5-2. In some embodiments, the shelf life is the period of time over which an agricultural composition can be stored and experience less than 50% loss of cell density in CFU/mL. In some embodiments, the shelf life is the period of time over which an agricultural composition can be stored and experience less than 90% loss of cell density in CFU/mL. In some embodiments, the shelf life is measured at room temperature. In some embodiments, the shelf life is measured at 4° C. In some embodiments, the temperature varies over the course of the period of storage over which shelf life is measured.

As used herein, a “seed treatment” refers to a substance that may be applied to agricultural seeds. The seed treatment may provide one or more benefits to the seed and/or plant resulting from the seed. Without limitation, seed treatments may include pesticides, herbicides, insecticides, nematicides, plant-growth promoting factors, fertilizers, compositions of the disclosure comprising diazotrophic microbes, and the like.

As used herein, the terms “formulation,” “composition,” “agricultural treatment,” and the like may be used interchangeably to refer to the agronomically stable liquid agricultural compositions of the present disclosure.

The term “colony forming unit” or “CFU” as used herein is a unit used to estimate the number of viable microbial cells in a sample. Viable is defined as the ability to multiply under the controlled conditions. Counting colony-forming units requires culturing the microbes and counting only viable cells, in contrast with microscopic examination which counts all cells, living or dead. The visual appearance of a colony in a cell culture requires significant growth and may result from the growth of individual or multiple viable cells.

As used herein, a “buffering agent,” “buffer solution,” or “buffer,” also known as a “pH buffer” or “hydrogen ion buffer,” is an aqueous solution consisting of a mixture of a weak acid and its conjugate base, or a weak base and its conjugate acid. Its pH changes very little when a small amount of strong acid or base is added to it. Buffering agents are used as a means of keeping pH at a nearly constant value, or within a certain pH range, over a period of time. Herein, “a buffering agent” may refer to either a chemical compound used to buffer a formulation or to a buffering system comprising a combination of acids, bases, and/or salts.

### Agronomically Stable Liquid Agricultural Compositions and Methods of Formulation Thereof

The present disclosure provides agronomically stable liquid agricultural compositions. The agricultural compositions comprise plant-beneficial, nitrogen-fixing microorganisms. The agricultural compositions also comprise one or more of buffering agents, microbial stabilizers, and physical stabilizers. The present disclosure also provides methods of formulating and improving the stability of liquid agricultural compositions comprising nitrogen-fixing microorganisms and one or more of buffering agents, microbial stabilizers, and physical stabilizers.

In some embodiments, the agricultural compositions are cost effective, scalable liquid formulations for providing nitrogen-fixing microorganisms to agricultural plants in the form most preferred within the agricultural industry with improved stability compared to existing liquid formulations. Unlike a dry powder, the agricultural composition does not require suspension or activation prior to use, thus allowing for improved consistency in quality and application results. In some embodiments, the agricultural composition is ready to use as-is. In some embodiments, the agricultural composition is concentrated and is diluted prior to use. When the agricultural composition is ready to use as-is or with dilution, in some embodiments, its use does not require consistent compliance in the activation steps taken by the user. In some embodiments, the ready to use formulation of the agricultural compositions decreases variance in the quality of the agricultural composition between different users.

In some embodiments, the present formulation methods produce agricultural compositions with improved shelf stability. In some embodiments, the agricultural composition has improved shelf stability compared to existing liquid formulations comprising nitrogen-fixing microorganisms. In some embodiments, this shelf stability is determined by measuring microbial viability as a function of time. In some embodiments, the agricultural composition is shelf stable for a period of three to six months. In some embodiments, the agricultural composition is shelf stable for a period of at least three months. In some embodiments, the agricultural composition is shelf stable for a period of at least four months. In some embodiments, the agricultural composition is shelf stable for a period of at least five months. In some embodiments, the agricultural composition is shelf stable for a period of at least six months.

Without wishing to be bound by theory, it is believed that the improved stability of the disclosed agricultural compositions is a result of the combination of formulation ingredients, e.g., as selected using a formulation method disclosed herein. In some embodiments, the agricultural composition components address different underlying causes of decay that lead to poor shelf stability for other liquid formulations. In some embodiments, the causes of decay are: high densities of microbial cells, toxins produced by the cells, and cells entering long-term stationary phase. In some embodiments, the present agricultural compositions address these causes of decay by comprising: an initial density of cells selected to provide a decreased decay rate; physical stabilizers that improve the uniform distribution of cells and decrease the local accumulation of high densities and/or toxin levels; microbial stabilizers that protect the cells, potentially by putting them into a semi-dormant state; and buffering agents that prevent pH fluctuations.

These components and the selection thereof for inclusion in the agricultural compositions of the disclosure are described in greater detail in the following sections.

### Microorganisms

The agricultural compositions of the present disclosure comprise plant-beneficial microorganisms, particularly nitrogen-fixing microorganisms. In some embodiments, microbes useful in the methods and agricultural compositions disclosed herein are obtained from any source. In some embodiments, microbes are bacteria, archaea, protozoa, algae, or fungi. In some embodiments, the microbes of this disclosure are nitrogen fixing microbes, for example nitrogen fixing bacteria, nitrogen fixing archaea, nitrogen fixing fungi, nitrogen fixing yeast, nitrogen fixing algae, or nitrogen fixing protozoa. In some embodiments, microbes useful in the methods and agricultural compositions disclosed herein are spore forming microbes, for example spore forming bacteria. In some embodiments, bacteria useful in the methods and agricultural compositions disclosed herein are Gram positive bacteria or Gram negative bacteria. In some embodiments, the bacteria are endospore forming bacteria of the Firmicute phylum. In some embodiments, the bacteria are diazotrophs. In some embodiments, the bacteria are not diazotrophs.

In some embodiments, the methods and agricultural compositions of the disclosure are used with an archaea, such as, for example, Methanothermobacter thermoautotrophicus, Methanosarcina barkeri, Methanospirillum hungatei, Methanobacterium bryantii, Methanococcus thermolithotrophicus, and Methanococcus maripaludis.

In some embodiments, bacteria which are useful for inclusion in the agricultural compositions of the disclosure include, but are not limited to, Agrobacterium radiobacter, Bacillus acidocaldarius, Bacillus acidoterrestris, Bacillus agri, Bacillus aizawai, Bacillus albolactis, Bacillus alcalophilus, Bacillus alvei, Bacillus aminoglucosidicus, Bacillus aminovorans, Bacillus amylolyticus (also known as Paenibacillus amylolyticus) Bacillus amyloliquefaciens, Bacillus aneurinolyticus, Bacillus atrophaeus, Bacillus azotoformans, Bacillus badius, Bacillus cereus (synonyms: Bacillus endorhythmos, Bacillus medusa), Bacillus chitinosporus, Bacillus circulans, Bacillus coagulans, Bacillus endoparasiticus Bacillus fastidiosus, Bacillus firmus, Bacillus kurstaki, Bacillus lacticola, Bacillus lactimorbus, Bacillus lactis, Bacillus laterosporus (also known as Brevibacillus laterosporus), Bacillus lautus, Bacillus lentimorbus, Bacillus lentus, Bacillus licheniformis, Bacillus maroccanus, Bacillus megaterium, Bacillus metiens, Bacillus mycoides, Bacillus natto, Bacillus nematocida, Bacillus nigrificans, Bacillus nigrum, Bacillus pantothenticus, Bacillus popillae, Bacillus psychrosaccharolyticus, Bacillus pumilus, Bacillus siamensis, Bacillus smithii, Bacillus sphaericus, Bacillus subtilis, Bacillus thuringiensis, Bacillus uniflagellatus, Bradyrhizobium japonicum, Brevibacillus brevis, Brevibacillus laterosporus (formerly Bacillus laterosporus), Chromobacterium subtsugae, Delftia acidovorans, Lactobacillus acidophilus, Lysobacter antibioticus, Lysobacter enzymogenes, Paenibacillus alvei, Paenibacillus polymyxa, Paenibacillus popilliae (formerly Bacillus popilliae), Pantoea agglomerans, Pasteuria penetrans (formerly Bacillus penetrans), Pasteuria usgae, Pectobacterium carotovorum (formerly Erwinia carotovora), Pseudomonas aeruginosa, Pseudomonas aureofaciens, Pseudomonas cepacia (formerly known as Burkholderia cepacia), Pseudomonas chlororaphis, Pseudomonas fluorescens, Pseudomonas proradix, Pseudomonas putida, Pseudomonas syringae, Serratia entomophila, Serratia marcescens, Streptomyces colombiensis, Streptomyces galbus, Streptomyces goshikiensis, Streptomyces griseoviridis, Streptomyces lavendulae, Streptomyces prasinus, Streptomyces saraceticus, Streptomyces venezuelae, Xanthomonas campestris, Xenorhabdus luminescens, Xenorhabdus nematophila, Rhodococcus globerulus AQ719 (NRRL Accession No. B-21663), Bacillus sp. AQ175 (ATCC Accession No. 55608), Bacillus sp. AQ 177 (ATCC Accession No. 55609), Bacillus sp. AQ178 (ATCC Accession No. 53522), and Streptomyces sp. strain NRRL Accession No. B-30145. In some embodiments, the bacterium is Azotobacter chroococcum, Methanosarcina barkeri, Klesiella pneumoniae, Azotobacter vinelandii, Rhodobacter spharoides, Rhodobacter capsulatus, Rhodobcter palustris, Rhodosporillum rubrum, Rhizobium leguminosarum or Rhizobium etli.

In some embodiments, the bacterium is a species of Clostridium, for example Clostridium pasteurianum, Clostridium beijerinckii, Clostridium perfringens, Clostridium tetani, Clostridium acetobutylicum.

In some embodiments, bacteria used with the methods and agricultural compositions of the present disclosure are cyanobacteria. Examples of cyanobacterial genuses include Anabaena (for example Anagaena sp. PCC7120), Nostoc (for example Nostoc punctiforme), or Synechocystis (for example Synechocystis sp. PCC6803).

In some embodiments, bacteria used with the methods and agricultural compositions of the present disclosure belong to the phylum Chlorobi, for example Chlorobium tepidum.

In some embodiments, microbes used with the methods and agricultural compositions of the present disclosure comprise a gene homologous to a known NifH gene. Sequences of known NifH genes may be found in, for example, the Zehr lab NifH database, (wwwzehr.pmc.ucsc.edu/nifH_Database_Public/, Apr. 4, 2014), or the Buckley lab NifH database (www.css.cornell.edu/faculty/buckley/nifh.htm, and Gaby, John Christian, and Daniel H. Buckley. “A comprehensive aligned nifH gene database: a multipurpose tool for studies of nitrogen-fixing bacteria.” Database 2014 (2014): bau001.). In some embodiments, microbes used with the methods and agricultural compositions of the present disclosure comprise a sequence which encodes a polypeptide with at least 60%, 70%, 80%, 85%, 90%, 95%, 96%, 96%, 98%, 99% or more than 99% sequence identity to a sequence from the Zehr lab NifH database, (wwwzehr.pmc.ucsc.edu/nifH_Database_Public/, Apr. 4, 2014). In some embodiments, microbes used with the methods and agricultural compositions of the present disclosure comprise a sequence which encodes a polypeptide with at least 60%, 70%, 80%, 85%, 90%, 95%, 96%, 96%, 98%, 99% or more than 99% sequence identity to a sequence from the Buckley lab NifH database, (Gaby, John Christian, and Daniel H. Buckley. “A comprehensive aligned nifH gene database: a multipurpose tool for studies of nitrogen-fixing bacteria.” Database 2014 (2014): bau001.).

In some embodiments, the methods and agricultural compositions described herein make use of bacteria that are able to self-propagate efficiently on the leaf surface, root surface, or inside plant tissues without inducing a damaging plant defense reaction, or bacteria that are resistant to plant defense responses. In some embodiments, the bacteria described herein are isolated by culturing a plant tissue extract or leaf surface wash in a medium with no added nitrogen.

In some embodiments, the bacteria described herein is an endophyte or an epiphyte or a bacterium inhabiting the plant rhizosphere (rhizospheric bacteria). Endophytes are organisms that enter the interior of plants without causing disease symptoms or eliciting the formation of symbiotic structures, and are of agronomic interest because they can enhance plant growth and improve the nutrition of plants (e.g., through nitrogen fixation). The bacteria can be a seed-borne endophyte. Seed-borne endophytes include bacteria associated with or derived from the seed of a grass or plant, such as a seed-borne bacterial endophyte found in mature, dry, undamaged (e.g., no cracks, visible fungal infection, or prematurely germinated) seeds. The seed-borne bacterial endophyte can be associated with or derived from the surface of the seed; alternatively, or in addition, it can be associated with or derived from the interior seed compartment (e.g., of a surface-sterilized seed). In some embodiments, a seed-borne bacterial endophyte is capable of replicating within the plant tissue, for example, the interior of the seed. Also, In some embodiments, the seed-borne bacterial endophyte is capable of surviving desiccation.

The bacterial used in methods or agricultural compositions of the disclosure, can comprise a plurality of different bacterial taxa in combination. By way of example, the bacteria may include Proteobacteria (such as Pseudomonas, Enterobacter, Stenotrophomonas, Burkholderia, Rhizobium, Herbaspirillum, Pantoea, Serratia, Rahnella, Azospirillum, Azorhizobium, Azotobacter, Duganella, Delftia, Bradyrhizobiun, Sinorhizobium and Halomonas), Firmicutes (such as Bacillus, Paenibacillus, Lactobacillus, Mycoplasma, and Acetabacterium), and Actinobacteria (such as Streptomyces, Rhodacoccus, Microbacterium, and Curtobacterium). The bacteria used in methods and agricultural compositions of this disclosure may include nitrogen fixing bacterial consortia of two or more species. In some embodiments, one or more bacterial species of the bacterial consortia may be capable of fixing nitrogen. In some embodiments, one or more species of the bacterial consortia facilitate or enhance the ability of other bacteria to fix nitrogen. The bacteria which fix nitrogen and the bacteria which enhance the ability of other bacteria to fix nitrogen may be the same or different. In some embodiments, a bacterial strain is able to fix nitrogen when in combination with a different bacterial strain, or in a certain bacterial consortia, but may be unable to fix nitrogen in a monoculture. Examples of bacterial genuses which may be found in a nitrogen fixing bacterial consortia include, but are not limited to, Herbaspirillum, Azospirillum, Enterobacter, and Bacillus.

Bacteria that can be used in the agricultural compositions and methods disclosed herein include Azotobacter sp., Bradyrhizobium sp., Klebsiella sp., and Sinorhizobium sp. In some embodiments, the bacteria are selected from the group consisting of: Azotobacter vinelandii, Bradyrhizobium japonicum, Klebsiella pneumoniae, and Sinorhizobium meliloti. In some embodiments, the bacteria are of the genus Enterobacter or Rahnella. In some embodiments, the bacteria are of the genus Frankia, or Clostridium. Examples of bacteria of the genus Clostridium include, but are not limited to, Clostridium acetobutilicum, Clostridium pasteurianum, Clostridium beijerinckii, Clostridium perfringens, and Clostridium tetani. In some embodiments, the bacteria are of the genus Paenibacillus, for example Paenibacillus azotofixans, Paenibacillus borealis, Paenibacillus durus, Paenibacillus macerans, Paenibacillus polymyxa, Paenibacillus alvei, Paenibacillus amylolyticus, Paenibacillus campinasensis, Paenibacillus chibensis, Paenibacillus glucanolyticus, Paenibacillus illinoisensis, Paenibacillus larvae subsp. Larvae, Paenibacillus larvae subsp. Pulvifaciens, Paenibacillus lautus, Paenibacillus macerans, Paenibacillus macquariensis, Paenibacillus macquariensis, Paenibacillus pabuli, Paenibacillus peoriae, or Paenibacillus polymyxa.

In some embodiments, bacteria for use in the present compositions and methods can be a member of one or more of the following taxa: Achromobacter, Acidithiobacillus, Acidovorax, Acidovoraz, Acinetobacter, Actinoplanes, Adlercreutzia, Aerococcus, Aeromonas, Afipia, Agromyces, Ancylobacter, Arthrobacter, Atopostipes, Azospirillum, Bacillus, Bdellovibrio, Beijerinckia, Bosea, Bradyrhizobium, Brevibacillus, Brevundimonas, Burkholderia, Candidatus Haloredivivus, Caulobacter, Cellulomonas, Cellvibrio, Chryseobacterium, Citrobacter, Clostridium, Coraliomargarita, Corynebacterium, Cupriavidus, Curtobacterium, Curvibacter, Deinococcus, Delftia, Desemzia, Devosia, Dokdonella, Dyella, Enhydrobacter, Enterobacter, Enterococcus, Erwinia, Escherichia, Escherichia Shigella, Exiguobacterium, Ferroglobus, Filimonas, Finegoldia, Flavisolibacter, Flavobacterium, Frigoribacterium, Gluconacetobacter, Hafnia, Halobaculum, Halomonas, Halosimplex, Herbaspirillum, Hymenobacter, Klebsiella, Kocuria, Kosakonia, Lactobacillus, Leclercia, Lentzea, Luteibacter, Luteimonas, Massilia, Mesorhizobium, Methylobacterium, Microbacterium, Micrococcus, Microvirga, Mycobacterium, Neisseria, Nocardia, Oceanibaculum, Ochrobactrum, Okibacterium, Oligotropha, Oryzihumus, Oxalophagus, Paenibacillus, Panteoa, Pantoea, Pelomonas, Perlucidibaca, Plantibacter, Polynucleobacter, Propionibacterium, Propioniciclava, Pseudoclavibacter, Pseudomonas, Pseudonocardia, Pseudoxanthomonas, Psychrobacter, Rahnella, Ralstonia, Rheinheimera, Rhizobium, Rhodococcus, Rhodopseudomonas, Roseateles, Ruminococcus, Sebaldella, Sediminibacillus, Sediminibacterium, Serratia, Shigella, Shinella, Sinorhizobium, Sinosporangium, Sphingobacterium, Sphingomonas, Sphingopyxis, Sphingosinicella, Staphylococcus, Stenotrophomonas, Strenotrophomonas, Streptococcus, Streptomyces, Stygiolobus, Sulfurisphaera, Tatumella, Tepidimonas, Thermomonas, Thiobacillus, Variovorax, WPS-2 genera incertae sedis, Xanthomonas, and Zimmermannella.

In some embodiments, the bacteria are gram-negative bacteria of a genus selected from the following list: Acetobacter, Achromobacter, Aerobacter, Anabaena, Azoarcus, Azomonas, Azorhizobium, Azospirillum, Azotobacter, Beijernickia, Bradyrhizobium, Burkholderia, Citrobacter, Derxia, Enterobacter, Herbaspirillum, Klebsiella, Kluyvera, Kosakonia, Nostoc, Mesorhizobium, Rahnella, Rhizobium, Rhodobacter, Rhodopseudomonas, Rhodospirillum, Serratia Sinorhizobium, Spirillum, Trichodesmium, and Xanthomonas.

In some embodiments, a bacterial species selected from at least one of the following genera are utilized: Enterobacter, Klebsiella, Kosakonia, and Rahnella. In some embodiments, a combination of bacterial species from the following genera are utilized: Enterobacter, Klebsiella, Kosakonia, and Rahnella. In some embodiments, the species utilized can be one or more of: Enterobacter sacchari, Klebsiella variicola, Kosakonia sacchari, and Rahnella aquatilis.

In some embodiments, a Gram positive microbe may have a Molybdenum-Iron nitrogenase system comprising: nifH, nifl), nifK, nifB, nifl, nifN, nifX, hesA, nifV, nifW, nifl), nifS, nifl1, and nif12. In some embodiments, a Gram positive microbe may have a vanadium nitrogenase system comprising: vnfDG, vnfK, vnfk, vnfN, vupC, vupB, vupA, vnfV, vnfR1, vnfH, vnfR2, vnfA (transcriptional regulator). In some embodiments, a Gram positive microbe may have an iron-only nitrogenase system comprising: anfK, anfG, anfD, anfH, anfA (transcriptional regulator). In some embodiments, a Gram positive microbe may have a nitrogenase system comprising glnB, and glnK (nitrogen signaling proteins). Some examples of enzymes involved in nitrogen metabolism in Gram positive microbes include glnA (glutamine synthetase), gdh (glutamate dehydrogenase), bdh (3-hydroxybutyrate dehydrogenase), glutaminase, gltAB/gltB/gltS (glutamate synthase), asnA/asnB (aspartate-ammonia ligase/asparagine synthetase), and ansA/ansZ (asparaginase). Some examples of proteins involved in nitrogen transport in Gram positive microbes include amtB (ammonium transporter), glnK (regulator of ammonium transport), glnPHQ/glnQHMP (ATP-dependent glutamine/glutamate transporters), glnT/alsT/yrbD/yflA (glutamine-like proton symport transporters), and gltP/gltT/yhcD/yflA (glutamate-like proton symport transporters).

Examples of Gram positive microbes for use within the present agricultural compositions include Paenibacillus polymixa, Paenibacillus riograndensis, Paenibacillus sp., Frankia sp., Heliobacterium sp., Heliobacterium chlorum, Heliobacillus sp., Heliophilum sp., Heliorestis sp., Clostridium acetobutylicum, Clostridium sp., Methanobacterium sp., Micrococcus sp., Mycobacterium flavum, Mycobacterium sp., Arthrobacter sp., Agromyces sp., Corynebacterium autitrophicum, Corynebacterium sp., Micromonospora sp., Propionibacteria sp., Streptomyces sp., and Microbacterium sp.

In some embodiments, microorganisms are present in agricultural compositions of the present disclosure at a concentration of between 10⁴ to 10¹² CFU/ml. In some embodiments, the microorganisms are at an initial concentration of 10⁴ to 10¹² CFU/ml. In some embodiments, the microorganisms are at an initial concentration of 10⁸ to 10¹⁰ CFU/ml. In some embodiments, the microorganisms are at an initial concentration of about 10⁴, 10⁵, 10⁶, 10⁷, 10⁸, 10⁹, 10¹⁰, 10¹¹, or 10¹² CFU/mL. In some embodiments, the microorganisms are at an initial concentration of about 10⁸. In some embodiments, the microorganisms are at an initial concentration of about 109. In some embodiments, the microorganisms are at an initial concentration of about 1010.

### Genetic Alterations

In some embodiments, the agricultural compositions of the present disclosure comprise a microbe capable of fixing nitrogen. In some embodiments, the microbe can naturally fix nitrogen. In some embodiments the microbe is genetically modified to fix nitrogen. In some embodiments, the organism is genetically modified to provide improved nitrogen fixation capabilities. Thus, in some embodiments, the microbes comprise one or more genetic variations introduced into one or more genes regulating nitrogen fixation. The genetic variation may be introduced into a gene selected from the group consisting of nifA, nifL, ntrB, ntrC, glutamine synthetase, glnA, glnB, glnK, draT, amtB, glutaminase, glnD, glnE, nifJ, nifH, nifD, nifK, nifY, nifE, nifN, nifU, nifS, nifV, nifW, nifZ, nifM, nifF, nifB, and nifQ. The genetic variation may be a variation in a gene encoding a protein with functionality selected from the group consisting of: glutamine synthetase, glutaminase, glutamine synthetase adenylyltransferase, transcriptional activator, anti-transcriptional activator, pyruvate flavodoxin oxidoreductase, flavodoxin, and NAD+-dinitrogen-reductase aDP-D-ribosyltransferase. The genetic variation may be a mutation that results in one or more of: increased expression or activity of nifA or glutaminase; decreased expression or activity of nifL, ntrB, glutamine synthetase, glnB, glnK, draT, amtB; decreased adenylyl-removing activity of GlnE; decreased expression of GlnD; or decreased uridylyl-removing activity of GlnD. The genetic variation may be a variation in a gene selected from the group consisting of: bcsii, bcsiii, yjbl, fhaB, pehA, otsB, treZ, glsA2, and combinations thereof.

In some embodiments, the microbe has a disrupted (e.g., deleted or partially deleted) nifL gene. In some embodiments, the microbe has a nifL gene that has been disrupted with the introduction of a promoter sequence that acts on the nifA gene. In some embodiments, e.g., when the microbe is a strain of K. variicola, the promoter is a K. variicola PinfC promoter. In some embodiments, e.g., when the microbe is a strain of K. sacchari, the promoter is a K. sacchari Prm5 promoter. In some embodiments, the microbe has a glnE gene that has been altered to remove the adenylyl-removing (AR) domain, while leaving the coding region for the adenyltransferase (AT) domain, which is functionally expressed. In some embodiments, the microbe has a deletion of the glnD gene.

The genetic variation introduced into one or more bacteria of the agricultural compositions disclosed herein may be a knock-out mutation or it may abolish a regulatory sequence of a target gene, or it may comprise insertion of a heterologous regulatory sequence, for example, insertion of a regulatory sequence found within the genome of the same bacterial species or genus. The regulatory sequence can be chosen based on the expression level of a gene in a bacterial culture or within plant tissue. The genetic variation may be produced by chemical mutagenesis. The plants grown may be exposed to biotic or abiotic stressors. However, in some embodiments, the methods disclosed herein also envision altering the impact of ATP or O₂ on the circuitry, or replacing the circuitry with other regulatory cascades in the cell, or altering genetic circuits other than nitrogen fixation. Gene clusters can be re-engineered to generate functional products under the control of a heterologous regulatory system. By eliminating native regulatory elements outside of, and within, coding sequences of gene clusters, and replacing them with alternative regulatory systems, the functional products of complex genetic operons and other gene clusters can be controlled and/or moved to heterologous cells, including cells of different species other than the species from which the native genes were derived. Once re-engineered, the synthetic gene clusters can be controlled by genetic circuits or other inducible regulatory systems, thereby controlling the products' expression as desired. The expression cassettes can be designed to act as logic gates, pulse generators, oscillators, switches, or memory devices. The controlling expression cassette can be linked to a promoter such that the expression cassette functions as an environmental sensor, such as an oxygen, temperature, touch, osmotic stress, membrane stress, or redox sensor.

As an example, the nifL, nifA, nifT, and nifX genes can be eliminated from the nif gene cluster. Synthetic genes can be designed by codon randomizing the DNA encoding each amino acid sequence. Codon selection is performed, specifying that codon usage be as divergent as possible from the codon usage in the native gene. Proposed sequences are scanned for any undesired features, such as restriction enzyme recognition sites, transposon recognition sites, repetitive sequences, sigma 54 and sigma 70 promoters, cryptic ribosome binding sites, and rho independent terminators. Synthetic ribosome binding sites are chosen to match the strength of each corresponding native ribosome binding site, such as by constructing a fluorescent reporter plasmid in which the 150 bp surrounding a gene's start codon (from −60 to +90) is fused to a fluorescent gene. This chimera can be expressed under control of the Ptac promoter, and fluorescence measured via flow cytometry. To generate synthetic ribosome binding sites, a library of reporter plasmids using 150 bp (−60 to +90) of a synthetic expression cassette is generated. Briefly, a synthetic expression cassette can consist of a random DNA spacer, a degenerate sequence encoding an RBS library, and the coding sequence for each synthetic gene. Multiple clones are screened to identify the synthetic ribosome binding site that best matched the native ribosome binding site. Synthetic operons that consist of the same genes as the native operons are thus constructed and tested for functional complementation. A further exemplary description of synthetic operons is provided in US20140329326.

Some examples of genetic alterations which may be made in Gram positive microbes include: deleting glnR to remove negative regulation of BNF in the presence of environmental nitrogen, inserting different promoters directly upstream of the nif cluster to eliminate regulation by GlnR in response to environmental nitrogen, mutating glnA to reduce the rate of ammonium assimilation by the GS-GOGAT pathway, deleting amtB to reduce uptake of ammonium from the media, mutating glnA so it is constitutively in the feedback-inhibited (FBI-GS) state, to reduce ammonium assimilation by the GS-GOGAT pathway.

In some embodiments, glnR is the main regulator of N metabolism and fixation in, e.g., Paenibacillus species. In some embodiments, the genome of a Paenibacillus species does not contain a gene to produce glnR. In some embodiments, the genome of a Paenibacillus species does not contain a gene to produce glnE or glnD. In some embodiments, the genome of a Paenibacillus species does contain a gene to produce glnB or glnK. For example, Paenibacillus sp. WLY78 doesn't contain a gene for glnB, or its homologs found in the archaeon Methanococcus maripaludis, nif11 and nif12. In some embodiments, the genomes of Paenibacillus species are variable. For example, Paenibacillus polymixa E681 lacks glnK and gdh, has several nitrogen compound transporters, but only amtB appears to be controlled by GlnR. In another example, Paenibacillus sp. JDR2 has glnK, gdh and most other central nitrogen metabolism genes, has many fewer nitrogen compound transporters, but does have glnPHQ controlled by GlnR. Paenibacillus riograndensis SBR5 contains a standard glnRA operon, an fdx gene, a main nif operon, a secondary nif operon, and an anf operon (encoding iron-only nitrogenase). Putative glnR/tnrA sites were found upstream of each of these operons. GlnR may regulate all of the above operons, except the anf operon. GlnR may bind to each of these regulatory sequences as a dimer.

Paenibacillus N-fixing strains may fall into two subgroups: Subgroup I, which contains only a minimal nif gene cluster and subgroup II, which contains a minimal cluster, plus an uncharacterized gene between nifX and hesA, and often other clusters duplicating some of the nif genes, such as nifH, nifHDK, nifBEN, or clusters encoding vanadaium nitrogenase (vnf) or iron-only nitrogenase (anf) genes.

In some embodiments, the genome of a Paenibacillus species may not contain a gene to produce glnB or ginK. In some embodiments, the genome of a Paenibacillus species may contain a minimal nif cluster with 9 genes transcribed from a sigma-70 promoter. In some embodiments, a Paenibacillus nif cluster is negatively regulated by nitrogen or oxygen. In some embodiments, the genome of a Paenibacillus species does not contain a gene to produce sigma-54. For example, Paenibacillus sp. WLY78 does not contain a gene for sigma-54. In some embodiments, a nif cluster is regulated by glnR, and/or TnrA. In some embodiments, activity of a nif cluster is altered by altering activity of glnR, and/or TnrA.

In Bacilli, glutamine synthetase (GS) is feedback-inhibited by high concentrations of intracellular glutamine, causing a shift in confirmation (referred to as FBI-GS). Nif clusters contain distinct binding sites for the regulators GlnR and TnrA in several Bacilli species. GlnR binds and represses gene expression in the presence of excess intracellular glutamine and AMP. A role of GlnR may be to prevent the influx and intracellular production of glutamine and ammonium under conditions of high nitrogen availability. TnrA may bind and/or activate (or repress) gene expression in the presence of limiting intracellular glutamine, and/or in the presence of FBI-GS. In some embodiments, the activity of a Bacilli nif cluster is altered by altering the activity of GlnR.

Feedback-inhibited glutamine synthetase (FBI-GS) may bind GlnR and stabilize binding of GlnR to recognition sequences. Several bacterial species have a GlnR/TnrA binding site upstream of the nif cluster. Altering the binding of FBI-GS and GlnR may alter the activity of the nif pathway.

Additional genetic modifications suitable for the microbes of the present disclosure may be found in International Patent Application No. PCT/US2019/039528, the contents of which are herein incorporated by reference in their entirety.

### Sources of Microbes

Microbes of the present disclosure can be obtained from any source, including environmental and commercial sources. The bacteria (or any microbe according to the disclosure) may be obtained from any general terrestrial environment, including its soils, plants, fungi, animals (including invertebrates) and other biota, including the sediments, water and biota of lakes and rivers; from the marine environment, its biota and sediments (for example, sea water, marine muds, marine plants, marine invertebrates (for example, sponges), marine vertebrates (for example, fish)); the terrestrial and marine geosphere (regolith and rock, for example, crushed subterranean rocks, sand and clays); the cryosphere and its meltwater; the atmosphere (for example, filtered aerial dusts, cloud and rain droplets); urban, industrial and other man-made environments (for example, accumulated organic and mineral matter on concrete, roadside gutters, roof surfaces, and road surfaces).

The plants from which the bacteria (or any microbe according to the disclosure) are obtained may be a plant having one or more desirable traits, for example a plant which naturally grows in a particular environment or under certain conditions of interest. By way of example, a certain plant may naturally grow in sandy soil or sand of high salinity, or under extreme temperatures, or with little water, or it may be resistant to certain pests or disease present in the environment, and it may be desirable for a commercial crop to be grown in such conditions, particularly if they are, for example, the only conditions available in a particular geographic location. By way of further example, the bacteria may be collected from commercial crops grown in such environments, or more specifically from individual crop plants best displaying a trait of interest amongst a crop grown in any specific environment: for example the fastest-growing plants amongst a crop grown in saline-limiting soils, or the least damaged plants in crops exposed to severe insect damage or disease epidemic, or plants having desired quantities of certain metabolites and other compounds, including fiber content, oil content, and the like, or plants displaying desirable colors, taste or smell. The bacteria may be collected from a plant of interest or any material occurring in the environment of interest, including fungi and other animal and plant biota, soil, water, sediments, and other elements of the environment as referred to previously.

The bacteria (or any microbe according to the disclosure) may be isolated from plant tissue. This isolation can occur from any appropriate tissue in the plant, including for example root, stem and leaves, and plant reproductive tissues. Non-limiting examples of plant tissues include a seed, seedling, leaf, cutting, plant, bulb, tuber, root, and rhizomes. In some embodiments, microorganisms are isolated from a seed. In some embodiments, microorganisms are isolated from a root.

Persons having skill in the art will be familiar with techniques for recovering microbes from various environmental sources. For example, microbes useful in the methods and agricultural compositions disclosed herein can be obtained by extracting microbes from surfaces or tissues of native plants; grinding seeds to isolate microbes; planting seeds in diverse soil samples and recovering microbes from tissues; or inoculating plants with exogenous microbes and determining which microbes appear in plant tissues. The parameters for processing samples may be varied to isolate different types of associative microbes, such as rhizospheric, epiphytes, or endophytes. By way of example, some methods for isolation from plants include the sterile excision of the plant material of interest (e.g. root or stem lengths, leaves), surface sterilization with an appropriate solution (e.g. 2% sodium hypochlorite), after which the plant material is placed on nutrient medium for microbial growth. Alternatively, the surface-sterilized plant material can be crushed in a sterile liquid (usually water) and the liquid suspension, including small pieces of the crushed plant material spread over the surface of a suitable solid agar medium, or media, which may or may not be selective (e.g. contain only phytic acid as a source of phosphorus). This approach is especially useful for bacteria which form isolated colonies and can be picked off individually to separate plates of nutrient medium, and further purified to a single species by well-known methods. Alternatively, the plant root or foliage samples may not be surface sterilized but only washed gently thus including surface-dwelling epiphytic microorganisms in the isolation process, or the epiphytic microbes can be isolated separately, by imprinting and lifting off pieces of plant roots, stem or leaves onto the surface of an agar medium and then isolating individual colonies as above. This approach is especially useful for bacteria, for example. Alternatively, the roots may be processed without washing off small quantities of soil attached to the roots, thus including microbes that colonize the plant rhizosphere. Otherwise, soil adhering to the roots can be removed, diluted and spread out onto agar of suitable selective and non-selective media to isolate individual colonies of rhizospheric bacteria.

Microbes may also be sourced from a repository, such as environmental strain collections, instead of initially isolating from a first plant. The microbes can be genotyped and phenotyped, via sequencing the genomes of isolated microbes; profiling the composition of communities in planta; characterizing the transcriptomic functionality of communities or isolated microbes; or screening microbial features using selective or phenotypic media (e.g., nitrogen fixation or phosphate solubilization phenotypes). Selected candidate strains or populations can be obtained via sequence data; phenotype data; plant data (e.g., genome, phenotype, and/or yield data); soil data (e.g., pH, N/P/K content, and/or bulk soil biotic communities); or any combination of these.

### Budapest Treaty on the International Recognition of the Deposit of Microorganisms for the Purpose of Patent Procedures

The microbial deposits of the present disclosure were made under the provisions of the Budapest Treaty on the International Recognition of the Deposit of Microorganisms for the Purpose of Patent Procedure (Budapest Treaty).

Applicants state that pursuant to 37 C.F.R. § 1.808 (a) (2) “all restrictions imposed by the depositor on the availability to the public of the deposited material will be irrevocably removed upon the granting of the patent.” This statement is subject to paragraph (b) of this section (i.e. 37 C.F.R. § 1.808 (b)).

The Enterobacter sacchari has now been reclassified as Kosakonia sacchari, the name for the organism may be used interchangeably throughout the present disclosure.

Some microbes of the present disclosure are derived from two wild-type strains. Strain CI006 is a bacterial species previously classified in the genus Enterobacter (see aforementioned reclassification into Kosakonia). Strain CI019 is a bacterial species classified in the genus Rahnella. The deposit information for the CI006 Kosakonia wild type (WT) and CI019 Rahnella WT are found in Table 1.

Some microorganisms described in this application were deposited on Jan. 6, 2017 or Aug. 11, 2017 with the Bigelow National Center for Marine Algae and Microbiota (NCMA), located at 60 Bigelow Drive, East Boothbay, Maine 04544, USA. As aforementioned, all deposits were made under the terms of the Budapest Treaty on the International Recognition of the Deposit of Microorganisms for the Purposes of Patent Procedure. The Bigelow National Center for Marine Algae and Microbiota accession numbers and dates of deposit for the aforementioned Budapest Treaty deposits are provided in Table 1.

Biologically pure cultures of Kosakonia sacchari (WT), Rahnella aquatilis (WT), and a variant/remodeled Kosakonia sacchari strain were deposited on Jan. 6, 2017 with the Bigelow National Center for Marine Algae and Microbiota (NCMA), located at 60 Bigelow Drive, East Boothbay, Maine 04544, USA, and assigned NCMA Patent Deposit Designation numbers 201701001, 201701003, and 201701002, respectively. The applicable deposit information is found below in Table 1.

Biologically pure cultures of variant/remodeled Kosakonia sacchari strains were deposited on Aug. 11, 2017 with the Bigelow National Center for Marine Algae and Microbiota (NCMA), located at 60 Bigelow Drive, East Boothbay, Maine 04544, USA, and assigned NCMA Patent Deposit Designation numbers 201708004, 201708003, and 201708002, respectively. The applicable deposit information is found below in Table 1.

A biologically pure culture of Klebsiella variicola (WT) was deposited on Aug. 11, 2017 with the Bigelow National Center for Marine Algae and Microbiota (NCMA), located at 60 Bigelow Drive, East Boothbay, Maine 04544, USA, and assigned NCMA Patent Deposit Designation number 201708001. Biologically pure cultures of two Klebsiella variicola variants/remodeled strains were deposited on Dec. 20, 2017 with the Bigelow National Center for Marine Algae and Microbiota (NCMA), located at 60 Bigelow Drive, East Boothbay, Maine 04544, USA, and assigned NCMA Patent Deposit Designation numbers 201712001 and 201712002, respectively. The applicable deposit information is found below in Table 1.

Biologically pure cultures of two Kosakonia sacchari variants/remodeled strains were deposited on Dec. 23, 2019 with the American Type Culture Collection (ATCC), located at 10801 University Boulevard, Manassas, Virginia 20110-2209, USA and assigned ATCC Patent Deposit Numbers PTA-126575 and PTA-126576. Biologically pure cultures of four Klebsiella variicola variants/remodeled strains were deposited on Dec. 23, 2019 with the American Type Culture Collection (ATCC), located at 10801 University Boulevard, Manassas, Virginia 20110-2209, USA and assigned ATCC Patent Deposit Numbers PTA-126577, PTA-126578, PTA-126579 and PTA-126580. A biologically pure culture of a Paenibacillus polymyxa (WT) strain was deposited on Dec. 23, 2019 with the American Type Culture Collection (ATCC), located at 10801 University Boulevard, Manassas, Virginia 20110-2209, USA and assigned ATCC Patent Deposit Number PTA-126581. A biologically pure culture of a Paraburkholderia tropica (WT) strain was deposited on Dec. 23, 2019 with the American Type Culture Collection (ATCC), located at 10801 University Boulevard, Manassas, Virginia 20110-2209, USA and assigned ATCC Patent Deposit Number PTA-126582. A biologically pure culture of a Herbaspirillum aquaticum (WT) strain was deposited on Dec. 23, 2019 with the American Type Culture Collection (ATCC), located at 10801 University Boulevard, Manassas, Virginia 20110-2209, USA and assigned ATCC Patent Deposit Number PTA-126583. Biologically pure cultures of four Metakosakonia intestini variants/remodeled strains were deposited on Dec. 23, 2019 with the American Type Culture Collection (ATCC), located at 10801 University Boulevard, Manassas, Virginia 20110-2209, USA and assigned ATCC Patent Deposit Numbers PTA-126584, PTA-126586, PTA-126587 and PTA-126588. A biologically pure culture of a Metakosakonia intestini (WT) strain was deposited on Dec. 23, 2019 with the American Type Culture Collection (ATCC), located at 10801 University Boulevard, Manassas, Virginia 20110-2209, USA and assigned ATCC Patent Deposit Number PTA-126585. A biologically pure culture of a Klebsiella variicola variant/remodeled strain was deposited on Mar. 25, 2020 with the American Type Culture Collection (ATCC), located at 10801 University Boulevard, Manassas, Virginia 20110-2209, USA and assigned ATCC Patent Deposit Number PTA-126740. A biologically pure culture of a Kosakonia sacchari variant/remodeled strain was deposited on Mar. 25, 2020 with the American Type Culture Collection (ATCC), located at 10801 University Boulevard, Manassas, Virginia 20110-2209, USA and assigned ATCC Patent Deposit Number PTA-126743. The applicable deposit information is found below in Table 1.

TABLE 1
Microorganisms Deposited under the Budapest Treaty

| | Pivot Strain | | | |
|---|---|---|---|---|
| | Designation | | | |
| | (some strains | | | |
| | have multiple | | Accession | |
| Depository | designations) | Taxonomy | Number | Date of Deposit |
| NCMA | CI006, | Kosakonia | NCMA | Jan. 6, 2017 |
| | PBC6.1 | sacchari (WT) | 201701001 | |
| NCMA | CI019 | Rahnella | NCMA | Jan. 6, 2017 |
| | | aquatilis (WT) | 201701003 | |
| NCMA | CM029 | Kosakonia | NCMA | Jan. 6, 2017 |
| | | sacchari | 201701002 | |
| NCMA | CM037 | Kosakonia | NCMA | Aug. 11, 2017 |
| | | sacchari | 201708004 | |
| NCMA | CM38, | Kosakonia | NCMA | Aug. 11, 2017 |
| | PBC6.38 | sacchari | 201708003 | |
| NCMA | CM094, | Kosakonia | NCMA | Aug. 11, 2017 |
| | PBC6.94 | sacchari | 201708002 | |
| NCMA | CI137, 137, | Klebsiella | NCMA | Aug. 11, 2017 |
| | PB137 | variicola (WT) | 201708001 | |
| NCMA | | Klebsiella | NCMA | Dec. 20, 2017 |
| | | variicola | 201712001 | |
| NCMA | | Klebsiella | NCMA | Dec. 20, 2017 |
| | | variicola | 201712002 | |
| ATCC | | Kosakonia | PTA- | Dec. 23, 2019 |
| | | sacchari | 126575 | |
| ATCC | | Kosakonia | PTA- | Dec. 23, 2019 |
| | | sacchari | 126576 | |
| ATCC | | Klebsiella | PTA- | Dec. 23, 2019 |
| | | variicola | 126577 | |
| ATCC | | Klebsiella | PTA- | Dec. 23, 2019 |
| | | variicola | 126578 | |
| ATCC | | Klebsiella | PTA- | Dec. 23, 2019 |
| | | variicola | 126579 | |
| ATCC | | Klebsiella | PTA- | Dec. 23, 2019 |
| | | variicola | 126580 | |
| ATCC | | Paenibacillus | PTA- | Dec. 23, 2019 |
| | | polymyxa (WT) | 126581 | |
| ATCC | CI8 | Paraburkholderia | PTA- | Dec. 23, 2019 |
| | | tropica (WT) | 126582 | |
| ATCC | CI3069 | Herbaspirillum | PTA- | Dec. 23, 2019 |
| | | aquaticum (WT) | 126583 | |
| ATCC | | Metakosakonia | PTA- | Dec. 23, 2019 |
| | | intestine | 126584 | |
| ATCC | CI910 | Metakosakonia | PTA- | Dec. 23, 2019 |
| | | intestini (WT) | 126585 | |
| ATCC | | Metakosakonia | PTA- | Dec. 23, 2019 |
| | | intestine | 126586 | |
| ATCC | | Metakosakonia | PTA- | Dec. 23, 2019 |
| | | intestine | 126587 | |
| ATCC | | Metakosakonia | PTA- | Dec. 23, 2019 |
| | | intestine | 126588 | |
| ATCC | | Klebsiella | PTA- | Mar. 25, 2020 |
| | | variicola | 126740 | |
| ATCC | | Kosakonia | PTA- | Mar. 25, 2020 |
| | | sacchari | 126743 | |

The present disclosure, in certain embodiments, provides isolated and biologically pure microorganisms that have applications, inter alia, in agriculture. The disclosed microorganisms can be utilized in their isolated and biologically pure states, as well as being formulated into compositions (see below section for exemplary composition descriptions). Furthermore, the disclosure provides microbial compositions containing at least two members of the disclosed isolated and biologically pure microorganisms, as well as methods of utilizing said microbial compositions. Furthermore, the disclosure provides for methods of modulating nitrogen fixation in plants via the utilization of the disclosed isolated and biologically pure microbes.

In some aspects, the isolated and biologically pure microorganisms of the disclosure are those from Table 1. In other aspects, the isolated and biologically pure microorganisms of the disclosure are derived from a microorganism of Table 1. For example, a strain, child, mutant, or derivative, of a microorganism from Table 1 are provided herein. The disclosure contemplates all possible combinations of microbes listed in Table 1, said combinations sometimes forming a microbial consortia. The microbes from Table 1, either individually or in any combination, can be combined with any plant, active molecule (synthetic, organic, etc.), adjuvant, carrier, supplement, or biological, mentioned in the disclosure.

### Initial Cell Density

In some embodiments, the present formulation methods comprise the step of selecting an initial cell density to provide an acceptable rate of microbial decay. In some embodiments, the initial cell density of the agricultural composition is varied to identify an initial cell density that lowers the rate of decay compared to an existing formulation. In some embodiments, the initial cell density of the agricultural composition is varied to identify an initial cell density that minimizes the rate of decay while maximizing the cell density. In some embodiments, the present agricultural compositions comprise an initial cell density with an acceptable rate of microbial decay, a rate of decay that is lower than existing formulations, or a rate of decay that is minimized while maximizing cell density.

In some embodiments, the initial cell density is selected to provide an acceptable rate of decay based on a target cell density at a later time point. For example, for an agricultural composition that is targeted to have at least a three-month shelf life, an acceptable rate of decay would be one that results in the three-month old agricultural composition comprising a microbial density above the target threshold given the value of the initial cell density.

To provide a non-limiting illustrative example of a calculation to identify an acceptable rate of decay, suppose the initial cell density is 1E10 CFU/mL; the acceptable threshold for cell density for the purposes of application to agricultural plant tissues or the environs thereof is 1E9 CFU/mL; and the target shelf life is at least three months. Then an acceptable rate of decay would be one that resulted in a composition having the cell density of 1E9 CFU/mL at the three month time point. Assuming that decay is approximately linear for the log of the cell density, this would be a decay rate that was less than or equal to the decay rate r that satisfied the equation log₁₀ T_{f}=log₁₀ Tᵢ₋r×t, where T_{f} is the final cell density threshold in CFU/mL at the target shelf-life time point, Tᵢ is the initial cell density in CFU/mL, r is the decay rate in log₁₀ loss of CFU/mL per day, and/is the number of days at the target shelf-life time point. For this example, that would translate to: log₁₀ 1E9=log₁₀ 1E10−r×90, which is satisfied when r is 1/90≈0.011 log₁₀ loss of CFU/mL per day.

In some embodiments, the method comprises testing multiple initial cell densities and monitoring microbial viability over a period of time. In some embodiments, this comprises generating a titration curve of initial cell density versus microbial decay rate. In some embodiments, the initial cell density is selected to be one associated with an acceptable rate of decay.

In some embodiments, the parameter of initial cell density is varied within the method and selected separately from the other parameters. In some embodiments, the parameter of initial cell density is varied at the same time as one or more other parameters, such as microbial stabilizer, physical stabilizer, or buffering agent.

### Buffering Agents

In some embodiments, the methods of the present disclosure comprise the step of selecting a buffering agent. In some embodiments, the agricultural compositions of the present disclosure comprise a buffering agent. In some embodiments, the buffering agent provides consistency in the pH of the agricultural composition. In some embodiments, the buffering agent prevents fluctuations in the pH of the agricultural composition that are detrimental to the nitrogen-fixing microorganisms comprised by the agricultural composition. In some embodiments, the buffering agent prevents toxic levels of acidity. In some embodiments, the buffering agent prevents toxic levels of basicity.

In some embodiments, the buffering agent is selected based on its ability to maintain the pH of the agricultural composition within an acceptable range for at least three months. In some embodiments, the buffering agent maintains the pH of the agricultural composition within an acceptable range for at least three months, at least four months, at least five months, or at least six months.

In some embodiments, the buffering agent maintains the pH of the agricultural composition in the pH range of pH 5-9, pH 5-8, pH 5-7, pH 5-6, pH 6-9, pH 6-8, pH 6-7, pH 7-9, or pH 7-8. In some embodiments, the buffering agent maintains the pH of the agricultural composition in the pH range of pH 6-8.

In some embodiments, the agricultural composition is buffered to the desired pH using conventional buffering agents. Non-limiting examples of buffering agents suitable for use within the disclosed agricultural compositions include sodium citrate, ascorbate, succinate, lactate, citric acid, boric acid, borax, hydrochloric acid, disodium hydrogen phosphate, acetic acid, formic acid, glycine, bicarbonate, phosphate, tartaric acid, Tris-glycine, Tris-NaCl, Tris-ethylenediamine tetraacetic acid (“EDTA”), Tris-borate, Tris-borate-EDTA, Tris-acteate-EDTA (“TAB”), Tris-buffered saline, 4-(2-hydroxyethyl)-1-piperazineethanesulfonic acid (“HEPES”), 3-(N-morpholino) propanesulfonic acid (“MOPS”), piperazine-1,4-bis(2-ethanesulfonic acid) (“PIPES”), 2-(N-morpholino) ethanesulfonic acid (“MES”), and phosphate buffered saline (“PBS”). Table 2 also provides exemplary buffering agents for use in the agricultural compositions of the disclosure, as well as their pKa values and useful pH ranges. In some embodiments, an agricultural composition of the present disclosure comprises a buffering agent disclosed in Table 2. In some embodiments, an agricultural composition of the present disclosure comprises one or more buffering agents disclosed in Table 2.

TABLE 2
Exemplary buffering agents

| | pKa at | Useful |
|---|---|---|
| Common name and/or chemical name | 25° C. | pH range |
| ACES | 6.78 | 6.1-7.5 |
| Acetic acid (Ethanoic acid) | 4.8 | 3.8-5.8 |
| ADA | 6.59 | 6.0-7.2 |
| AMP | 9.7 | 9.0-10.5 |
| AMPD | 8.8 | 7.8-9.7 |
| AMPSO | 9 | 8.3-9.7 |
| BES | 7.09 | 6.4-7.8 |
| Bicine (2-(bis(2-hydroxyethyl) | 8.35 | 7.6-9.0 |
| amino)acetic acid) | | |
| Bis-Tris | 6.5 | 5.8-7.2 |
| Bis-Tris Propane | 6.8, 9.0 | 6.3-9.5 |
| Boric acid | 9.24 | 8.25-10.25 |
| CABS | 10.7 | 10.0-11.4 |
| Cacodylate (dimethylarsenic acid) | 6.27 | 5.0-7.4 |
| CAPS | 10.4 | 9.7-11.1 |
| CAPSO | 9.6 | 8.9-10.3 |
| CHES (N-Cyclohexyl-2- | 9.3 | 8.3-10.3 |
| aminoethanesulfonic acid) | | |
| Citric acid (2-Hydroxypropane- | 3.13, 4.76, | 2.1-7.4 |
| 1,2,3-tricarboxylic acid) | 6.40 | |
| DIPSO | 7.6 | 7.0-8.2 |
| EPPS | 8 | 7.3-8.7 |
| Gly-Gly | 8.2 | 7.5-8.9 |
| HEPBS | 8.3 | 7.6-9.0 |
| HEPES (4-(2-hydroxyethyl)-1- | 7.48 | 2.5-3.5 or |
| piperazineethanesulfonic acid) | | 6.8-8.2 |
| HEPPSO | 7.8 | 7.1-8.5 |
| KH2PO4 | 7.2 | 6.2-8.2 |
| (Monopotassium phosphate) | | |
| MES | 6.15 | 5.5-6.7 |
| (2-(N-morpholino)ethanesulfonic acid) | | |
| MOBS | 7.6 | 6.9-8.3 |
| MOPS | 7.2 | 6.5-7.9 |
| (3-(N-morpholino)propanesulfonic acid) | | |
| MOPSO | 6.9 | 6.2-7.6 |
| PBS or high buffering capacity PBS | | 5.8-8.0 |
| PIPES | 6.76 | 6.1-7.5 |
| PIPES (piperazine-N,N′-bis(2- | 6.76 | 6.1-7.5 |
| ethanesulfonic acid)) | | |
| POPSO | 7.8 | 7.2-8.5 |
| TABS | 8.9 | 8.2-9.6 |
| TAPS ([tris(hydroxymethyl) | 8.43 | 7.7-9.1 |
| methylamino]propanesulfonic acid) | | |
| TAPSO (3-[N-tris(hydroxymethyl) | 7.635 | 7.0-8.2 |
| methylamino]-2-hydroxypropane- | | |
| sulfonic acid) | | |
| TEA | 7.8 | 7.3-8.3 |
| TES (2-[[1,3-dihydroxy-2-(hydroxymethyl) | 7.4 | 6.8-8.2 |
| propan-2-yl]amino]ethanesulfonic acid) | | |
| Tricine (N-[tris(hydroxymethyl)methyl] | 8.05 | 7.4-8.8 |
| glycine) | | |
| Tris (tris(hydroxymethyl)aminomethane) | 8.07 | 7.1-9.1 |
| or (2-amino-2-(hydroxymethyl)propane- | | |
| 1,3-diol) | | |

Additional buffers and instructions on how to prepare them can be found in, e.g., “Common Buffers and Stock Solutions” (2011) Current Protocols in Nucleic Acid Chemistry, A.2A.1-A.2A.14 and in the Sigma Aldrich “Buffer Reference Center” www.sigmaaldrich.com/life-science/core-bioreagents/biological-buffers/learning-center/buffer-reference-center.html, the contents of each of which are incorporated herein in their entirety.

In some embodiments, the buffering agent is one with a high buffering capacity. In some embodiments, the buffering agent is a modified, high buffering capacity version of any one of the buffering agents disclosed herein. In some embodiments, the buffering agent is PBS. In some embodiments, the buffering agent is modified, high buffering capacity PBS as described in the Examples herein.

In some embodiments, the formulation method comprises the step of varying the buffering agent. In some embodiments, the formulation method comprises the step of varying the concentration/molarity of the buffering agent.

### Microbial Stabilizers

In some embodiments, the method of formulation comprises the step of selecting a microbial stabilizer. In some embodiments, the agricultural composition comprises a microbial stabilizer. A microbial stabilizer is an agent that acts to stabilize the microorganism population within the agricultural composition. In some embodiments, the microbial stabilizer decreases or slows the decay rate of the microbial population. In some embodiments, the microbial stabilizer accomplishes this change in the decay rate by maintaining the microorganisms in a semi-dormant state. In a semi-dormant state, microorganisms do not respond to environmental conditions as rapidly as they would in an active state.

In some embodiments, the microbial stabilizer improves microbial survival rate, decreases microbial decay, improves microbial metabolic activity, improves microbial catabolic gene expression, improves the microbial colonization rate, or decreases toxin accumulation within the agricultural composition after 1-6 months of storage compared to the agricultural composition without the microbial stabilizer.

In some embodiments, the microbial stabilizer increases the survival rate of microbial cells comprised by the agricultural composition after storage, e.g., after 1, 2, 3, 4, 5, or 6 months of storage. In some embodiments, the log loss of CFU/mL of microbes after the storage period is less than 1. In some embodiments, the log loss is less than 1, 0.9, 0.8, 0.7, 0.6, 0.5, 0.4, 0.3, or 0.2.

In some embodiments, the microbial stabilizer improves the metabolic activity and/or catabolic gene expression of the microorganisms comprised by the agricultural composition after the storage period. In some embodiments, the microbes are more metabolically and/or catabolically active than microbes from the agricultural composition without the microbial stabilizer.

In some embodiments, the microbial stabilizer improves the colonization rate of the microorganisms in the agricultural plant after the storage period compared to the agricultural composition minus the microbial stabilizer. In some embodiments, microbial colonization is unaffected by the storage period for the agricultural composition comprising the microbial stabilizer.

In some embodiments, the microbial stabilizer decreases toxin accumulation. In some embodiments, the toxin is a direct product or byproduct of nitrogen fixation. In some embodiments, the toxin is ammonia or ammonium. In some embodiments, the toxin is produced during cell growth/division.

In some embodiments, the microbial stabilizer decreases toxin accumulation at least two-fold over the target time period, e.g., three months, compared to the agricultural composition absent the microbial stabilizer. In some embodiments, the microbial stabilizer decreases toxin accumulation at least two-fold to at least ten-fold compared to the agricultural composition without the microbial stabilizer. In some embodiments, the microbial stabilizer decreases toxin accumulation at least two-fold, at least three-fold, at least four-fold, at least five-fold, at least six-fold, at least seven-fold, at least eight-fold, at least nine-fold, or at least ten-fold. In some embodiments, the microbial stabilizer decreases toxin accumulation about two-fold, about three-fold, about four-fold, about five-fold, about six-fold, about seven-fold, about eight-fold, about nine-fold, or about ten-fold.

In some embodiments, the microbial stabilizer is a sugar. In some embodiments, the microbial stabilizer is a non-reducing sugar. In some embodiments, the microbial stabilizer is a monosaccharide. Monosaccharides suitable for use include, but are not limited to, glucose and fructose. In some embodiments, the microbial stabilizer is fructose. In some embodiments, the microbial stabilizer is a disaccharide. Monosaccharides suitable for use include, but are not limited to, trehalose, sucrose, lactose, melibiose, and lactulose. In some embodiments, the microbial stabilizer is trehalose. In some embodiments, the microbial stabilizer is a polysaccharide. Polysaccharides suitable for use include, but are not limited to, maltodextrin, microcrystalline cellulose, and dextran. Additional carbohydrates suitable for use as microbial stabilizers within the agricultural compositions of the present disclosure include, but are not limited to, pentoses (e.g., ribose, xylose), hexoses (e.g., mannose, sorbose), oligosaccharides (e.g., raffinose), and oligofructoses. In some embodiments, the microbial stabilizer is a sugar alcohol. Sugar alcohols suitable for use include, but are not limited to, glycerol, mannitol, and sorbitol.

In some embodiments, the microbial stabilizer is an amino acid. In some embodiments, the microbial stabilizer is glycine, proline, glutamate, or cysteine. In some embodiments, the microbial stabilizer is a protein or protein hydrolysate. Proteins or protein hydrolysates suitable for use as microbial stabilizers within the agricultural composition of the present disclosure include, but are not limited to, malt extract, milk powder, casein, whey powder, and yeast extract. In some embodiments, the microbial stabilizer is skimmed milk, starch, humic acid, chitosan, CMC, corn steep liquor, molasses, paraffin, pinolene, NFSM, MgSO₄, liquid growth medium, horse serum, or Ficoll.

In some embodiments, the microbial stabilizer is a desiccant. As used herein, a “desiccant” can include any compound or mixture of compounds that can be classified as a desiccant regardless of whether the compound or compounds are used in such concentrations that they in fact have a desiccating effect on the liquid inoculant. Such desiccants are ideally compatible with the microbial population used, and should promote the ability of the microbial population to survive application on the agricultural plant tissues or the environs thereof and to survive desiccation. Examples of suitable desiccants include one or more of trehalose, sucrose, glycerol, and methylene glycol. Other suitable desiccants include, but are not limited to, non-reducing sugars and sugar alcohols (e.g., mannitol or sorbitol).

In some embodiments, the microbial stabilizer comprised by the agricultural composition also acts as a physical stabilizer. In some embodiments, the substance acting as a microbial stabilizer within the agricultural composition has properties of a thickening agent and therefore also acts as a physical stabilizer. In some embodiments, an agricultural composition of the present disclosure comprising both a physical and a microbial stabilizer does so by comprising the same agent that has characteristics of both types of stabilizer.

In some embodiments, the concentration of microbial stabilizer comprised by the agricultural composition ranges from about 0.1% w/v to about 20% w/v. In some embodiments, the concentration of microbial stabilizer comprised by the agricultural composition is in the range of 0.1-1.0% w/v, 1.0-5.0% w/v, 5.0-10% w/v, or 10-20% w/v. In some embodiments, the microbial stabilizer is present at a concentration of about 0.5-10% w/v. In some embodiments, the microbial stabilizer is present at a concentration of about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0% w/v. In some embodiments, the microbial stabilizer is present at a concentration of about 1.0% w/v. In some embodiments, the microbial stabilizer is present at a concentration of about 1.3% w/v. In some embodiments, the microbial stabilizer is present at a concentration of about 2.5% w/v.

### Physical Stabilizers

In some embodiments, the method of formulation comprises the step of selecting a physical stabilizer. In some embodiments, the agricultural composition comprises a physical stabilizer. As used herein, a “physical stabilizer” refers to a substance that improves the homogeneity of the agricultural composition, such that the microbial cells are at a similar density throughout the liquid composition. By increasing homogeneity, the physical stabilizer prevents high concentrations of cells and/or toxins from accumulating in any one sub-volume of the agricultural composition.

In some embodiments, the physical stabilizer increases the viscosity of the liquid agricultural composition. In some embodiments, the physical stabilizer is a thickening agent. In some embodiments, the physical stabilizer is an anti-settling agent. In some embodiments, the physical stabilizer is a suspension aid. In some embodiments, the physical stabilizer acts to maintain microbial cells in suspension, improving the cell's resistance to settle statically and flow under shear or rheological shear-thinning. In some embodiments, a physical stabilizer may also have properties of a microbial stabilizer and vice versa. In some embodiments, the agricultural composition comprises more than one physical stabilizer.

In some embodiments, the physical stabilizer is a polysaccharide. Polysaccharides suitable for use as physical stabilizers include, but are not limited to, polyethylene glycol (PEG), xanthan gum, pectin, and alginates. In some embodiments, the physical stabilizer is xanthan gum. In some embodiments, the physical stabilizer is a protein or protein hydrolysate. Proteins or protein hydrolysates suitable for use as physical stabilizers within the agricultural composition of the present disclosure include, but are not limited to, gluten, collagen, gelatin, elastin, keratin, and albumin. In some embodiments, the physical stabilizer is a polymer. Polymers suitable for use as physical stabilizers include, but are not limited to, Carbopol® (CBP) polymers, methylene glycol, polyvinyl alcohol (PVA), polyvinylpyrrolidone (PVP), poyacrylate, hydroxyethyl cellulose, or hydroxypropyl methylcellulose. In some embodiments, the physical stabilizer is a gum or its derivative. Gums and their derivatives suitable for use as physical stabilizers within the agricultural composition of the present disclosure include, but are not limited to, guar gum, gum Arabic, gum tragacanth, xanthan gum, derivitized guar, hydroxypropyl guar, and polysaccharide gums. In some embodiments, the physical stabilizer is a CBP polymer.

In some embodiments, the physical stabilizer is a suspension aid. Suitable suspension aids for use as physical stabilizers in the agricultural compositions of the present disclosure include, but are not limited to, water soluble polymers such as acrylamide homo- and copolymers, acrylic acid homo- and copolymer, cellulose, methyl cellulose, ethyl cellulose, carboxymethyl cellulose (sodium and other salts), carboxymethyl hydroxyethyl cellulose, hydroxyethyl cellulose, hydrophobically modified hydroxyethyl cellulose, hydroxypropyl cellulose, hydroxypropyl methylcellulose, water-soluble cellulose ethers, carboxy-vinyl copolymers, alginic acid, polyacrylic acid, sodium polyacrylate, partially and fully hydrolyzed polyvinyl alcohols, partially neutralized polyacrylic acid, polyalkylene glycol, polyvinylpyrrolidone and derivatives, starch and its derivatives, vinylpyrrolidone homo- and copolymers, polyacrylamide, attapulgite, montmorillonite, organically modified montmorillonite clays, alumina, precipitated silica, or any mixture thereof.

In some embodiments, the concentration of physical stabilizer comprised by the agricultural composition ranges from about 0.01% w/v to about 30% w/v. In some embodiments, the concentration of physical stabilizer comprised by the agricultural composition is in the range of 0.01-0.1% w/v, 0.1-1.0% w/v, 1.0-5.0% w/v, 5.0-10% w/v, 10-15%, 15-20%, 20-25%, or 25-30% w/v. In some embodiments, the physical stabilizer is present at a concentration of about 0.01-2.0% w/v. In some embodiments, the physical stabilizer is present at a concentration of about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0% w/v. In some embodiments, the physical stabilizer is present at a concentration of about 0.1% w/v. In some embodiments, the physical stabilizer is present at a concentration of about 0.2% w/v.

Additional Agricultural Composition Components and/or Treatments

The present liquid agricultural compositions comprise plant-beneficial, nitrogen-fixing microorganisms and one or more of a buffering agent, a microbial stabilizer, and a physical stabilizer. In some embodiments, the formulation methods of the present disclosure comprise the step of selecting additional components. In some embodiments, the agricultural compositions of the present disclosure comprise additional components. In some embodiments, the additional ingredients are separately added to the liquid agricultural composition. In some embodiments, the agricultural plant tissues or environs thereof of the present disclosure are separately treated with an additional component before, during, or after application of the agricultural compositions of the present disclosure. These additional components may include protectants and beneficial ingredients including but not limited to animal and bird repellants, attractants, baits, herbicides, herbicide safeners, antidessicants, antitranspirants, frost prevention aids, inoculants, dyes, brighteners, markers, synergists, pigments, UV protectants, antioxidants, leaf polish, pigmentation stimulants and inhibitors, surfactants, moisture retention aids, humic acids and humates, lignins and lignates, bitter flavors, irritants, malodorous ingredients, molluscicides (e.g., slugs and snails), nematicides, rodenticides, defoliants, desiccants, sticky traps, IPM (integrated pest management) lures, chemosterilants, plant defense boosters (harpin protein and chitosan), and other beneficial or detrimental agents applied to the surface of the plant tissue or the environs thereof. In some embodiments, multiple active agents are readily formulated within a given agricultural composition, for example, multiple active agents may include two or more of any of the following fungicides, fertilizers, pesticides, herbicides, and any type of active ingredient or class of active ingredient.

Suitable additional ingredients for the agricultural compositions of the present disclosure include, but are not limited to, the following:

Insecticides: A1) the class of carbamates consisting of aldicarb, alanycarb, benfuracarb, carbaryl, carbofuran, carbosulfan, methiocarb, methomyl, oxamyl, pirimicarb, propoxur and thiodicarb; A2) the class of organophosphates consisting of acephate, azinphos-ethyl, azinphos-methyl, chlorfenvinphos, chlorpyrifos, chlorpyrifos-methyl, demeton-S-methyl, diazinon, dichlorvos/DDVP, dicrotophos, dimethoate, disulfoton, ethion, fenitrothion, fenthion, isoxathion, malathion, methamidaphos, methidathion, mevinphos, monocrotophos, oxymethoate, oxydemeton-methyl, parathion, parathion-methyl, phenthoate, phorate, phosalone, phosmet, phosphamidon, pirimiphos-methyl, quinalphos, terbufos, tetrachlorvinphos, triazophos and trichlorfon; A3) the class of cyclodiene organochlorine compounds such as endosulfan; A4) the class of fiproles consisting of ethiprole, fipronil, pyrafluprole and pyriprole; A5) the class of neonicotinoids consisting of acetamiprid, chlothianidin, dinotefuran, imidacloprid, nitenpyram, thiacloprid and thiamethoxam; A6) the class of spinosyns such as spinosad and spinetoram; A7) chloride channel activators from the class of mectins consisting of abamectin, emamectin benzoate, ivermectin, lepimectin and milbemectin; A8) juvenile hormone mimics such as hydroprene, kinoprene, methoprene, fenoxycarb and pyriproxyfen; A9) selective homopteran feeding blockers such as pymetrozine, flonicamid and pyrifluquinazon; A10) mite growth inhibitors such as clofentezine, hexythiazox and etoxazole; A11) inhibitors of mitochondrial ATP synthase such as diafenthiuron, fenbutatin oxide and propargite; uncouplers of oxidative phosphorylation such as chlorfenapyr; A12) nicotinic acetylcholine receptor channel blockers such as bensultap, cartap hydrochloride, thiocyclam and thiosultap sodium; A13) inhibitors of the chitin biosynthesis type 0 from the benzoylurea class consisting of bistrifluron, diflubenzuron, flufenoxuron, hexaflumuron, lufenuron, novaluron and teflubenzuron; A14) inhibitors of the chitin biosynthesis type 1 such as buprofezin; A15) moulting disruptors such as cyromazine; A16) ecdyson receptor agonists such as methoxyfenozide, tebufenozide, halofenozide and chromafenozide; A17) octopamin receptor agonists such as amitraz; A18) mitochondrial complex electron transport inhibitors pyridaben, tebufenpyrad, tolfenpyrad, flufenerim, cyenopyrafen, cyflumetofen, hydramethylnon, acequinocyl or fluacrypyrim; A19) voltage-dependent sodium channel blockers such as indoxacarb and metaflumizone; A20) inhibitors of the lipid synthesis such as spirodiclofen, spiromesifen and spirotetramat; A21) ryanodine receptor-modulators from the class of diamides consisting of flubendiamide, the phthalamide compounds (R)-3-Chlor-N1-{2-methyl-4-[1,2,2,2-tetrafluor-1-(trifluormethyl)ethyl]phenyl}-N2-(1-methyl-2-methyl sulfonylethyl) phthalamid and (S)-3-Chlor-N1-{2-methyl-441,2,2,2-tetrafluor-1-(trifluormethyl)ethyl]phenyl}-N2-(1-methyl-2-methylsulfonylethyl) phthalamid, chloranthraniliprole and cyantraniliprole; A22) compounds of unknown or uncertain mode of action such as azadirachtin, amidoflumet, bifenazate, fluensulfone, piperonyl butoxide, pyridalyl, sulfoxaflor; or A23) sodium channel modulators from the class of pyrethroids consisting of acrinathrin, allethrin, bifenthrin, cyfluthrin, gamma-cyhalothrin, lambda-cyhalothrin, cypermethrin, alpha-cypermethrin, beta-cypermethrin, zeta-cypermethrin, deltamethrin, esfenvalerate, etofenprox, fenpropathrin, fenvalerate, flucythrinate, tau-fluvalinate, permethrin, silafluofen, tefluthrin and tralomethrin and any suitable combinations thereof.

Fungicides: B1) azoles selected from the group consisting of bitertanol, bromuconazole, cyproconazole, difenoconazole, diniconazole, enilconazole, epoxiconazole, fluquinconazole, fenbuconazole, flusilazole, flutriafol, hexaconazole, imibenconazole, ipconazole, metconazole, myclobutanil, penconazole, propiconazole, prothioconazole, simeconazole, triadimefon, triadimenol, tebuconazole, tetraconazole, triticonazole, prochloraz, pefurazoate, imazalil, triflumizole, cyazofamid, benomyl, carbendazim, thia-bendazole, fuberidazole, ethaboxam, etridiazole and hymexazole, azaconazole, diniconazole-M, oxpoconazol, paclobutrazol, uniconazol, 1-(4-chloro-phenyl)-2-([1,2,4]triazol-1-yl)-cycloheptanol and imazalilsulfphate; B2) strobilurins selected from the group consisting of azoxystrobin, dimoxystrobin, enestroburin, fluoxastrobin, kresoxim-methyl, methominostrobin, orysastrobin, picoxystrobin, pyraclostrobin, trifloxystrobin, methyl (2-chloro-541-(3-methylbenzyloxyimino)ethyl]benzyl) carbamate, methyl (2-chloro-5-[1-(6-methylpyridin-2-ylmethoxyimino)ethyl]benzyl) carbamate and methyl 2-(ortho-(2,5-dimethylphenyloxymethylene)-phenyl)-3-methoxyacrylate, 2-(2-(6-(3-chloro-2-methyl-phenoxy)-5-fluoro-pyrimidin-4-yloxy)-phenyl)-2-methoxyimino-N-methyl-acetamide and 3-methoxy-2-(2-(N-(4-methoxy-phenyl)-cyclopropanecarboximidoylsulfanylmethyl)-phenyl)-acrylic acid methyl ester; B3) carboxamides selected from the group consisting of carboxin, benalaxyl, benalaxyl-M, fenhexamid, flutolanil, furametpyr, mepronil, metalaxyl, mefenoxam, ofurace, oxadixyl, oxycarboxin, penthiopyrad, isopyrazam, thifluzamide, tiadinil, 3,4-dichloro-N-(2-cyanophenyl) isothiazole-5-carboxamide, dimethomorph, flumorph, flumetover, fluopicolide (picobenzamid), zoxamide, carpropamid, diclocymet, mandipropamid, N-(2-(443-(4-chlorophenyl) prop-2-ynyloxy]-3-methoxyphenyl)ethyl)-2-methanesulfonyl-amino-3-methylbutyramide, N-(2-(4-[3-(4-chloro-phenyl) prop-2-ynyloxy]-3-methoxy-phenyl)ethyl)-2-ethanesulfonylamino-3-methylbutyramide, methyl 3-(4-chlorophenyl)-3-(2-isopropoxycarbonyl-amino-3-methyl-butyrylamino) propionate, N-(4′-bromobiphenyl-2-yl)-4-difluoromethylA-methylthiazole-6-carboxamide, N-(4′-trifluoromethyl-biphenyl-2-yl)-4-difluoromethyl-2-methylthiazole-5-carboxamide, N-(4′-chloro-3′-fluorobiphenyl-2-yl)-4-difluoromethyl-2-methyl-thiazole-5-carboxamide, N-(3′,4′-dichloro-4-fluorobiphenyl-2-yl)-3-difluoro-methyl-1-methyl-pyrazole-4-carboxamide, N-(3′,4′-dichloro-5-fluorobiphenyl-2-yl)-3-difluoromethyl-1-methylpyrazole-4-carboxamide, N-(2-cyano-phenyl)-3,4-dichloroisothiazole-5-carboxamide, 2-amino-4-methyl-thiazole-5-carboxanilide, 2-chloro-N-(1,1,3-trimethyl-indan-4-yl)-nicotinamide, N-(2-(1,3-dimethylbutyl)-phenyl)-1,3-dimethyl-5-fluoro-1H-pyrazole-4-carboxamide, N-(4′-chloro-3′,5-difluoro-biphenyl-2-yl)-3-difluoromethyl-1-methyl-1H-pyrazole-4-carboxamide, N-(4′-chloro-3′, 5-difluoro-biphenyl-2-yl)-3-trifluoromethyl-1-methyl-1H-pyrazole-4-carboxamide, N-(3′, 4′-dichloro-5-fluoro-biphenyl-2-yl)-3-trifluoromethyl-1-methyl-1H-pyrazole-4-carboxamide, N-(3′, 5-difluoro-4′-methyl-biphenyl-2-yl)-3-difluoromethyl-1-methyl-1H-pyrazole-4-carboxamide, N-(3′, 5-difluoro-4′-methyl-biphenyl-2-yl)-3-trifluoromethyl-1-methyl-1H-pyrazole-4-carboxamide, N-(cis-2-bicyclopropyl-2-yl-phenyl)-3-difluoromethyl-1-methyl-1H-pyrazole-4-carboxamide, N-(trans-2-bicyclopropyl-2-yl-phenyl)-3-difluoro-methyl-1-methyl-1H-pyrazole-4-carboxamide, fluopyram, N-(3-ethyl-3,5-5-trimethyl-cyclohexyl)-3-formylamino-2-hydroxy-benzamide, oxytetracyclin, silthiofam, N-(6-methoxy-pyridin-3-yl) cyclopropanecarboxamide, 2-iodo-N-phenyl-benzamide, N-(2-bicyclo-propyl-2-yl-phenyl)-3-difluormethyl-1-methylpyrazol-4-ylcarboxamide, N-(3′,4′,5′-trifluorobiphenyl-2-yl)-1,3-dimethylpyrazol-4-ylcarboxamide, N-(3′,4′,5′-trifluorobiphenyl-2-yl)-1,3-dimethyl-5-fluoropyrazol-4-yl-carboxamide, N-(3′,4′,5′-trifluorobiphenyl-2-yl)-5-chloro-1,3-dimethyl-pyrazol-4-ylcarboxamide, N-(3′,4′,5′-trifluorobiphenyl-2-yl)-3-fluoromethyl-1-methylpyrazol-4-ylcarboxamide, N-(3′,4′,5′-trifluorobiphenyl-2-yl)-3-(chlorofluoromethyl)-1-methylpyrazol-4-ylcarboxamide, N-(3′,4′,5′-trifluorobiphenyl-2-yl)-3-difluoromethyl-1-methylpyrazol-4-ylcarboxamide, N-(3′,4′,5′-trifluorobiphenyl-2-yl)-3-difluoromethyl-5-fluoro-1-methylpyrazol-4-ylcarboxamide, N-(3′,4′,5′-trifluorobiphenyl-2-yl)-5-chloro-3-difluoromethyl-1-methylpyrazol-4-ylcarboxamide, N-(3′,4′,5′-trifluorobiphenyl-2-yl)-3-(chlorodifluoromethyl)-1-methylpyrazol-4-ylcarboxamide, N-(3′,4′,5′-trifluorobiphenyl-2-yl)-1-methyl-3-trifluoromethylpyrazol-4-ylcarboxamide, N-(3′,4′,5′-trifluorobiphenyl-2-yl)-5-fluoro-1-methyl-3-trifluoromethylpyrazol-4-ylcarboxamide, N-(3′,4′,5′-trifluorobiphenyl-2-yl)-5-chloro-1-methyl-3-trifluoromethylpyrazol-4-ylcarboxamide, N-(2′,4′,5′-trifluorobiphenyl-2-yl)-1,3-dimethylpyrazol-4-ylcarboxamide, N-(2′,4′,5′-trifluorobiphenyl-2-yl)-1,3-dimethyl-5-fluoropyrazol-4-ylcarboxamide, N-(2′,4′,5′-trifluorobiphenyl-2-yl)-5-chloro-1,3-dimethylpyrazol-4-ylcarboxamide, N-(2′,4′,5′-trifluorobiphenyl-2-yl)-3-fluoromethyl-1-methylpyrazol-4-ylcarboxamide, N-(2′,4′,5′-trifluorobiphenyl-2-yl)-3-(chlorofluoromethyl)-1-methylpyrazol-4-ylcarboxamide, N-(2′,4′,5′-trifluorobiphenyl-2-yl)-3-difluoromethyl-1-methylpyrazol-4-ylcarboxamide, N-(2′,4′,5′-trifluorobiphenyl-2-yl)-3-difluoromethyl-5-fluoro-1-methylpyrazol-4-ylcarboxamide, N-(2′,4′,5′-trifluorobiphenyl-2-yl)-5-chloro-3-difluoromethyl-1-methylpyrazol-4-ylcarboxamide, N-(2′,4′,5′-trifluorobiphenyl-2-yl)-3-(chlorodifluoromethyl)-1-methylpyrazol-4-ylcarboxamide, N-(2′,4′,5′-trifluorobiphenyl-2-yl)-1-methyl-3-trifluoromethylpyrazol-4-ylcarboxamide, N-(2′,4′,5′-trifluorobiphenyl-2-yl)-5-fluoro-1-methyl-3-trifluoromethylpyrazol-4-ylcarboxamide, N-(2′,4′,5′-trifluorobiphenyl-2-yl)-5-chloro-1-methyl-3-trifluoromethylpyrazol-4-ylcarboxamide, N-(3′,4′-dichloro-3-fluorobiphenyl-2-yl)-1-methyl-3-trifluoromethyl-1H-pyrazole-4-carboxamide, N-(3′,4′-dichloro-3-fluorobiphenyl-2-yl)-1-methyl-3-difluoromethyl-1H-pyrazole-4-carboxamide, N-(3′,4′-difluoro-3-fluorobiphenyl-2-yl)-1-methyl-3-trifluoromethyl-1H-pyrazole-4-carboxamide, N-(3′,4′-difluoro-3-fluorobiphenyl-2-yl)-1-methyl-S-difluoromethyl-1H-pyrazole-4-carboxamide, N-(3′-chloro-4′-fluoro-3-fluorobiphenyl-2-yl)-1-methyl-3-difluoromethyl-1H-pyrazole-4-carboxamide, N-(3′,4′-dichloro-4-fluorobiphenyl-2-yl)-1-methyl-3-trifluoromethyl-1H-pyrazole-4-carboxamide, N-(3′,4′-difluoro-4-fluorobiphenyl-2-yl)-1-methyl-S-trifluoromethyl-1H-pyrazole-4-carboxamide, N-(3′,4′-dichloro-4-fluorobiphenyl-2-yl)-1-methyl-3-difluoromethyl-1H-pyrazole-4-carboxamide, N-(3′,4′-difluoro-4-fluorobiphenyl-2-yl)-1-methyl-3-difluoromethyl-1H-pyrazole-4-carboxamide, N-(3′-chloro-4′-fluoro-4-fluorobiphenyl-2-yl)-1-methyl-S-difluoromethyl-1H-pyrazole-4-carboxamide, N-(3′,4′-dichloro-5-fluorobiphenyl-2-yl)-1-methyl-3-trifluoromethyl-1H-pyrazole-4-carboxamide, N-(3′,4′-difluoro-5-fluorobiphenyl-2-yl)-1-methyl-3-trifluoromethyl-1H-pyrazole-4-carboxamide, N-(3′,4′-dichloro-5-fluorobiphenyl-2-yl)-1-methyl-S-difluoromethyl-1H-pyrazole-carboxamide, N-(3′,4′-difluoro-5-fluorobiphenyl-2-yl)-1-methyl-3-difluoromethyl-1H-pyrazole-4-carboxamide, N-(3′,4′-dichloro-5-fluorobiphenyl-2-yl)-1,3-dimethyl-1H-pyrazole-4-carboxamide, N-(3′-chloro-4′-fluoro-5-fluorobiphenyl-2-yl)-1-methyl-3-difluoromethyl-1H-pyrazole-4-carboxamide, N-(4′-fluoro-4-fluorobiphenyl-2-yl)-1-methyl-3-trifluoromethyl-1H-pyrazole-4-carboxamide, N-(4′-fluoro-5-fluorobiphenyl-2-yl)-1-methyl-3-trifluoromethyl-1H-pyrazole-4-carboxamide, N-(4′-chloro-5-fluorobiphenyl-2-yl)-1-methyl-3-trifluoromethyl-1H-pyrazole-4-carboxamide, N-(4′-methyl-5-fluorobiphenyl-2-yl)-1-methyl-3-trifluoromethyl-1H-pyrazole-4-carboxamide, N-(4′-fluoro-5-fluorobiphenyl-2-yl)-1,3-dimethyl-1H-pyrazole-4-carboxamide, N-(4′-chloro-5-fluorobiphenyl-2-yl)-1,3-dimethyl-1H-pyrazole-4-carboxamide, N-(4′-methyl-5-fluorobiphenyl-2-yl)-1,3-dimethyl-1H-pyrazole-4-carboxamide, N-(4′-fluoro-6-fluorobiphenyl-2-yl)-1-methyl-3-trifluoromethyl-1H-pyrazole-4-carboxamide, N-(4′-chloro-6-fluorobiphenyl-2-yl)-1-methyl-3-trifluoromethyl-1H-pyrazole-4-carboxamide, N-[2-(1,1,2,3,3,3-hexafluoropropoxy)-phenyl]-3-difluoromethyl-1-methyl-1H-pyrazole-4-carboxamide, N-[4′-(trifluoromethylthio)-biphenyl-2-yl]-3-difluoromethyl-1-methyl-1H-pyrazole-4-carboxamide and N44′-(trifluoromethylthio)-biphenyl-2-yl]-1-methyl-3-trifluoromethyl-1-methyl-1H-pyrazole-4-carboxamide; B4) heterocyclic compounds selected from the group consisting of fluazinam, pyrifenox, bupirimate, cyprodinil, fenarimol, ferimzone, mepanipyrim, nuarimol, pyrimethanil, triforine, fenpiclonil, fludioxonil, aldimorph, dodemorph, fenpropimorph, tridemorph, fenpropidin, iprodione, procymidone, vinclozolin, famoxadone, fenamidone, octhilinone, proben-azole, 5-chloro-7-(4-methyl-piperidin-1-yl)-6-(2,4,6-trifluorophenyl) 41,2,4]triazolo[1,5-a]pyrimidine, anilazine, diclomezine, pyroquilon, proquinazid, tricyclazole, 2-butoxy-6-iodo-3-propylchromen-4-one, acibenzolar-S-methyl, captafol, captan, dazomet, folpet, fenoxanil, quinoxyfen, N,N-dimethyl-3-(3-bromo-6-fluoro-2-methylindole-1-sulfonyl)-[1,2,4]triazole-1-sulfonamide, 5-ethyl-6-octyl41,2,4]triazolo[1 2,3,5,6-tetrachloro-4-methanesulfonyl-pyridine, 3,4,5-trichloro-pyridine-2,6-di-carbonitrile, N-(1-(5-bromo-3-chloro-pyridin-2-yl)-ethyl)-2,4-dichloro-nicotinamide, N-((5-bromo-3-chloro pyridin-2-yl)-methyl)-2,4-dichloro-nicotinamide, diflumetorim, nitrapyrin, dodemorphacetate, fluoroimid, blasticidin-S, chinomethionat, debacarb, difenzoquat, difenzoquat-methylsulphat, oxolinic acid and piperalin; B5) carbamates selected from the group consisting of mancozeb, maneb, metam, methasulphocarb, metiram, ferbam, propineb, thiram, zineb, ziram, diethofencarb, iprovalicarb, benthiavalicarb, propamocarb, propamocarb hydrochlorid, 4-fluorophenyl N-(1-(1-(4-cyanophenyl)-ethanesulfonyl) but-2-yl) carbamate, methyl 3-(4-chloro-phenyl)-3-(2-isopropoxycarbonylamino-3-methyl-butyrylamino) propanoate; or B6) other fungicides selected from the group consisting of guanidine, dodine, dodine free base, iminoctadine, guazatine, antibiotics: kasugamycin, streptomycin, polyoxin, validamycin A, nitrophenyl derivatives: binapacryl, dinocap, dinobuton, sulfur-containing heterocyclyl compounds: dithianon, isoprothiolane, organometallic compounds: fentin salts, organophosphorus compounds: edifenphos, iprobenfos, fosetyl, fosetyl-aluminum, phosphorous acid and its salts, pyrazophos, tolclofos-methyl, organochlorine compounds: dichlofluanid, flusulfamide, hexachloro-benzene, phthalide, pencycuron, quintozene, thiophanate-methyl, tolylfluanid, others: cyflufenamid, cymoxanil, dimethirimol, ethirimol, furalaxyl, metrafenone and spiroxamine, guazatine-acetate, iminoc-tadine-triacetate, iminoctadine-tris(albesilate), kasugamycin hydrochloride hydrate, dichlorophen, pentachlorophenol and its salts, N-(4-chloro-2-nitro-phenyl)-N-ethyl-4-methyl-benzenesulfonamide, did nitrothal-isopropyl, tecnazen, biphenyl, bronopol, diphenylamine, mildiomycin, oxincopper, prohexadione calcium, N-(cyclopropylmethoxyimino-(6-difluoromethoxy-2,3-difluoro-phenyl)-methyl)-2-phenyl acetamide, N′-(4-(4-chloro-3-trifluoromethyl-phenoxy)-2,5-dimethyl-phenyl)-N-ethyl-N-methyl formamidine, N′-(4-(4-fluoro-3-trifluoromethyl-phenoxy)-2,5-dimethyl-phenyl)-N-ethyl-N-methyl formamidine, N′-(2-methyl-5-trifluormethyl-4-(3-trimethylsilanyl-propoxy)-phenyl)-N-ethyl-N-methylformamidine and N′-(5-difluormethyl-2-methyl-4-(3-trimethylsilanyl-propoxy)-phenyl)-N-ethyl-N-methyl formamidine, and any combinations thereof.

Herbicides: C1) acetyl-CoA carboxylase inhibitors (ACC), for example cyclohexenone oxime ethers, such as alloxydim, clethodim, cloproxydim, cycloxydim, sethoxydim, tralkoxydim, butroxydim, clefoxydim or tepraloxydim; phenoxyphenoxypropionic esters, such as clodinafop-propargyl, cyhalofop-butyl, diclofop-methyl, fenoxaprop-ethyl, fenoxaprop-P-ethyl, fenthiapropethyl, fluazifop-butyl, fluazifop-P-butyl, haloxyfop-ethoxyethyl, haloxyfop-methyl, haloxyfop-P-methyl, isoxapyrifop, propaquizafop, quizalofop-ethyl, quizalofop-P-ethyl or quizalofop-tefuryl; or arylaminopropionic acids, such as flamprop-methyl or flamprop-isopropyl; C2 acetolactate synthase inhibitors (ALS), for example imidazolinones, such as imazapyr, imazaquin, imazamethabenz-methyl (imazame), imazamox, imazapic or imazethapyr; pyrimidyl ethers, such as pyrithiobac-acid, pyrithiobac-sodium, bispyribac-sodium. KIH-6127 or pyribenzoxym; sulfonamides, such as florasulam, flumetsulam or metosulam; or sulfonylureas, such as amidosulfuron, azimsulfuron, bensulfuron-methyl, chlorimuron-ethyl, chlorsulfuron, cinosulfuron, cyclosulfamuron, ethametsulfuron-methyl, ethoxysulfuron, flazasulfuron, halosulfuron-methyl, imazosulfuron, metsulfuron-methyl, nicosulfuron, primisulfuron-methyl, prosulfuron, pyrazosulfuron-ethyl, rimsulfuron, sulfometuron-methyl, thifensulfuron-methyl, triasulfuron, tribenuron-methyl, triflusulfuron-methyl, tritosulfuron, sulfosulfuron, foramsulfuron or iodosulfuron; C3) amides, for example allidochlor (CDAA), benzoylprop-ethyl, bromobutide, chiorthiamid. diphenamid, etobenzanidibenzchlomet), fluthiamide, fosamin or monalide; C4) auxin herbicides, for example pyridinecarboxylic acids, such as clopyralid or picloram; or 2,4-D or benazolin; C5) auxin transport inhibitors, for example naptalame or diflufenzopyr; C6) carotenoid biosynthesis inhibitors, for example benzofenap, clomazone (dimethazone), diflufenican, fluorochloridone, fluridone, pyrazolynate, pyrazoxyfen, isoxaflutole, isoxachlortole, mesotrione, sulcotrione (chlormesulone), ketospiradox, flurtamone, norflurazon or amitrol; C7) enolpyruvylshikimate-3-phosphate synthase inhibitors (EPSPS), for example glyphosate or sulfosate; C8) glutamine synthetase inhibitors, for example bilanafos (bialaphos) or glufosinate-ammonium; C9) lipid biosynthesis inhibitors, for example anilides, such as anilofos or mefenacet; chloroacetanilides, such as dimethenamid, S-dimethenamid, acetochlor, alachlor, butachlor, butenachlor, diethatyl-ethyl, dimethachlor, metazachlor, metolachlor, S-metolachlor, pretilachlor, propachlor, prynachlor, terbuchlor, thenylchlor or xylachlor; thioureas, such as butylate, cycloate, di-allate, dimepiperate, EPTC. esprocarb, molinate, pebulate, prosulfocarb, thiobencarb (benthiocarb), tri-allate or vemolate; or benfuresate or perfluidone; C10) mitosis inhibitors, for example carbamates, such as asulam, carbetamid, chlorpropham, orbencarb, pronamid (propyzamid), propham or tiocarbazil; dinitroanilines, such as benefin, butralin, dinitramin, ethalfluralin, fluchloralin, oryzalin, pendimethalin, prodiamine or trifluralin; pyridines, such as dithiopyr or thiazopyr; or butamifos, chlorthal-dimethyl (DCPA) or maleic hydrazide; C11) protoporphyrinogen IX oxidase inhibitors, for example diphenyl ethers, such as acifluorfen, acifluorfen-sodium, aclonifen, bifenox, chlomitrofen (CNP), ethoxyfen, fluorodifen, fluoroglycofen-ethyl, fomesafen, furyloxyfen, lactofen, nitrofen, nitrofluorfen or oxyfluorfen; oxadiazoles, such as oxadiargyl or oxadiazon; cyclic imides, such as azafenidin, butafenacil, carfentrazone-ethyl, cinidon-ethyl, flumiclorac-pentyl, flumioxazin, flumipropyn, flupropacil, fluthiacet-methyl, sulfentrazone or thidiazimin; or pyrazoles, such as ET-751.JV 485 or nipyraclofen; C12) photosynthesis inhibitors, for example propanil, pyridate or pyridafol; benzothiadiazinones, such as bentazone; dinitrophenols, for example bromofenoxim, dinoseb, dinoseb-acetate, dinoterb or DNOC; dipyridylenes, such as cyperquat-chloride, difenzoquat-methyl sulfate, diquat or paraquat-dichloride; ureas, such as chlorbromuron, chlorotoluron, difenoxuron, dimefuron, diuron, ethidimuron, fenuron, fluometuron, isoproturonisouron, linuron, methabenzthiazuron, methazole, metobenzuron, metoxuron, monolinuron, neburon, siduron or tebuthiuron; phenols, such as bromoxynil or ioxynil; chloridazon; triazines, such as ametryn, atrazine, cyanazine, desmein, dimethamethryn, hexazinone, prometon, prometryn, propazine, simazine, simetryn, terbumeton, terbutryn, terbutylazine or trietazine; triazinones, such as metamitron or metribuzin; uracils, such as bromacil, lenacil or terbacil; or biscarbamates, such as desmedipham or phenmedipham; C13) synergists, for example oxiranes, such as tridiphane; C14) CIS cell wall synthesis inhibitors, for example isoxaben or dichlobenil; C16) various other herbicides, for example dichloropropionic acids, such as dalapon; dihydrobenzofurans, such as ethofumesate; phenylacetic acids, such as chlorfenac (fenac); or aziprotryn, barban, bensulide, benzthiazuron, benzofluor, buminafos, buthidazole, buturon, cafenstrole, chlorbufam, chlorfenprop-methyl, chloroxuron, cinmethylin, cumyluron, cycluron, cyprazine, cyprazole, dibenzyluron, dipropetryn, dymron, eglinazin-ethyl, endothall, ethiozin, flucabazone, fluorbentranil, flupoxam, isocarbamid, isopropalin, karbutilate, mefluidide, monuron, napropamide, napropanilide, nitralin, oxaciclomefone, phenisopham, piperophos, procyazine, profluralin, pyributicarb, secbumeton, sulfallate (CDEC), terbucarb, triaziflam, triazofenamid or trimeturon; or their environmentally compatible salts or combinations thereof.

Nematicides: Benomyl, cloethocarb, aldoxycarb, tirpate, diamidafos, fenamiphos, cadusafos, dichlofenthion, ethoprophos, fensulfothion, fosthiazate, heterophos, isamidofof, isazofos, phosphocarb, thionazin, imicyafos, mecarphon, acetoprole, benclothiaz, chloropicrin, dazomet, fluensulfone, oxamyl, terbufos and suitable combinations thereof.

Plant Growth Regulators: D1) Antiauxins, such as clofibric acid, 2,3,5-triiodobenzoic acid; D2) Auxins such as 4-CPA, 2,4-D, 2,4-DB, 2,4-DEP, dichlorprop, fenoprop, IAA, IBA, naphthaleneacetamide, α-naphthaleneacetic acids, 1-naphthol, naphthoxyacetic acids, potassium naphthenate, sodium naphthenate, 2,4,5-T; D3) cytokinins, such as 21P, benzyladenine, 4-hydroxyphenethyl alcohol, kinetin, zeatin; D4) defoliants, such as calcium cyanamide, dimethipin, endothal, ethephon, merphos, metoxuron, pentachlorophenol, thidiazuron, tribufos; D5) ethylene inhibitors, such as aviglycine, 1-methylcyclopropene; D6) ethylene releasers, such as ACC, etacelasil, ethephon, glyoxime; D7) gametocides, such as fenridazon, maleic hydrazide; D8) gibberellins, such as gibberellins, gibberellic acid; D9) growth inhibitors, such as abscisic acid, ancymidol, butralin, carbaryl, chlorphonium, chlorpropham, dikegulac, flumetralin, fluoridamid, fosamine, glyphosine, isopyrimol, jasmonic acid, maleic hydrazide, mepiquat, piproctanyl, prohydrojasmon, propham, tiaojiean, 2,3,5-tri-iodobenzoic acid; D10) morphactins, such as chlorfluren, chlorflurenol, dichlorflurenol, flurenol; D11) growth retardants, such as chlormequat, daminozide, flurprimidol, mefluidide, paclobutrazol, tetcyclacis, uniconazole; D12) growth stimulators, such as brassinolide, brassinolide-ethyl, DCPTA, forchlorfenuron, hymexazol, prosuler, triacontanol; D13) unclassified plant growth regulators, such as bachmedesh, benzofluor, buminafos, carvone, choline chloride, ciobutide, clofencet, cyanamide, cyclanilide, cycloheximide, cyprosulfamide, epocholeone, ethychlozate, ethylene, fuphenthiourea, furalane, heptopargil, holosulf, inabenfide, karetazan, lead arsenate, methasulfocarb, prohexadione, pydanon, sintofen, triapenthenol, trinexapac.

### Fertilizers, Nitrogen Stabilizers, and Urease Inhibitors

The agricultural compositions of the disclosure, which may comprise any microbe taught herein, may be combined with one or more of a: fertilizer, nitrogen stabilizer, or urease inhibitor.

In some embodiments, fertilizers are used in combination with the methods and bacteria of the present disclosure. Fertilizers include anhydrous ammonia, urea, ammonium nitrate, and urea-ammonium nitrate (UAN) compositions, among many others. In some embodiments, pop-up fertilization and/or starter fertilization is used in combination with the methods and bacteria of the present disclosure.

In some embodiments, nitrogen stabilizers are used in combination with the methods and bacteria of the present disclosure. Nitrogen stabilizers include nitrapyrin, 2-chloro-6-(trichloromethyl)pyridine, N-SERVE 24, INSTINCT, dicyandiamide (DCD).

In some embodiments, urease inhibitors are used in combination with the methods and bacteria of the present disclosure. Urease inhibitors include N-(n-butyl)-thiophosphoric triamide (NBPT), AGROTAIN, AGROTAIN PLUS, and AGROTAIN PLUS SC. Further, the disclosure contemplates utilization of AGROTAIN ADVANCED 1.0, AGROTAIN DRI-MAXX, and AGROTAIN ULTRA.

Further, stabilized forms of fertilizer can be used. For example, a stabilized form of fertilizer is SUPER U, containing 46% nitrogen in a stabilized, urea-based granule, SUPERU contains urease and nitrification inhibitors to guard from denitrification, leaching, and volatilization. Stabilized and targeted foliar fertilizer such as NITAMIN may also be used herein.

Pop-up fertilizers are commonly used in corn fields. Pop-up fertilization comprises applying a few pounds of nutrients with the seed at planting. Pop-up fertilization is used to increase seedling vigor.

Slow- or controlled-release fertilizer that may be used herein entails: A fertilizer containing a plant nutrient in a form which delays its availability for plant uptake and use after application, or which extends its availability to the plant significantly longer than a reference ‘rapidly available nutrient fertilizer’ such as ammonium nitrate or urea, ammonium phosphate or potassium chloride. Such delay of initial availability or extended time of continued availability may occur by a variety of mechanisms. These include controlled water solubility of the material by semi-permeable coatings, occlusion, protein materials, or other chemical forms, by slow hydrolysis of water-soluble low molecular weight compounds, or by other unknown means.

Stabilized nitrogen fertilizer that may be used herein entails: A fertilizer to which a nitrogen stabilizer has been added. A nitrogen stabilizer is a substance added to a fertilizer which extends the time the nitrogen component of the fertilizer remains in the soil in the urea-N or ammoniacal-N form.

Nitrification inhibitor that may be used herein entails: A substance that inhibits the biological oxidation of ammoniacal-N to nitrate-N. Some examples include: (1) 2-chloro-6-(trichloromethyl-pyridine), common name Nitrapyrin, manufactured by Dow Chemical; (2) 4-amino-1,2,4-6-triazole-HCl, common name ATC, manufactured by Ishihada Industries; (3) 2,4-diamino-6-trichloro-methyltriazine, common name CI-1580, manufactured by American Cyanamid; (4) Dicyandiamide, common name DCD, manufactured by Showa Denko; (5) Thiourea, common name TU, manufactured by Nitto Ryuso; (6) 1-mercapto-1,2,4-triazole, common name MT, manufactured by Nippon; (7) 2-amino-4-chloro-6-methyl-pyramidine, common name AM, manufactured by Mitsui Toatsu; (8) 3,4-dimethylpyrazole phosphate (DMPP), from BASF; (9) 1-amide-2-thiourea (ASU), from Nitto Chemical Ind.; (10) Ammoniumthiosulphate (ATS); (11) 1H-1,2,4-triazole (HPLC); (12) 5-ethylene oxide-3-trichloro-methly 1,2,4-thiodiazole (Terrazole), from Olin Mathieson; (13) 3-methylpyrazole (3-MP); (14) 1-carbamoyle-3-methyl-pyrazole (CMP); (15) Neem; and (16) DMPP.

Urease inhibitor that may be used herein entails: A substance that inhibits hydrolytic action on urea by the enzyme urease. Thousands of chemicals have been evaluated as soil urease inhibitors (Kiss and Simihaian, 2002). However, only a few of the many compounds tested meet the necessary requirements of being nontoxic, effective at low concentration, stable, and compatible with urea (solid and solutions), degradable in the soil and inexpensive. They can be classified according to their structures and their assumed interaction with the enzyme urease (Watson, 2000, 2005). Four main classes of urease inhibitors have been proposed: (a) reagents which interact with the sulphydryl groups (sulphydryl reagents), (b) hydroxamates, (c) agricultural crop protection chemicals, and (d) structural analogues of urea and related compounds. N-(n-Butyl) thiophosphoric triamide (NBPT), phenylphosphorodiamidate (PPD/PPDA), and hydroquinone are probably the most thoroughly studied urease inhibitors (Kiss and Simihaian, 2002). Research and practical testing has also been carried out with N-(2-nitrophenyl) phosphoric acid triamide (2-NPT) and ammonium thiosulphate (ATS). The organo-phosphorus compounds are structural analogues of urea and are some of the most effective inhibitors of urease activity, blocking the active site of the enzyme (Watson, 2005).

In some embodiments, compositions are supplemented with trace metal ions, such as molybdenum ions, iron ions, manganese ions, or combinations of these ions. The concentration of ions in examples of compositions as described herein may between about 0.1 mM and about 50 mM.

Some examples of agricultural compositions may also include additional carriers, besides those involved in the formulation process. Additional carriers may include beta-glucan, carboxylmethyl cellulose (CMC), bacterial extracellular polymeric substance (EPS), sugar, trehalose, maltose, animal milk, milk powder, or other suitable carriers. In some embodiments, peat or planting materials can be used as a carrier, or biopolymers in which a composition is entrapped in the biopolymer can be used as a carrier.

Agricultural compositions described herein may include additional agriculturally acceptable carriers, in addition to the microbial stabilizers, physical stabilizers, and/or buffering agents included in the formulation process. Additional ingredients useful for these embodiments may include at least one member selected from the group consisting of a tackifier, a fungicide, an antibacterial agent, a preservative, a stabilizer, a surfactant, an anti-complex agent, an herbicide, a nematicide, an insecticide, a plant growth regulator, a fertilizer, a rodenticide, a desiccant, a bactericide, a nutrient, or any combination thereof. For example, any of the agricultural compositions described herein can include an agriculturally acceptable carrier (e.g., one or more of a fertilizer such as a non-naturally occurring fertilizer, an adhesion agent such as a non-naturally occurring adhesion agent, and a pesticide such as a non-naturally occurring pesticide). A non-naturally occurring adhesion agent can be, for example, a polymer, copolymer, or synthetic wax. For example, any of the coated plant tissues or the environs thereof described herein can contain such an agriculturally acceptable carrier in their coating. In any of the agricultural compositions or methods described herein, an agriculturally acceptable carrier can be or can include a non-naturally occurring compound (e.g., a non-naturally occurring fertilizer, a non-naturally occurring adhesion agent such as a polymer, copolymer, or synthetic wax, or a non-naturally occurring pesticide). Non-limiting examples of agriculturally acceptable carriers are described below. Additional examples of agriculturally acceptable carriers are known in the art.

In some cases, microbes are mixed with an additional agriculturally acceptable carrier. The carrier can be a solid carrier or liquid carrier, and in various forms including microspheres, powders, emulsions and the like. The carrier may be any one or more of a number of carriers that confer a variety of properties, such as increased stability, wettability, or dispersability. Wetting agents such as natural or synthetic surfactants, which can be nonionic or ionic surfactants, or a combination thereof can be included in the agricultural composition. Suitable formulations that may be prepared include wettable powders, granules, gels, agar strips or pellets, thickeners, and the like, microencapsulated particles, and the like, liquids such as aqueous flowables, aqueous suspensions, water-in-oil emulsions, etc. The formulation may include grain or legume products, for example, ground grain or beans, broth or flour derived from grain or beans, starch, sugar, or oil.

In some embodiments, the agricultural carrier is a soil or a plant growth medium. Other agricultural carriers that may be used include water, fertilizers, plant-based oils, humectants, or combinations thereof. Alternatively, the agricultural carrier may be a solid, such as diatomaceous earth, loam, silica, alginate, clay, bentonite, vermiculite, seed cases, other plant and animal products, or combinations, including granules, pellets, or suspensions. Mixtures of any of the aforementioned ingredients are also contemplated as carriers, such as but not limited to, pesta (flour and kaolin clay), agar or flour-based pellets in loam, sand, or clay, etc. Agricultural compositions may include food sources for the bacteria, such as barley, rice, or other biological materials such as seed, plant parts, sugar cane bagasse, hulls or stalks from grain processing, ground plant material or wood from building site refuse, sawdust or small fibers from recycling of paper, fabric, or wood.

For example, a fertilizer can be used to help promote the growth or provide nutrients to a plant tissue, e.g., a seed, seedling, or plant. Non-limiting examples of fertilizers include nitrogen, phosphorous, potassium, calcium, sulfur, magnesium, boron, chloride, manganese, iron, zinc, copper, molybdenum, and selenium (or a salt thereof). Additional examples of fertilizers include one or more amino acids, salts, carbohydrates, vitamins, glucose, NaCl, yeast extract, NH₄H₂PO₄, (NH₄)₂SO₄, glycerol, valine, L-leucine, lactic acid, propionic acid, succinic acid, malic acid, citric acid, KH tartrate, xylose, lyxose, and lecithin. In one embodiment, the agricultural composition can include a tackifier or adherent (referred to as an adhesive agent) to help bind other active agents to a substance (e.g., a surface of a plant tissue or the environs thereof). Such agents are useful for combining microbes with carriers that can contain other compounds (e.g., control agents that are not biologic), to yield a coating composition. Such compositions help create coatings around the plant tissues or the environs thereof to maintain contact between the microbe and other agents with the plant tissues or the environs thereof. In one embodiment, adhesives are selected from the group consisting of: alginate, gums, starches, lecithins, formononetin, polyvinyl alcohol, alkali formononetinate, hesperetin, polyvinyl acetate, cephalins, Gum Arabic, Xanthan Gum, Mineral Oil, Polyethylene Glycol (PEG), Polyvinyl pyrrolidone (PVP), Arabino-galactan, Methyl Cellulose, PEG 400, Chitosan, Polyacrylamide, Polyacrylate, Polyacrylonitrile, Glycerol, Triethylene glycol, Vinyl Acetate, Gellan Gum, Polystyrene, Polyvinyl, Carboxymethyl cellulose, Gum Ghatti, and polyoxyethylene-polyoxybutylene block copolymers.

In some embodiments, the adhesives can be, e.g. a wax such as carnauba wax, beeswax, Chinese wax, shellac wax, spermaceti wax, candelilla wax, castor wax, ouricury wax, and rice bran wax, a polysaccharide (e.g., starch, dextrins, maltodextrins, alginate, and chitosans), a fat, oil, a protein (e.g., gelatin and zeins), gum arables, and shellacs. Adhesive agents can be non-naturally occurring compounds, e.g., polymers, copolymers, and waxes. For example, non-limiting examples of polymers that can be used as an adhesive agent include: polyvinyl acetates, polyvinyl acetate copolymers, ethylene vinyl acetate (EVA) copolymers, polyvinyl alcohols, polyvinyl alcohol copolymers, celluloses (e.g., ethylcelluloses, methylcelluloses, hydroxymethylcelluloses, hydroxypropylcelluloses, and carboxymethylcelluloses), polyvinylpyrolidones, vinyl chloride, vinylidene chloride copolymers, calcium lignosulfonates, acrylic copolymers, polyvinylacrylates, polyethylene oxide, acylamide polymers and copolymers, polyhydroxyethyl acrylate, methylacrylamide monomers, and polychloroprene.

In some embodiments, one or more of the adhesion agents, anti-fungal agents, growth regulation agents, and pesticides (e.g., insecticide) are non-naturally occurring compounds (e.g., in any combination). Additional examples of agriculturally acceptable carriers include dispersants (e.g., polyvinylpyrrolidone/vinyl acetate PVPIVA S-630), surfactants, binders, and filler agents.

The agricultural composition can also contain a surfactant. Non-limiting examples of surfactants include nitrogen-surfactant blends such as Prefer 28 (Cenex), Surf-N (US), Inhance (Brandt), P-28 (Wilfarm) and Patrol (Helena); esterified seed oils include Sun-It II (AmCy), MSO (UAP), Scoil (Agsco), Hasten (Wilfarm) and Mes-100 (Drexel); and organo-silicone surfactants include Silwet L77 (UAP), Silikin (Terra), Dyne-Amic (Helena), Kinetic (Helena), Sylgard 309 (Wilbur-Ellis) and Century (Precision). In one embodiment, the surfactant is present at a concentration of between 0.01% v/v to 10% v/v. In another embodiment, the surfactant is present at a concentration of between 0.1% v/v to 1% v/v.

In some embodiments, a fungicide includes a compound or agent, whether chemical or biological, that can inhibit the growth of a fungus or kill a fungus. In some embodiments, a fungicide includes compounds that may be fungistatic or fungicidal. In some embodiments, a fungicide is a protectant, or agent that is effective predominantly on the surface of plant tissues or the environs thereof. In some embodiments, a fungicide is a protectant, or agent that is effective predominantly on the seed surface, providing protection against seed surface-borne pathogens and providing some level of control of soil-borne pathogens. Non-limiting examples of protectant fungicides include captan, maneb, thiram, or fludioxonil.

In some embodiments, fungicide can be a systemic fungicide, which can be absorbed into the emerging seedling and inhibit or kill the fungus inside host plant tissues. Systemic fungicides used for agricultural treatment include, but are not limited to the following: azoxystrobin, carboxin, mefenoxam, metalaxyl, thiabendazole, trifloxystrobin, and various triazole fungicides, including difenoconazole, ipconazole, tebuconazole, and triticonazole. Mefenoxam and metalaxyl are primarily used to target the water mold fungi Pythium and Phytophthora. Some fungicides are preferred over others, depending on the plant species, either because of subtle differences in sensitivity of the pathogenic fungal species, or because of the differences in the fungicide distribution or sensitivity of the plants. In some embodiments, fungicide can be a biological control agent, such as a bacterium or fungus. Such organisms may be parasitic to the pathogenic fungi, or secrete toxins or other substances which can kill or otherwise prevent the growth of fungi. Any type of fungicide, particularly ones that are commonly used on plants, can be used as a control agent in an agricultural composition.

In some embodiments, the agricultural composition comprises a control agent which has antibacterial properties. In one embodiment, the control agent with antibacterial properties is selected from the compounds described herein elsewhere. In another embodiment, the compound is Streptomycin, oxytetracycline, oxolinic acid, or gentamicin. Other examples of antibacterial compounds which can be used as part of an agricultural composition include those based on dichlorophene and benzylalcohol hemi formal (Proxel® from ICI or Acticide® RS from Thor Chemie and Kathon® MK 25 from Rohm & Haas) and isothiazolinone derivatives such as alkylisothiazolinones and benzisothiazolinones (Acticide® MBS from Thor Chemie).

In some embodiments, growth regulator is selected from the group consisting of: Abscisic acid, amidochlor, ancymidol, 6-benzylaminopurine, brassinolide, butralin, chlormequat (chlormequat chloride), choline chloride, cyclanilide, daminozide, dikegulac, dimethipin, 2,6-dimethylpuridine, ethephon, flumetralin, flurprimidol, fluthiacet, forchlorfenuron, gibberellic acid, inabenfide, indole-3-acetic acid, maleic hydrazide, mefluidide, mepiquat (mepiquat chloride), naphthaleneacetic acid, N-6-benzyladenine, paclobutrazol, prohexadione phosphorotrithioate, 2,3,5-tri-iodobenzoic acid, trinexapac-ethyl and uniconazole. Additional non-limiting examples of growth regulators include brassinosteroids, cytokinines (e.g., kinetin and zeatin), auxins (e.g., indolylacetic acid and indolylacetyl aspartate), flavonoids and isoflavanoids (e.g., formononetin and diosmetin), phytoaixins (e.g., glyceolline), and phytoalexin-inducing oligosaccharides (e.g., pectin, chitin, chitosan, polygalacuronic acid, and oligogalacturonic acid), and gibellerins. Such agents are ideally compatible with the agricultural plant tissues or the environs thereof onto which the agricultural composition is applied (e.g., it should not be deleterious to the growth or health of the plant). Furthermore, the agent is ideally one which does not cause safety concerns for human, animal or industrial use (e.g., no safety issues, or the compound is sufficiently labile that the commodity plant product derived from the plant contains negligible amounts of the compound).

Some examples of nematode-antagonistic biocontrol agents include ARF18; 30 Arthrobotrys spp.; Chaetomium spp.; Cylindrocarpon spp.; Exophilia spp.; Fusarium spp.; Gliocladium spp.; Hirsutella spp.; Lecanicillium spp.; Monacrosporium spp.; Myrothecium spp.; Neocosmospora spp.; Paecilomyces spp.; Pochonia spp.; Stagonospora spp.; vesicular-arbuscular mycorrhizal fungi, Burkholderia spp.; Pasteuria spp., Brevibacillus spp.; Pseudomonas spp.; and Rhizobacteria. Particularly preferred nematode-antagonistic biocontrol agents include ARF18, Arthrobotrys oligospora, Arthrobotrys dactyloides, Chaetomium globosum, Cylindrocarpon heteronema, Exophilia jeanselmei, Exophilia pisciphila, Fusarium aspergilus, Fusarium solani, Gliocladium catenulatum, Gliocladium roseum, Gliocladium vixens, Hirsutella rhossiliensis, Hirsutella minnesotensis, Lecanicillium lecanii, Monacrosporium drechsleri, Monacrosporium gephyropagum, Myrotehcium verrucaria, Neocosmospora vasinfecta, Paecilomyces lilacinus, Pochonia chlamydosporia, Stagonospora heteroderae, Stagonospora phaseoli, vesicular-arbuscular mycorrhizal fungi, Burkholderia cepacia, Pasteuria penetrans, Pasteuria thornei, Pasteuria nishizawae, Pasteuria ramosa, Pastrueia usage, Brevibacillus laterosporus strain G4, Pseudomonas fluorescens and Rhizobacteria.

Some examples of nutrients can be selected from the group consisting of a nitrogen fertilizer including, but not limited to Urea, Ammonium nitrate, Ammonium sulfate, Non-pressure nitrogen solutions, Aqua ammonia, Anhydrous ammonia, Ammonium thiosulfate, Sulfur-coated urea, Urea-formaldehydes, IBDU, Polymer-coated urea, Calcium nitrate, Ureaform, and Methylene urea, phosphorous fertilizers such as Diammonium phosphate, Monoammonium phosphate, Ammonium polyphosphate, Concentrated superphosphate and Triple superphosphate, and potassium fertilizers such as Potassium chloride, Potassium sulfate, Potassium-magnesium sulfate, Potassium nitrate. Such compositions can exist as free salts or ions within the agricultural composition. Alternatively, nutrients/fertilizers can be complexed or chelated to provide sustained release over time.

Some examples of rodenticides may include selected from the group of substances consisting of 2-isovalerylindan-1,3-dione, 4-(quinoxalin-2-ylamino) benzenesulfonamide, alpha-chlorohydrin, aluminum phosphide, antu, arsenous oxide, barium carbonate, bisthiosemi, brodifacoum, bromadiolone, bromethalin, calcium cyanide, chloralose, chlorophacinone, cholecalciferol, coumachlor, coumafuryl, coumatetralyl, crimidine, difenacoum, difethialone, diphacinone, ergocalciferol, flocoumafen, fluoroacetamide, flupropadine, flupropadine hydrochloride, hydrogen cyanide, iodomethane, lindane, magnesium phosphide, methyl bromide, norbormide, phosacetim, phosphine, phosphorus, pindone, potassium arsenite, pyrinuron, scilliroside, sodium arsenite, sodium cyanide, sodium fluoroacetate, strychnine, thallium sulfate, warfarin and zinc phosphide.

In the liquid form, for example, solutions or suspensions, bacterial populations can be mixed or suspended in suitable liquid carriers. Suitable liquid diluents or carriers include water, buffered solvents, oils, petroleum distillates, or other liquid carriers.

Solid compositions can be prepared by dispersing the bacterial populations in and on an appropriately divided solid carrier, such as peat, wheat, bran, vermiculite, clay, talc, bentonite, diatomaceous earth, fuller's earth, pasteurized soil, and the like. When such formulations are used as wettable powders, biologically compatible dispersing agents such as non-ionic, anionic, amphoteric, or cationic dispersing and emulsifying agents can be used.

The solid carriers used upon formulation include, for example, mineral carriers such as kaolin clay, pyrophyllite, bentonite, montmorillonite, diatomaceous earth, acid white soil, vermiculite, and pearlite, and inorganic salts such as ammonium sulfate, ammonium phosphate, ammonium nitrate, urea, ammonium chloride, and calcium carbonate. Also, organic fine powders such as wheat flour, wheat bran, and rice bran may be used. The liquid carriers include vegetable oils such as soybean oil and cottonseed oil, glycerol, ethylene glycol, polyethylene glycol, propylene glycol, polypropylene glycol, etc.

The agricultural composition can be fabricated in bioreactors such as continuous stirred tank reactors, batch reactors, and on the farm. In some embodiments, compositions can be stored in a container, such as a jug or in mini bulk. In some embodiments, compositions are stored within an object selected from the group consisting of a bottle, jar, ampule, package, vessel, bag, box, bin, envelope, carton, container, silo, shipping container, truck bed, and/or case.

Additional agricultural composition components for inclusion in the compositions disclosed herein may be found in International Patent Application No. PCT/US2019/039217, the contents of which are herein incorporated by reference in their entirety.

### Methods of Formulating Agronomically Stable Liquid Agricultural Compositions

As referenced in the foregoing sections, the present disclosure provides methods of formulating the disclosed agronomically stable liquid agricultural compositions, e.g., methods of improving the stability of liquid agricultural compositions comprising nitrogen-fixing microorganisms. These methods comprise the steps of varying and/or optimizing the agricultural composition along different parameters. In some embodiments, these parameters include the selection of an initial cell density, the choice of microbial stabilizer, the choice of physical stabilizer, and the choice of buffering agent, each of which components is described in detail in its respective section.

In some embodiments, the selection of the initial cell density with an acceptable decay rate, the selection of the buffering agent, the selection of the microbial stabilizer, and the selection of the physical stabilizer are performed in any order. In some embodiments, these selection steps are performed in parallel. In some embodiments, these selection steps are performed serially. In some embodiments, the selection of the initial cell density with an acceptable decay rate is performed first. Selection of the initial cell density is described in detail in the present application disclosure.

In some embodiments, the selections of the buffering agent, microbial stabilizer, and physical stabilizer are performed in tandem through screening assays comprising different combinations. In some embodiments, the selections of any two of the buffering agent, microbial stabilizer, and physical stabilizer are performed in tandem. In some embodiments, when tested in tandem, the method comprises selecting combinations that have an additive or synergistic effect on microbial stability. In some embodiments, the selections of the buffering agent, microbial stabilizer, and physical stabilizer are performed separately through screening assays varying each parameter individually. In some embodiments, the method comprises varying each parameter by assaying two or more possible components of each type. In some embodiments, the method comprises varying each parameter by assaying two or more concentrations of each component.

In some embodiments, the method comprises comparing possible components and/or concentrations of components against each other in one or more screening assays. In some embodiments, the agricultural composition is screened for microbial viability. In some embodiments, microbial viability is measured in CFU/mL via a standard plating assay. In some embodiments, the agricultural composition is screened for colonization potential. In some embodiments, the agricultural composition is screened for colonization potential in log 10 copies per gram of fresh weight via a root colonization assay, as describe in Example 1. In some embodiments, the agricultural composition is screened for toxin accumulation. In some embodiments, the composition is screened for toxin concentrations at a given time point, e.g., the target shelf life time point. In some embodiments, the results of any one or more screening assays are used to select the buffering agent, microbial stabilizer, and physical stabilizer for inclusion in the agricultural composition.

In terms of selection of the buffering agent, in some embodiments, a screening assay compares different buffering agents, different pH levels of buffering agents, different buffering capacities of a given buffering agent, and/or different molarities of buffering agents. In terms of selection of the microbial stabilizer, in some embodiments, a screening assay compares different microbial stabilizers and/or different concentrations of a given microbial stabilizer. In terms of selection of the physical stabilizer, in some embodiments, a screening assay compares different physical stabilizers and/or different concentrations of a given physical stabilizer. In some embodiments, the physical stabilizers and microbial stabilizers are assayed in tandem.

In some embodiments, the method improves the shelf life of the agricultural composition at least 2-fold, at least 3-fold, or at least 4-fold. In some embodiments, the method produces a liquid agricultural composition with a shelf life of at least 30 days, at least 2 months, at least 3 months, at least 4 months, at least 5 months, or at least 6 months.

### Methods of Application

The present disclosure provides methods for improving one or more aspects of agricultural plant characteristics through the application of the disclosed agronomically stable agricultural compositions to agricultural plant tissues or the environs thereof. More generally, provided herein are methods of applying the disclosed agricultural compositions to agricultural plant tissues or the environs thereof. Such application may improve one or more characteristics of the agricultural plant. In some embodiments, the methods are used for improving the health, yield, yield variance, stress resistance, growth, or agronomic characteristics of a plant, the methods comprising contacting the plant tissues or the environs thereof before or during planting with the disclosed agronomically stable liquid agricultural compositions.

In some embodiments, provided are methods for providing a diazotrophic bacterium to agricultural plant tissues or the environs thereof, the method comprising formulating an agricultural composition according to the present disclosure and applying said agricultural composition to said agricultural plant tissues or the environs thereof. In some embodiments, the agricultural composition is applied in-furrow. In some embodiments, the agricultural composition is applied as a seed coat. In some embodiments, the present methods are used to increase agricultural plant crop yield and/or decrease agricultural plant crop yield variance.

In some embodiments, the methods comprise applying one or more of the disclosed agricultural compositions to agricultural plant tissues or the environs thereof. In some embodiments, one or more compositions are applied to the surface of a seed, seedling, plant, plant part, or the environs thereof. In some embodiments, one or more compositions are applied as a seed coat on a seed. In some embodiments, one or more compositions are applied as a layer above a surface of a seed, seedling, plant, plant part, or the environs thereof. In some embodiments, one or more compositions is applied to a seed, seedling, plant, plant part, or the environs thereof by spraying, immersing, coating, misting, sprinkling, rolling and/or encapsulating the seed, seedling, plant, plant part, or the environs thereof with the one or more compositions. Similarly, applying the composition to the seed, seedling, plant, plant part, or the environs thereof comprises any means of application, including dipping, rolling, spraying, shaking, immersing, flowing, misting, painting, brushing, and washing. As used herein, the term “applying” or “application” refers to placing or distributing an agricultural composition of the present disclosure onto an area, volume, or quantity of agricultural plant tissues or the environs thereof. For example, application may be accomplished by hand broadcast, machine spreading, brushing, spraying, machine broadcasting, irrigating, top dressing vehicle, and the like, onto agricultural plant tissues or the environs thereof.

In some embodiments, the methods provide an effective amount of a disclosed composition to plant tissues or the environs thereof. In general, an effective amount is an amount sufficient to result in plants with improved traits (e.g. a desired level of nitrogen fixation). An effective amount of the agricultural composition can be used to populate the sub-soil region around seeds, seedlings, plants, or plant parts with viable bacterial growth, or populate the seeds, seedlings, plants, or plant parts with viable bacterial growth.

In some embodiments, the present methods result in a higher concentration of microbes surviving through storage, delivery, and/or transport until planting.

In some embodiments, the agricultural compositions disclosed herein may be applied to any plant part or the environs thereof. Non-limiting examples of plant tissues include a seed, seedling, leaf, cutting, plant, bulb, tuber, root, and rhizomes. In some embodiments, agricultural compositions are applied to a seed. In some embodiments, agricultural compositions are applied to a seedling. In some embodiments, agricultural compositions are applied to plant roots. In some embodiments, agricultural compositions are applied in-furrow.

### Concentrations and Rates of Application of Agricultural Compositions

As aforementioned, the agricultural compositions of the present disclosure, which comprise a nitrogen-fixing microorganism, can be applied as an agricultural composition to a seed, seedling, plant, plant part, or the environs thereof.

The microbes of the disclosure can be present on the seed, seedling, plant, plant part, or the environs thereof in a variety of concentrations. In some embodiments, the agricultural composition is applied as a seed treatment in a variety of concentrations. For example, the microbes can be found in a seed treatment at a CFU concentration per seed of: 1×10¹, 1×10², 1×10³, 1×10⁴, 1×10⁵, 1×10⁶, 1×10⁷, 1×10⁸, 1×10⁹, 1×10¹⁰, or more. In particular aspects, the seed treatment compositions comprise about 1×10⁴ to about 1×10⁸ CFU per seed. In other particular aspects, the seed treatment compositions comprise about 1×10⁵ to about 1×10⁷ CFU per seed. In other aspects, the seed treatment compositions comprise about 1×10⁶ CFU per seed.

In the United States, about 10% of corn acreage is planted at a seed density of above about 36,000 seeds per acre; ⅓ of the corn acreage is planted at a seed density of between about 33,000 to 36,000 seeds per acre; ⅓ of the corn acreage is planted at a seed density of between about 30,000 to 33,000 seeds per acre, and the remainder of the acreage is variable. See, “Corn Seeding Rate Considerations,” written by Steve Butzen, available at: www.pioneer.com/home/site/us/agronomy/library/corn-seeding-rate-considerations/

Table 3 below utilizes various CFU concentrations per seed in a contemplated seed treatment embodiment (rows across) and various seed acreage planting densities (1^{st} column: 15K-41K) to calculate the total amount of CFU per acre, which would be utilized in various agricultural scenarios (i.e. seed treatment concentration per seed×seed density planted per acre). Thus, if one were to utilize a seed treatment with 1×10⁶ CFU per seed and plant 30,000 seeds per acre, then the total CFU content per acre would be 3×10¹⁰ (i.e. 30K*1×10⁶).

TABLE 3
Total CFU Per Acre Calculation for Seed Treatment Embodiments

| Corn Population | | | | | | | | |
|---|---|---|---|---|---|---|---|---|
| (i.e. seeds per | | | | | | | | |
| acre) | 1.00E+02 | 1.00E+03 | 1.00E+04 | 1.00E+05 | 1.00E+06 | 1.00E+07 | 1.00E+08 | 1.00E+09 |
| 15,000 | 1.50E+06 | 1.50E+07 | 1.50E+08 | 1.50E+09 | 1.50E+10 | 1.50E+11 | 1.50E+12 | 1.50E+13 |
| 16,000 | 1.60E+06 | 1.60E+07 | 1.60E+08 | 1.60E+09 | 1.60E+10 | 1.60E+11 | 1.60E+12 | 1.60E+13 |
| 17,000 | 1.70E+06 | 1.70E+07 | 1.70E+08 | 1.70E+09 | 1.70E+10 | 1.70E+11 | 1.70E+12 | 1.70E+13 |
| 18,000 | 1.80E+06 | 1.80E+07 | 1.80E+08 | 1.80E+09 | 1.80E+10 | 1.80E+11 | 1.80E+12 | 1.80E+13 |
| 19,000 | 1.90E+06 | 1.90E+07 | 1.90E+08 | 1.90E+09 | 1.90E+10 | 1.90E+11 | 1.90E+12 | 1.90E+13 |
| 20,000 | 2.00E+06 | 2.00E+07 | 2.00E+08 | 2.00E+09 | 2.00E+10 | 2.00E+11 | 2.00E+12 | 2.00E+13 |
| 21,000 | 2.10E+06 | 2.10E+07 | 2.10E+08 | 2.10E+09 | 2.10E+10 | 2.10E+11 | 2.10E+12 | 2.10E+13 |
| 22,000 | 2.20E+06 | 2.20E+07 | 2.20E+08 | 2.20E+09 | 2.20E+10 | 2.20E+11 | 2.20E+12 | 2.20E+13 |
| 23,000 | 2.30E+06 | 2.30E+07 | 2.30E+08 | 2.30E+09 | 2.30E+10 | 2.30E+11 | 2.30E+12 | 2.30E+13 |
| 24,000 | 2.40E+06 | 2.40E+07 | 2.40E+08 | 2.40E+09 | 2.40E+10 | 2.40E+11 | 2.40E+12 | 2.40E+13 |
| 25,000 | 2.50E+06 | 2.50E+07 | 2.50E+08 | 2.50E+09 | 2.50E+10 | 2.50E+11 | 2.50E+12 | 2.50E+13 |
| 26,000 | 2.60E+06 | 2.60E+07 | 2.60E+08 | 2.60E+09 | 2.60E+10 | 2.60E+11 | 2.60E+12 | 2.60E+13 |
| 27,000 | 2.70E+06 | 2.70E+07 | 2.70E+08 | 2.70E+09 | 2.70E+10 | 2.70E+11 | 2.70E+12 | 2.70E+13 |
| 28,000 | 2.80E+06 | 2.80E+07 | 2.80E+08 | 2.80E+09 | 2.80E+10 | 2.80E+11 | 2.80E+12 | 2.80E+13 |
| 29,000 | 2.90E+06 | 2.90E+07 | 2.90E+08 | 2.90E+09 | 2.90E+10 | 2.90E+11 | 2.90E+12 | 2.90E+13 |
| 30,000 | 3.00E+06 | 3.00E+07 | 3.00E+08 | 3.00E+09 | 3.00E+10 | 3.00E+11 | 3.00E+12 | 3.00E+13 |
| 31,000 | 3.10E+06 | 3.10E+07 | 3.10E+08 | 3.10E+09 | 3.10E+10 | 3.10E+11 | 3.10E+12 | 3.10E+13 |
| 32,000 | 3.20E+06 | 3.20E+07 | 3.20E+08 | 3.20E+09 | 3.20E+10 | 3.20E+11 | 3.20E+12 | 3.20E+13 |
| 33,000 | 3.30E+06 | 3.30E+07 | 3.30E+08 | 3.30E+09 | 3.30E+10 | 3.30E+11 | 3.30E+12 | 3.30E+13 |
| 34,000 | 3.40E+06 | 3.40E+07 | 3.40E+08 | 3.40E+09 | 3.40E+10 | 3.40E+11 | 3.40E+12 | 3.40E+13 |
| 35,000 | 3.50E+06 | 3.50E+07 | 3.50E+08 | 3.50E+09 | 3.50E+10 | 3.50E+11 | 3.50E+12 | 3.50E+13 |
| 36,000 | 3.60E+06 | 3.60E+07 | 3.60E+08 | 3.60E+09 | 3.60E+10 | 3.60E+11 | 3.60E+12 | 3.60E+13 |
| 37,000 | 3.70E+06 | 3.70E+07 | 3.70E+08 | 3.70E+09 | 3.70E+10 | 3.70E+11 | 3.70E+12 | 3.70E+13 |
| 38,000 | 3.80E+06 | 3.80E+07 | 3.80E+08 | 3.80E+09 | 3.80E+10 | 3.80E+11 | 3.80E+12 | 3.80E+13 |
| 39,000 | 3.90E+06 | 3.90E+07 | 3.90E+08 | 3.90E+09 | 3.90E+10 | 3.90E+11 | 3.90E+12 | 3.90E+13 |
| 40,000 | 4.00E+06 | 4.00E+07 | 4.00E+08 | 4.00E+09 | 4.00E+10 | 4.00E+11 | 4.00E+12 | 4.00E+13 |
| 41,000 | 4.10E+06 | 4.10E+07 | 4.10E+08 | 4.10E+09 | 4.10E+10 | 4.10E+11 | 4.10E+12 | 4.10E+13 |

For in-furrow embodiments, in some embodiments, the microbes of the disclosure are applied at a CFU concentration per acre of about 1E9-1E13 CFU/acre. In some embodiments, the microbes of the disclosure are applied at a CFU concentration per acre of about: 3E9, 1.5E10, 3E10, 1.5E11, 3E11, 8E11, 1.5E12, 3E12, or more. In some embodiments, the liquid in-furrow compositions are applied at a concentration of between about 3E9 to about 3E12 CFU per acre.

In some aspects, the in-furrow compositions are contained in a liquid agricultural composition. In the liquid in-furrow embodiments, the microbes can be present at a CFU concentration per milliliter of: 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, or more. In certain aspects, the liquid in-furrow compositions comprise microbes at a concentration of about 1×10⁶ to about 1×10¹¹ CFU per milliliter. In other aspects, the liquid in-furrow compositions comprise microbes at a concentration of about 1×10⁷ to about 1×10¹⁰ CFU per milliliter. In other aspects, the liquid in-furrow compositions comprise microbes at a concentration of about 1×10⁸ to about 1×10⁹ CFU per milliliter. In other aspects, the liquid in-furrow compositions comprise microbes at a concentration of up to about 1×10¹³ CFU per milliliter.

### Improvement of Plant Traits

Methods and agricultural compositions of the present disclosure may be employed to introduce or improve one or more of a variety of desirable traits in a plant through application of the disclosed compositions to a seed, seedling, plant, plant part, or the environs thereof prior to or during planting. Examples of traits that may be introduced or improved include: root biomass, root length, height, shoot length, leaf number, water use efficiency, overall biomass, yield, fruit size, grain size, photosynthesis rate, tolerance to drought, heat tolerance, salt tolerance, resistance to nematode stress, resistance to a fungal pathogen, resistance to a bacterial pathogen, resistance to a viral pathogen, level of a metabolite, and proteome expression. The desirable traits, including height, overall biomass, root and/or shoot biomass, seed germination, seedling survival, photosynthetic efficiency, transpiration rate, seed/fruit number or mass, plant grain or fruit yield, leaf chlorophyll content, photosynthetic rate, root length, or any combination thereof, can be used to measure growth, and compared with the growth rate of reference agricultural plants (e.g., plants without the improved traits) grown under identical conditions. In some embodiments, the methods and agricultural compositions described herein can improve plant traits, such as promoting plant growth, maintaining high chlorophyll content in leaves, increasing fruit or seed numbers, and increasing fruit or seed unit weight. In some embodiments, the plant has improved health, yield, stress resistance, growth, or agronomic characteristics relative to a control plant.

A preferred trait to be introduced or improved is nitrogen fixation, as described herein. A second preferred trait to be introduced or improved is colonization potential, as described herein. In some embodiments, a plant resulting from the methods described herein exhibits a difference in the trait that is at least about 5% greater, for example at least about 5%, at least about 8%, at least about 10%, at least about 15%, at least about 20%, at least about 25%, at least about 30%, at least about 40%, at least about 50%, at least about 60%, at least about 75%, at least about 80%, at least about 80%, at least about 90%, or at least 100%, at least about 200%, at least about 300%, at least about 400% or greater than a reference agricultural plant grown under the same conditions in the soil. In additional examples, a plant resulting from the methods described herein exhibits a difference in the trait that is at least about 5% greater, for example at least about 5%, at least about 8%, at least about 10%, at least about 15%, at least about 20%, at least about 25%, at least about 30%, at least about 40%, at least about 50%, at least about 60%, at least about 75%, at least about 80%, at least about 80%, at least about 90%, or at least 100%, at least about 200%, at least about 300%, at least about 400% or greater than a reference agricultural plant grown under similar conditions in the soil.

The trait to be improved may be assessed under conditions including the application of one or more biotic or abiotic stressors. Examples of stressors include abiotic stresses (such as heat stress, salt stress, drought stress, cold stress, and low nutrient stress) and biotic stresses (such as nematode stress, insect herbivory stress, fungal pathogen stress, bacterial pathogen stress, and viral pathogen stress).

The trait improved by methods and agricultural compositions of the present disclosure may be nitrogen fixation, including in a plant not previously capable of nitrogen fixation. In some embodiments, bacteria isolated according to a method described herein produce 1% or more (e.g. 2%, 3%, 4%, 5%, 6%, 7%, 8%, 9%, 10%, 15%, 20%, or more) of a plant's nitrogen, which may represent an increase in nitrogen fixation capability of at least 2-fold (e.g. 3-fold, 4-fold, 5-fold, 6-fold, 7-fold, 8-fold, 9-fold, 10-fold, 20-fold, 50-fold, 100-fold, 1000-fold, or more) as compared to bacteria isolated from the first plant before introducing any genetic variation. In some embodiments, the bacteria produce 5% or more of a plant's nitrogen. The desired level of nitrogen fixation may be achieved after repeating the steps of introducing genetic variation, exposure to a plurality of plants, and isolating bacteria from plants with an improved trait one or more times (e.g. 1, 2, 3, 4, 5, 10, 15, 25, or more times). In some embodiments, enhanced levels of nitrogen fixation are achieved in the presence of fertilizer supplemented with glutamine, ammonia, or other chemical source of nitrogen. Methods for assessing degree of nitrogen fixation are known and may be employed to assess the methods described herein.

### Agricultural Plant Tissue Compositions

In one aspect, the present disclosure provides agricultural plant tissues, e.g., seeds, comprising the disclosed compositions. In some embodiments, the plant tissues have been subject to a method of application disclosed herein. The methods and agricultural compositions described herein are suitable for plant tissues from any of a variety of plants, such as plants in the genera Hordeum, Oryza, Zea, and Triticeae. Other non-limiting examples of suitable plants include mosses, lichens, and algae. In some embodiments, the plants have economic, social and/or environmental value, such as food crops, fiber crops, oil crops, plants in the forestry or pulp and paper industries, feedstock for biofuel production and/or ornamental plants. In some embodiments, plants are used to produce economically valuable products such as a grain, a flour, a starch, a syrup, a meal, an oil, a film, a packaging, a nutraceutical product, a pulp, an animal feed, a fish fodder, a bulk material for industrial chemicals, a cereal product, a processed human-food product, a sugar, an alcohol, and/or a protein. Non-limiting examples of crop plants include maize, rice, wheat, barley, sorghum, millet, oats, rye triticale, buckwheat, sweet corn, sugar cane, onions, tomatoes, strawberries, asparagus, canola, soybean, potato, vegetables, cereals, and oilseeds. Plant tissues as provided herein can be genetically modified organisms (GMO), non-GMO, organic, or conventional. In some embodiments, the methods and agricultural compositions described herein are suitable for plant tissues from any of a variety of transgenic plants, non-transgenic plants, and hybrid plants thereof.

In some embodiments, plant tissues that are treated using the methods and agricultural composition disclosed herein include plant tissues from plants that are important or interesting for agriculture, horticulture, biomass for the production of biofuel molecules and other chemicals, and/or forestry. Some examples of these plants may include pineapple, banana, coconut, lily, grasspeas and grass; and dicotyledonous plants, such as, for example, peas, alfalfa, tomatillo, melon, chickpea, chicory, clover, kale, lentil, soybean, tobacco, potato, sweet potato, radish, cabbage, rape, apple trees, grape, cotton, sunflower, thale cress, canola, citrus (including orange, mandarin, kumquat, lemon, lime, grapefruit, tangerine, tangelo, citron, and pomelo), pepper, bean, lettuce, Panicum virgatum (switch), Sorghum bicolor (sorghum, sudan), Miscanthus giganteus (miscanthus), Saccharum sp. (energycane), Populus balsamifera (poplar), Zea mays (corn), Glycine max (soybean), Brassica napus (canola), Triticum aestivum (wheat), Gossypium hirsutum (cotton), Oryza sativa (rice), Helianthus annuus (sunflower), Medicago sativa (alfalfa), Beta vulgaris (sugarbeet), Pennisetum glaucum (pearl millet), Panicum spp. Sorghum spp., Miscanthus spp., Saccharum spp., Erianthus spp., Populus spp., Secale cereale (rye), Salix spp. (willow), Eucalyptus spp. (eucalyptus), Triticosecale spp. (triticum-25 wheat X rye), Bamboo, Carthamus tinctorius (safflower), Jatropha curcas (Jatropha), Ricinus communis (castor), Elaeis guineensis (oil palm), Phoenix dactylifera (date palm), Archontophoenix cunninghamiana (king palm), Syagrus romanzoffiana (queen palm), Linum usitatissimum (flax), Brassica juncea, Manihot esculenta (cassaya), Lycopersicon esculentum (tomato), Lactuca saliva (lettuce), Musa paradisiaca (banana), Solanum tuberosum (potato), Brassica oleracea (broccoli, cauliflower, brussel sprouts), Camellia sinensis (tea), Fragaria ananassa (strawberry), Theobroma cacao (cocoa), Coffea arabica (coffee), Vitis vinifera (grape), Ananas comosus (pineapple), Capsicum annum (hot & sweet pepper), Allium cepa (onion), Cucumis melo (melon), Cucumis sativus (cucumber), Cucurbita maxima (squash), Cucurbita moschata (squash), Spinacea oleracea (spinach), Citrullus lanatus (watermelon), Abelmoschus esculentus (okra), Solanum melongena (eggplant), Papaver somniferum (opium poppy), Papaver orientale, Taxus baccata, Taxus brevifolia, Artemisia annua, Cannabis saliva, Camptotheca acuminate, Catharanthus roseus, Vinca rosea, Cinchona officinalis, Coichicum autumnale, Veratrum californica, Digitalis lanata, Digitalis purpurea, Dioscorea 5 spp., Andrographis paniculata, Atropa belladonna, Datura stomonium, Berberis spp., Cephalotaxus spp., Ephedra sinica, Ephedra spp., Erythroxylum coca, Galanthus wornorii, Scopolia spp., Lycopodium serratum (Huperzia serrata), Lycopodium spp., Rauwolfia serpentina, Rauwolfia spp., Sanguinaria canadensis, Hyoscyamus spp., Calendula officinalis, Chrysanthemum parthenium, Coleus forskohlii, Tanacetum parthenium, Parthenium argentatum (guayule), Hevea spp. (rubber), Mentha spicata (mint), Mentha piperita (mint), Bixa orellana, Alstroemeria spp., Rosa spp. (rose), Dianthus caryophyllus (carnation), Petunia spp. (petunia), Poinsettia pulcherrima (poinsettia), Nicotiana tabacum (tobacco), Lupinus albus (lupin), Uniola paniculata (oats), Hordeum vulgare (barley), and Lolium spp. (rye).

In some embodiments, plant tissues or plant parts, e.g., seeds, from a monocotyledonous plant are treated. Monocotyledonous plants belong to the orders of the Alismatales, Arales, Arecales, Bromeliales, Commelinales, Cyclanthales, Cyperales, Eriocaulales, Hydrocharitales, Juncales, Lilliales, Najadales, Orchidales, Pandanales, Poales, Restionales, Triuridales, Typhales, and Zingiberales. Plants belonging to the class of the Gymnospermae are Cycadales, Ginkgoales, Gnetales, and Pinales. In some embodiments, the monocotyledonous plant can be selected from the group consisting of a maize, rice, wheat, barley, and sugarcane.

In some embodiments, plant tissues or plant parts, e.g., seeds, from a dicotyledonous plant are treated, including those belonging to the orders of the Aristochiales, Asterales, Batales, Campanulales, Capparales, Caryophyllales, Casuarinales, Celastrales, Cornales, Diapensales, Dilleniales, Dipsacales, Ebenales, Ericales, Eucomiales, Euphorbiales, Fabales, Fagales, Gentianales, Geraniales, Haloragales, Hamamelidales, Middles, Juglandales, Lamiales, Laurales, Lecythidales, Leitneriales, Magniolales, Malvales, Myricales, Myrtales, Nymphaeales, Papeverales, Piperales, Plantaginales, Plumb aginales, Podostemales, Polemoniales, Polygalales, Polygonales, Primulales, Proteales, Rafflesiales, Ranunculales, Rhamnales, Rosales, Rubiales, Salicales, Santales, Sapindales, Sarraceniaceae, Scrophulariales, Theales, Trochodendrales, Umbellales, Urticales, and Violates. In some embodiments, the dicotyledonous plant can be selected from the group consisting of cotton, soybean, pepper, and tomato.

In some embodiments, the plant to be improved is not readily amenable to experimental conditions. For example, a crop plant may take too long to grow enough to practically assess an improved trait serially over multiple iterations. Accordingly, a first plant from which bacteria are initially isolated, and/or the plurality of plants to which genetically manipulated bacteria are applied may be a model plant, such as a plant more amenable to evaluation under desired conditions. Non-limiting examples of model plants include Setaria, Brachypodium, and Arabidopsis. Ability of bacteria isolated according to a method of the disclosure using a model plant may then be applied to plant tissues, e.g., seeds, of a plant of another type (e.g. a crop plant) to confirm conferral of the improved trait.

Traits that may be improved by the methods and agricultural compositions disclosed herein include any observable characteristic of the seed or the plant resulting therefrom, including, for example, growth rate, height, weight, color, taste, smell, changes in the production of one or more compounds by the plant (including for example, metabolites, proteins, drugs, carbohydrates, oils, and any other compounds). Selecting agricultural plant tissues based on genotypic information is also envisaged (for example, including the pattern of plant gene expression in response to the bacteria, or identifying the presence of genetic markers, such as those associated with increased nitrogen fixation). Plants from which the plant tissues are obtained may also be selected based on the absence, suppression or inhibition of a certain feature or trait (such as an undesirable feature or trait) as opposed to the presence of a certain feature or trait (such as a desirable feature or trait). Additional plants and seeds acceptable for use within the methods and compositions of the present disclosure may be found in International Application Nos. PCT/US2019/039528 and PCT/US2019/039217, the contents of each of which are herein incorporated by reference in their entirety.

### Non-Genetically Modified Maize

The methods and agricultural compositions described herein are suitable for any of a variety of non-genetically modified maize plants or parts thereof. In some embodiments, the corn is organic. The methods and agricultural compositions described herein are suitable for plant tissues, e.g., seeds, or for in-furrow application, to any non-genetically modified hybrids, varieties, lineages, etc. In some embodiments, corn varieties generally fall under six categories: sweet corn, flint corn, popcorn, dent corn, pod corn, and flour corn.

### Genetically Modified Maize

The methods and agricultural compositions described herein are suitable for plant tissues, e.g., seeds, or for in-furrow application, of any of a hybrid, variety, lineage, etc. of genetically modified maize plants or part thereof.

Furthermore, the methods and agricultural compositions described herein are suitable for any of the following genetically modified maize events, which have been approved in one or more countries, or any new genetically modified corn event, which may include Bt traits, glufosinate resistance, glyphosate resistance, etc.: 32138 (32138 SPT Maintainer), 3272 (ENOGEN), 3272×Bt11, 3272×bt11×GA21, 3272×Bt11×MIR604, 3272×Bt11×MIR604×GA21, 3272×Bt11×MIR604×TC1507×5307×GA21, 3272×GA21, 3272×MIR604, 3272×MIR604×GA21, 4114, 5307 (AGRISURE Duracade), 5307×GA21, 5307×MIR604×Bt11×TC1507×GA21 (AGRISURE Duracade 5122), 5307×MIR604×Bt11×TC1507×GA21×MIR162 (AGRISURE Duracade 5222), 59122 (Herculex RW), 59122×DAS40278, 59122×GA21, 59122×MIR604, 59122×MIR604×GA21, 59122×MIR604×TC1507, 59122×MIR604×TC1507×GA21, 59122×MON810, 59122×MON810×MIR604, 59122×MON810×NK603, 59122×MON810×NK603×MIR604, 59122×MON88017, 59122×MON88017×DAS40278, 59122×NK603 (Herculex RW ROUNDUP READY 2), 59122×NK603×MIR604, 59122×TC1507×GA21, 676, 678, 680, 3751 IR, 98140, 98140×59122, 98140×TC1507, 98140×TC1507×59122, Bt10 (Bt10), Bt11 [X4334CBR, X4734CBR] (AGRISURE CB/LL), Bt11×5307, Bt11×5307×GA21, Bt11×59122×MIR604, Br11×59122×MIR604×GA21, Bt11×59122×MIR604×TC1507, M53, M56, DAS-59122-7, Bt11×59122×MIR604×TC1507×GA21, Bt11×59122×TC1507, TC1507×DAS-59122-7, Bt11×59122×TC1507×GA21, Bt11×GA21 (AGRISURE GT/CB/LL), Bt11×MIR162 (AGRISURE Viptera 2100), BT11×MIR162×5307, Bt11×MIR162×5307×GA21, Bt11×MIR162×GA21 (AGRISURE Viptera 3110), Bt11×MIR162×MIR604 (AGRISURE Viptera 3100), Bt11×MIR162×MIR604×5307, Bt11×MIR162×MIR604×5307×GA21, Bt11×MIR162×MIR604×GA21 (AGRISURE Viptera 3111/AGRISURE Viptera 4), Bt11, MIR162×MIR604×MON89034×5307×GA21, Bt11×MIR162×MIR604×TC1507, Bt11×MIR162×MIR604×TC1507×5307, Bt11×MIR162×MIR604×TC1507×GA21, Bt11×MIR162×MON89034, Bt11×MIR162×MON89034×GA21, Bt11×MIR162×TC1507, Bt11×MIR162×TC1507×5307, Bt11×MIR162×TC1507×5307×GA21, Bt11×MR162×TC1507×GA21 (AGRISURE Viptera 3220), BT11×MIR604 (Agrisure BC/LL/RW), Bt11×MIR604×5307, Bt11×MIR604×5307×GA21, Bt11×MIR604×GA21, Bt11×MIR604×TC1507, Bt11×MIR604×TC1507×5307, Bt11×MIR604×TC1507×GA21, Bt11×MON89034×GA21, Bt11×TC1507, Bt11×TC1507×5307, Bt11×TC1507×GA21, Bt176 (NaturGard KnockOut/Maximizer), BVLA430101, CBH-351 (STARLINK Maize), DAS40278 (ENLIST Maize), DAS40278×NK603, DBT418 (Bt Xtra Maize), DLL25 [B16], GA21 (ROUNDUP READY Maize/AGRISURE GT), GA21×MON810 (ROUNDUP READY Yieldgard Maize), GA21×T25, HCEM485, LY038 (MAVERA Maize), LY038×MON810 (MAVERA Yieldgard Maize), MIR162 (AGRISURE Viptera), MIR162×5307, MIR162×5307×GA21, MIR162×GA21, MIR162×MIR604, MIR162×MIR604×5307, MIR162×MIR604×5307×GA21, MIR162×MIR604×GA21, MIR162×MIR604×TC1507×5307, MIR162×MIR604×TC1507×5307×GA21, MIR162×MIR604×TC1507×GA21, MIR162×MON89034, MIR162×NK603, MIR162×TC1507, MIR162×TC1507×5307, MIR162×TC1507×5307×GA21, MIR162×TC1507×GA21, MIR604 (AGRISURE RW), MIR604×5307, MIR604×5307×GA21, MIR604×GA21 (AGRISURE GT/RW), MIR604×NK603, MIR604×TC1507, MIR604×TC1507×5307, MIR604×TC1507×5307×GA21, MIR604×TC1507×GA21, MON801 [MON80100], MON802, MON809, MON810 (YIELDGARD, MAIZEGARD), MON810×MIR162, MON810×MIR162×NK603, MON810×MIR604, MON810×MON88017 (YIELDGARD VT Triple), MON810×NK603×MIR604, MON832 (ROUNDUP READY Maize), MON863 (YIELDGARD Rootworm RW, MAXGARD), MON863×MON810 (YIELDGARD Plus), MON863×MON810×NK603 (YIELDGARD Plus with RR), MON863×NK603 (YIELDGARD RW+RR), MON87403, MON87411, MON87419, MON87427 (ROUNDUP READY Maize), MON87427×59122, MON87427×MON88017, MON87427×MON88017×59122, MON87427×MON89034, MON87427×MON89034×59122, MON87427×MON89034×MIR162×MON87411, MON87427×MON89034×MON88017, MON87427×MON89034×MON88017×59122, MON87427×MON89034×NK603, MON87427×MON89034×TC1507, MON87427×MON89034×TC1507×59122, MON87427×MON89034×TC1507×MON87411×59122, MON87427×MON89034×TC1507×MON87411×59122×DAS40278, MON87427×MON89034×TC1507×MON88017, MON87427×MON89034×MIR162×NK603, MON87427×MON89034×TC1507×MON88017×59122, MON87427×TC1507, MON87427×TC1507×59122, MON87427×TC1507×MON88017, MON87427×TC1507×MON88017×59122, MON87460 (GENUITY DROUGHTGARD), MON87460×MON88017, MON87460×MON89034×MON88017, MON87460×MON89034×NK603, MON87460×NK603, MON88017, MON88017×DAS40278, MON89034, MON89034×59122, MON89034×59122×DAS40278, MON89034×59122×MON88017, MON89034×59122×MON88017×DAS40278, MON89034×DAS40278, MON89034×MON87460, MON89034×MON88017 (GENUITY VT Triple Pro), MON89034×MON88017×DAS40278, MON89034×NK603 (GENUITY VT Double Pro), MON89034×NK603×DAS40278, MON89034×TC1507, MON89034×TC1507×59122, MON89034×TC1507×59122×DAS40278, MON89034×TC1507×DAS40278, MON89034×TC1507×MON88017, MON89034×TC1507×MON88017×59122 (GENUITY SMARTSTAX), MON89034×TC1507×MON88017×59122×DAS40278, MON89034×TC1507×MON88017×DAS40278, MON89034×TC1507×NK603 (POWER CORE), MON89034×TC1507×NK603×DAS40278, MON89034×TC1507×NK603×MIR162, MON89034×TC1507×NK603×MIR162×DAS40278, MON89034×GA21, MS3 (INVIGOR Maize), MS6 (INVIGOR Maize), MZHGOJG, MZIR098, NK603 (ROUNDUP READY 2 Maize), NK603×MON810×4114×MIR604, NK603×MON810 (YIELDGARD CB+RR), NK603×T25 (ROUNDUP READY LIBERTY LINK Maize), T14 (LIBERTY LINK Maize), T25 (LIBERTY LINK Maize), T25×MON810 (LIBERTY LINK YIELDGARD Maize), TC1507 (HERCULEX I, HERCULEX CB), TC1507×59122×MON810×MIR604×NK603 (OPTIMUM INTRASECT XTREME), TC1507×MON810×MIR604×NK603, TC1507×5307, TC1507×5307×GA21, TC1507×59122 (HERCULEX XTRA), TC1507×59122×DAS40278, TC1507×59122×MON810, TC1507×59122×MON810×MIR604, TC1507×59122×MON810×NK603 (OPTIMUM INTRASECT XTRA), TC1507×59122×MON88017, TC1507×59122×MON88017×DAS40278, TC1507×59122×NK603 (HERCULEX XTRA RR), TC1507×59122×NK603×MIR604, TC1507×DAS40278, TC1507×GA21, TC1507×MIR162×NK603, TC1507×MIR604×NK603 (OPTIMUM TRISECT), TC1507×MON810, TC1507×MON810×MIR162, TC1507×MON810×MIR162×NK603, TC1507×MON810×MIR604, TC1507×MON810×NK603 (OPTIMUM INTRASECT), TC1507×MON810×NK603×MIR604, TC1507×MON88017, TC1507×MON88017×DAS40278, TC1507×NK603 (HERCULEX I RR), TC1507×NK603×DAS40278, TC6275, and VCO-01981-5.

### Remodeled Microbes of the Disclosure

The present disclosure provides engineered microbes for use in the disclosed agricultural compositions and methods. In some embodiments, the microbes are non-intergeneric remodeled microbes. The term “non-intergeneric” indicates that the genetic variations introduced into the host do not contain nucleic acid sequences from outside the host genus (e.g., no transgenic DNA). Therefore, in some embodiments, the microbes are not transgenic. For example, for non-transgenic microbes with varied promoters, promoters for promoter swapping are selected from within the microbe's genome, or genus.

Exemplary non-intergeneric genetic variations include a mutation in the gene of interest that may improve the function of the protein encoded by the gene; a constitutionally active promoter that can replace the endogenous promoter of the gene of interest to increase the expression of the gene; a mutation that will inactivate the gene of interest; the insertion of a promoter from within the host's genome into a heterologous location, e.g. insertion of the promoter into a gene that results in inactivation of said gene and upregulation of a downstream gene; and the like. The mutations can be point mutations, insertions, and/or deletions (full or partial deletion of the gene). For example, in some embodiments, to improve the nitrogen fixation activity of the host microbe, a genetic variation may comprise an inactivating mutation of the nifL gene (negative regulator of nitrogen fixation pathway) and/or comprise replacing the endogenous promoter of the nifA and/or nifH gene (nitrogenase iron protein that catalyzes a key reaction to fix atmospheric nitrogen) with a constitutionally active promoter that will drive the expression of the nifA and/or nifH gene constitutively. Additional genetic variations of interest are described further in the foregoing “Genetic alterations” section.

Exemplary wild-type and modified sequences of microbes for use in the agricultural compositions and methods of the present disclosure are provided in Table 4.

TABLE 4
Wild-type and modified sequences for use in microbes of the disclosure

| SEQ ID | | |
|---|---|---|
| NO | Genotype | Description |
| SEQ ID | 16S | N/A |
| NO 1 | | |
| SEQ ID | nifH | N/A |
| NO 2 | | |
| SEQ ID | nifD1 | 1 of 2 unique genes annotated as nifD in 63 genome |
| NO 3 | | |
| SEQ ID | nifD2 | 2 of 2 unique genes annotated as nifD in 63 genome |
| NO 4 | | |
| SEQ ID | nifK1 | 1 of 2 unique genes annotated as nifK in 63 genome |
| NO 5 | | |
| SEQ ID | nifK2 | 2 of 2 unique genes annotated as nifK in 63 genome |
| NO 6 | | |
| SEQ ID | nifL | N/A |
| NO 7 | | |
| SEQ ID | nifA | N/A |
| NO 8 | | |
| SEQ ID | glnE | N/A |
| NO 9 | | |
| SEQ ID | amtB | N/A |
| NO 10 | | |
| SEQ ID | PinfC | 500 bp immediately upstream of the ATG start codon |
| NO 11 | | of the infC gene |
| SEQ ID | 16S | N/A |
| NO 12 | | |
| SEQ ID | nifH1 | 1 of 2 unique genes annotated as nifH in 137 genome |
| NO 13 | | |
| SEQ ID | nifH2 | 2 of 2 unique genes annotated as nifH in 137 genome |
| NO 14 | | |
| SEQ ID | nifD1 | 1 of 2 unique genes annotated as nifD in 137 genome |
| NO 15 | | |
| SEQ ID | nifD2 | 2 of 2 unique genes annotated as nifD in 137 genome |
| NO 16 | | |
| SEQ ID | nifK1 | 1 of 2 unique genes annotated as nifK in 137 genome |
| NO 17 | | |
| SEQ ID | nifK2 | 2 of 2 unique genes annotated as nifK in 137 genome |
| NO 18 | | |
| SEQ ID | nifL | N/A |
| NO 19 | | |
| SEQ ID | nifA | N/A |
| NO 20 | | |
| SEQ ID | glnE | N/A |
| NO 21 | | |
| SEQ ID | PinfC | 500 bp immediately upstream of the TTG start codon of |
| NO 22 | | infC |
| SEQ ID | amtB | N/A |
| NO 23 | | |
| SEQ ID | Prm8.2 | internal promoter located in nlpI gene; 299 bp starting |
| NO 24 | | at 81 bp after the A of the ATG of the nlpI gene |
| SEQ ID | Prm6.2 | 300 bp upstream of the secE gene starting at 57 bp |
| NO 25 | | upstream of the A of the ATG of secE |
| SEQ ID | Prm1.2 | 400 bp immediately upstream of the ATG of cspE gene |
| NO 26 | | |
| SEQ ID | 16S | N/A |
| NO 27 | | |
| SEQ ID | nifH | N/A |
| NO 28 | | |
| SEQ ID | nifD1 | 1 of 2 unique genes annotated as nifD in 728 genome |
| NO 29 | | |
| SEQ ID | nifD2 | 2 of 2 unique genes annotated as nifD in 728 genome |
| NO 30 | | |
| SEQ ID | nifK1 | 1 of 2 unique genes annotated as nifK in 728 genome |
| NO 31 | | |
| SEQ ID | nifK2 | 2 of 2 unique genes annotated as nifK in 728 genome |
| NO 32 | | |
| SEQ ID | nifL | N/A |
| NO 33 | | |
| SEQ ID | nifA | N/A |
| NO 34 | | |
| SEQ ID | glnE | N/A |
| NO 35 | | |
| SEQ ID | amtB | N/A |
| NO 36 | | |
| SEQ ID | 16S | N/A |
| NO 37 | | |
| SEQ ID | 16S | N/A |
| NO 38 | | |
| SEQ ID | 16S | N/A |
| NO 39 | | |
| SEQ ID | 16S | N/A |
| NO 40 | | |
| SEQ ID | nifH | N/A |
| NO 41 | | |
| SEQ ID | Dinitrogenase | N/A |
| NO 42 | iron- | |
| | molybdenum | |
| | cofactor CDS | |
| SEQ ID | nifD1 | N/A |
| NO 43 | | |
| SEQ ID | nifD2 | N/A |
| NO 44 | | |
| SEQ ID | nifK1 | N/A |
| NO 45 | | |
| SEQ ID | nifK2 | N/A |
| NO 46 | | |
| SEQ ID | nifL | N/A |
| NO 47 | | |
| SEQ ID | nifA | N/A |
| NO 48 | | |
| SEQ ID | glnE | N/A |
| NO 49 | | |
| SEQ ID | amtB | N/A |
| NO 50 | | |
| SEQ ID | PinfC | 498 bp immediately upstream of the ATG of the infC |
| NO 51 | | gene |
| SEQ ID | 16S | N/A |
| NO 52 | | |
| SEQ ID | nifH | N/A |
| NO 53 | | |
| SEQ ID | nifD1 | 1 of 2 unique genes annotated as nifD in 126585 |
| NO 54 | | genome |
| SEQ ID | nifD2 | 2 of 2 unique genes annotated as nifD in 126585 |
| NO 55 | | genome |
| SEQ ID | nifK1 | 1 of 2 unique genes annotated as nifK in 126585 |
| NO 56 | | genome |
| SEQ ID | nifK2 | 2 of 2 unique genes annotated as nifK in 126585 |
| NO 57 | | genome |
| SEQ ID | nifL | N/A |
| NO 58 | | |
| SEQ ID | nifA | N/A |
| NO 59 | | |
| SEQ ID | glnE | N/A |
| NO 60 | | |
| SEQ ID | amtB | N/A |
| NO 61 | | |
| SEQ ID | PinfC | 500 bp immediately upstream of the ATG start codon |
| NO 62 | | of the infC gene |
| SEQ ID | Prm1 | 348 bp includes the 319 bp immediately upstream of the |
| NO 63 | | ATG start codon of the lpp gene and the first 29 bp of |
| | | the lpp gene |
| SEQ ID | Prm7 | 339 bp upstream of the sspA gene, ending at 46 bp |
| NO 64 | | upstream of the ATG of the sspA gene |
| SEQ ID | 16S | N/A |
| NO 65 | | |
| SEQ ID | nifH | N/A |
| NO 66 | | |
| SEQ ID | nifD1 | 1 of 2 unique genes annotated as nifD in 1113 genome |
| NO 67 | | |
| SEQ ID | nifD2 | 2 of 2 unique genes annotated as nifD in 1113 genome |
| NO 68 | | |
| SEQ ID | nifK | N/A |
| NO 69 | | |
| SEQ ID | nifL | N/A |
| NO 70 | | |
| SEQ ID | nifA partial gene | due to a gap in the sequence assembly, we can only |
| NO 71 | | identify a partial gene from the 1113 genome |
| SEQ ID | glnE | N/A |
| NO 72 | | |
| SEQ ID | 16S | |
| NO 73 | | |
| SEQ ID | nifH | |
| NO 74 | | |
| SEQ ID | nifD1 | 1 of 2 unique genes annotated as nifD in 1116 genome |
| NO 75 | | |
| SEQ ID | nifD2 | 2 of 2 unique genes annotated as nifD in 1116 genome |
| NO 76 | | |
| SEQ ID | nifK1 | 1 of 2 unique genes annotated as nifK in 1116 genome |
| NO 77 | | |
| SEQ ID | nifK2 | 2 of 2 unique genes annotated as nifK in 1116 genome |
| NO 78 | | |
| SEQ ID | nifL | N/A |
| NO 79 | | |
| SEQ ID | nifA | N/A |
| NO 80 | | |
| SEQ ID | glnE | N/A |
| NO 81 | | |
| SEQ ID | amtB | N/A |
| NO 82 | | |
| SEQ ID | 16S | N/A |
| NO 83 | | |
| SEQ ID | nifH | N/A |
| NO 84 | | |
| SEQ ID | nifD1 | 1 of 2 unique genes annotated as nifD in 1293 genome |
| NO 85 | | |
| SEQ ID | nifD2 | 2 of 2 unique genes annotated as nifD in 1293 genome |
| NO 86 | | |
| SEQ ID | nifK | 1 of 2 unique genes annotated as nifK in 1293 genome |
| NO 87 | | |
| SEQ ID | nifK1 | 2 of 2 unique genes annotated as nifK in 1293 genome |
| NO 88 | | |
| SEQ ID | nifA | N/A |
| NO 89 | | |
| SEQ ID | glnE | N/A |
| NO 90 | | |
| SEQ ID | amtB1 | 1 of 2 unique genes annotated as amtB in 1293 genome |
| NO 91 | | |
| SEQ ID | amtB2 | 2 of 2 unique genes annotated as amtB in 1293 genome |
| NO 92 | | |
| SEQ ID | ΔnifL::PinfC | starting at 24 bp after the A of the ATG start codon, |
| NO 93 | | 1375 bp of nifL have been deleted and replaced with |
| | | the 1021 PinfC promoter sequence |
| SEQ ID | ΔnifL::PinfC | starting at 24 bp after the A of the ATG start codon, |
| NO 94 | with 500 bp flank | 1375 bp of nifL have been deleted and replaced with |
| | | the 1021 PinfC promoter sequence; 500 bp flanking the |
| | | nifL gene upstream and downstream are included |
| SEQ ID | glnEΔAR-2 | glnE gene with 1673 bp immediately downstream of the |
| NO 95 | | ATG start codon deleted, resulting in a truncated glnE |
| | | protein lacking the adenylyl-removing (AR) domain |
| SEQ ID | glnEAAR-2 with | glnE gene with 1673 bp immediately downstream of the |
| NO 96 | 500 bp flank | ATG start codon deleted, resulting in a truncated glnE |
| | | protein lacking the adenylyl-removing (AR) domain; |
| | | 500 bp flanking the glnE gene upstream and |
| | | downstream are included |
| SEQ ID | ΔnifL::Prm1 | starting at 24 bp after the A of the ATG start codon, |
| NO 97 | | 1375 bp of nifL have been deleted and replaced with |
| | | the 1021 Prm1 promoter sequence |
| SEQ ID | ΔnifL::Prm1 | starting at 24 bp after the A of the ATG start codon, |
| NO 98 | with 500 bp flank | 1375 bp of nifL have been deleted and replaced with |
| | | the 1021 rml promoter sequence; 500 bp flanking the |
| | | nifL gene upstream and downstream are included |
| SEQ ID | glnEΔAR-2 | glnE gene with 1673 bp immediately downstream of the |
| NO 99 | | ATG start codon deleted, resulting in a truncated glnE |
| | | protein lacking the adenylyl-removing (AR) domain |
| SEQ ID | glnEΔAR-2 with | glnE gene with 1673 bp immediately downstream of the |
| NO 100 | 500 bp flank | ATG start codon deleted, resulting in a truncated glnE |
| | | protein lacking the adenylyl-removing (AR) domain; |
| | | 500 bp flanking the glnE gene upstream and |
| | | downstream are included |
| SEQ ID | ΔnifL::Prm1 | starting at 24 bp after the A of the ATG start codon, |
| NO 101 | | 1375 bp of nifL have been deleted and replaced with |
| | | the 1021 Prm1 promoter sequence |
| SEQ ID | ΔnifL::Prm1 | starting at 24 bp after the A of the ATG start codon, |
| NO 102 | with 500 bp flank | 1375 bp of nifL have been deleted and replaced with |
| | | the 1021 rm1 promoter sequence; 500 bp flanking the |
| | | nifL gene upstream and downstream are included |
| SEQ ID | glnEΔAR-2 | glnE gene with 1673 bp immediately downstream of the |
| NO 103 | | ATG start codon deleted, resulting in a truncated glnE |
| | | protein lacking the adenylyl-removing (AR) domain |
| SEQ ID | glnEΔAR-2 with | glnE gene with 1673 bp immediately downstream of the |
| NO 104 | 500 bp flank | ATG start codon deleted, resulting in a truncated glnE |
| | | protein lacking the adenylyl-removing (AR) domain; |
| | | 500 bp flanking the glnE gene upstream and |
| | | downstream are included |
| SEQ ID | ΔnifL::Prm7 | starting at 24 bp after the A of the ATG start codon, |
| NO 105 | | 1375 bp of nifL have been deleted and replaced with |
| | | the 1021 Prm7 promoter sequence |
| SEQ ID | ΔnifL::Prm7 | starting at 24 bp after the A of the ATG start codon, |
| NO 106 | with 500 bp flank | 1375 bp of nifL have been deleted and replaced with |
| | | the 1021 rm7 promoter sequence; 500 bp flanking the |
| | | nifL gene upstream and downstream are included |
| SEQ ID | glnEΔAR-2 | glnE gene with 1290 bp immediately downstream of the |
| NO 107 | | ATG start codon deleted, resulting in a truncated glnE |
| | | protein lacking the adenylyl-removing (AR) domain |
| SEQ ID | glnEΔAR-2 with | glnE gene with 1290 bp immediately downstream of the |
| NO 108 | 500 bp flank | ATG start codon deleted, resulting in a truncated glnE |
| | | protein lacking the adenylyl-removing (AR) domain; |
| | | 500 bp flanking the glnE gene upstream and |
| | | downstream are included |
| SEQ ID | ΔnifL::PinfC | starting at 24 bp after the A of the ATG start codon, |
| NO 109 | | 1372 bp of nifL have been deleted and replaced with |
| | | the 137 PinfC promoter sequence |
| SEQ ID | ΔnifL::PinfC | starting at 24 bp after the A of the ATG start codon, |
| NO 110 | with 500 bp flank | 1372 bp of nifL have been deleted and replaced with |
| | | the 137 PinfC promoter sequence; 500 bp flanking the |
| | | nifL gene upstream and downstream are included |
| SEQ ID | glnEAAR-2 | glnE gene with 1290 bp immediately downstream of the |
| NO 111 | 36 bp deletion | ATG start codon deleted AND 36 bp deleted beginning |
| | | at 1472 bp downstream of the start codon, resulting in a |
| | | truncated glnE protein lacking the adenylyl-removing |
| | | (AR) domain |
| SEQ ID | glnEAAR-2 | glnE gene with 1290 bp immediately downstream of the |
| NO 112 | 36 bp deletion | ATG start codon deleted AND 36 bp deleted beginning |
| | | at 1472 bp downstream of the start codon, resulting in a |
| | | truncated glnE protein lacking the adenylyl-removing |
| | | (AR) domain; 500 bp flanking the nifL gene upstream |
| | | and downstream are included |
| SEQ ID | ΔnifL::Prm8.2 | starting at 24 bp after the A of the ATG start codon, |
| NO 113 | | 1372 bp of nifL have been deleted and replaced with |
| | | the 137 Prm8.2 promoter sequence |
| SEQ ID | ΔnifL::Prm8.2 | starting at 24 bp after the A of the ATG start codon, |
| NO 114 | with 500 bp flank | 1372 bp of nifL have been deleted and replaced with |
| | | the 137 Prm8.2 promoter sequence; 500 bp flanking the |
| | | nifL gene upstream and downstream are included |
| SEQ ID | glnEΔAR-2 | glnE gene with 1290 bp immediately downstream of the |
| NO 115 | 36 bp deletion | ATG start codon deleted AND 36 bp deleted beginning |
| | | at 1472 bp downstream of the start codon, resulting in a |
| | | truncated glnE protein lacking the adenylyl-removing |
| | | (AR) domain |
| SEQ ID | glnEΔAR-2 | glnE gene with 1290 bp immediately downstream of the |
| NO 116 | 36 bp deletion | ATG start codon deleted AND 36 bp deleted beginning |
| | | at 1472 bp downstream of the start codon, resulting in a |
| | | truncated glnE protein lacking the adenylyl-removing |
| | | (AR) domain; 500 bp flanking the nifL gene upstream |
| | | and downstream are included |
| SEQ ID | ΔnifL::Prm6.2 | starting at 24 bp after the A of the ATG start codon, |
| NO 117 | | 1372 bp of nifL have been deleted and replaced with |
| | | the 137 Prm6.2 promoter sequence |
| SEQ ID | ΔnifL::Prm6.2 | starting at 24 bp after the A of the ATG start codon, |
| NO 118 | with 500 bp flank | 1372 bp of nifL have been deleted and replaced with |
| | | the 137 Prm6.2 promoter sequence; 500 bp flanking the |
| | | nifL gene upstream and downstream are included |
| SEQ ID | ΔnifL::Prm1.2 | starting at 24 bp after the A of the ATG start codon, |
| NO 119 | | 1372 bp of nifL have been deleted and replaced with |
| | | the 137 Prm1.2 promoter sequence |
| SEQ ID | ΔnifL::Prm1.2 | starting at 24 bp after the A of the ATG start codon, |
| NO 120 | with 500 bp flank | 1372 bp of nifL have been deleted and replaced with |
| | | the 137 Prm1.2 promoter sequence; 500 bp flanking the |
| | | nifL gene upstream and downstream are included |
| SEQ ID | glnEΔAR-2 | glnE gene with 1290 bp immediately downstream of the |
| NO 121 | 36 bp deletion | ATG start codon deleted AND 36 bp deleted beginning |
| | | at 1472 bp downstream of the start codon, resulting in a |
| | | truncated glnE protein lacking the adenylyl-removing |
| | | (AR) domain |
| SEQ ID | glnEΔAR-2 | glnE gene with 1290 bp immediately downstream of the |
| NO 122 | 36 bp deletion | ATG start codon deleted AND 36 bp deleted beginning |
| | | at 1472 bp downstream of the start codon, resulting in a |
| | | truncated glnE protein lacking the adenylyl-removing |
| | | (AR) domain; 500 bp flanking the nifL gene upstream |
| | | and downstream are included |
| SEQ ID | ΔnifL::PinfC | starting at 24 bp after the A of the ATG start codon, |
| NO 123 | | 1372 bp of nifL have been deleted and replaced with |
| | | the 137 PinfC promoter sequence |
| SEQ ID | AnifL::PinfC | starting at 24 bp after the A of the ATG start codon, |
| NO 124 | with 500 bp flank | 1372 bp of nifL have been deleted and replaced with |
| | | the 137 PinfC promoter sequence; 500 bp flanking the |
| | | nifL gene upstream and downstream are included |
| SEQ ID | glnEΔAR-2 | glnE gene with 1290 bp immediately downstream of the |
| NO 125 | | ATG start codon deleted, resulting in a truncated glnE |
| | | protein lacking the adenylyl-removing (AR) domain |
| SEQ ID | glnEΔAR-2 with | glnE gene with 1290 bp immediately downstream of the |
| NO 126 | 500 bp flank | ATG start codon deleted, resulting in a truncated glnE |
| | | protein lacking the adenylyl-removing (AR) domain; |
| | | 500 bp flanking the glnE gene upstream and |
| | | downstream are included |
| SEQ ID | glnEΔAR-2 | glnE gene with 1650 bp immediately downstream of the |
| NO 127 | | ATG start codon deleted, resulting in a truncated glnE |
| | | protein lacking the adenylyl-removing (AR) domain |
| SEQ ID | glnEΔAR-2 with | glnE gene with 1650 bp immediately downstream of the |
| NO 128 | 500 bp flank | ATG start codon deleted, resulting in a truncated glnE |
| | | protein lacking the adenylyl-removing (AR) domain; |
| | | 500 bp flanking the glnE gene upstream and |
| | | downstream are included |
| SEQ ID | ΔnifL::Prm6.1 | starting at 221 bp after the A of the ATG start codon, |
| NO 129 | | 845 bp of nifL have been deleted and replaced with the |
| | | NCMA 201701003 Prm6.1 promoter sequence |
| SEQ ID | ΔnifL::Prm6.1 | starting at 221 bp after the A of the ATG start codon, |
| NO 130 | with 500 bp flank | 845 bp of nifL have been deleted and replaced with the |
| | | NCMA 201701003 Prm6.1 promoter sequence; 500 bp |
| | | flanking the nifL gene upstream and downstream are |
| | | included |
| SEQ ID | ΔnifL::Prm6.1 | starting at 221 bp after the A of the ATG start codon, |
| NO 131 | | 845 bp of nifL have been deleted and replaced with the |
| | | NCMA 201701003 Prm6.1 promoter sequence |
| SEQ ID | ΔnifL::Prm6.1 | starting at 221 bp after the A of the ATG start codon, |
| NO 132 | with 500 bp flank | 845 bp of nifL have been deleted and replaced with the |
| | | NCMA 201701003 Prm6.1 promoter sequence; 500 bp |
| | | flanking the nifL gene upstream and downstream are |
| | | included |
| SEQ ID | ΔnifL::Prm7.1 | starting at 221 bp after the A of the ATG start codon, |
| NO 133 | | 845 bp of nifL have been deleted and replaced with the |
| | | NCMA 201701003 Prm7.1 promoter sequence |
| SEQ ID | ΔnifL::Prm7.1 | starting at 221 bp after the A of the ATG start codon, |
| NO 134 | with 500 bp flank | 845 bp of nifL have been deleted and replaced with the |
| | | NCMA 201701003 Prm76.1 promoter sequence; |
| | | 500 bp flanking the nifL gene upstream and |
| | | downstream are included |
| SEQ ID | ΔnifL::Prm1.2 | starting at 221 bp after the A of the ATG start codon, |
| NO 135 | | 845 bp of nifL have been deleted and replaced with the |
| | | NCMA 201701003 Prm1.2 promoter sequence |
| SEQ ID | ΔnifL::Prm1.2 | starting at 221 bp after the A of the ATG start codon, |
| NO 136 | with 500 bp flank | 845 bp of nifL have been deleted and replaced with the |
| | | NCMA 201701003 Prm1.2 promoter sequence; 500 bp |
| | | flanking the nifL gene upstream and downstream are |
| | | included |
| SEQ ID | ΔnifL::Prm1.2 | starting at 221 bp after the A of the ATG start codon, |
| NO 137 | | 845 bp of nifL have been deleted and replaced with the |
| | | NCMA 201701003 Prm1.2 promoter sequence |
| SEQ ID | ΔnifL::Prm1.2 | starting at 221 bp after the A of the ATG start codon, |
| NO 138 | with 500 bp flank | 845 bp of nifL have been deleted and replaced with the |
| | | NCMA 201701003 Prm1.2 promoter sequence; 500 bp |
| | | flanking the nifL gene upstream and downstream are |
| | | included |
| SEQ ID | glnEΔAR-2 | glnE gene with 1650 bp immediately downstream of the |
| NO 139 | | ATG start codon deleted, resulting in a truncated glnE |
| | | protein lacking the adenylyl-removing (AR) domain |
| SEQ ID | glnEΔAR-2 with | glnE gene with 1650 bp immediately downstream of the |
| NO 140 | 500 bp flank | ATG start codon deleted, resulting in a truncated glnE |
| | | protein lacking the adenylyl-removing (AR) domain; |
| | | 500 bp flanking the glnE gene upstream and |
| | | downstream are included |
| SEQ ID | ΔnifL::Prm3.1 | starting at 221 bp after the A of the ATG start codon, |
| NO 141 | | 845 bp of nifL have been deleted and replaced with the |
| | | NCMA 201701003 Prm3.1 promoter sequence |
| SEQ ID | ΔnifL::Prm3.1 | starting at 221 bp after the A of the ATG start codon, |
| NO 142 | with 500 bp flank | 845 bp of nifL have been deleted and replaced with the |
| | | NCMA 201701003 Prm3.1 promoter sequence; 500 bp |
| | | flanking the nifL gene upstream and downstream are |
| | | included |
| SEQ ID | glnEΔAR-2 | glnE gene with 1650 bp immediately downstream of the |
| NO 143 | | ATG start codon deleted, resulting in a truncated glnE |
| | | protein lacking the adenylyl-removing (AR) domain |
| SEQ ID | glnEΔAR-2 with | glnE gene with 1650 bp immediately downstream of the |
| NO 144 | 500 bp flank | ATG start codon deleted, resulting in a truncated glnE |
| | | protein lacking the adenylyl-removing (AR) domain; |
| | | 500 bp flanking the glnE gene upstream and |
| | | downstream are included |
| SEQ ID | ΔnifL::PinfC | starting at 24 bp after the A of the ATG start codon, |
| NO 145 | | 1375 bp of nifL have been deleted and replaced with |
| | | the 63 PinfC promoter sequence |
| SEQ ID | ΔnifL::PinfC | starting at 24 bp after the A of the ATG start codon, |
| NO 146 | with 500 bp flank | 1375 bp of nifL have been deleted and replaced with |
| | | the 63 PinfC promoter sequence; 500 bp flanking the |
| | | nifL gene upstream and downstream are included |
| SEQ ID | ΔnifL::Prm5 | starting at 31 bp after the A of the ATG start codon, |
| NO 147 | | 1375 bp of nifL have been deleted and replaced with |
| | | the NCMA 201701001 Prm5 promoter sequence |
| SEQ ID | ΔnifL::Prm5 | starting at 31 bp after the A of the ATG start codon, |
| NO 148 | with 500 bp flank | 1375 bp of nifL have been deleted and replaced with |
| | | the NCMA 201701001 Prm5 promoter sequence; |
| | | 500 bp flanking the nifL gene upstream and |
| | | downstream are included |
| SEQ ID | ΔnifL::Prm1 | starting at 31 bp after the A of the ATG start codon, |
| NO 149 | | 1375 bp of nifL have been deleted and replaced with |
| | | the NCMA 201701001 Prm1 promoter sequence |
| SEQ ID | ΔnifL::Prm1 | starting at 31 bp after the A of the ATG start codon, |
| NO 150 | with 500 bp flank | 1375 bp of nifL have been deleted and replaced with |
| | | the NCMA 201701001 Prm1 promoter sequence; |
| | | 500 bp flanking the nifL gene upstream and |
| | | downstream are included |
| SEQ ID | ΔnifL::Prm1 | starting at 31 bp after the A of the ATG start codon, |
| NO 151 | | 1375 bp of nifL have been deleted and replaced with |
| | | the NCMA 201701001 Prm1 promoter sequence |
| SEQ ID | AnifL::Prm1 | starting at 31 bp after the A of the ATG start codon, |
| NO 152 | with 500 bp flank | 1375 bp of nifL have been deleted and replaced with |
| | | the NCMA 201701001 Prm1 promoter sequence; |
| | | 500 bp flanking the nifL gene upstream and |
| | | downstream are included |
| SEQ ID | glnEΔAR-2 | glnE gene with 1644 bp immediately downstream of the |
| NO 153 | | ATG start codon deleted, resulting in a truncated glnE |
| | | protein lacking the adenylyl-removing (AR) domain |
| SEQ ID | glnEΔAR-2 with | glnE gene with 1644 bp immediately downstream of the |
| NO 154 | 500 bp flank | ATG start codon deleted, resulting in a truncated glnE |
| | | protein lacking the adenylyl-removing (AR) domain; |
| | | 500 bp flanking the glnE gene upstream and |
| | | downstream are included |
| SEQ ID | glnEΔAR-1 | glnE gene with 1287 bp immediately downstream of the |
| NO 155 | | ATG start codon deleted, resulting in a truncated glnE |
| | | protein lacking the adenylyl-removing (AR) domain |
| SEQ ID | ΔnifL::Prm1 | starting at 31 bp after the A of the ATG start codon, |
| NO 156 | | 1375 bp of nifL have been deleted and replaced with |
| | | the NCMA 201701001 Prm1 promoter sequence |
| SEQ ID | ΔnifL::Prm1 | starting at 31 bp after the A of the ATG start codon, |
| NO 157 | with 500 bp flank | 1375 bp of nifL have been deleted and replaced with |
| | | the NCMA 201701001 Prm1 promoter sequence; |
| | | 500 bp flanking the nifL gene upstream and |
| | | downstream are included |
| SEQ ID | glnEΔAR-1 with | glnE gene with 1287 bp immediately downstream of the |
| NO 158 | 500 bp flank | ATG start codon deleted, resulting in a truncated glnE |
| | | protein lacking the adenylyl-removing (AR) domain; |
| | | 500 bp flanking the glnE gene upstream and |
| | | downstream are included |
| SEQ ID | glnEΔAR-1 | glnE gene with 1287 bp immediately downstream of the |
| NO 159 | | ATG start codon deleted, resulting in a truncated glnE |
| | | protein lacking the adenylyl-removing (AR) domain |
| SEQ ID | glnEΔAR-1 with | glnE gene with 1287 bp immediately downstream of the |
| NO 160 | 500 bp flank | ATG start codon deleted, resulting in a truncated glnE |
| | | protein lacking the adenylyl-removing (AR) domain; |
| | | 500 bp flanking the glnE gene upstream and |
| | | downstream are included |
| SEQ ID | ΔnifL::Prm5 | starting at 31 bp after the A of the ATG start codon, |
| NO 161 | | 1375 bp of nifL have been deleted and replaced with |
| | | the NCMA 201701001 Prm5 promoter sequence |
| SEQ ID | ΔnifL::Prm5 | starting at 31 bp after the A of the ATG start codon, |
| NO 162 | with 500 bp flank | 1375 bp of nifL have been deleted and replaced with |
| | | the NCMA 201701001 Prm5 promoter sequence; |
| | | 500 bp flanking the nifL gene upstream and |
| | | downstream are included |
| SEQ ID | ΔnifL::Prm1 | starting at 31 bp after the A of the ATG start codon, |
| NO 163 | | 1375 bp of nifL have been deleted and replaced with |
| | | the NCMA 201701001 Prm1 promoter sequence |
| SEQ ID | ΔnifL::Prm1 | starting at 31 bp after the A of the ATG start codon, |
| NO 164 | with 500 bp flank | 1375 bp of nifL have been deleted and replaced with |
| | | the NCMA 201701001 Prm1 promoter sequence; |
| | | 500 bp flanking the nifL gene upstream and |
| | | downstream are included |
| SEQ ID | glnEΔAR-2 | glnE gene with 1644 bp immediately downstream of the |
| NO 165 | | ATG start codon deleted, resulting in a truncated glnE |
| | | protein lacking the adenylyl-removing (AR) domain |
| SEQ ID | glnEAAR-2 with | glnE gene with 1644 bp immediately downstream of the |
| NO 166 | 500 bp flank | ATG start codon deleted, resulting in a truncated glnE |
| | | protein lacking the adenylyl-removing (AR) domain; |
| | | 500 bp flanking the glnE gene upstream and |
| | | downstream are included |
| SEQ ID | ΔamtB | First 1088 bp of amtB gene and 4 bp upstream of start |
| NO 167 | | codon deleted; 199 bp of gene remaining lacks a start |
| | | codon; no amtB protein is translated |
| SEQ ID | ΔamtB with | First 1088 bp of amtB gene and 4 bp upstream of start |
| NO 168 | 500 bp flank | codon deleted; 199 bp of gene remaining lacks a start |
| | | codon; no amtB protein is translated |
| SEQ ID | glnEΔAR-1 | glnE gene with 1287 bp immediately downstream of the |
| NO 169 | | ATG start codon deleted, resulting in a truncated glnE |
| | | protein lacking the adenylyl-removing (AR) domain |
| SEQ ID | glnEΔAR-1 with | glnE gene with 1287 bp immediately downstream of the |
| NO 170 | 500 bp flank | ATG start codon deleted, resulting in a truncated glnE |
| | | protein lacking the adenylyl-removing (AR) domain; |
| | | 500 bp flanking the glnE gene upstream and |
| | | downstream are included |
| SEQ ID | ΔnifL::Prm1 | starting at 31 bp after the A of the ATG start codon, |
| NO 171 | | 1375 bp of nifL have been deleted and replaced with |
| | | the NCMA 201701001 Prm1 promoter sequence |
| SEQ ID | ΔnifL::Prm1 | starting at 31 bp after the A of the ATG start codon, |
| NO 172 | with 500 bp flank | 1375 bp of nifL have been deleted and replaced with |
| | | the NCMA 201701001 Prm1 promoter sequence; |
| | | 500 bp flanking the nifL gene upstream and |
| | | downstream are included |
| SEQ ID | ΔamtB | First 1088 bp of amtB gene and 4 bp upstream of start |
| NO 173 | | codon deleted; 199 bp of gene remaining lacks a start |
| | | codon; no amtB protein is translated |
| SEQ ID | ΔamtB with | First 1088 bp of amtB gene and 4 bp upstream of start |
| NO 174 | 500 bp flank | codon deleted; 199 bp of gene remaining lacks a start |
| | | codon; no amtB protein is translated |
| SEQ ID | ΔnifL::PinfC | starting at 20 bp after the A of the ATG start codon, |
| NO 175 | | 1379 bp of nifL have been deleted and replaced with |
| | | the PTA-126585 PinfC promoter sequence |
| SEQ ID | ΔnifL::PinfC | starting at 20 bp after the A of the ATG start codon, |
| NO 176 | with 500 bp flank | 1379 bp of nifL have been deleted and replaced with |
| | | the PTA-126585 PinfC promoter sequence; 500 bp |
| | | flanking the nifL gene upstream and downstream are |
| | | included |
| SEQ ID | 16S-1 | 1 of 3 unique 16S rDNA genes in the NCMA |
| NO 177 | | 201701001 genome |
| SEQ ID | 16S-2 | 2 of 3 unique 16S rDNA genes in the NCMA |
| NO 178 | | 201701001 genome |
| SEQ ID | nifH | N/A |
| NO 179 | | |
| SEQ ID | nifD2 | 2 of 2 unique genes annotated as nifD in NCMA |
| NO 180 | | 201701001 genome |
| SEQ ID | nifK2 | 2 of 2 unique genes annotated as nifK in NCMA |
| NO 181 | | 201701001 genome |
| SEQ ID | nifL | N/A |
| NO 182 | | |
| SEQ ID | nifA | N/A |
| NO 183 | | |
| SEQ ID | glnE | N/A |
| NO 184 | | |
| SEQ ID | 16S-3 | 3 of 3 unique 16S rDNA genes in the NCMA |
| NO 185 | | 201701001 genome |
| SEQ ID | nifD1 | 1 of 2 unique genes annotated as nifD in NCMA |
| NO 186 | | 201701001 genome |
| SEQ ID | nifK1 | 1 of 2 unique genes annotated as nifK in NCMA |
| NO 187 | | 201701001 genome |
| SEQ ID | amtB | N/A |
| NO 188 | | |
| SEQ ID | Prm1 | 348 bp includes the 319 bp immediately upstream of the |
| NO 189 | | ATG start codon of the lpp gene and the first 29 bp of |
| | | the lpp gene |
| SEQ ID | Prm5 | 313 bp starting at 432 bp upstream of the ATG start |
| NO 190 | | codon of the ompX gene and ending 119 bp upstream |
| | | of the ATG start codon of the ompX gene |
| SEQ ID | nifL | N/A |
| NO 191 | | |
| SEQ ID | nifA | N/A |
| NO 192 | | |
| SEQ ID | 16S-1 | 1 of 7 unique 16S rDNA genes in the NCMA |
| NO 193 | | 201701003 genome |
| SEQ ID | 16S-2 | 2 of 7 unique 16S rDNA genes in the NCMA |
| NO 194 | | 201701003 genome |
| SEQ ID | 16S-3 | 3 of 7 unique 16S rDNA genes in the NCMA |
| NO 195 | | 201701003 genome |
| SEQ ID | 16S-4 | 4 of 7 unique 16S rDNA genes in the NCMA |
| NO 196 | | 201701003 genome |
| SEQ ID | 16S-5 | 5 of 7 unique 16S rDNA genes in the NCMA |
| NO 197 | | 201701003 genome |
| SEQ ID | 16S-6 | 6 of 7 unique 16S rDNA genes in the NCMA |
| NO 198 | | 201701003 genome |
| SEQ ID | 16S-7 | 7 of 7 unique 16S rDNA genes in the NCMA |
| NO 199 | | 201701003 genome |
| SEQ ID | nifH1 | 1 of 2 unique genes annotated as nifH in NCMA |
| NO 200 | | 201701003 genome |
| SEQ ID | nifH2 | 2 of 2 unique genes annotated as nifH in NCMA |
| NO 201 | | 201701003 genome |
| SEQ ID | nifD1 | 1 of 2 unique genes annotated as nifD in NCMA |
| NO 202 | | 201701003 genome |
| SEQ ID | nifD2 | 2 of 2 unique genes annotated as nifD in NCMA |
| NO 203 | | 201701003 genome |
| SEQ ID | nifK1 | 1 of 2 unique genes annotated as nifK in NCMA |
| NO 204 | | 201701003 genome |
| SEQ ID | nifK2 | 2 of 2 unique genes annotated as nifK in NCMA |
| NO 205 | | 201701003 genome |
| SEQ ID | glnE | N/A |
| NO 206 | | |
| SEQ ID | Prm4 | 449 bp immediately upstream of the ATG of the dscC 2 |
| NO 207 | | gene |
| SEQ ID | Prm1.2 | 500 bp immediately upstream of the TTG start codon of |
| NO 208 | | the infC gene |
| SEQ ID | Prm3.1 | 170 bp immediately upstream of the ATG start codon |
| NO 209 | | of the rplN gene |
| SEQ ID | Prm6.1 | 142 bp immediately upstream of the ATG of a highly - |
| NO 210 | | expressed hypothetical protein (annotated as |
| | | PROKKA_00662 in NCMA 201701003 assembly 82) |
| SEQ ID | Prm7.1 | 293 bp immediately upstream of the ATG of the lpp |
| NO 211 | | gene |
| SEQ ID | glnEΔAR-2 | glnE gene with 1650 bp immediately downstream of the |
| NO 212 | | ATG start codon deleted, resulting in a truncated glnE |
| | | protein lacking the adenylyl-removing (AR) domain |
| SEQ ID | glnEΔAR-2 with | glnE gene with 1650 bp immediately downstream of the |
| NO 213 | 500 bp flank | ATG start codon deleted, resulting in a truncated glnE |
| | | protein lacking the adenylyl-removing (AR) domain; |
| | | 500 bp flanking the glnE gene upstream and |
| | | downstream are included |
| SEQ ID | ΔnifL::null-v1 | starting at 221 bp after the A of the ATG start codon, |
| NO 214 | | 845 bp of nifL have been deleted and replaced with the |
| | | 31 bp sequence |
| | | “GGAGTCTGAACTCATCCTGCGATGGGGGCTG” |
| SEQ ID | ΔnifL::null-v1 | starting at 221 bp after the A of the ATG start codon, |
| NO 215 | with 500 bp flank | 845 bp of nifL have been deleted and replaced with the |
| | | 31 bp sequence |
| | | “GGAGTCTGAACTCATCCTGCGATGGGGGCTG”; |
| | | 500 bp flanking the nifL gene upstream and |
| | | downstream are included |
| SEQ ID | ΔnifL::null-v2 | starting at 221 bp after the A of the ATG start codon, |
| NO 216 | | 845 bp of nifL have been deleted and replaced with the |
| | | 5 bp sequence “TTAAA” |
| SEQ ID | ΔnifL::null-v2 | starting at 221 bp after the A of the ATG start codon, |
| NO 217 | with 500 bp flank | 845 bp of nifL have been deleted and replaced with the |
| | | 5 bp sequence “TTAAA”; 500 bp flanking the nifL gene |
| | | upstream and downstream are included |
| SEQ ID | ΔnifL::Prm4 | starting at 221 bp after the A of the ATG start codon, |
| NO 218 | | 845 bp of nifL have been deleted and replaced with the |
| | | CI19 Prm4 sequence |
| SEQ ID | ΔnifL::Prm4 | starting at 221 bp after the A of the ATG start codon, |
| NO 219 | with 500 bp flank | 845 bp of nifL have been deleted and replaced with the |
| | | CI19 Prm4 sequence; 500 bp flanking the nifL gene |
| | | upstream and downstream are included |
| SEQ ID | glnD with | glnD gene with upstream and downstream flanking |
| NO 220 | flanking | sequences included |
| | sequences | |
| SEQ ID | ΔnifL::Prm5 | Retaining the first 30 bp and the last 83 bp, the middle |
| NO 221 | | region of nifL is deleted and replaced with the NCMA |
| | | 201701001 Prm5 promoter sequence. |
| SEQ ID | glnEΔAR | Deletion of the first 1287 bp after the ATG start codon |
| NO 222 | | of the glnE gene. Resulting GlnE protein lacks AR |
| | | domain, but expresses ATase domain. |
| SEQ ID | ΔglnD with | Deletion of glnD gene by removing all 2676 |
| NO 223 | flanking | nucleotides, including start and stop codons. Upstream |
| | sequences | and downstream flanking sequences are included. |
| | ΔnifL::PinfC | Deletion of the nifL gene from 20 bp after the ATG |
| | | (start) to 87 bp before the TGA (stop) of the gene. A |
| | | 500 bp fragment of the region upstream of the infC |
| | | gene was inserted (PinfC) upstream of nifA replacing |
| | | the deleted portion. |
| | ΔglnE_{AR}-KO2 | Deletion of 1647 bp after the start codon of the glnE |
| | | gene. |
| | ΔnifL::PinfC | Deletion of the nifL gene from 20 bp after the ATG |
| | | (start) to 87 bp before the TGA (stop) of the gene. A |
| | | 500 bp fragment of the region upstream of the infC |
| | | gene was inserted (PinfC) upstream of nifA replacing |
| | | the deleted portion. |
| | glnE_{AR}-DxD | Modification of the “GAT” found 513 bp after the start |
| | | codon of glnE to a “GCG” codon. |
| | ΔnifL::Prm8.2 | Deletion of the nifL gene from 20 bp after the ATG |
| | | (start) to 87 bp before the TGA (stop) of the gene. A |
| | | 299 bp fragment (Prm8.2), found 77 bp after the start |
| | | codon of nlpI to 376 bp after the start codon of nlpI was |
| | | inserted upstream of nifA replacing the deleted portion. |
| | ΔglnE_{AR}-KO2 | Deletion of 1647 bp after the start codon of the glnE |
| | | gene. |
| | ΔnifL::PinfC | Deletion of the nifL gene from 20 bp after the ATG |
| | | (start) to 87 bp before the TGA (stop) of the gene. A |
| | | 500 bp fragment of the region upstream of the infC |
| | | gene was inserted (PinfC) upstream of nifA replacing |
| | | the deleted portion. |
| | ΔglnE_{AR}-KO2 | Deletion of 1647 bp after the start codon of the glnE |
| | | gene. |
| | ΔnifL::Prm1.2 | Deletion of the nifL gene from 20 bp after the ATG |
| | | (start) to 87 bp before the TGA (stop) of the gene. A |
| | | 400 bp fragment from the region upstream of the cspE |
| | | gene was inserted (Prm1.2) upstream of nifA replacing |
| | | the deleted portion. |
| | ΔglnE_{AR}-KO2 | Deletion of 1647 bp after the start codon of the glnE |
| | | gene. |
| | ΔnifL::Prm1.2 | Deletion of the nifL gene from 20 bp after the ATG |
| | | (start) to 87 bp before the TGA (stop) of the gene. A |
| | | 400 bp fragment from the region upstream of the cspE |
| | | gene was inserted (Prm1.2) upstream of nifA replacing |
| | | the deleted portion. |
| | ΔglnE_{AR}-KO2 | Deletion of 1647 bp after the start codon of the glnE |
| | | gene. |
| | rpoN-Prm8.2 | Deletion of the 47bo between ibtB2 and rpoN and |
| | | insertion of a fragment (Prm8.2), found 77 bp after the |
| | | start codon of nlpI to 376 bp after the start codon of |
| | | nlpI, directly upstream of rpoN. |
| | ΔnifL::Prm1.2 | Deletion of the nifL gene from 20 bp after the ATG |
| | | (start) to 87 bp before the TGA (stop) of the gene. A |
| | | 400 bp fragment from the region upstream of the cspE |
| | | gene was inserted (Prm1.2) upstream of nifA replacing |
| | | the deleted portion. |
| | ΔglnE_{AR}-KO2 | Deletion of 1647 bp after the start codon of the glnE |
| | | gene. |
| | ΔglnD_{ACT1/2} | Deletion of the 546 bp before the stop codon of the |
| | | glnD gene. |

TABLE 5
Further Descriptions of Deposited Strains from the Disclosure

| | Mutagenic DNA | |
|---|---|---|
| Strain ID | Description | Genotype |
| NCMA | Wildtype parent Kosakonia | WT |
| 201701001 | sacchari | |
| NCMA | Wildtype parent Klebsiella | WT |
| 201708001 | variicola | |
| PTA-126585 | Wildtype parent | WT |
| | Metakosakonia intestini | |
| PTA-126582 | Wildtype parent | WT |
| | Paraburkholderia tropica | |
| PTA-126581 | Wildtype parent | WT |
| | Paenibacillus polymyxa | |
| PTA-126583 | Wildtype parent | WT |
| | Herbaspirillum aquaticum | |
| NCMA | Disruption of nifL gene with | ΔnifL::Prm1, ΔglnE- |
| 201708004 | a fragment of the region | AR_KO2 |
| | upstream of the lpp gene | |
| | inserted (Prm1) upstream of | |
| | nifA. Deletion of the 1647 bp | |
| | after the start codon of the | |
| | glnE gene containing the | |
| | adenylyl-removing domain | |
| | of glutamate-ammonia-ligase | |
| | adenylyltransferase (ΔglnE- | |
| | AR_KO2). | |
| PTA-126575 | Disruption of nifL gene with | ΔnifL::Prm1, |
| | a fragment of the region | ΔglnD_UT_truncation |
| | upstream of the lpp gene | |
| | inserted (Prm1) upstream of | |
| | nifA. Deletion of the 987 bp | |
| | after the start codon of the | |
| | glnD gene containing the | |
| | uridylyltransferase (UT) | |
| | domain of the bifunctional | |
| | uridylyltransferase/uridylyl- | |
| | removing enzyme (ΔglnD- | |
| | UT_truncation) | |
| PTA-126576 | Disruption of nifL gene with | ΔnifL::Prm1, |
| | a fragment of the region | ΔglnD_UT_deactivation |
| | upstream of the lpp gene | |
| | inserted (Prm1) upstream of | |
| | nifA. Deactivation of the | |
| | uridylyltransferase (UT) | |
| | domain of the bifunctional | |
| | uridylyltransferase/uridylyl- | |
| | removing enzyme, glnD, by | |
| | mutating amino acid | |
| | residues 90 and 91 from GG | |
| | to DV as well as residue | |
| | 104 from D to A. | |
| NCMA | Deletion of the 1647 bp after | ΔglnE-AR_KO2 |
| 201712001 | the start codon of the glnE | |
| | gene containing the | |
| | adenylyl-removing domain | |
| | of glutamate-ammonia-ligase | |
| | adenylyltransferase (ΔglnE- | |
| | AR_KO2). | |
| PTA-126577 | Deletion of the native dctA1 | ΔnifL::P8.2, ΔglnE- |
| | promoter and insertion of a | AR_KO2 |
| | fragment (Prm8.2), found | |
| | 77 bp after the start codon of | |
| | nipI to 376 bp after the start | |
| | codon of nlpI, directly | |
| | upstream of dctA1. Deletion | |
| | of the 1647 bp after the start | |
| | codon of the glnE gene | |
| | containing the adenylyl- | |
| | removing domain of | |
| | glutamate-ammonia-ligase | |
| | adenylyltransferase (ΔglnE- | |
| | AR_KO2). | |
| PTA-126578 | Disruption of nifL gene with | ΔnifL::Prm1.2 ΔglnE- |
| | a fragment of the region | AR_KO2, |
| | upstream of the cspE gene | ΔglnD_ACT 12_ |
| | inserted (Prm1.2) upstream | truncation |
| | of nifA. Deletion of the | |
| | 1647 bp after the start codon | |
| | of the glnE gene containing | |
| | the adenylyl-removing | |
| | domain of glutamate- | |
| | ammonia-ligase | |
| | adenylyltransferase (ΔglnE- | |
| | AR_KO2). Deletion of the | |
| | 546 bp before the stop codon | |
| | of the glnD gene containing | |
| | the ACT1/2 domain of the | |
| | bifunctional | |
| | uridylyltransferase/uridylyl- | |
| | removing enzyme (ΔglnD- | |
| | ACT12_truncation) | |
| PTA-126579 | Disruption of nifL gene with | ΔnifL::Prm1.2, ΔglnE- |
| | a fragment of the region | AR_KO2, glsA2::Prm1.2 |
| | upstream of the espE gene | |
| | inserted (Prm1.2) upstream | |
| | of nifA. Deletion of the | |
| | 1647 bp after the start codon | |
| | of the glnE gene containing | |
| | the adenylyl-removing | |
| | domain of glutamate- | |
| | ammonia-ligase | |
| | adenylyltransferase (ΔglnE- | |
| | AR_KO2). Deletion of the | |
| | native glsA2 promoter and | |
| | insertion of a fragment | |
| | (Prm1.2) directly upstream | |
| | of the glsA2 CDS. | |
| PTA-126580 | Disruption of nifL gene with | ΔnifL::Prm1.2, ΔglnE- |
| | a fragment of the region | AR_KO2, rpoN::Prm1.2 |
| | upstream of the espE gene | |
| | inserted (Prm1.2) upstream | |
| | of nifA. Deletion of the | |
| | 1647 bp after the start codon | |
| | of the glnE gene containing | |
| | the adenylyl-removing | |
| | domain of glutamate- | |
| | ammonia-ligase | |
| | adenylyltransferase (ΔglnE- | |
| | AR_KO2). Deletion of the | |
| | native rpoN promoter and | |
| | insertion of a fragment | |
| | (Prm1.2) directly upstream | |
| | of the rpoN CDS. | |
| PTA-126588 | Disruption of nifL gene with | ΔnifL::Prm2.1, ΔglnE- |
| | a fragment of the region | AR_KO2, glsA2::Prm1.1 |
| | upstream of the rmF gene | |
| | inserted (Prm2.1) upstream | |
| | of nifA. Deletion of the | |
| | 1647 bp after the start codon | |
| | of the glnE gene containing | |
| | the adenylyl-removing | |
| | domain of glutamate- | |
| | ammonia-ligase | |
| | adenylyltransferase (ΔglnE- | |
| | AR_KO2). Deletion of the | |
| | native glsA2 promoter and | |
| | insertion of a fragment | |
| | upstream of the csrA gene | |
| | (Prm1.1) directly upstream | |
| | of the glsA2 CDS. | |
| PTA-126586 | Disruption of nifL gene with | ΔnifL::Prm2.1, ΔglnE- |
| | a fragment of the region | AR_KO2, |
| | upstream of the rmF gene | ΔglnD_UT_truncation |
| | inserted (Prm2.1) upstream | |
| | of nifA. Deletion of the | |
| | 1647 bp after the start codon | |
| | of the glnE gene containing | |
| | the adenylyl-removing | |
| | domain of glutamate- | |
| | ammonia-ligase | |
| | adenylyltransferase (ΔglnE- | |
| | AR_KO2). Deletion of the | |
| | 987 bp after the start codon | |
| | of the glnD gene containing | |
| | the uridylyltransferase (UT) | |
| | domain of the bifunctional | |
| | uridylyltransferase/uridylyl- | |
| | removing enzyme (ΔglnD- | |
| | UT_truncation) | |
| PTA-126584 | Disruption of nifL gene with | ΔnifL::Prm2.1, ΔglnE- |
| | a fragment of the region | AR_KO2 |
| | upstream of the rmF gene | |
| | inserted (Prm2.1) upstream | |
| | of nifA. Deletion of the | |
| | 1647 bp after the start codon | |
| | of the glnE gene containing | |
| | the adenylyl-removing | |
| | domain of glutamate- | |
| | ammonia-ligase | |
| | adenylyltransferase (ΔglnE- | |
| | AR_KO2). | |
| PTA-126587 | Disruption of nifL gene with | ΔnifL::Prm2.1, |
| | a fragment of the region | ΔglnD_UT_truncation |
| | upstream of the rmF gene | |
| | inserted (Prm2.1) upstream | |
| | of nifA. Deletion of the | |
| | 987 bp after the start codon | |
| | of the glnD gene containing | |
| | the uridylyltransferase (UT) | |
| | domain of the bifunctional | |
| | uridylyltransferase/uridylyl- | |
| | removing enzyme (ΔglnD- | |
| | UT_truncation). | |
| PTA-126743 | Disruption of nifL gene with | ΔnifL::Prm5, glnEΔAR, |
| | a promoter that natively | ΔglnD |
| | drives ompX (Prm5) inserted | |
| | upstream of nifA. Deletion | |
| | of the 1287 bp after the start | |
| | codon of the glnE gene | |
| | containing the adenylyl- | |
| | removing domain of | |
| | glutamate-ammonia-ligase | |
| | adenylyltransferase Deletion | |
| | of entire glnD gene. | |

### EXAMPLES

### Example 1: Exemplary Method for Improving Stability of Liquid Agricultural Composition Comprising Nitrogen-Fixing Gram Negative Bacteria

To overcome the limitations of existing dry and liquid formulations of nitrogen-fixing microorganisms, novel formulation methods were developed to produce agronomically stable agricultural compositions for delivering nitrogen-fixing microorganism to agricultural plant tissues or the environs thereof. An exemplary implementation of a method according to the present disclosure involved varying four different parameters of the liquid agricultural composition, each of which proved to contribute to the stability of the composition. The four parameters were: (1) cell density of the nitrogen-fixing microorganism at time of formulation and selection and inclusion of (2) a microbial stabilizer, (3) a physical stabilizer, and (4) a buffering agent. The combination of the microbial stabilizer and the physical stabilizer was observed to have a direct impact on toxin accumulation.

In this example, the nitrogen-fixing microorganism was the diazotrophic bacterium Klebsiella variicola strain NCMA 201712002.

### Cell Density at Time of Formulation

To determine the effects of initial cell density on the stability of the bacteria within the liquid agricultural composition, a titration curve was created to measure the relationship between initial cell density and decay rate. Cellular densities between ˜9.4 and ˜10.85 (given in log CFU/mL) were tested with water used as a diluent. The decay rate for each sample was determined by taking measurements of the initial cellular density and the cellular density at the 30 day time point, using standard plating methods, which measurements were used to determine an overall average rate of decay, given in log loss of CFU/mL per day).

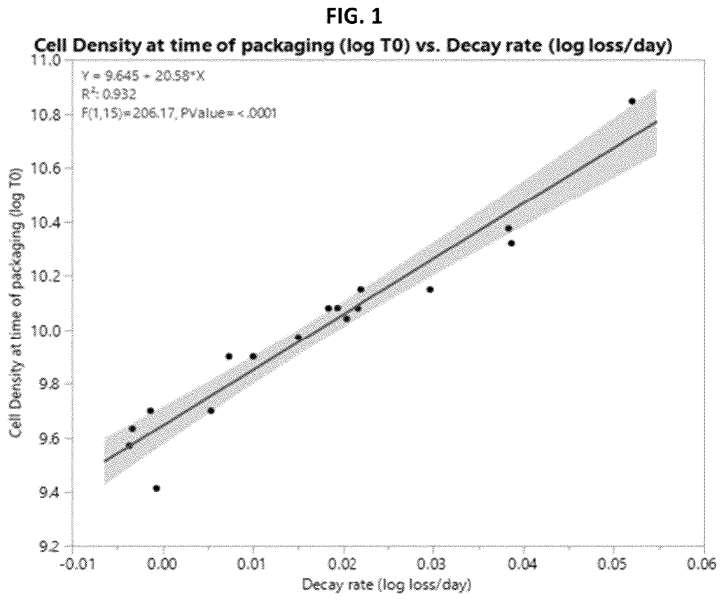

FIG. 1 shows the results of this assay, demonstrating that initial cellular densities between ˜9.4 and ˜9.7 log CFU/mL did not experience any average measurable decay over the 30 day period of observation, while higher initial cellular densities led to decay, with an initial cellular density of ˜10.85, for example, leading to a decay rate of ˜0.055 log loss of CFU/mL per day. This data shows a significant correlation (p<0.0001) between the cell density of the nitrogen-fixing microorganisms at the time of formulation of the agricultural composition and the decay rate of the microorganisms. Because of this, initial dilution had a significant impact on improving product stability while decreasing the required fermentation volume. Since the typical bacterial titer at the end of fermentation was around 1.5E10 CFU/mL, and the target cell density for the agricultural composition based on these results was around 4.5E9-5.5E9 CFU/mL, this provided the ability to dilute the fermentation volume 3-fold, thereby decreasing the fermentation needs 3-fold as well.

However, while packaging density was observed to affect the stability of the agricultural composition, other factors also affected stability beyond 30 days.

### Ammonia Toxin Accumulation Over Time

The inventors theorized that in addition to the effects of cellular density, another key mechanism of failure in the stability of the agricultural composition over time could be a high level of toxin accumulation during storage. Ammonia is a byproduct of media breakdown during fermentation and may also accumulate during storage due to nitrogen fixation by the bacteria. Ammonia is toxic to Klebsiella variicola NCMA 201712002 at concentrations above 50 mM, as determined by measuring the final cellular OD in a metabolite screen, results shown in Table 6.

TABLE 6
Ammonia toxicity in
Klebsiella variicola NCMA 201712002.

| [Ammonia] mM | Final OD |
|---|---|
| 10 | 2.0874 |
| 50 | 2.1558 |
| 100 | 0.4435 |

To test the possibility that ammonia accumulation could be linked to the deterioration of the agricultural composition's stability over time, average ammonia concentrations (averaged across triplicate samples) were measured in the supernatant of samples stored under different conditions for a period of 90 days, with measurements taken at 0, 35, and 90 days post-harvest. The conditions that varied were:
Dilution level: normal fermentation broth at 1E10 CFU/mL (“broth”) versus half broth/half PBS at 5E9 CFU/mL (“halfbroth”), neither one with stabilizers present;
Storage container: bladder (“bladder”) versus bottle (“bottle”); and
Storage temperature: 4° C. (“4C”) versus room temperature (“RT”), with one sample testing the condition of moving from 4° C. to RT at 35 days (“4CtoRT”).

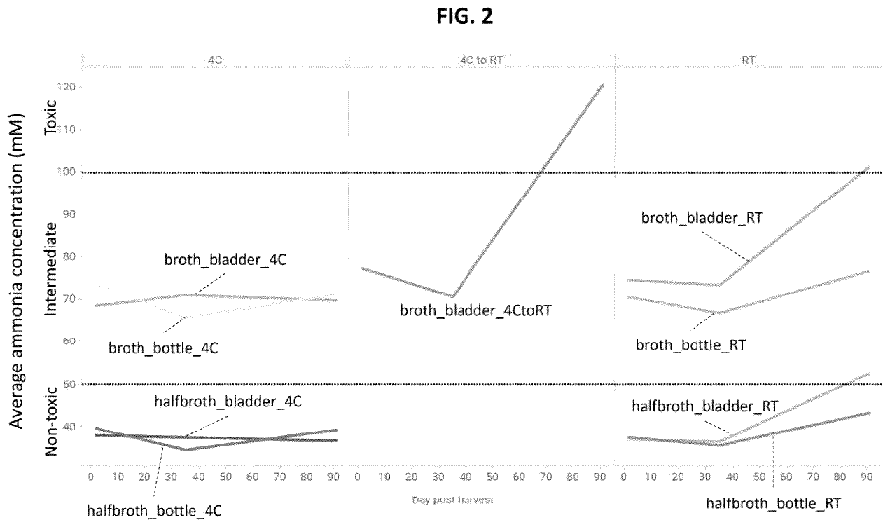

FIG. 2 shows the results of this experiment. In all room temperature samples, ammonia accumulated over time, with the average ammonia concentration at 90 days exceeding the starting value. This ammonia accumulation suggests that cells are metabolically active during storage in room temperature samples. On the other hand, samples stored at 4° C. did not experience a significant increase in ammonia concentration over the 90 day period. The greatest ammonia accumulation was observed in the sample that was moved from 4° C. to room temperature, which may indicate that the temperature shift upregulates cell metabolism.

### Microbial Stabilizer & Physical Stabilizer

To improve the stability of the agricultural composition beyond the 30 day period, 82 combinations of microbial and physical stabilizers were explored. The samples comprised fermentation broth at two different initial cell densities (2× and 3× dilution), all in high buffering capacity PBS, along with the combination of stabilizers. The samples were stored at room temperature. Stability and functionality were monitored for a period of 5 months. The stability of the product was measured using a room temperature measurement of cellular viability in units of CFU/mL.

During this time, various stabilizer combinations resulted in different levels of stability, with fructose and xanthan gum at a final concentration of 1.3% and 0.1%, respectively, producing the best results in terms of long term stability: this combination resulted in an agricultural composition that maintained its bacterial viability for the full 3 month period, with less than 0.2 log loss of CFU/mL over the 3 month time frame.

Four agricultural compositions were also tested in an in planta microbial colonization assay at the three month time point. The four compositions comprised the following combinations of microbial and physical stabilizers: (A) 100 mM PBS, no stabilizers; (B) 1.3% fructose+0.2% CBP; (C) 1.3% fructose+0.1% xanthan gum, and (D) HEPES, no stabilizers. Most of the other combinations of microbial and physical stabilizers produced results below the target of 1E9 CFU/mL at 3 months' time.

The root colonization assay was performed as in International Patent Application No. PCT/US2019/039528, the contents of which are incorporated herein by reference in their entirety. Briefly, four days after planting, 1 ml of a bacterial overnight culture (approximately 109 CFU) was applied to the soil above the planted seed. Seedlings were fertilized three times weekly with 25 ml modified Hoagland's solution supplemented with 0.5 mM ammonium nitrate. Four weeks after planting, root samples were collected and the total genomic DNA (gDNA) was extracted. Root colonization was quantified using qPCR with primers designed to amplify unique regions of either the wild type or derivative strain genome. QPCR reaction efficiency was measured using a standard curve generated from a known quantity of gDNA from the target genome. Data was normalized to genome copies per g fresh weight using the tissue weight and extraction volume. The 3-month old A-D samples were compared to a negative control (untreated control seedlings, no microbe added) and a positive control (fresh culture without stabilizers or buffers).

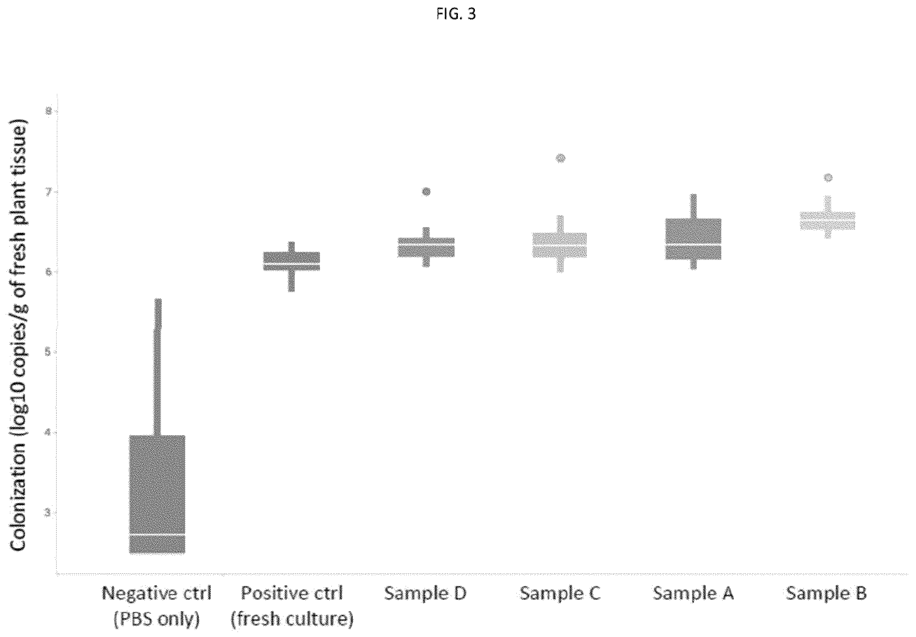

FIG. 3 and Table 7 show the results of this assay: all four 3-month old compositions had the same colonization performance as a fresh culture of bacteria.

TABLE 7
Colonization performance of 3-month
old agricultural compositions comprising high-
performing combinations of physical and microbial stabilizers.

| | Negative | Positive | Sample | Sample | Sample | Sample |
|---|---|---|---|---|---|---|
| Condition | control | control | D | C | A | B |
| No. of | 11 | 12 | 11 | 12 | 12 | 12 |
| replicates | | | | | | |
| Avg. | 3.29 | 6.10 | 6.34 | 6.40 | 6.41 | 6.68 |
| colonization | | | | | | |
| (log10 | | | | | | |
| copies/g | | | | | | |
| fresh weight) | | | | | | |

In addition to testing the colonization potential of the top-performing agricultural compositions, the supernatants of six different formulation conditions were assayed for soluble ammonia accumulation at the three month time point and compared to a control sample without stabilizers. These six formulations and control formulation comprised the following combinations of physical and microbial stabilizers:
(Sample 1) physical stabilizer: 0.1% xanthan gum; microbial stabilizer: 1.3% fructose;
(Sample 2) physical stabilizer: 0.1% xanthan gum; microbial stabilizer: 2.5% trehalose;
(Sample 3) physical stabilizer: 0.1% xanthan gum; microbial stabilizer: none;
(Sample 4) physical stabilizer: 0.2% CBP; microbial stabilizer: 1% fructose;
(Sample 5) physical stabilizer: 0.2% CBP; microbial stabilizer: 2.5% trehalose; and
(Sample 6) physical stabilizer: 0.2% CBP; microbial stabilizer: none.
(Control) physical stabilizer: none; microbial stabilizer: none.

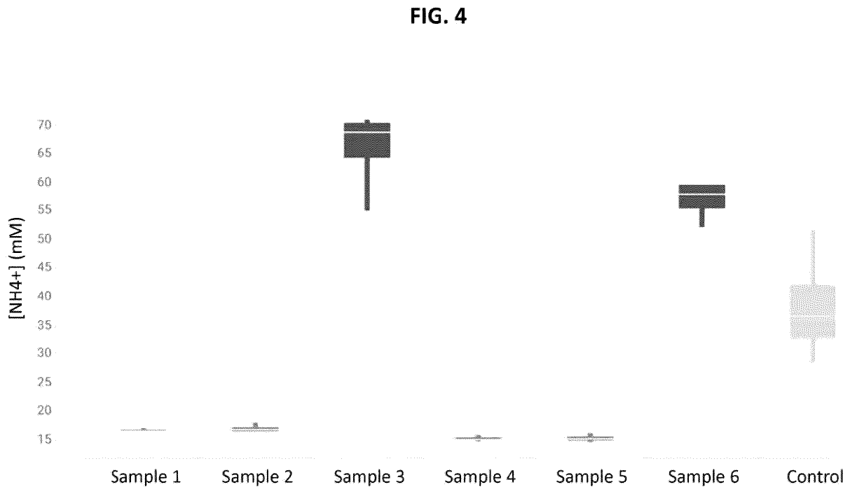

The results of this assay are shown in FIG. 4 and Table 8. These results show that the physical stabilizer alone was not sufficient to decrease ammonia accumulation over the 3-month time frame; in fact, the 0.1% xanthan gum alone condition and the 0.2% CBP alone condition both led to increased soluble ammonia production compared to the control condition without stabilizers. By contrast, the combinations of physical and microbial stabilizers reduced soluble ammonia accumulation four-fold compared to the physical stabilizers alone. In the presence of these combinations, the soluble ammonia accumulation dropped to non-toxic levels, well below the 50 mM threshold. In fact, a comparison of the results in FIG. 4 with those shown in FIG. 2 demonstrates that these combinations of stabilizers (in high buffering capacity PBS with an initial cell density selected to provide a lower decay rate) brought ammonia concentrations down to below 20 mM levels as compared to levels above 75 mM for the full broth, room temperature conditions shown in FIG. 2, a more than 3-fold decrease in ammonia concentration.

TABLE 8
Soluble ammonia accumulation
in 3-month old agricultural compositions.

| | Sam- | Sam- | Sam- | Sam- | Sam- | Sam- | |
|---|---|---|---|---|---|---|---|
| | ple | ple | ple | ple | ple | ple | |
| Condition | 1 | 2 | 3 | 4 | 5 | 6 | Control |
| Median | 16.51 | 16.70 | 68.77 | 15.02 | 15.08 | 57.94 | 36.53 |
| [NH4+] | | | | | | | |
| (mM) | | | | | | | |

### Buffering Agent

In addition to testing different combinations of stabilizers, various buffers were also assayed within the agricultural composition formulation to determine the effect of the choice of buffer on microbial stability. The buffers included: phosphate buffered saline (PBS); modified, high buffering capacity PBS; 3-Morpholinopropane-1-sulfonic acid (MOPS); 2-[4-(2-hydroxyethyl)piperazin-1-yl]ethanesulfonic acid (HEPES); and reverse osmosis water. The modified, high buffering capacity PBS had a higher concentration of KH₂PO₄ than the standard PBS recipe, with the following overall composition: 23 g/L KH₂PO₄, 7.5 g/L NaCl, 0.2 g/L KCl, and 1.4 g/L dibasic Na₂HPO₄. Each of the buffers was tested alone within the agricultural composition and in combination with various stabilizers. The samples all comprised the same initial cellular density of 3-5E9 CFU/mL selected to decrease the decay rate based on the results of the assay shown in FIG. 1.

The stability of the agricultural compositions was measured over time via room temperature measurements of cellular viability (CFU/mL). The 1-month time point was chosen as the deselection timeline, at which point it was determined that a target cell density of 3-5E9 CFU/mL was required for a stable product. For samples within that target cell density range at time of packaging, at the one month time point, the microbial stability was the same across all samples, regardless of the buffer or stabilizers comprised by the composition. At the two month time point, the combination of a buffer and a microbial stabilizer provided improved stability compared to conditions without those agents (regardless of the physical stabilizer). At the three month time point, the importance of the physical stabilizer type became apparent, and the fructose and xanthan gum combination of stabilizers outperformed other stabilizer combinations for preserving bacterial viability within the composition. Finally, at the four month time point, the combination of all four components was able to preserve bacterial viability: an initial cell density between 3-5E9 CFU/mL, high buffering capacity PBS, 0.1% xanthan gum and 1.3% fructose.

### Results

The inventors of the present application sought to identify novel liquid agricultural compositions of nitrogen-fixing microbes with improved stability at room temperature compared to existing formulations, ideally for a period of at least three months. To do so, four different parameters of the composition were varied: initial cell density, microbial stabilizer, physical stabilizer, and buffering agent. Addressing only a single variable did not lead to the maximal period of microbial stability. Instead, the final agricultural composition achieved greater than four months of bacterial stability at room temperature by addressing all four parameters: selection of an initial cellular density chosen to decrease microbial decay rate; presence of a microbial stabilizer (fructose 1.3%); presence of a physical stabilizer (xanthan gum 0.1%); and selection of a buffering system (high buffering capacity modified PBS).

This exemplary method of modifying the aforementioned combination of four parameters is able to improve microbial stability and decrease accumulation of toxic metabolites produced during storage of an agricultural composition comprising nitrogen-fixing microorganisms. Selecting the proper cell density at time of packaging was a component in producing a stable product. Next, addition of a microbial and physical stabilizer, as well as having a buffer with a high buffering capacity, were all elements in providing long term shelf life. Microbial stabilizers protect the cells by potentially putting them in a semi-dormant state, so that the cells do not respond to environmental conditions as rapidly as they would in an active state; an appropriate buffering system prevents deleterious pH fluctuations; and physical stabilizers create homogeneous solutions, where the cells are at similar density throughout the liquid. Each component individually functioned to improve stability to some extent, but it was the combination of all four factors that provided the most significant long-term improvement in microbial stability.

### Example 2: Formulation of Exemplary Agronomically Stable Liquid Agricultural Composition

The present example provides the formulation steps and components for the exemplary agricultural composition developed according to Example 1.

### Fermentation, Formulation, and Packaging

Fermentation: Klebsiella variicola NCMA 201712002 was fermented at 30° C. in stirred tank reactors (STR) to generate a high cell density robust broth by batch process. Cells were harvested at a cell density of 1.2 to 2.0E10 CFU/mL.

Formulation: After fermentation, cells were diluted in modified, high buffering capacity PBS to a cellular density of 4.5-5.5E9 CFU/mL with fructose and xanthan gum added to final concentrations of 1.3% and 0.1%, respectively. The concentrations of the chemical components of the final product are provided in Table 9.

TABLE 9
Components and concentrations in
final liquid agricultural composition product

| | Concentration |
|---|---|
| Description | (% in final product) |
| Fructose | 1.3 |
| Xanthan gum | 0.1 |
| monobasic KH₂PO₄ | 1.53 |
| NaCl | 0.5 |
| KCl | 0.013 |
| dibasic Na₂HPO₄ | 0.09 |

Packaging: product was packaged and stored in breathable bags/bladders with 20% headspace.

### Bacterial Stability

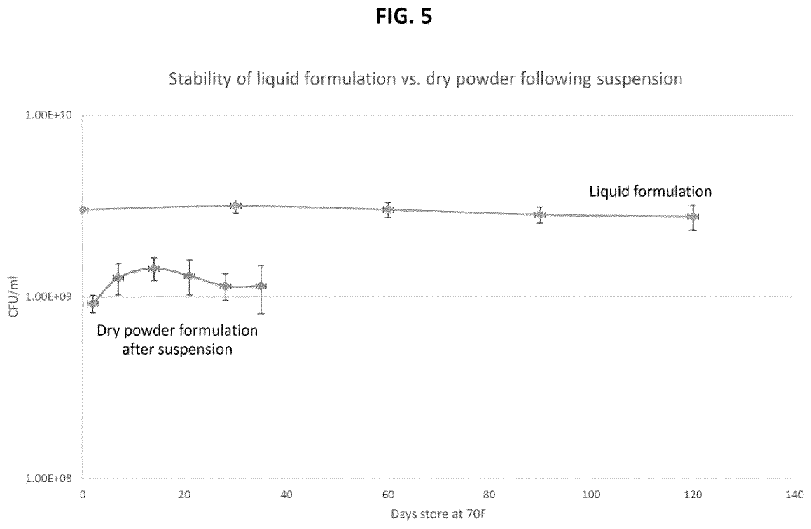

The microbial stability within the liquid agricultural composition was compared to a dry formulation (Pivot Bio PROVEN™ 2019 dry formulation) comprising the same bacterial strain. The liquid agricultural composition was prepared according to the foregoing formulation steps, while the dry formulation was suspended and activated in a liquid medium according to packaging instructions. The microbial stability of the liquid formulation was monitored on a monthly basis for four months, while the bacterial propagation of the activated and suspended dry formulation was monitored for a period of 35 days. FIG. 5 shows the results of this comparison. The liquid formulation was stable, with almost no change in bacterial viability over the entire tested period of four months, while the bacteria within the suspended dry formulation initially grow after activation, but then start to slowly decay after more than 30 days. This is consistent with the packaging instructions for the dry formulation, which recommend use within 30 days after activation. By contrast, the liquid formulation provides a longer stability timeframe within its liquid form, allowing for greater ease of storage and delivery, without the need for activation and use within a short period.

### Bacterial Titer

The improved stability and functionality of the agronomically stable liquid agricultural composition also allowed for an increased bacterial titer within the final product as compared to the dry formulation after liquid suspension. The liquid agricultural composition had a bacterial titer high enough to provide sufficient microbes for 20 acres of land with only 8 L of product-four-fold higher than for the suspended dry powder formulation, which provided sufficient microbes for only 5 acres of land with 8 L of product.

### Example 3: Formulation of Exemplary Agronomically Stable Agricultural Composition Comprising Alternative Bacterium

The formulation ingredients selected in Example 1 and the formulation steps carried out in Example 2 were applied with a different strain of bacterium, Kosakonia sacchari PTA-126743, with the aim of improving microbial stability and shelf life, and decreasing toxin accumulation for the agricultural composition comprising that bacterium.

The PTA-126743 strain agricultural composition was formulated with a 3× diluted initial cell density of about 3E9 CFU/mL, 1.3% fructose, and 0.1% xanthan gum in high buffering capacity PBS. This agricultural composition was compared to the normal fermentation broth in terms of ammonia accumulation, bacterial stability, and pH stability. All samples were tested in triplicate, and the results were averaged.

Average ammonia concentration was measured at the 0 and 3 month time points, with results shown in Table 10. The results demonstrate that the agricultural composition had lower average ammonia concentrations at both time points, with less than half the amount of ammonia accumulation over the course of three months compared to the non-optimized fermentation broth.

TABLE 10
Ammonia accumulation in broth vs. agricultural
composition comprising Kosakonia sacchari PTA-126743.

| | | Initial | 3 Mo. | | 3 Mo. |
|---|---|---|---|---|---|
| | | Avg. | Avg. | 3 Mo. | Change |
| | | [NH4+] | [NH4+] | Std. | [NH4+] |
| Formulation | Strain | (mM) | (mM) | Dev. | (mM) |
| Broth | PTA-126743 | 78.7 | 95.8 | 2.3 | 17.2 |
| Agricultural | PTA-126743 | 20.7 | 28.6 | 4.3 | 7.8 |
| composition | | | | | |

The bacterial stability was also compared between the broth and the agricultural composition, results shown in Table 11. Cell density was measured at 0, 1, and 3 months, and the 1 month log loss and 3 month log loss were computed based on these values. The initial cell density of the agricultural composition was one third that of the broth because of the dilution step within the formulation of the agricultural composition. At 1 month, the broth experienced 0.93 log loss of CFU/mL, while the agricultural composition experienced only 0.22 log loss of CFU/mL. At 3 months, the broth experienced 1.28 log loss of CFU/mL compared to the 0.69 log loss of CFU/mL for the agricultural composition, such that the agricultural composition experienced roughly 50% less viability loss over the 3 month time period. By the 3 month time point, the cell density of the agricultural composition was higher than that of the broth, even though it started out as a one third dilution, because of the much lower decay rate. In fact, due to some measurement uncertainty, it is believed that the cell density of the agricultural composition at 3 months was even higher than reported.

TABLE 11
Bacterial stability in broth vs. agricultural composition
comprising Kosakonia sacchari PTA-126743.

| | | | | | 1 | | 3 |
|---|---|---|---|---|---|---|---|
| | | Stor- | | | month | | month |
| For- | | age | Initial | 1 month | log | 3 month | log |
| mulation | Strain | Temp | CFU/ml | CFU/ml | loss | CFU/ml | loss |
| Broth | PTA- | RT | 7.83E+09 | 9.24E+08 | 0.93 | 4.12E+08 | 1.28 |
| | 126743 | | | | | | |
| Agricul- | PTA- | RT | 2.72E+09 | 1.64E+09 | 0.22 | 5.55E+08 | 0.69 |
| tural | 126743 | | | | | | |
| com- | | | | | | | |
| position | | | | | | | |

The pH stability of the agricultural composition over the 3 month time period was also compared to that of the broth, results shown in Table 12. Overall, the pH of the agricultural composition changed by less than 0.3 at either time point compared to the original pH, while the pH of the broth continued to increase over the 3 month window, with a final difference of 1.31 compared to the initial pH measurement.

TABLE 12
Bacterial stability in broth vs. agricultural composition
comprising Kosakonia sacchari PTA-126743.

| | | | | | | | 3 |
|---|---|---|---|---|---|---|---|
| | | Stor- | | 1 | 1 month | 3 | month |
| For- | | age | Initial | month | pH | month | pH |
| mulation | Strain | Temp | pH | pH | change | pH | change |
| Broth | PTA- | RT | 6.74 | 7.72 | 0.98 | 8.05 | 1.31 |
| | 126743 | | | | | | |
| Agricultural | PTA- | RT | 6.52 | 6.25 | −0.27 | 6.73 | 0.21 |
| composition | 126743 | | | | | | |

These results show that the agricultural composition maintained its microbial viability, pH level, and low ammonia concentration better than the non-optimized fermentation broth over the course of 3 months.

### Example 4: Field Application of Exemplary Liquid Agricultural Composition

The exemplary agronomically stable liquid agricultural composition developed according to the method of Example 1 and prepared according to the formulation steps of Example 2 was evaluated in eight corn fields.

### Data Collection

Corn yields with the grower standard nitrogen fertilization practice were compared to corn yields with the liquid agricultural composition added to the system as an in-furrow application at planting. These trials took place in 4 different states: 3 in Illinois, 2 in Missouri, 2 in Minnesota, and 1 in Iowa. Farmers were instructed to split fields in three with the liquid agricultural composition on one third of the field, the Grower Standard Practice (untreated control) on the next third, and a suspended and activated dry formulation (Pivot Bio PROVEN™, as referenced in Example 2) on the remaining third.

Trial participants provided digital as-planted (planter monitor) and harvest (combine yield monitor) maps identifying the two treatment zones and the control zone. ArcGIS software was used to analyze data and compare yield differences between the zones.

The yield data from the harvest combine monitors were used to examine changes in yield variability between liquid formulation-treated field areas, suspended dry formulation-treated field areas, and untreated control areas by analyzing the yield homogeneity of variance and standard deviation.

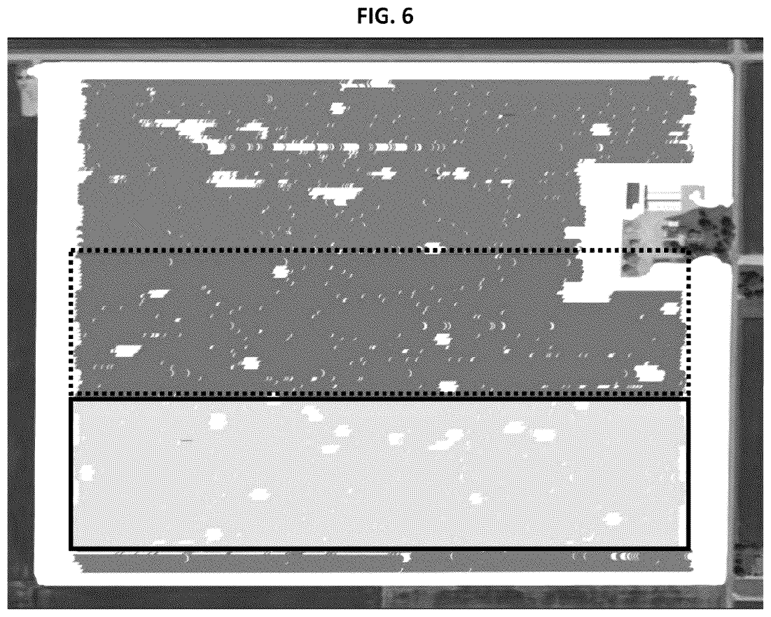

Additional QA/QC procedures were applied to combine data ensuring representative comparisons from both treated and untreated field regions. Header rows, which are typically lower in yield, more prone to damage and have a varying incident solar radiation profile, were removed from field data sets. This can be seen in FIG. 6, which shows the data collection and quality control for an exemplary corn field. In this figure, the perimeter of the field is white, showing areas that were excluded from the data analysis. In addition, the most reliable data from combine harvest monitors occurs in areas where the combine is moving at a steady velocity. Thus, data points were removed with automated filters in areas where the combine was accelerating or where the combine had to slow down to pass obstacles like field drains or terraces—these are shown as white patches within the illustrative field depicted in FIG. 6. A total of 54,623 yield harvest data points total were analyzed in this field. Table 13 contains the summary of data points and acreage collected for the treatment and control areas across all 8 trials.

TABLE 13
Data points by treatment type across the 8 field trials.

| | Untreated | Suspended dry | Liquid | |
|---|---|---|---|---|
| | Control | formulation | formulation | Total |
| Total monitor | 385,361 | 188,451 | 182,231 | 756,043 |
| data points | | | | |
| Total area | 637 | 293 | 272 | 1,202 |
| (acres) | | | | |

### Results

The performance of the exemplary liquid agricultural composition was evaluated along two metrics: average yield within a field and yield consistency across the field.

Increasing average yield in a field increases the overall production. Furthermore, uniform crop development is an important factor in maximizing yields and an important driver of within field yield variance. Corn is more responsive to nitrogen than other nutrients. Consequently, differences in nitrogen availability within fields contribute greatly to yield variance. The agronomically stable liquid agricultural compositions of the present disclosure can serve as a baseline nitrogen source that doesn't leach from the soil and that delivers nitrogen to the corn plant in a more consistent and reliable manner compared to traditional synthetic nitrogen sources.

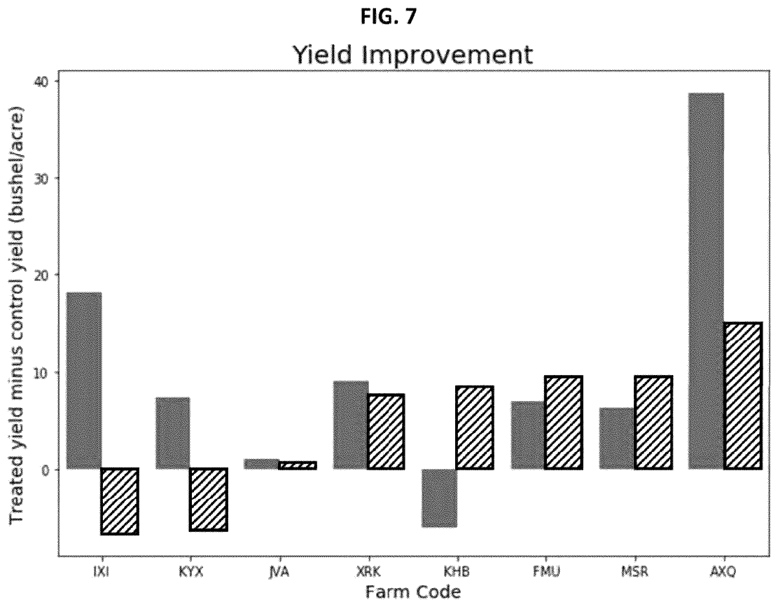

In terms of average yield, the dry formulation has shown a 76% win rate for increasing yields across 37 large scale field trials, with an average improvement of 5.8 bushels/acre. The exemplary liquid agricultural composition was tested in a smaller set of 8 field trials side by side with the dry formulation. It showed a similar win rate of 75% and an average improvement of 4.7 bushels/acre, as shown in FIG. 7. The activated dry formulation had a yield improvement of 10.2 bushels/acre and a win rate of 88% in the same subset of trials. This shows that the liquid agricultural composition achieved a similar win rate and average yield improvement compared to the overall win rate and average yield improvement for the commercially available dry formulation, while having improved liquid shelf stability and ease of transportation, storage, and use.

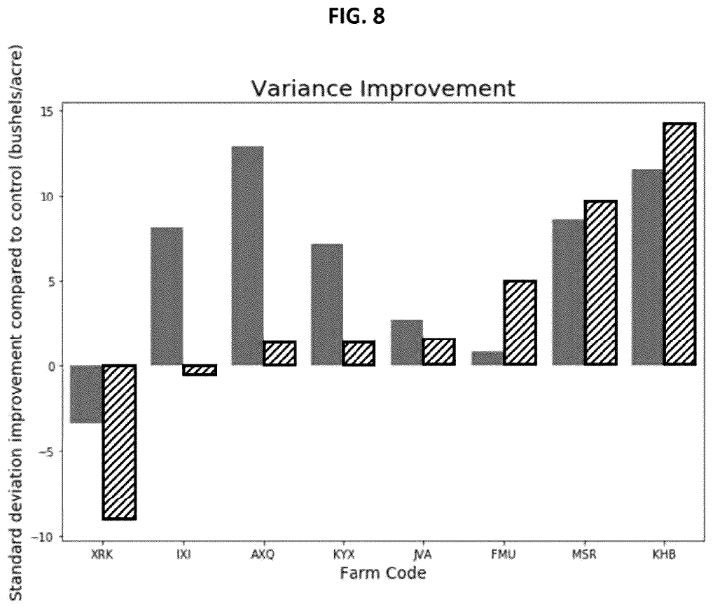

In terms of yield consistency, the dry formulation has improved uniform yield consistency in 70% of the 37 large scale field trials by reducing the standard deviation of the yield points in treated areas. FIG. 8 shows a similar yield variance improvement for the exemplary liquid agricultural composition, which achieved a win rate of 75% and a variance improvement of 2.9 bushels/acre in the 8 trials. The dry formulation showed a variance improvement of 6.1 bushels/acre and a win rate of 88% in the same trials. This comparison shows that the liquid agricultural composition achieved a higher win rate for yield variance improvement than the historical average for the commercially available dry formulation.

Overall, in its 8 field trials, the liquid agricultural composition performed comparably to the historical average for the commercially available dry formulation comprising the same strain of nitrogen-fixing gram negative bacteria. These similar results were achieved in spite of the fact that the liquid agricultural composition was two-months old upon application to the fields compared to the suspended dry formulation, which was freshly activated. These trial demonstrate the suitability of the liquid agricultural composition for use in improving the average yield and yield consistency of corn crops. Furthermore, it accomplished these results without the need for activation and suspension by the farmers, and with much greater shelf stability in liquid form.

### Example 5: Wheat Field Application of Exemplary Liquid Agricultural Composition

The exemplary agronomically stable liquid agricultural composition developed according to the method of Example 1 and prepared according to the formulation steps of Example 2 was evaluated in ten trial locations in common spring wheat production areas.

### Data Collection

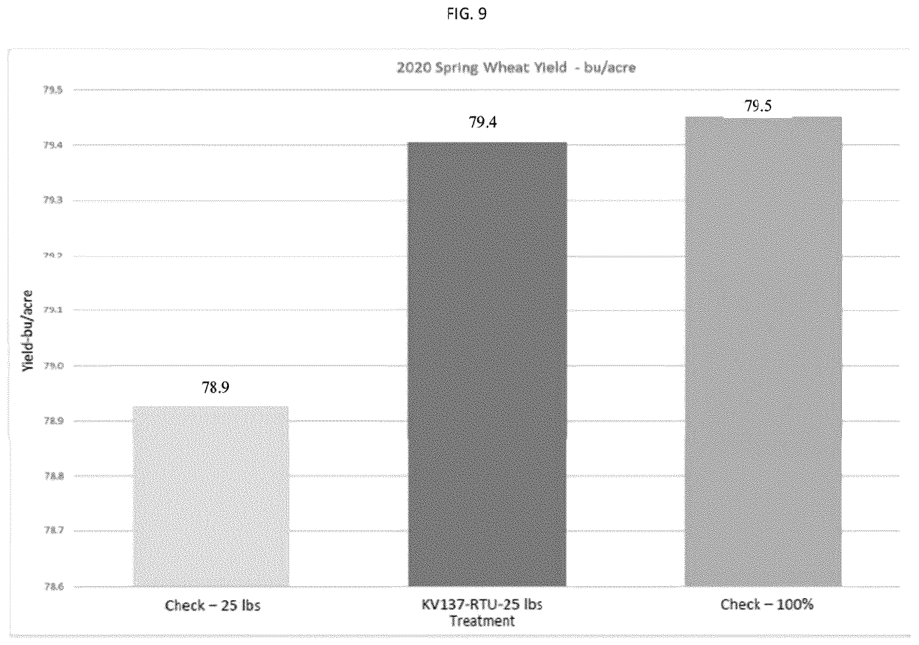

Wheat yields with the grower standard nitrogen fertilization practice were compared to wheat yields with the liquid agricultural composition added to the system as an in-furrow application at planting. The trial was a large replicated complete block design with four repetitions. There was one untreated control which received 100% of the recommended nitrogen rate (labeled “check 100%” in FIG. 9). The other treatments, included a reduced nitrogen control, in which nitrogen was reduced by 25 pounds and a experimental treatment in which the reduced nitrogen control sample was supplemented with the liquid agricultural composition (nontreated control labeled “check −25 lbs” and treated labeled “KV137-RTU-25 lbs” in FIG. 9).

### Results

The performance of the exemplary liquid agricultural composition was evaluated by average yield in bushels per acre (FIG. 9). In the reduced nitrogen fertilizer setting, KV137-RTU had an average yield response 0.6 bu/acre higher compared to the reduced nitrogen “check-25 lbs” and was within 0.1 bu/acre of wheat treated with the grower standard nitrogen fertilization practice.

Overall, these trials demonstrate the suitability of the liquid agricultural composition for use in producing wheat with reduced nitrogen fertilizer.

### Example 6: Sorghum Field Application of Exemplary Liquid Agricultural Composition

The exemplary agronomically stable liquid agricultural composition developed according to the method of Example 1 and prepared according to the formulation steps of Example 2 was evaluated in ten trial locations in common grain sorghum production areas.

### Data Collection

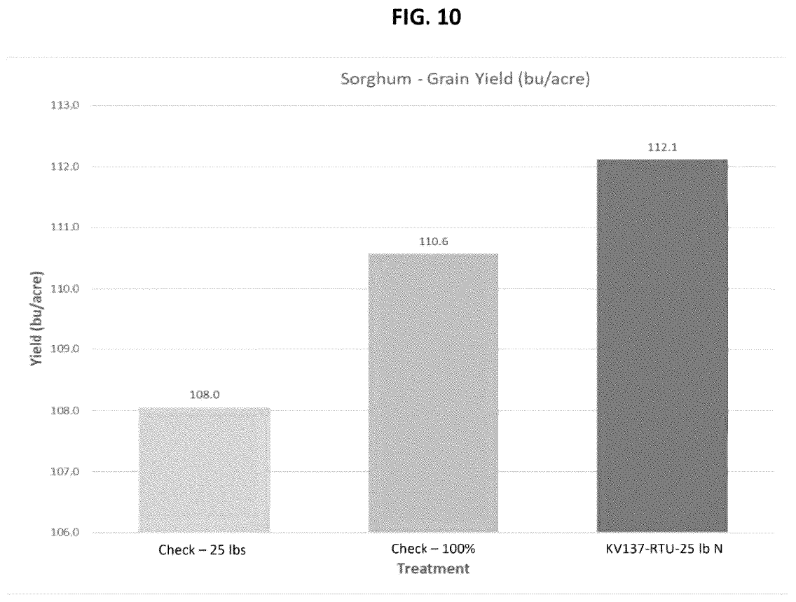

Sorghum yields with the grower standard nitrogen fertilization practice were compared to sorghum yields with the liquid agricultural composition added to the system as an in-furrow application at planting. The trial was a large replicated complete block design with four repetitions. There was one untreated control which received 100% of the recommended nitrogen rate (labeled “check 100%” in FIG. 10). The other treatments, included a nontreated control and an experimental treatment, were reduced by 25 pounds of nitrogen alone, or in the case of the experimental treatment, were supplemented with the liquid nutritional composition (nontreated control labeled “check-25 lbs” and treated labeled “KV137-RTU-25 lbs” in FIG. 10).

### Results

The performance of the exemplary liquid agricultural composition was evaluated by average yield in bushels per acre (FIG. 10). In the reduced nitrogen fertilizer setting, KV137-RTU had an average yield increase of 4.1 bu/acre over the “check-25 lbs” with a win rate of 71% and a 1.5 bu/acre advantage over the sorghum treated with the grower standard nitrogen fertilization practice with a win rate of 30%.

Overall, these trials demonstrate the suitability of the liquid agricultural composition for use in producing sorghum with reduced nitrogen fertilizer.

### Example 7: Formulation of Exemplary Agronomically Stable Liquid Agricultural Composition

The present example provides the formulation components for an exemplary agricultural composition developed with K. sacchari PTA-126743.

### Fermentation, Formulation, and Packaging

Fermentation: K. sacchari PTA-126743 was fermented at 30° C. in stirred tank reactors (STR) to generate a high cell density robust broth by batch process.

Formulation: After fermentation, cells were diluted in modified, high buffering capacity PBS. Formulations were generated with different combinations of stabilizers (3 stabilizers each at 2 concentrations), one physical stabilizer and 2 carrier buffers (water as control and optimized PBS buffer). Specifically, combinations of fructose (1.3%), xanthan gum (0.1%), sucrose (1.3% or 2.5%), inulin (1.3% or 2.5%), and 200 mM phosphate, all in comparison with a control having no buffer or stabilizer.

Packaging: product was packaged and stored in breathable bags/bladders with 20% headspace.

### Bacterial Stability

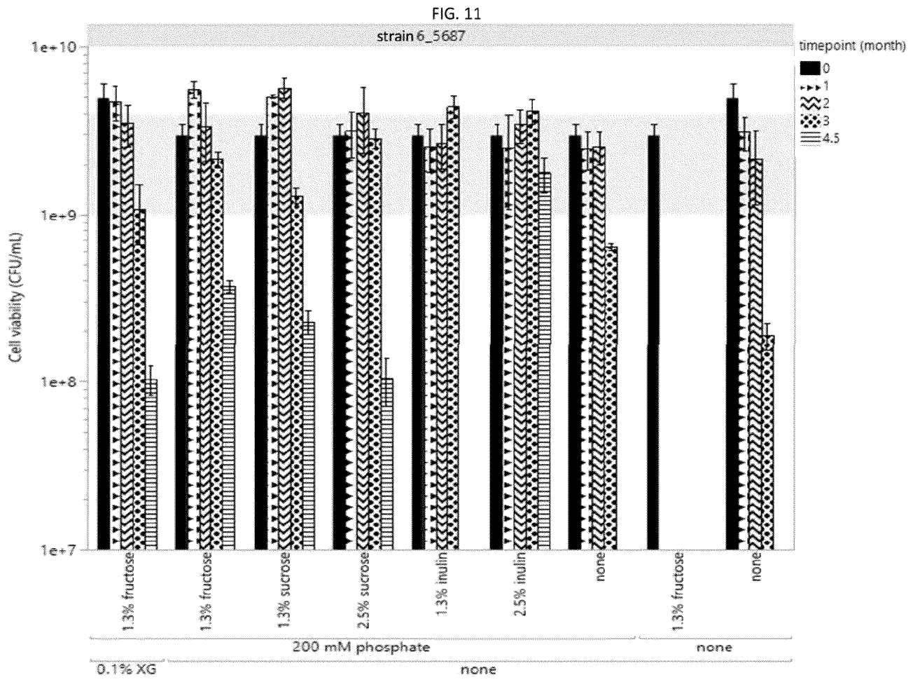

Stability of formulated material was monitored at room temperature for up to 4.5 months with monthly sample taken and analyzed for viability, pH and purity (cell viability (CFU/mL) shown in FIG. 11). The results indicate that a microbial stabilizer and buffer can maintain viability of the product beyond 3 months.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

### NUMBERED EMBODIMENTS OF THE INVENTION

Notwithstanding the appended claims, the disclosure sets forth the following numbered embodiments:

1. An agronomically stable liquid agricultural composition, comprising:
a) a diazotrophic bacterium;
b) a buffering agent;
c) a microbial stabilizer; and
d) a physical stabilizer,
wherein the composition has a room temperature shelf life of at least 30 days.

2. The composition of embodiment 1, wherein the microbial stability of the composition is greater than the microbial stability of the composition absent one or more of the buffering agent, microbial stabilizer, and physical stabilizer.

3. The composition of any one of embodiments 1-2, wherein the shelf life is at least two months, at least three months, at least four months, or at least five months.

4. The composition of any one of embodiments 1-3, wherein the shelf life is at least three months.

5 The composition of any one of embodiments 1-4, wherein the log loss of CFU/mL over the shelf life of the composition is less than 0.2.

6. The composition of any one of embodiments 1-5, wherein the bacterium is present at a cellular density that provides an acceptable rate of decay over the shelf life of the composition.

7. The composition of any one of embodiments 1-6, wherein the bacterium is present at a cellular density that minimizes the rate of decay over the shelf life of the composition.

8. The composition of any one of embodiments 1-7, wherein the bacterium is present at a cellular density that provides a reduced, but not minimized rate of decay.

9. The composition of any one of embodiments 1-8, wherein the bacterium is present at a cellular density that provides a rate of decay of less than 1.0 log loss of CFU/mL over 30 days in the agricultural composition absent the buffering agent, microbial stabilizer, and physical stabilizer.

10. The composition of any one of embodiments 1-9, wherein the bacterium is present at a cellular density of about 3E9-6E9 CFU/mL.

11. The composition of any one of embodiments 1-10, wherein the buffering agent maintains the pH of the composition over the shelf life of the composition.

12. The composition of any one of embodiments 1-11, wherein the buffering agent maintains the pH of the composition at about pH 6-8 over the shelf life of the composition.

13. The composition of any one of embodiments 1-12, wherein the buffering agent maintains the pH of the composition at about pH 6.5 over the shelf life of the composition.

14. The composition of any one of embodiments 1-13, wherein the buffering agent is selected from the list consisting of phosphate buffered saline (PBS); modified, high buffering capacity PBS; 3-Morpholinopropane-1-sulfonic acid (MOPS); and 2-[4-(2-hydroxyethyl)piperazin-1-yl]ethanesulfonic acid (HEPES).

15. The composition of any one of embodiments 1-14, wherein the buffering agent is modified, high buffering capacity PBS.

16. The composition of any one of embodiments 1-15, wherein the microbial stabilizer slows the doubling rate of the diazotrophic bacterium.

17. The composition of any one of embodiments 1-16, wherein the microbial stabilizer slows the toxin accumulation rate within the composition.

18. The composition of any one of embodiments 1-17, wherein the microbial stabilizer is a monosaccharide, disaccharide, polysaccharide, pentose, hexose, oligosaccharide, oligofructose, sugar alcohol, amino acid, protein or protein hydrolysate, or polymer.

19. The composition of any one of embodiments 1-18, wherein the microbial stabilizer is a monosaccharide or a disaccharide selected from the list consisting of glucose, fructose, trehalose, sucrose, lactose, melibiose, and lactulose.

20. The composition of any one of embodiments 1-19, wherein the microbial stabilizer is fructose or trehalose.

21. The composition of any one of embodiments 1-20, wherein the microbial stabilizer is fructose.

22. The composition of any one of embodiments 1-21, wherein the microbial stabilizer is fructose and is present in the composition at a concentration of about 0.5-2.5% w/v.

23. The composition of any one of embodiments 1-22, wherein the microbial stabilizer is fructose and is present in the composition at a concentration of about 1.3% w/v.

24. The composition of any one of embodiments 1-23, wherein the physical stabilizer decreases the local density of the diazotrophic bacterium within the composition.

25. The composition of any one of embodiments 1-24, wherein the physical stabilizer is a polysaccharide, protein or protein hydrolysate, polymer, or a natural gum or its derivative.

26. The composition of any one of embodiments 1-25, wherein the physical stabilizer is a polysaccharide.

27. The composition of any one of embodiments 1-26, wherein the physical stabilizer is a polysaccharide selected from the list consisting of maltodextrin, polyethylene glycol (PEG), xanthan gum, pectin, alginates, microcrystalline cellulose, and dextran.

28. The composition of any one of embodiments 1-27, wherein the physical stabilizer is xanthan gum.

29. The composition of any one of embodiments 1-28, wherein the physical stabilizer is xanthan gum and is present in the composition at a concentration of about 0.001-0.2% w/v.

30. The composition of any one of embodiments 1-29, wherein the physical stabilizer is xanthan gum and is present in the composition at a concentration of about 0.1% w/v.

31. The composition of any one of embodiments 1-30, wherein the bacterium is a gram-negative bacterium.

32. The composition of any one of embodiments 1-31, wherein the bacterium is of a genus selected from the group consisting of: Acetobacter, Achromobacter, Aerobacter, Anabaena, Azoarcus, Azomonas, Azorhizobium, Azospirillum, Azotobacter, Beijernickia, Bradyrhizobium, Burkholderia, Citrobacter, Derxia, Enterobacter, Herbaspirillum, Klebsiella, Kluyvera, Kosakonia, Mesorhizobium, Metakosakonia, Paraburkholderia, Nostoc, Rahnella, Rhizobium, Rhodobacter, Rhodopseudomonas, Rhodospirillum, Serratia Sinorhizobium, Spirillum, Trichodesmium, and Xanthomonas.

33. The composition of any one of embodiments 1-32, wherein the bacterium is of a species selected from the group consisting of: Achromobacter marplatensis, Achromobacter spiritinus, Azospirillum lipoferum, Enterobacter sacchari, Herbaspirillum aquaticum, Klebsiella variicola, Kluyvera intermedia, Kosakonia pseudosacchari, Kosakonia sacchari, Metakosakonia intestini, Paraburkholderia tropica, Rahnella aquatilis, and combinations thereof.

34. The composition of any one of embodiments 1-33, wherein the bacterium is a gram-positive bacterium.

35. The composition of any one of embodiments 1-34, wherein the bacterium is of a genus selected from the group consisting of: Arthrobacter, Agromyces, Bacillus, Clostridium, Corynebacterium, Frankia, Heliobacillus, Heliobacterium, Heliophilum, Heliorestis Methanobacterium, Microbacterium, Micrococcus, Micromonospora, Mycobacterium, Paenibacillus, Propionibacterium, and Streptomyces.

36. The composition of any one of embodiments 1-35, wherein the bacterium is of a species selected from the group consisting of: Bacillus amyloliquefaciens, Bacillus macerans, Bacillus pumilus, Bacillus thuringiensis, Clostridium acetobutylicum, Corynebacterium autitrophicum Methanobacterium formicicum, Methanobacterium omelionski, Microbacterium murale, Mycobacterium flavum, Paenibacillus polymyxa, Paenibacillus riograndensis, Propionibacterium acidipropio, Propionibacterium freudenreichii, Streptococcus lactis, Streptomyces griseus, and combinations thereof.

37. The composition of any one of embodiments 1-36, wherein the bacterium is of the genus Klebsiella.

38. The composition of any one of embodiments 1-37, wherein the bacterium is of the species Klebsiella variicola.

39. The composition of any one of embodiments 1-38, wherein the bacterium is of the strain Klebsiella variicola NCMA 201712002.

40. The composition of any one of embodiments 1-39, wherein the bacterium is of the genus Kosakonia.

41. The composition of any one of embodiments 1-40, wherein the bacterium is of the species Kosakonia sacchari.

42. The composition of any one of embodiments 1-41, wherein the bacterium is of the strain Kosakonia sacchari PTA-126743.

43. The composition of any one of embodiments 1-42, wherein the bacterium is endophytic, epiphytic, or rhizospheric.

44. The composition of any one of embodiments 1-43, wherein the bacterium is a wild type bacterium.

45. The composition of any one of embodiments 1-44, wherein the bacterium is an engineered bacterium.

46. The composition of any one of embodiments 1-45, wherein the bacterium is a transgenic bacterium.

47. The composition of any one of embodiments 1-46, wherein the bacterium is an intragenic bacterium.

48. The composition of any one of embodiments 1-47, wherein the bacterium is a remodeled bacterium.

49 The composition of any one of embodiments 1-48, wherein the bacterium comprises a non-intergeneric genomic modification.

50. The composition of any one of embodiments 1-49, wherein the bacterium is an engineered bacterium capable of fixing atmospheric nitrogen in the presence of exogenous nitrogen.

51. The composition of any one of embodiments 1-50, wherein the bacterium is an engineered bacterium comprising at least one genetic variation introduced into at least one gene, or non-coding polynucleotide, of the nitrogen fixation or assimilation genetic regulatory network.

52. The composition of any one of embodiments 1-51, wherein the bacterium is an engineered bacterium comprising an introduced control sequence operably linked to at least one gene of the nitrogen fixation or assimilation genetic regulatory network.

53. The composition of any one of embodiments 1-52, wherein the bacterium is an engineered bacterium comprising a heterologous promoter operably linked to at least one gene of the nitrogen fixation or assimilation genetic regulatory network.

54. The composition of any one of embodiments 1-53, wherein the bacterium is an engineered bacterium comprising at least one genetic variation selected from the group consisting of: nifA, nifL, ntrB, ntrC, polynucleotide encoding glutamine synthetase, glnA, glnB, glnK, drat, amtB, polynucleotide encoding glutaminase, glnD, glnE, nifJ, nifH, nifD, nifK, nifY, nifE, nifN, nifU, nifS, nifV, nifW, nifZ, nifM, nifF, nifB, nifQ, a gene associated with biosynthesis of a nitrogenase enzyme, and combinations thereof.

55. The composition of any one of embodiments 1-54, wherein the bacterium is an engineered bacterium comprising at least one genetic variation introduced into at least one gene, or non-coding polynucleotide, of the nitrogen fixation or assimilation genetic regulatory network that results in one or more of: increased expression or activity of NifA or glutaminase; decreased expression or activity of NifL, NtrB, glutamine synthetase, GlnB, GlnK, DraT, AmtB; decreased adenylyl-removing activity of GlnE; or decreased expression or uridylyl-removing activity of GlnD.

56. The composition of any one of embodiments 1-55, wherein the bacterium is an engineered bacterium comprising a mutated nifL gene that has been altered to comprise a heterologous promoter inserted into said nifL gene.

57. The composition of any one of embodiments 1-56, wherein the bacterium is an engineered bacterium comprising a mutated glnE gene that results in a truncated GlnE protein lacking an adenylyl-removing (AR) domain.

58. The composition of any one of embodiments 1-57, wherein the bacterium is an engineered bacterium comprising a mutated glnD gene that results in the lack of expression of said glnD gene.

59. The composition of any one of embodiments 1-58, wherein the bacterium is an engineered bacterium comprising a mutated amtB gene that results in the lack of expression of said amtB gene.

60 The composition of any one of embodiments 1-59, wherein the bacterium is an engineered bacterium comprising at least one of: a mutated nifL gene that has been altered to comprise a heterologous promoter inserted into said nifL gene; a mutated glnE gene that results in a truncated GlnE protein lacking an adenylyl-removing (AR) domain; a mutated amtB gene that results in the lack of expression of said amtB gene; a mutated glnD gene that results in the lack of expression of said glnD gene; and combinations thereof.

61. The composition of any one of embodiments 1-60, wherein the bacterium is an engineered bacterium comprising at least one genetic variation introduced into genes involved in a pathway selected from the group consisting of: exopolysaccharide production, endo-polygalaturonase production, trehalose production, and glutamine conversion.

62. The composition of any one of embodiments 1-61, wherein the bacterium is an engineered bacterium comprising at least one genetic variation introduced into genes selected from the group consisting of: bcsii, bcsiii, yjbE, fhaB, pehA, otsB, treZ, glsA2, and combinations thereof.

63. The composition of any one of embodiments 1-62, wherein the bacterium is selected from Table 1, or a variant, mutant, or derivative thereof.

64. The composition of any one of embodiments 1-63, wherein the bacterium comprises a nucleic acid sequence that shares at least about 90%, 95%, or 99% sequence identity to a nucleic acid sequence selected from SEQ ID NOs: 1-223.

65. The composition of any one of embodiments 1-64, wherein the bacterium comprises a nucleic acid sequence selected from SEQ ID NOs: 1-223.

66. An agronomically stable liquid agricultural composition with a room temperature shelf life of at least 3 months, comprising:
a) a diazotrophic bacterium at a cellular density that provides a decay rate of less than 0.2 log loss CFU/mL over the shelf life of the composition;
b) a buffering agent that maintains the pH of the composition around pH 6.7 over the shelf life of the composition;
c) a microbial stabilizer that slows the doubling rate of the diazotrophic bacterium; and
d) a physical stabilizer that decreases the local density of the diazotrophic bacterium within the composition,
wherein the stability of the composition is greater than, and the presence of toxic byproducts is less than, the composition absent one or more of the buffering agent, microbial stabilizer, and physical stabilizer.

67. An agronomically stable liquid agricultural composition with a room temperature shelf life of at least 3 months, comprising:
a) Klebsiella variicola at a concentration of at least about 4.5E9 CFU/mL;
b) modified, high buffering capacity PBS;
c) fructose at a concentration of at least about 1% w/v; and
d) xanthan gum at a concentration of at least about 0.05% w/v.

68. An agronomically stable liquid agricultural composition with a room temperature shelf life of at least 3 months, comprising:
a) Kosakonia sacchari at a concentration of about 3E9 CFU/mL;
b) modified, high buffering capacity PBS; and
c) fructose at a concentration of at least about 1% w/v.

69. The composition of any one of embodiments 1-68, wherein the bacterium is a non-intergeneric remodeled bacterium.

70. Agricultural plant tissue comprising the agronomically stable liquid agricultural composition of any of embodiments 1-69.

71. The tissue of embodiment 70, wherein the agricultural plant is a legume or cereal grain.

72. The tissue of embodiment 70 or 71, wherein the agricultural plant is alfalfa, clover, bean, pea, chickpea, lentil, lupin, mesquite, carob, soybean, peanut, rooibos, or tamarind.

73. The tissue of any one of embodiments 70-72, wherein the agricultural plant is corn, rice, wheat, barley, sorghum, millet, oats, or rye.

74. The tissue of any one of embodiments 70-73, wherein the agricultural plant is corn.

75. A method for applying a diazotrophic bacterium to agricultural plant tissues comprising applying the composition of any one of embodiments 1-69 to agricultural plant tissues or the environs thereof.

76. A method for maintaining a population of a diazotrophic bacterium on an agricultural plant tissue comprising applying the composition of any one of embodiments 1-69 to said plant tissue or the environs thereof.

77. A method of increasing agricultural plant crop yield comprising applying the composition of any one of embodiments 1-69 to the agricultural plant tissues or the environs thereof prior to, during, or immediately following planting, thereby increasing the crop yield of the agricultural plant once planted.

78. A method of providing fixed atmospheric nitrogen to a cereal plant, comprising applying the composition of any one of embodiments 1-69 to the cereal plant tissues or the environs thereof.

79. A method of providing fixed atmospheric nitrogen to a corn plant that eliminates the need for the addition of in-season exogenous nitrogen application, comprising applying the composition of any one of embodiments 1-69 to the corn plant tissues or the environs thereof.

80. A method for increasing corn yield per acre, comprising applying the composition of any one of embodiments 1-69 to the corn plant tissues or the environs thereof.

81. A method for reducing infield variability for corn yield per acre, comprising applying the composition of any one of embodiments 1-69 to the corn plant tissues or the environs thereof, wherein the standard deviation of corn mean yield measured in bushels per acre is lower than for control plants to which the composition has not been applied.

82. The method of any one of embodiments 75-81, wherein the method comprises:
a) applying the composition to a locus; and
b) providing to the locus a plurality of the plants.

83. The method of any one of embodiments 75-82, wherein the composition comprises a plurality of said diazotrophic bacteria, wherein the diazotrophic bacteria comprise engineered bacteria, and wherein the engineered bacteria colonize the root surface of said plurality of plants and supply the plants with fixed nitrogen.

84. The method of any one of embodiments 75-83, wherein the composition comprises a plurality of said diazotrophic bacteria, wherein the diazotrophic bacteria comprise engineered bacteria, and wherein the engineered bacteria colonize the root surface of said plurality of plants and supply the plants with fixed nitrogen, and wherein the plurality of engineered bacteria produce in the aggregate at least about 15 pounds of fixed nitrogen per acre over the course of at least about 10 days to about 60 days.

85. The method of any one of embodiments 75-84, wherein exogenous nitrogen is not applied to the plant tissues or the environs thereof after the composition is applied.

86. The method of any one of embodiments 75-85, wherein the agricultural plant is a legume or cereal grain.

87. The method of any one of embodiments 75-86, wherein the agricultural plant is alfalfa, clover, bean, pea, chickpea, lentil, lupin, mesquite, carob, soybean, peanut, rooibos, or tamarind.

88. The method of any one of embodiments 75-87, wherein the agricultural plant is corn, rice, wheat, barley, sorghum, millet, oats, or rye.

89. The method of any one of embodiments 75-88, wherein the agricultural plant is corn.

90. The method of any one of embodiments 75-89, wherein the method increases the crop yield of the agricultural plant.

91. The method of any one of embodiments 75-90, wherein the method increases the crop yield of the agricultural plant with a win rate of greater than 65%.

92. The method of any one of embodiments 75-91, wherein the method increases the crop yield of the agricultural plant with a win rate of about 75%.

93. The method of any one of embodiments 75-92, wherein the method increases the crop yield of the agricultural plant by more than 3 bushels/acre.

94. The method of any one of embodiments 75-93, wherein the method increases the crop yield of the agricultural plant by about 3 bushels/acre.

95. The method of any one of embodiments 75-94, wherein the method reduces infield variability for the agricultural plant crop yield per acre.

96. The method of any one of embodiments 75-95, wherein the method reduces infield variability for the agricultural plant crop yield per acre with a win rate of greater than 65%.

97. The method of any one of embodiments 75-96, wherein the method reduces infield variability for the agricultural plant crop yield per acre with a win rate of about 75%.

98. The method of any one of embodiments 75-97, wherein the method reduces infield variability for the agricultural plant crop yield per acre with a variance improvement of greater than 2 bushels/acre.

99. A method of preparing an agronomically stable liquid agricultural composition comprising a diazotrophic bacterium, the method comprising the steps of:
a) providing a diazotrophic bacterium;
b) selecting for inclusion in the composition a cellular density of the diazotrophic bacterium that provides an acceptable rate of decay of the bacterium;
c) selecting a buffering agent for inclusion in the composition;
d) selecting a microbial stabilizer for inclusion in the composition; and
e) selecting a physical stabilizer for inclusion in the composition,
wherein the composition is stable at room temperature for a period of more than 30 days, and wherein the stability of the composition is greater than the composition absent one or more of the buffering agent, microbial stabilizer, an physical stabilizer.

100. A method for improving the stability of a liquid agricultural composition comprising a diazotrophic bacterium, the method comprising the steps of
a) providing a diazotrophic bacterium;
b) selecting for inclusion in the composition a cellular density of the diazotrophic bacterium that provides an acceptable rate of decay of the bacterium;
c) selecting a buffering agent for inclusion in the composition;
d) selecting a microbial stabilizer for inclusion in the composition; and
e) selecting a physical stabilizer for inclusion in the composition, wherein the composition has a room temperature shelf life of at least 30 days.

101. The method of embodiment 99 or 100, wherein step (b) comprises generating a titration curve to determine cellular density versus decay rate of the diazotrophic bacterium and selecting a cellular density that provides an acceptable decay rate.

102. The method of any one of embodiments 99-101, wherein steps (c)-(e) can be performed in any order.

103. The method of any one of embodiments 99-102, wherein any subset of steps (c)-(e) can be performed serially or in parallel.

104. The method of any one of embodiments 99-103, wherein step (c) comprises selecting a buffering agent that improves microbial stability when included in the agricultural composition, either in the presence or absence of the microbial and/or physical stabilizer.

105. The method of any one of embodiments 99-104, wherein step (c) comprises comparing two or more buffering agents and selecting the buffering agent that provides greater improvement to the microbial stability when included in the agricultural composition, either in the presence or absence of the microbial and/or physical stabilizer.

106. The method of any one of embodiments 99-105, wherein step (d) comprises selecting a microbial stabilizer that improves microbial stability when included in the agricultural composition, either in the presence or absence of the buffering agent and/or physical stabilizer

107. The method of any one of embodiments 99-106, wherein step (d) comprises comparing two or more microbial stabilizers and selecting the microbial stabilizer that provides greater improvement to the microbial stability when included in the agricultural composition, either in the presence or absence of the buffering agent and/or physical stabilizer.

108. The method of any one of embodiments 99-107, wherein step (e) comprises selecting a physical stabilizer that improves microbial stability when included in the agricultural composition, either in the presence or absence of the buffering agent and/or microbial stabilizer.

109. The method of any one of embodiments 99-108, wherein step (e) comprises comparing two or more physical stabilizers and selecting the physical stabilizer that provides greater improvement to the microbial stability when included in the agricultural composition, either in the presence or absence of the buffering agent and/or microbial stabilizer.

110. The method of any one of embodiments 99-109, wherein the selections of the microbial stabilizer and the physical stabilizer in steps (d) and (e) are performed simultaneously.

111. The method of any one of embodiments 99-110, wherein the method increases the shelf life of the liquid agricultural composition by a factor of at least 2, at least 3, or at least 4.

112. The method of any one of embodiments 99-111, wherein any one of steps (b)-(e) alone provides less improvement to the microbial stability of the composition than all of the steps together.

113. The method of any one of embodiments 99-112, wherein the composition is for application to agricultural plant tissues or the environs thereof.

114. The method of any one of embodiments 99-113, wherein the method decreases the accumulation of toxic byproducts in the composition over the course of its shelf life.

115. The method of any one of embodiments 99-114, wherein the method decreases the accumulation of ammonia in the composition over the course of its shelf life.

116. The method of any one of embodiments 99-115, wherein the method decreases the accumulation of ammonia in the composition by at least two-fold over the course of its shelf life as compared to an agricultural composition absent the microbial stabilizer and buffering agent.

117. The method of any one of embodiments 99-116, wherein the composition at the end of its' shelf life has a colonization potential approximately equal to the colonization potential of the composition when freshly formulated.

118. The method of any one of embodiments 99-117, wherein the microbial stability of the composition is greater than the microbial stability of the composition absent one or more of the buffering agent, microbial stabilizer, and physical stabilizer.

119. The method of any one of embodiments 99-118, wherein the composition has a shelf life of at least two months, at least three months, at least four months, or at least five months.

120. The method of any one of embodiments 99-119, wherein the composition has a shelf life of at least three months.

121. The method of any one of embodiments 99-120, wherein the log loss of CFU/mL over the shelf life of the composition is less than 0.2.

122. The method of any one of embodiments 99-121, wherein the cellular density of the bacterium minimizes the rate of decay over the shelf life of the composition.

123. The method of any one of embodiments 99-122, wherein the cellular density of the bacterium provides a reduced, but not minimized rate of decay.

124. The method of any one of embodiments 99-123, wherein the cellular density of the bacterium provides a rate of decay of less than 1.0 log loss of CFU/mL over 30 days in the agricultural composition absent the buffering agent, microbial stabilizer, and physical stabilizer.

125. The method of any one of embodiments 99-124, wherein the bacterium is present at a cellular density of about 3E9-6E9 CFU/mL.

126. The method of any one of embodiments 99-125, wherein the buffering agent maintains the pH of the composition over the shelf life of the composition.

127. The method of any one of embodiments 99-126, wherein the buffering agent maintains the pH of the composition at about pH 6-8 over the shelf life of the composition.

128. The method of any one of embodiments 99-127, wherein the buffering agent maintains the pH of the composition at about pH 6.5 over the shelf life of the composition.

129. The method of any one of embodiments 99-128, wherein the buffering agent is selected from the list consisting of phosphate buffered saline (PBS); modified, high buffering capacity PBS; 3-Morpholinopropane-1-sulfonic acid (MOPS); and 2-[4-(2-hydroxyethyl)piperazin-1-yl]ethanesulfonic acid (HEPES).

130. The method of any one of embodiments 99-129, wherein the buffering agent is modified, high buffering capacity PBS.

131. The method of any one of embodiments 99-130, wherein the microbial stabilizer slows the doubling rate of the diazotrophic bacterium.

132. The method of any one of embodiments 99-131, wherein the microbial stabilizer slows the toxin accumulation rate within the composition.

133. The method of any one of embodiments 99-132, wherein the microbial stabilizer is a monosaccharide, disaccharide, polysaccharide, pentose, hexose, oligosaccharide, oligofructose, sugar alcohol, amino acid, protein or protein hydrolysate, or polymer.

134. The method of any one of embodiments 99-133, wherein the microbial stabilizer is a monosaccharide or a disaccharide selected from the list consisting of glucose, fructose, trehalose, sucrose, lactose, melibiose, and lactulose.

135. The method of any one of embodiments 99-134, wherein the microbial stabilizer is fructose or trehalose.

136. The method of any one of embodiments 99-135, wherein the microbial stabilizer is fructose.

137. The method of any one of embodiments 99-136, wherein the microbial stabilizer is fructose and is selected for inclusion in the composition at a concentration of about 0.5-2.5% w/v.

138. The method of any one of embodiments 99-137, wherein the microbial stabilizer is fructose and is selected for inclusion in the composition at a concentration of about 1.3% w/v.

139. The method of any one of embodiments 99-138, wherein the physical stabilizer decreases the local density of the diazotrophic bacterium within the composition.

140. The method of any one of embodiments 99-139, wherein the physical stabilizer is a polysaccharide, protein or protein hydrolysate, polymer, or a natural gum or its derivative.

141. The method of any one of embodiments 99-140, wherein the physical stabilizer is a polysaccharide.

142. The method of any one of embodiments 99-141, wherein the physical stabilizer is a polysaccharide selected from the list consisting of maltodextrin, polyethylene glycol (PEG), xanthan gum, pectin, alginates, microcrystalline cellulose, and dextran.

143. The method of any one of embodiments 99-142, wherein the physical stabilizer is xanthan gum.

144. The method of any one of embodiments 99-143, wherein the physical stabilizer is xanthan gum and is selected for inclusion in the composition at a concentration of about 0.001-0.2% w/v.

145. The method of any one of embodiments 99-144, wherein the physical stabilizer is xanthan gum and is selected for inclusion in the composition at a concentration of about 0.1% w/v.

146. The method of any one of embodiments 99-145, wherein the bacterium is a gram-negative bacterium.

147. The method of any one of embodiments 99-146, wherein the bacterium is of a genus selected from the group consisting of: Acetobacter, Achromobacter, Aerobacter, Anabaena, Azoarcus, Azomonas, Azorhizobium, Azospirillum, Azotobacter, Beijernickia, Bradyrhizobium, Burkholderia, Citrobacter, Derxia, Enterobacter, Herbaspirillum, Klebsiella, Kluyvera, Kosakonia, Mesorhizobium, Metakosakonia, Paraburkholderia, Nostoc, Rahnella, Rhizobium, Rhodobacter, Rhodopseudomonas, Rhodospirillum, Serratia Sinorhizobium, Spirillum, Trichodesmium, and Xanthomonas.

148. The method of any one of embodiments 99-147, wherein the bacterium is of a species selected from the group consisting of: Achromobacter marplatensis, Achromobacter spiritinus, Azospirillum lipoferum, Enterobacter sacchari, Herbaspirillum aquaticum, Klebsiella variicola, Kluyvera intermedia, Kosakonia pseudosacchari, Kosakonia sacchari, Metakosakonia intestini, Paraburkholderia tropica, Rahnella aquatilis, and combinations thereof.

149. The method of any one of embodiments 99-148, wherein the bacterium is a gram-positive bacterium.

150. The method of any one of embodiments 99-149, wherein the bacterium is of a genus selected from the group consisting of: Arthrobacter, Agromyces, Bacillus, Clostridium, Corynebacterium, Frankia, Heliobacillus, Heliobacterium, Heliophilum, Heliorestis Methanobacterium, Microbacterium, Micrococcus, Micromonospora, Mycobacterium, Paenibacillus, Propionibacterium, and Streptomyces.

151. The method of any one of embodiments 99-150, wherein the bacterium is of a species selected from the group consisting of: Bacillus amyloliquefaciens, Bacillus macerans, Bacillus pumilus, Bacillus thuringiensis, Clostridium acetobutylicum, Corynebacterium autitrophicum Methanobacterium formicicum, Methanobacterium omelionski, Microbacterium murale, Mycobacterium flavum, Paenibacillus polymyxa, Paenibacillus riograndensis, Propionibacterium acidipropio, Propionibacterium freudenreichii, Streptococcus lactis, Streptomyces griseus, and combinations thereof.

152. The method of any one of embodiments 99-151, wherein the bacterium is of the genus Klebsiella.

153. The method of any one of embodiments 99-152, wherein the bacterium is of the species Klebsiella variicola.

154. The method of any one of embodiments 99-153, wherein the bacterium is of the strain Klebsiella variicola NCMA 201712002.

155. The method of any one of embodiments 99-154, wherein the bacterium is of the genus Kosakonia.

156. The method of any one of embodiments 99-155, wherein the bacterium is of the species Kosakonia sacchari.

157. The method of any one of embodiments 99-156, wherein the bacterium is of the strain Kosakonia sacchari PTA-126743.

158. The method of any one of embodiments 99-157, wherein the bacterium is endophytic, epiphytic, or rhizospheric.

159. The method of any one of embodiments 99-158, wherein the bacterium is a wild type bacterium.

160. The method of any one of embodiments 99-159, wherein the bacterium is an engineered bacterium.

161. The method of any one of embodiments 99-160, wherein the bacterium is a transgenic bacterium.

162. The method of any one of embodiments 99-161, wherein the bacterium is an intragenic bacterium.

163. The method of any one of embodiments 99-162, wherein the bacterium is a remodeled bacterium.

164. The method of any one of embodiments 99-163, wherein the bacterium comprises a non-intergeneric genomic modification.

165. The method of any one of embodiments 99-164, wherein the bacterium is an engineered bacterium capable of fixing atmospheric nitrogen in the presence of exogenous nitrogen.

166. The method of any one of embodiments 99-165, wherein the bacterium is an engineered bacterium comprising at least one genetic variation introduced into at least one gene, or non-coding polynucleotide, of the nitrogen fixation or assimilation genetic regulatory network.

167. The method of any one of embodiments 99-166, wherein the bacterium is an engineered bacterium comprising an introduced control sequence operably linked to at least one gene of the nitrogen fixation or assimilation genetic regulatory network.

168. The method of any one of embodiments 99-167, wherein the bacterium is an engineered bacterium comprising a heterologous promoter operably linked to at least one gene of the nitrogen fixation or assimilation genetic regulatory network.

169. The method of any one of embodiments 99-168, wherein the bacterium is an engineered bacterium comprising at least one genetic variation selected from the group consisting of: nifA, nifL, ntrB, ntrC, polynucleotide encoding glutamine synthetase, glnA, glnB, glnK, drat, amtB, polynucleotide encoding glutaminase, glnD, glnE, nifJ, nifH, nifD, nifK, nifY, nifE, nifN, nifU, nifS, nifV, nifW, nifZ, nifM, nifF, nifB, nifQ, a gene associated with biosynthesis of a nitrogenase enzyme, and combinations thereof.

170. The method of any one of embodiments 99-169, wherein the bacterium is an engineered bacterium comprising at least one genetic variation introduced into at least one gene, or non-coding polynucleotide, of the nitrogen fixation or assimilation genetic regulatory network that results in one or more of: increased expression or activity of NifA or glutaminase; decreased expression or activity of NifL, NtrB, glutamine synthetase, GlnB, GlnK, DraT, AmtB; decreased adenylyl-removing activity of GlnE; or decreased expression or uridylyl-removing activity of GlnD.

171. The method of any one of embodiments 99-170, wherein the bacterium is an engineered bacterium comprising a mutated nifL gene that has been altered to comprise a heterologous promoter inserted into said nifL gene.

172. The method of any one of embodiments 99-171, wherein the bacterium is an engineered bacterium comprising a mutated glnE gene that results in a truncated GlnE protein lacking an adenylyl-removing (AR) domain.

173. The method of any one of embodiments 99-172, wherein the bacterium is an engineered bacterium comprising a mutated glnD gene that results in the lack of expression of said glnD gene.

174. The method of any one of embodiments 99-173, wherein the bacterium is an engineered bacterium comprising a mutated amtB gene that results in the lack of expression of said amtB gene.

175. The method of any one of embodiments 99-174, wherein the bacterium is an engineered bacterium comprising at least one of: a mutated nifL gene that has been altered to comprise a heterologous promoter inserted into said nifL gene; a mutated glnE gene that results in a truncated GlnE protein lacking an adenylyl-removing (AR) domain; a mutated amtB gene that results in the lack of expression of said amtB gene; a mutated glnD gene that results in the lack of expression of said glnD gene; and combinations thereof.

176. The method of any one of embodiments 99-175, wherein the bacterium is an engineered bacterium comprising at least one genetic variation introduced into genes involved in a pathway selected from the group consisting of: exopolysaccharide production, endo-polygalaturonase production, trehalose production, and glutamine conversion.

177. The method of any one of embodiments 99-176, wherein the bacterium is an engineered bacterium comprising at least one genetic variation introduced into genes selected from the group consisting of: bcsii, bcsiii, yjbE, fhaB, pehA, otsB, treZ, glsA2, and combinations thereof.

178. The method of any one of embodiments 99-177, wherein the bacterium is selected from Table 1, or a variant, mutant, or derivative thereof.

179. The method of any one of embodiments 99-178, wherein the bacterium comprises a nucleic acid sequence that shares at least about 90%, 95%, or 99% sequence identity to a nucleic acid sequence selected from SEQ ID NOs: 1-223.

180. The method of any one of embodiments 99-179, wherein the bacterium comprises a nucleic acid sequence selected from SEQ ID NOs: 1-223.

181. The method of any one of embodiments 99-180, wherein the bacterium is a non-intergeneric remodeled bacterium.

What is claimed is:

1. An agronomically stable liquid agricultural composition, comprising:
a) diazotrophic bacteria;
b) a buffering agent;
c) a microbial stabilizer comprising a monosaccharide or disaccharide; and
d) a physical stabilizer comprising a polysaccharide selected from maltodextrin, pectin, an alginate, microcrystalline cellulose, and dextran, a protein or protein hydrolysate, a polymer, or a natural gum,
wherein the composition has a log loss of 0.2 or less over the shelf life of the composition.

2. The composition of claim 1, wherein the composition has a soluble ammonia concentration of less than 20 mM after 90 days storage at room temperature.

3. The composition of claim 1, wherein the bacteria are present at a cellular density of about 3E9 CFU/mL to about 6E9 CFU/mL.

4. The composition of claim 1, wherein the buffering agent is selected from phosphate buffered saline (PBS); modified, high buffering capacity PBS; 3-morpholinopropane-1-sulfonic acid; and 2-[4-(2-hydroxyethyl)piperazin-1-yl]ethanesulfonic acid.

5. The composition of claim 1, wherein the buffering agent is modified, high buffering capacity PBS.

6. The composition of claim 1, wherein the microbial stabilizer is a monosaccharide selected from glucose and fructose or a disaccharide selected from trehalose, sucrose, lactose, melibiose, and lactulose.

7. The composition of claim 1, wherein the microbial stabilizer is fructose or trehalose.

8. The composition of claim 1, wherein the microbial stabilizer is fructose and is present in the composition at a concentration of about 0.5% w/v to about 2.5% w/v.

9. The composition of claim 1, wherein the physical stabilizer is xanthan gum.

10. The composition of claim 1, wherein the physical stabilizer is xanthan gum and is present in the composition at a concentration of about 0.001% w/v to about 0.2% w/v.

11. The composition of claim 1, wherein the bacteria is of a genus selected from: Acetobacter, Achromobacter, Aerobacter, Anabaena, Azoarcus, Azomonas, Azorhizobium, Azospirillum, Azotobacter, Beijernickia, Bradyrhizobium, Burkholderia, Citrobacter, Derxia, Enterobacter, Herbaspirillum, Klebsiella, Kluyvera, Kosakonia, Mesorhizobium, Metakosakonia, Paraburkholderia, Nostoc, Rahnella, Rhizobium, Rhodobacter, Rhodopseudomonas, Rhodospirillum, Serratia Sinorhizobium, Spirillum, Trichodesmium, and Xanthomonas.

12. The composition of claim 1, wherein the bacteria is of a species selected from: Achromobacter marplatensis, Achromobacter spiritinus, Azospirillum lipoferum, Enterobacter sacchari, Herbaspirillum aquaticum, Klebsiella variicola, Kluyvera intermedia, Kosakonia pseudosacchari, Kosakonia sacchari, Metakosakonia intestini, Paraburkholderia tropica, Rahnella aquatilis, and combinations thereof.

13. The composition of claim 1, wherein the bacteria is of a genus selected from: Arthrobacter, Agromyces, Bacillus, Clostridium, Corynebacterium, Frankia, Heliobacillus, Heliobacterium, Heliophilum, Heliorestis Methanobacterium, Microbacterium, Micrococcus, Micromonospora, Mycobacterium, Paenibacillus, Propionibacterium, and Streptomyces.

14. The composition of claim 1, wherein the bacteria is of a species selected from: Bacillus amyloliquefaciens, Bacillus macerans, Bacillus pumilus, Bacillus thuringiensis, Clostridium acetobutylicum, Corynebacterium autitrophicum Methanobacterium formicicum, Methanobacterium omelionski, Microbacterium murale, Mycobacterium flavum, Paenibacillus polymyxa, Paenibacillus riograndensis, Propionibacterium acidipropio, Propionibacterium freudenreichii, Streptococcus lactis, Streptomyces griseus, and combinations thereof.

15. The composition of claim 1, wherein the bacteria is a species of Klebsiella, Kosakonia, or a combination thereof.

16. The composition of claim 1, wherein the bacteria is a strain of Klebsiella variicola, a strain of Kosakonia sacchari, or a combination thereof.

17. The composition of claim 1, wherein the bacteria is Klebsiella variicola NCMA 201712002, Kosakonia sacchari PTA-126743, or a combination thereof.

18. The composition of claim 1, wherein the bacteria comprise an engineered bacterium capable of fixing atmospheric nitrogen in the presence of exogenous nitrogen.

19. The composition of claim 1, wherein the bacteria comprise an engineered bacterium comprising a heterologous promoter operably linked to at least one gene of a nitrogen fixation or assimilation genetic regulatory network.

20. The composition of claim 1, wherein the bacteria comprise a nucleic acid sequence that shares at least about 90%, 95%, or 99% sequence identity to a nucleic acid sequence selected from any one of SEQ ID NOs: 1-223.

21. An agronomically stable liquid agricultural composition with a room temperature shelf life of at least 3 months, comprising:
a) Klebsiella at a concentration of at least about 4.5E9 CFU/mL;
b) modified, high buffering capacity phosphate buffered saline;
c) fructose at a concentration of at least 1% w/v; and
d) xanthan gum at a concentration of at least 0.05% w/v.

22. An agronomically stable liquid agricultural composition with a room temperature shelf life of at least 3 months, comprising:
a) Kosakonia at a concentration of about 3E9 CFU/mL;
b) modified, high buffering capacity phosphate buffered saline; and
c) fructose at a concentration of at least 1% w/v.

23. A method of providing fixed atmospheric nitrogen to a cereal plant, said method comprising applying the composition of claim 1 to the cereal plant, a cereal plant tissue, or an environ thereof.


SEQUENCE LISTING (223 sequences)

SEQ ID NO: 1 - DNA, 1270 bp, Rahnella aquatilis
  gene 1..1270: note = 16S
  variation 186: note = n is a, c, t, g, unknown or other
  variation 458: note = n is a, c, t, g, unknown or other
  variation 1041: note = n is a, c, t, g, unknown or other
  variation 1131..1132: note = n is a, c, t, g, unknown or other
  variation 1135: note = n is a, c, t, g, unknown or other
  variation 1139..1140: note = n is a, c, t, g, unknown or other
  source 1..1270: mol_type = genomic DNA
attgaagagtttgatcatggctcagattgaacgctggcggcaggcctaacacatgcaagtcgagcggcagcgggaagtagcttgctactttgccggcgagcggcggacgggtgagtaatgtctgggaaactgcctgatggagggggataactactggaaacggtagctaataccgcatgacctcgnaagagcaaagtgggggatcttcggacctcacgccatcggatgtgcccagatgggattagctagtaggtgaggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttagggttgtaaagcactttcagcgaggaggaaggcatcanacttaatacgtgtggtgattgacgttactcgcagaagaagcaccggctaactccgtgccagcagccgcggtaatacggagggtgcaagcgttaatcggaattactgggcgtaaagcgcacgcaggcggtttgttaagtcagatgtgaaatccccgcgcttaacgtgggaactgcatttgaaactggcaagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccacggaattcgccagagatggcttagtgccttcgggaaccgtganacaggtgctgcatggctgtcgtcagctcgtgttgtgaaatgttgggttaagtcccgcaacgagcgcaacccttatcctttgttgccagcnngtnatgnngggaactcaaaggagactgccggtgataaaccggaggaaggtggggatgacgtcaagtcatcatggcccttacgagtagggctacacacgtgctacaatggcatatacaaagagaagcgaactcgcgagg

SEQ ID NO: 2 - DNA, 876 bp, Rahnella aquatilis
  gene 1..876: note = nifH
  source 1..876: mol_type = genomic DNA
atggcaatgcgtcaatgcgcaatctacgggaaagggggtattgggaaatccaccactacccaaaaccttgtagcggctctggccgaaatgaataagaaggtcatgatcgtcggctgtgaccctaaggctgattcaacccgcctcattctgcatgcgaaagcacagaacaccatcatggaaatggccgctgaagtgggctccgtggaagatctggagctggaagatgtgatgcaaatcggctatggcggcgtgcgctgtgcggaatcaggcggccctgagcctggtgtgggttgtgccggacgcggggtgatcaccgccatcaacttcctcgaagaagaaggcgcgtatgtgccggatctggattttgtgttttacgacgtattgggcgatgtggtctgtggcggtttcgcgatgccaattcgcgaaaacaaagcgcaggaaatctacatcgtgtgctccggtgaaatgatggcgatgtatgccgccaacaacatttccaaaggcatcgtgaaatacgcgaaatcgggcaaagttcgcctggccgggctgatctgtaactcccgccagacggatcgcgaagatgaactgatcatcgcgctggctgaaaaacttggcacgcaaatgatccacttcgtgccgcgtgacaacattgtgcaacgcgctgaaatccgccgcatgacggtcatcgaatacgacccgacttgtgcgcaggcagatcagtatcgtgcactggcgaacaaaatcgtcaacaacaccaaaatggtggtgccgacaccggtcaccatggatgagctggaagccctgttaatggaatttggcattatggaagaagaagacctggccatcgtcggtcgtaccgccgccgaagaggcgtga

SEQ ID NO: 3 - DNA, 1374 bp, Rahnella aquatilis
  gene 1..1374: note = nifD1
  source 1..1374: mol_type = genomic DNA
atgaaggcaaaagagattctggcgctgattgatgagccagcctgtgagcataaccacaagcagaagtcgggttgcagcctgccgaaaccgggcgcgacggcaggcggttgtgcgtttgatggcgcgcagattgcgctgctgccggtcgcggacgtcgcgcatctggtgcacggcccgattggctgtaccggcagttcatgggacaaccgtggcagccgcagttccgggccttccatcaaccgcatgggcttcaccaccgacatgagcgagcaggatgtgattatggggcgcggcgagcgacgcttatttcacgccgtgcagcacatcgtcagccattaccatccggtggcggtctttatttacaacacctgcgtacccgcgatggaaggggatgacgttgaagccgtgtgtcgcgccgcatcggccgctgccggtgtgccggttatttcagtcgatgccgccggtttctacggcagcaaaaatctcggtaaccgcattgccggggacgtgatggtcaaaaaggtgatcggccagcgcgaacccgcgccgtggccggaaaactcaccgatccccgccggacaccgccacagcatcagcctgattggcgaattcaatattgccggcgagttctggcacgttctgccgctgctcgatgagctcgggatccgcgtgctgtgcagcctttccggggattcccgttttgctgaaatccagactatgcaccgtggcgaagccaacatgctggtgtgctcgcgggcgctgatcaacgtcgcccgaaaaatggaagagcgttaccagatcccatggtttgaaggcagtttttatggcctgcgttccatggctgattccctgcgcacgatcgccgtgctgctcaaagacccggatttacaggcgcgcacagaacgtctgattgagcgcgaggaggcggcgacacatcttgcgcttgcgccttaccgtgcgcggctcagcgggcgcaaggcgctgctgtataccggtggcgtgaaatcctggtcggtggtctcggcgttacaggatttaggcatcacggtggtggcgaccggcacccgaaaatcaaccgaagaagacaagcagcgtattcgcgaactgatgggtgaagacgtgctgatgctcgacgaaggcaatgccagaaccttgctcgacaccctctatcgtttcggcggcgacatcatgatcgccgggggccgcaacatgtataccgcgtacaaagcccgcctgccgttcctggatatcaatcaggagcgcgagcatgcgtttgccggatatcacgggctggtaaatctggccgaacagttgtgtatcaccctggaaagcccggtctgggcgcaggtcaaccgtctggcgccgtggcgctaa

SEQ ID NO: 4 - DNA, 1449 bp, Rahnella aquatilis
  gene 1..1449: note = nifD2
  source 1..1449: mol_type = genomic DNA
atgaccagtgaaacacgcgaacgtaacgaggcattgatccaggaagtgctggagatcttccccgagaaggcgcttaaagatcgtaagaaacacatgatgaccaccgacccggcgatggaatctgtcggcaagtgtattgtctcaaaccgcaaatcacagccgggcgtgatgaccgtgcgaggctgcgcttacgccggttccaaaggcgtggtctttggcccgatcaaagacatggcgcatatctcccacggcccggttggttgcggccagtattcccgtgccggacgccgtaactattacaccggctggagcggcgtgaacagctttggcaccctcaacttcaccagtgattttcaggaacgggacatcgtatttggcggcgataaaaagctcgacaaattgatcgatgaactggagatgttgttcccgctgagcaaaggcatttcggtgcagtcggaatgtccggtcggtctgatcggcgatgacatttctgccgtcgccaaagccagcagcgccaaaatcggtaagccggtcgtgccggtacgctgcgaggggttccgcggtgtgtcgcaatcgctcggccatcacattgctaacgatgtcatccgcgactgggtgctggataaccgcgaaggcaatgaatttgaaaccacgccttacgacgtggcgattatcggcgactacaacatcggcggtgacgcctgggcctcacgtattctgctcgaagaaatggggctgcgcgtggtggcgcagtggtccggcgacggcacgctggtggagatggaaaacaccccgaaagtcgcgctcaatctggtgcactgctaccgctcgatgaactacatctcccgtcatatggaagaaaaacacggcattccgtggatggaatacaacttctttggcccgaccaaaattgcggaatctctgcgcgaaatcgcggcgcgttttgacgataccatccggaaaaacgccgaagcggtgattgaaaaatatcaggcgcaaacgcaggcggtgatcgacaaataccgtccgcgtctggaaggcaaaaaggtgctgttgtatctcggcggtttacgtccgcgccacatcatcggggcgtatgaagatctgggaatggaaatcatcggtaccggctatgaattcggtcataacgatgattacgaccgcaccttaccgatgctcaaagaaggcacgttgctgttcgatgacctgagcagttatgagctggaagcgttcgttaaagcgctgaaaccggatcttgtcgggtcaggtatcaaagaaaaatacattttccagaaaatgggcgtgccgttccgccagatgcactcctgggattattccggcccttatcacggctacgacggtttcggcatttttgcccgtgacatggacatgacgctgaacaatccgggctggagtcagctgaccgccccctggttgaaaacggcctga

SEQ ID NO: 5 - DNA, 1386 bp, Rahnella aquatilis
  gene 1..1386: note = nifK1
  source 1..1386: mol_type = genomic DNA
atggctcaaattctgcgtaatgccaagccgcttgccaccacgcctgtcaaaagcgggcaaccgctcggggcgatcctggccagtcaggggctggaaaattgcatcccgctggttcacggcgcgcaaggttgtagcgcgttcgccaaagttttcttcatccagcattttcacgatccgatcccgttgcagtccacggcgatggaatcgaccacgactatcatgggctcggatggcaacgtcagtactgcgttgaccacgttgtgtcagcgcagtaatccaaaagccattgtgattttgagcaccggactgtcagaagcgcagggcagtgatttgtcgatggcgctgcgtgagtttcgcgacaaagaaccgcgctttaatgccatcgctattctgaccgttaacacgccggatttttacggctcgctggaaaacggctacagcgcgctgatggaaagcgtgatcactcagtgggtgccggaaaagccgccgaccggcatgcgtaacaagcgcgtgaacctgctggtgagccatctgctgacgccgggggatctggaattactgcgcagctatgtcgaagcctttggcctgcaaccggtgatcctgccggatttatcacagtcgctggacggacatctggcgaatggcgatttcaatccggtcacgcagggcggcacgtcgcaacgccagattgaacaaatggggcagagcctgaccaccattaccattggcagttcgctcaactgcgccgccagtctgatggcgatgcgcagccgtggcatggcgctgaacctgccgcacctgatgacgctggaaaacatggacagtctgatccgccatctgcatcaggtgtcaggccgcgaggtaccggcatggattgagcgccagcgcgggcaactgctggacgccatgatcgactgccatacctggctgcagtcacagcgtattgcgctggcggcagaagcggatttgctggtggcgtggtgcgatttcgctcagagccagggaatgcgcgtcgggccggtgattgcgccggttaatcagcagtcactggccgggctgccggtcgaacaggtggtgatcggcgatctggaagatttacaaacccggctcgacagctacccggtttcactgctggtggcgaactcccacgctgcaccactggcggaaaaaaacggtatcgcgctggtacgtgccggtttcccgctttacgaccgtctcggggaatttcgccgcgtgcggcagggctatgcgggtattcgcgacaccttgttcgaactcgcgaacctgatgcaggcgcgccatcacatgctgacggcgtatcactcaccgcttaggcaggtgttcggcctgagcccggtaccggaggccagtcatgaggcgcgctaa

SEQ ID NO: 6 - DNA, 1569 bp, Rahnella aquatilis
  gene 1..1569: note = nifK2
  source 1..1569: mol_type = genomic DNA
atgagtcaagatcttggcaccccaaaatcctgtttcccgctgttcgagcaggatgaataccagaatatgtttacccacaaacgcgcgctggaagaagcacacggcgaggcgaaagtgcgggaagtgtttgaatggaccaccacgcaggaatatcaggatctgaacttctcgcgtgaagcgctgaccgtcgacccggcgaaagcctgccagccgttaggcgcggtactttgcgcgctgggttttaccaacacgttgccgtatgtccatggttcacaaggctgtgtggcgtatttccgtacctattttaatcgtcatttcaaagagccggtggcctgtgtttccgactcaatgaccgaagatgccgccgtttttggcggaaataacaacatgaatgtcggtctggaaaacgccagcgcgctgtacaagccggaaattattgcggtctccaccacctgtatggcggaagtgatcggtgatgacctgcaggcttttatcgccaacgccaaaaaagacggatttgtggatgccggtatgccaatcccgtatgcccatacaccgagttttctgggcagtcatgtcaccggctgggacaacatgtttgaaggcttcgcccgtacctttaccaccgacgccacgcgggaatatcagccgggcaaacttgccaaactgaacgtggtgaccggttttgaaacttatctcggcaactaccgggttattcaccgcatgatgagccagatgggggtcgaatgcagcgtcttgtccgatccgtctgaagtgctcgacaccccggctgacggccaataccgcatgtatgccggcggcaccacgcaaaccgaaatgcgtgatgcaccggatgccatcgacaccttgctgctgcaaccgtggcaattacagaaaaccaaaaaggtggtgcagggcgactggaatcagccgggcaccgaagtcagtgtaccgattggcctggcggcgaccgatgccttgctgatgacggtaagcgaactgaccggcaaaccgatagctgacgcgctggcgactgaacgtggccgtctggtggacatgatgctcgattctcacacctggctgcacggcaagcgtttcggtctctacggtgacccggattttgtgatgggcatgaccgcattcctgctggaactgggctgtgaaccgaccaccattctcagccataacggcaacaaacgctggcagaaagccatgaagaaaatgctggctgattcgccttacggacaggacagcgaagtgtatgtgaactgcgatctgtggcatttccgctcgctgatgtttacccgtaaaccggactttatgatcggcaactcttacggaaaattcattcagcgtgacacgctggccaaaggcgaacagttcgaagtgccgctgatccgtatcggattcccgatttttgaccggcaccatttgcaccgtcagaccacctggggatacgagggcgcgatgagcatcctgacgcaactggtgaatgcggtgctcgaacagctggatcgcgaaaccatgaagctcggcaaaaccgactacaacttcgatctgatccgctaa

SEQ ID NO: 7 - DNA, 1488 bp, Rahnella aquatilis
  gene 1..1488: note = nifL
  source 1..1488: mol_type = genomic DNA
atgagcatcacggcgttatcagcatcatttcctgaggggaatatcgccagccgcttgtcgctgcaacatccttcactgttttataccgtggttgaacaatcatcggtggcgatttcgctgaccgatccgcaggcgcgcatttgttatgccaatccggcattctgccgccagacgggttttgcacttgagacacttttgggcgagaaccaccgtctgctggccagccagcagacgccgaaacatatctatgacgaaatgtggcgcactttgttgcagggcaaatcctggaacggccaactgatcaaccggcgtaataaccgttcgctttatctggcggatgtcactatcacgcctgttttaggcgcggacgggcaggtggagcattacctcggcatgcacaaagatatcagcgagaaatacgcgctggagcagcggttgcgcaaccacatcaccttgttcacggaggtgctgaacaatattcccgccgccgtggtggtggtggatgagcaggacaatgtggtgatggacaatctggcctacaaaaccctgtgcgctgactgcggcggaaaagagctgttggccgaaatgggctatccgcaactcaaagagatgctcaacagtggcgaaccggtgccggtttccatgcgcggcaacgtacgctggttttctttcggtcagtggtcattgcagggcgttaatgaagaggccagccgcttttttaccggcattaccgcgccgggaaaactgattgttctcaccgactgcaccgatcagcatcaccggcagcagcagggttatcttgaccggctcaagcaaaaacttaccaacggcaaattgctggcagccatccgcgagtcgcttgatgccgcgctgattcagctcaacgggccaattaatatgctggcggctgcgcgtcgtcttaacggcgaagaaggcaacaacatggcgctggaattcgcctggcgcgaaggcgagcaggcggtgagtcgcttacaggcctgccgtccgtcgctggattttgagccgcaggcagaatggccggtcagtgaattcttcgacgatctgagcgcgctgtacgacagccattttctcagtgacggtgaattgcgttacgtggtcatgccatctgatctgcacgctgtcgggcaacgaacgcaaatccttaccgcgctgagcttatggattgatcacacgctgtcacaggcgcaggccatgccgtctctgaagctctcggtgaacattgttgcgaagcaggatgcgagctggttgtgttttgacattaccgataatgtgccgcgtgaacgggtgcgttatgcccgcccggaagcggcgttttcccgtccggggaatggcatggagctgcgccttatccagacgctgatcgcccatcatcgcggttctttagatctctcggtccgccctgatggcggcaccttgctgacgttacgcctgccggtacagcaggttatcaccggaggcttaaaatga

SEQ ID NO: 8 - DNA, 1557 bp, Rahnella aquatilis
  gene 1..1557: note = nifA
  source 1..1557: mol_type = genomic DNA
atgacccagttacctaccgcgggcccggttatccggcgctttgatatgtctgcccagtttacggcgctttatcgcatcagcgtggcgctgagtcaggaaagcaataccgcgcgcgcactggcggcgatcctcgaagtgcttcacgatcatgcatttatgcaatacggcatggtgtgtctgttcgataaagaacgcaatgcactgtttgtggaatccctgcatggcatcgacggcgaaaggaaaaaagagacccgccatgtccgttaccgcatgggggaaggcgtgatcggcgcggtgatgagccagcgtcaggcgctggtgttaccgcgcatttcagacgatcagcgttttctcgaccgcctgaatatttacgattacagcctgccgctgattggtgtgccgatccccggtgcggataatcagcctgcgggtgtgctggtggcacagccgatggcgttgcacgaagaccggctggctgccagtacgcggtttttagaaatggtcgccaatctcatcagccagccactgcgttctgccacgcccccggaatcattgcctgctcaaacgccggtccggtgcagtgttccgcgccagtttggttttgagcagatggtcgggaaaagtcaggcgatgcgccagacgatggacattttacggcaggtttccaaatgggataccacggttctggtgcgtggtgaaagcggcaccggcaaggaacttatcgccaatgccattcattacaactcaccccgtgcggccgcgccatttgtgaaattcaactgcgccgcgctgccggataacctgctggaaagcgaactgttcggtcatgaaaaaggggccttcaccggcgctatacgcacccgaaaaggccgctttgaactggcggacgggggcacgttattcctcgatgaaatcggcgaatcgagcgcgtcgtttcaggccaaattgctgcgcattttgcaggaaggtgaaatggaacgggtcggcggcgataccacgctgaaagttgatgtgcgcattattgctgccaccaaccgtaatcttgaggaggaagtgcgtgccgggaattttcgcgaagacctgtattatcgcctgaacgtgatgccggtttcgctgcctgcactgcgtgaaaggctggatgatatcgccgatctggcgccgtttctggtcaaaaagattgcgctgcgtcaggggcgggaactgcgcatcagtgatggtgcggtgcgtctgctgatgacctacagctggccaggcaacgtgcgtgaactggaaaactgcctcgaacgggcgtcggtaatgaccgatgaagggctgatcgaccgcgacgtgatcctgttcaatcaccatgagtccccggcgctgtccgtcaaacccggtctgccgctggcgacagatgaaagctggctggatcaggaactcgacgaacgccagcgggtgattgctgcactggagaaaaccggctgggtgcaggccaaagcggcccgactgctgggcatgacaccgcgccagattgcctaccgtatccagattatggacatcaacatgcaccgtatctga

SEQ ID NO: 9 - DNA, 2838 bp, Rahnella aquatilis
  gene 1..2838: note = glnE
  source 1..2838: mol_type = genomic DNA
atgttgccactttcttctgttttgcaaagccacgcgcagagtttgcctgaacgctggcatgaacatcctgaaaacctgcccctccccgatgatgaacagctggctgtgctgagcagcagtgaattcatgacggacagtttgctggcttttccgcagtggtggcatgaaattgtccaaaatccccctcaggcgcaggagtggcaactttaccgtcagtggctggatgaatcgctgacgcaggtgactgacgaagccgggttaatgaaagctttgcgtctgttccgccgccgtattctgacccgcattgcgtggtcacagtccgcgcaaaccagcgaagcaaaagatacgcttcaccagctgagtgaactggcggaattattgattgtcagcgcccgtgactggctgtatgccgcttgctgtcgcgagttcggtacgccggtcaatgccgcaggggaaccgcagagaatgctgatcctcgggatgggcaaactcggcggtggcgagctgaatttctcatcggacatcgacctgatttttgcttatccggaaaatggccagacacgcggcggtcggcgtgaactggataacgcacaatttttcacccggctcggccagcgtctgatcaaagcgctggatcagcccactatcgacggttttgtctatcgcgtggacatgcgtttgcgtccgttcggcgacagtggcccgctggtgatgagcttcccggcactggaagattattatcaggaacaggggcgcgactgggaacgctacgcaatggtgaaagcgcgtctgatgggcggcgcggaggacatcagcagtcaggaattgcgtaaaatgctgatgccttttgtcttccgccgttatatcgatttcagtgtgatccagtccctgcgtaacatgaaaggcatgatcgcccgcgaagtacgccgccgtggtctgaaagacaacatcaaactcggcgcaggcggtattcgtgaaattgaatttatcgtgcaggtatttcagctgatccgtggcggtcgtgaaccggcattgcagcagcgtgcgttgttgccaacgcttcaggcgctggaaaatctggggctgctgccggtagagcaggtgttgcagttgcgtaacagctatctgttcctgcgacgtctggaaaacctgttgcaggccattgctgacgagcaaacgcaaaccttaccgtccgatgagctgaatcaggcgcgtctggcgtgggggatgaattacgctggctggccgcagcttctggatgcagtgaatgctcacatgcaggccgtacgcgcggtatttaacgatctgattggcgatgacacgccagatgccgaagatgacgtgcaactctcccggttcagcagtttatggattgatacgcttgagcctgacgagctggctccgctggtgccgcaacttgacgaaaatgcgcaacggcatgttttacatcagattgctgattttcgccgtgacgtggataaacgcacgatagggccacgtgggcgtgatcagttggatttgctgatgccgcgtttactggcccaggtctgcacctataaaaatgcggatgtgacgttacagcgcctgatgcagttgctgctcaatatcgtcacgcgcacgacgtatatcgagctgctggtggaatatcccggtgcgctcaaacagttaatacgtctgtgcgctgcctcgccgatggtggcgacgcaacttgcgcgtcatcctttattgctcgacgaactgctcgacccgcgcacgctttaccagccgattgagccgggcgcgtaccgtgatgaactgcggcaatatctgatgcgggtgccaaccgaagacgaagaacaacagcttgaagccgtgcgccagttcaaacaggcacagcatttacgtattgcggccggggatatttccggtgcgttgccggtgatgaaagtcagtgaccatttaacctaccttgcggaggccattctcgacgttgtggtgcaacaggcgtgggaacaaatggtcgtaaaatacggtcagccaacccatcttcagcaccgtaaagggcgcggttttgccgtggtgggttacggaaaactcggtggctgggagctgggttacagctcggatctggatctggtcttcctgctcgattgcgcgccggaagtcatgaccgacggcgaacgcagcattgacgggcgtcagttttatctgcggctggcgcagcgcatcatgcatttattcagcacccgtacgtcgtcaggcattctttatgaggttgacccgcgtctgcggccttccggtgcttccggcatgctggtcagcaccatcgaagcttttgcggattatcaggccaacgaagcctggacatgggagcatcaggcgctggttcgcgcgcgtgtggtttatggtgatccgcaactgacgcagcaatttaatgccacgcgtcgcgacattctttgccgccagcgcgatgccgacggcttgcgtaaggaagtccgtgaaatgcgcgagaaaatgtatgcccatctgggcagcaaaagagccgacgagtttgatctgaaagccgatccgggtggcataacggatattgaattcatcgcacaatatctggttctgcgtttcgcgcatgatgagccgaagctgacccgctggtctgataacgtgcggattttcgaactgatggcgcgacatgacatcatgccggaagaggaagcacgccatctgacgcaggcttacgtgacattgcgcgatgaaattcatcatctggcgttgcaggaacacagcgggaaagtggccgcagacagctttgccactgagcgcgcgcaaatccgcgccagctgggcaaactggcttggctga

SEQ ID NO: 10 - DNA, 1290 bp, Rahnella aquatilis
  gene 1..1290: note = amtB
  source 1..1290: mol_type = genomic DNA
atgaaaaaacttttatccatgatggggcttggtgcagtggctttgctaccttcgcttgccatggcagcagcaccagcagcggcaaacggtgctgataacgcctttatgatgatttgtaccgcgctggtattgttcatgaccgtacccggtgtggcgttgttctacggcggcttactgcgttctaaaaacgttttgtccatgctgactcaggttattgttacctttgctctggtctgcgtcctgtggatcctctacggttacagccttgccttcagtgaaggtaacgcgttcttcggtggtttcagcaacgtaatgatgaaaggcattggcctggattctgtgactggcaccttctcgcagatgatccacgttgcattccagtgttcatttgcctgcatcactgtagcgctgatcgtaggtggtattgctgaacgtgtgcgtttctcagcagttctgattttcactgtgatctggctgactttctcttatattccgatggctcacatggtatgggcaggcggtttcctggctgctgacggtgcgctggactttgccggtggtaccgttgttcatatcaatgccgcaattgctggcctggtaggggcttatctgctgggtaaacgcgccggttttggcaaagaagctttcaaaccacacaacctgccaatggtcttcactggcgcctcaatcctgtatgtgggctggttcggcttcaatgcgggttcagcaagtgccgcaagctctgttgccgcgctggctttcctgaacactgtcattgctactgctggcgcaatcctgtcctggacgctggttgagtggatggtgcgcggtaagccctcactgctgggcgcaagctccggtgctatcgcaggtctggtggctatcacgcctgcatgtggtacggtcggcgtaggtggtgctctgattatcggtctggtaggcggtatcactggtctgtggggggttgttaccctgaaaaaatggctgcgtgttgatgacacctgtgatgtgttcggtgttcatggcgtgtgcggtatcgtaggttgtctgctgacgggtgtattcacgtccagttcacttggcggcgtgggctacgcagaaggcgtgaccatgggccatcaggtttgggtgcagttcttcagcgtgtgcgtaacattggtctggtcaggcgttgttgccttcatcggttacaaagtggctgacatgatcgtaggtctgcgtgttcctgaagaacaagaacgcgaaggtctggacgttaacagccacggcgaaaacgcttacaaccaataa

SEQ ID NO: 11 - DNA, 500 bp, Rahnella aquatilis
  gene 1..500: note = PinfC
  source 1..500: mol_type = genomic DNA
tgaatatcactgactcacaagctacctatgtcgaagaattaactaaaaaactgcaagatgcaggcattcgcgttaaagccgacttgagaaatgagaagattggctttaaaattcgcgaacacacgctacgccgtgttccttatatgttagtttgtggcgataaagaggtcgaagcaggcaaagttgctgttcgtactcgtcgcggcaaagacttaggaagcatggatgttagcgaagtcgttgacaaactgctggcggaaatccgcagcagaagtcatcatcaactggaggaataaagtattaaaggcggaaaacgagttcaaccggcgcgtcctaatcgcattaacaaagagattcgcgcgcaagaagttcgcctcaccggcgtcgatggcgagcagattggtattgtcagtctgaatgaagctcttgaaaaagctgaggaagcgggcgtcgatttagtagaaatcagtccgaatgccgagccgccagtttgtcgaatc

SEQ ID NO: 12 - DNA, 1085 bp, Klebsiella variicola
  gene 1..1085: note = 16S
  source 1..1085: mol_type = genomic DNA
attgaagagtttgatcatggctcagattgaacgctggcggcaggcctaacacatgcaagtcgagcggtagcacagagagcttgctctcgggtgacgagcggcggacgggtgagtaatgtctgggaaactgcctgatggagggggataactactggaaacggtagctaataccgcataacgtcgcaagaccaaagtgggggaccttcgggcctcatgccatcagatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttcgggttgtaaagcactttcagcggggaggaaggcggtgaggttaataacctcaccgattgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcattcgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgatttggaggttgtgcccttgaggcgtggcttccggagctaacgcgttaaatcgaccgcctggggagtacggccgcaaggttaaaactcaaatgaattgacgggggcccgcacaagcggtggagcatgtggtttaattcgatgcaacgcgaagaaccttacctggtcttgacatccacagaactttccagagatggattggtgccttcgggaactgtgagacaggtgctgcatggctgtcgtcagctcgtgttgtgaaatgttggg

SEQ ID NO: 13 - DNA, 882 bp, Klebsiella variicola
  gene 1..882: note = nifH1
  source 1..882: mol_type = genomic DNA
atgaccatgcgtcaatgcgctatctacggtaaaggcggtatcggtaaatccaccaccacccagaatctcgtcgcggccctcgccgagatgggtaagaaagtgatgatcgtcggctgcgatccgaaagcggattccacccgtctgatcctccacgctaaagcccagaacaccatcatggagatggcggcggaagtgggctcggtcgaggatctggagctcgaagacgttctgcaaatcggctatggcgatgtccgttgcgccgaatccggcggcccggagccaggcgtcggctgcgccggacgcggggtgatcaccgccatcaacttcctcgaggaagaaggcgcctatgaagaagatttggatttcgtcttctatgacgtcctcggcgacgtggtctgcggcggcttcgctatgccgatccgcgaaaacaaagcccaggagatctacatcgtctgctccggcgagatgatggcgatgtatgccgccaacaatatctccaaagggatcgtgaagtacgccaaatccggcaaggtgcgcctcggcggcctgatctgtaactcgcgcaaaaccgaccgggaagacgaactgatcatcgccctggcggagaagcttggcacgcagatgatccacttcgttccccgcgacaacattgtgcagcgcgcggagatccgccggatgacggtgatcgagtacgacccgacctgtcagcaggcgaatgaatatcgtcaactggcgcagaagatcgtcaataacaccaaaaaagtggtgccgacgccgtgcaccatggacgagctggaatcgctgctgatggagttcggcatcatggaagaagaagacaccagcatcattggtaaaaccgccgctgaagaaaacgcggcctga

SEQ ID NO: 14 - DNA, 1113 bp, Klebsiella variicola
  gene 1..1113: note = nifH2
  source 1..1113: mol_type = genomic DNA
atggttaggaaaagtagaagtaaaaatacaaatatagaactaactgaacatgaccatttattaataagtcaaataaaaaagcttaaaacacaaaccacttgcttttttaataataaaggaggggttgggaagactacattagtagcaaatttaggagcagagctatcaataaactttagtgcaaaagttcttattgtggatgccgaccctcaatgtaatctcacgcagtatgtattaagtgatgaagaaactcaggacttatatgggcaagaaaatccagatagtatttatacagtaataagaccactatcctttggtaaaggatatgaaagtgacctccctataaggcatgtagagaatttcggttttgacataattgtcggtgaccctagacttgctttacaggaagaccttttagctggagactggcgagatgccaaaggcggtgggatgcgaggaattaggacaacttttgtatttgcagagttaattaagaaagctcgtgagctaaattatgattttgttttctttgacatgggaccatcattaggcgcaatcaacagggcagtattactggcaatggaattctttgtcgtcccaatgtcaatcgatgtattttcactatgggctattaaaaatattggctccacggtttcaatatggaaaaaagaattagacacagggattcggctctcagaggaacctagcgaattatcacaattatcacctcaaggaaaactaaagtttctcggttacgtcacccaacaacataaagaacgctctggatacgatacaattcagcttgagaatactgaggaagaaataaaatcgaaacgtcgggtaaaggcgtatgaagacattggagaggtgtttccttctaaaattactgagcatctttctaaactttatgcatcaaaagatatgaacccacaccttggagatatacgtcatttaggtagtttagctccgaaatcacaatcacaacacgttccgatgatatcagtgtctggtacaggaaattacaccagacttagaaaaagcgcgcgtgaactttatcgagatattgcaagaagatacttagagaacattcagactgctaatggcgagaaatag

SEQ ID NO: 15 - DNA, 1374 bp, Klebsiella variicola
  gene 1..1374: note = nifD1
  source 1..1374: mol_type = genomic DNA
atgaagggaaaggaaattctggcgctgctggacgaacccgcctgcgagcacaaccagaagcaaaaatccggctgcagcgcccctaagcccggcgctaccgccggcggttgcgccttcgacggcgcgcagataacgctcctgcccatcgccgacgtcgcgcacctggtgcacggccccatcggctgcgcgggcagctcgtgggataaccgcggcagcgtcagcgccggcccggccctcaaccggctcggctttaccaccgatcttaacgaacaggatgtgattatgggccgcggcgaacgccgcctgttccacgccgtgcgtcacatcgtcgaccgctatcatccggcggcggtctttatctacaacacctgcgtaccggcgatggagggcgatgacatcgaggcggtctgccaggccgcacagaccgccaccggcgtcccggtcatcgctattgacgccgccggtttctacggcagtaaaaatcttggcaaccgaatggcgggcgacgtgatgctcaggcaggtgattggccagcgcgaaccggccccgtggccagacaacacgccctttgccccggcccagcgccacgatatcggcctgattggcgaattcaatatcgccggcgagttctggcaggtccagccgctgctcgacgagctggggatccgcgtcctcggcagcctctccggcgacggccgctttgccgagatccagaccctgcaccgggcgcaggccaatatgctggtgtgctcgcgcgcgctgatcaacgtcgcccgggggctggagctgcgctacggcacgccgtggtttgaaggcagcttctacgggatccgcgccacctccgacgccttgcgccagctggcgacgctgctgggggatgacgacctgcgccgccgcaccgaggcgctgatcgcccgcgaagagcaggcggcggagcaggctcttgcgccgtggcgtgagcagctccgcgggcgcaaagtgctgctctataccggcggcgtgaaatcctggtcggtggtatcggccctgcaggatctcggcatgaccgtggtggccaccggcacgcgcaaatccaccgaggaggacaaacagcggatccgtgagctgatgggcgacgaggcggtgatgcttgaggagggcaatgcccgcaccctgctcgacgtggtgtaccgctatcaggccgacctgatgatcgccggcggacgcaatatgtacaccgcctggaaagcccggctgccgtttctcgatatcaatcaggagcgcgagcacgcctacgccggctatcagggcatcatcaccctcgcccgccagctctgtctgaccctcgccagccccgtctggccgcaaacgcatacccgcgccccgtggcgctag

SEQ ID NO: 16 - DNA, 1449 bp, Klebsiella variicola
  gene 1..1449: note = nifD2
  source 1..1449: mol_type = genomic DNA
atgaccaacgcaacaggcgaacgtaaccttgcgctcatccaggaagtcctggaggtgtttcccgaaaccgcgcgcaaagagcgcagaaagcacatgatgatcagcgatccgcagatggagagcgtcggcaagtgcattatctcgaaccgtaaatcgcagcccggggtgatgaccgtgcgcggctgcgcctatgcgggctcgaaaggggtggtgtttgggccaatcaaagacatggcccatatctcgcacggccccatcggctgcggccagtattcccgcgccggacggcgcaactactataccggcgtcagcggtgtcgacagcttcggcaccctgaacttcacctctgattttcaggagcgcgatattgttttcggcggcgataaaaagctgaccaaactgatcgaagagatggagctgctgttcccgctgaccaaagggatcaccatccagtcggagtgcccggtgggcctgatcggcgatgacatcagcgccgtagccaacgccagcagcaaggcgctggataaaccggtgatcccggtgcgctgcgaaggctttcgcggcgtatcgcaatcgctgggccaccatatcgccaacgacgtggtgcgcgactgggtgctgaacaatcgcgaagggcagccgtttgccagcaccccgtacgatgttgccatcattggcgattacaacatcggcggcgacgcctgggcctcgcgcattctgctggaagagatggggctgcgcgtagtggcgcagtggtccggcgacggcaccctggtggagatggagaacaccccattcgttaagcttaacctcgtccactgctaccgttcgatgaactatatcgcccgccatatggaggagaaacatcagatcccatggatggaatataacttcttcggcccgaccaaaatcgccgaatcgctgcgcaagatcgccgatcaatttgatgacaccattcgcgccaatgcggaagcggtgatcgccaaatatgaggggcagatggcggccatcatcgccaaatatcgcccgcggctggaggggcgcaaagtgctgctgtacatgggggggctgcggccgcgccacgtcatcggcgcctatgaggatctcgggatggagatcatcgccgccggctacgagtttgcccataacgatgattacgaccgcaccctgccggacctgaaagagggcaccctgctgtttgacgatgccagcagctatgagctggaggccttcgtcaaagcgctgaaacctgacctcatcggctccgggatcaaagagaaatatatcttccagaaaatgggggtgccgttccgccagatgcactcctgggactattccggcccctatcacggctatgacggcttcgccatctttgcccgcgatatggatatgaccctgaacaatccggcgtggaacgaactgactgccccgtggctgaagtctgcgtga

SEQ ID NO: 17 - DNA, 1386 bp, Klebsiella variicola
  gene 1..1386: note = nifK1
  source 1..1386: mol_type = genomic DNA
atggcagatattatccgcagtgaaaaaccgctggcggtgagcccgattaaaaccgggcaaccgctcggggcgatcctcgccagcctcgggctggcccaggccatcccgctggtccacggcgcccagggctgcagcgccttcgccaaagttttctttattcagcatttccatgacccggtgccgctgcagtcgacggccatggatccgaccgccacgatcatgggggccgacggcaatatcttcaccgcgctcgacaccctctgccagcgccacagcccgcaggccatcgtgctgctcagcaccggtctggcggaagcgcagggcagcgatatcgcccgggtggtgcgccagtttcgcgaggcgcatccgcgccataacggcgtggcgatcctcaccgtcaataccccggatttttttggctctatggaaaacggctacagcgcggtgatcgagagcgtgatcgagcagtgggtcgcgccgacgccgcgtccggggcagcggccccggcgggtcaacctgctggtcagccacctctgttcgccaggggatatcgaatggctgggccgctgcgtggaggcctttggcctgcagccggtgatcctgccggacctctcgcagtcaatggatggccacctcggtgaaggggattttacgcccctgacccagggcggcgcctcgctgcgccagattgcccagatgggccagagtctgggcagcttcgccattggcgtgtcgctccagcgggcggcatcgctcctgacccaacgcagccgcggcgacgtgatcgccctgccgcatctgatgaccctcgaccattgcgatacctttatccatcagctggcgaagatgtccggacgccgcgtaccggcctggattgagcgccagcgtggccagctgcaggatgcgatgatcgactgccatatgtggcttcagggccagcgcatggcgatggcggcggagggcgacctgctggcggcgtggtgtgatttcgcccgcagccaggggatgcagcccggcccgctggtcgcccccaccagccaccccagcctgcgccagctgccggtcgagcaagtcgtgccgggggatcttgaggatctgcagcagctgctgagccaccaacccgccgatctgctggtggctaactctcacgcccgcgatctggcggagcagtttgccctgccgctgatccgcgtcggttttcccctcttcgaccggctcggtgagtttcgtcgcgtccgccaggggtacgccggtatgcgagatacgctgtttgaactggccaatctgctgcgcgaccgccatcaccacaccgccctctaccgctcgccgcttcgccagggcgccgacccccagccggcttcaggagacgcttatgccgcccattaa

SEQ ID NO: 18 - DNA, 1563 bp, Klebsiella variicola
  gene 1..1563: note = nifK2
  source 1..1563: mol_type = genomic DNA
atgagccaaacgatcgataaaattcacagctgttatccgctgtttgaacaggatgaataccagaccctgttccagaataaaaagacccttgaagaggcgcacgacgcgcagcgtgtgcaggaggtttttgcctggaccaccaccgccgagtatgaagcgctgaacttccagcgcgaggcgctgaccgtcgacccggccaaagcctgccagccgctcggcgccgtactctgcgcgctggggttcgccggcaccctgccctacgtgcacggctcccagggctgcgtcgcctattttcgcacctactttaaccgccattttaaagagccggtcgcctgcgtctccgactccatgaccgaggacgcggcggtgttcggcggcaacaacaacatgaatctgggcctgcagaatgccagcgcgctgtataaacccgagattatcgccgtctccaccacctgtatggccgaggtgatcggcgacgatctgcaggcgtttatcgccaacgccaaaaaagagggatttgttgacgaccgcatcgccattccttacgcccatacccccagctttatcggcagccatgtcaccggctgggacaatatgttcgaagggttcgcgaagacctttaccgctgactacgccgggcagccgggcaaacagcaaaagctcaatctggtgaccggatttgagacctatctcggcaacttccgcgtgctgaagcggatgatggcgcagatggatgtcccgtgcagcctgctctccgacccatcagaggtgctcgacacccccgccgacggccattaccggatgtacgccggcggcaccagccagcaggagatcaaaaccgcgccggacgccattgacaccctgctgctgcagccgtggcagctggtgaaaagcaaaaaggtggttcaggagatgtggaaccagcccgccaccgaggtggccgttccgctgggcctggccgccaccgacgcgctgctgatgaccgtcagtcagctgaccggcaaaccgatcgccgacgctctgaccctggagcgcggccggctggtcgacatgatgctggattcccacacctggctgcatggcaaaaaattcggcctctacggcgatccggatttcgtgatggggctgacgcgcttcctgctggagctgggctgcgagccgacggtgatcctcagtcataacgccaataaacgctggcaaaaagcgatgaagaaaatgctcgatgcctcgccgtacggtcaggaaagcgaagtgttcatcaactgcgacctgtggcacttccggtcgctgatgttcacccgtcagccggactttatgatcggtaactcctacggcaagtttatccagcgcgataccctggcaaagggcaaagccttcgaagtgccgctgatccgtctgggctttccgctgttcgaccgccatcatctgcaccgccagaccacctggggctatgaaggcgcaatgaacatcgtcacgacgctggtgaacgccgtgctggaaaaactggaccacgacaccagccagttgggcaaaaccgattacagcttcgacctcgttcgttaa

SEQ ID NO: 19 - DNA, 1485 bp, Klebsiella variicola
  gene 1..1485: note = nifL
  source 1..1485: mol_type = genomic DNA
atgaccctgaatatgatgctcgataacgccgcgccggaggccatcgccggcgcgctgactcaacaacatccggggctgttttttaccatggtggaacaggcctcggtggccatctccctcaccgatgccagcgccaggatcatttacgccaacccggcgttttgccgccagaccggctattcgctggcgcaattgttaaaccagaacccgcgcctgctggccagcagccagacgccgcgcgagatctatcaggagatgtggcataccctgctccagcgtcagccctggcgcggtcagctgattaatcagcgtcgggacggcggcctgtacctggtggagattgacatcaccccggtgcttagcccgcaaggggaactggagcattatctggcgatgcagcgggatatcagcgtcagctacaccctcgaacagcggctgcgcaaccatatgaccctgatggaggcggtgctgaataatatccccgccgccgtggtagtggtggacgagcaggatcgggtggtgatggacaacctcgcctacaaaaccttctgcgctgactgcggcggccgggagctgctcaccgagctgcaggtctcccctggccggatgacgcccggcgtggaggcgatcctgccggtggcgctgcgcggggccgcgcgctggctgtcggtaacctgctggccgttgcccggcgtcagtgaagaggccagccgctactttatcgacagcgcgctggcgcggaccctggtggtgatcgccgactgtacccagcagcgtcagcagcaggagcaagggcgccttgaccggctgaagcagcaaatgaccgccggcaagctgctggcggcgatccgcgagtcgctggacgccgcgctgatccagctgaactgcccgattaatatgctggcggcagcccgtcggctgaacggcgagggaagcgggaatgtggcgctggaggccgcctggcgtgaaggggaagaggcgatggcgcggctccagcgctgtcgcccatcgctggaactcgaaaaccccgccgtctggccgctgcagccctttttcgacgatctgtgcgccctctaccgtacacgcttcgatcccgacgggctgcaggtcgacatggcctcaccgcatctgatcggctttggccagcgcaccccactgctggcgtgcttaagcctgtggctcgatcgcaccctggccctcgccgccgaactcccctccgtgccgctggcgatgcagctctacgccgaggagaacgacggctggctgtcgctgtatctgactgacaacgtaccgctgctgcaggtgcgctacgctcactcccccgacgcgctgaactcgccgggcaaaggcatggagctgcggctgatccagaccctggtggcgcaccatcgcggggccattgagctggcttcccgaccgcagggcggcacctgcctgaccctgcgtttcccgctgtttaacaccctgaccggaggtgaagcatga

SEQ ID NO: 20 - DNA, 1575 bp, Klebsiella variicola
  gene 1..1575: note = nifA
  source 1..1575: mol_type = genomic DNA
atgatccctgaatccgacccggacaccaccgtcagacgcttcgacctctctcagcagttcaccgccatgcagcggataagcgtggtgctgagccgggccaccgaggccagcaaaacgctgcaggaggtgctcagcgtattacacaacgatgcctttatgcagcacgggatgatctgcctgtacgacagcgagcaggagatcctcagtatcgaagcgctgcagcaaaccggccagcagcccctccccggcagcacgcagatccgctatcgccccggcgagggactggtggggaccgtgctggcccaggggcagtcgctggtgctgccccgggtcgccgacgatcagcgttttctcgaccgcctgagcctctacgattacgatctgccgtttatcgccgtaccgttgatggggcccaacgcccggccaataggggtgctggcggcccagccgatggcgcgccaggaagagcggctgccggcctgcacccgttttctcgaaaccgtcgccaacctcgtcgcccagaccatccggctgatgatccttccggcctcacccgccctgtcgagccgccagccgccgaaggtggaacggccgccggcctgctcgtcgtcgcgcggcgtgggccttgacaatatggtcggcaagagcccggcgatgcgccagatcgtggaggtgatccgtcaggtttcgcgctgggacaccaccgtgctggtacgcggcgaaagcggcaccgggaaagagctgatcgccaacgccatccatcaccattcgccacgggctggcgccgccttcgtcaaatttaactgcgcggcgctgccggacaccctgctggaaagcgaactgttcggccatgagaaaggcgcctttaccggggcggtgcgtcagcgtaaaggacgttttgagctggcggatggcggcaccctgttcctcgatgagattggtgaaagcagcgcctcgttccaggccaagctgctgcgtatcctccaggagggggagatggagcgggtcggcggcgatgagaccctgcgggtgaatgtccgcatcatcgccgccaccaaccgtcacctggaggaggaggtccggctgggccatttccgcgaggatctctactatcgtctgaacgtgatgcccatcgccctgcccccgctgcgcgagcgtcaggaggacatcgccgagctggcgcacttcctggtgcgcaaaatcggccagcatcaggggcgcacgctgcggatcagcgagggcgcgatccgcctgctgatggagtacagctggccgggtaacgttcgcgaactggagaactgcctcgaacgatcggcggtgatgtcggagagtggcctgatcgatcgcgacgtgatcctcttcactcaccaggatcgtcccgccaaagccctgcctgccagcgggccagcggaagacagctggctggacaacagcctggacgaacgtcagcgactgatcgccgcgctggaaaaagccggctgggtgcaggccaaggcggcacggctgctggggatgacgccgcgccaggtcgcttatcggatccagatcatggatatcaccctgccgcgtctgtag

SEQ ID NO: 21 - DNA, 2838 bp, Klebsiella variicola
  gene 1..2838: note = glnE
  source 1..2838: mol_type = genomic DNA
atgatgccgctttctccgcaattacagcagcactggcagacggtcgctgaccgtctgccagcggattttcccattgccgaactgagcccacaggccaggtcggtcatggcgttcagcgattttgtcgaacagagtgtgatcgcccagccgggctggctgaatgagcttgcggactcctcgccggaggcggaagagtggcggcattacgaggcctggctgcaggatcgcctgcaggccgtcactgacgaagcggggttgatgcgagagctgcgtctcttccgccgccagatgatggtccgcatcgcctgggcgcaggcgctgtcgctggtgagcgaagaagagactctgcagcagctgagcgtcctggcggagaccctgattgtcgccgcccgcgactggctgtacgccgcctgctgtaaggagtggggaacgccatgcaatgccgagggccagccgcagccgctgctgatcctcgggatgggaaagctgggcggcggcgagctgaacttctcttccgatatcgatctgatctttgcctggcctgagcatggcgccacccgcggcggccgccgcgagctggataacgcccagttctttacccgtctggggcagcggctgatcaaggcccttgaccagccgacgcaggacggctttgtctatcgggttgacatgcgcctgcggccgtttggcgacagtgggccgctggtactcagttttgcggcgctggaagattattaccaggagcagggtcgggactgggaacgctatgcgatggtgaaagcgcggatcatgggcgataacgacggcgtgtacgccagcgagttgcgcgcgatgctccgtcctttcgtcttccgccgttatatcgacttcagcgtgatccagtcgctgcgtaacatgaaaggcatgatcgcccgcgaagtgcggcgtcgcgggctgaaagacaacatcaagctcggcgccggcgggatccgtgaaattgagtttatcgttcaggtctttcaactgatccgcggtggtcgcgaacctgcactgcagcagcgcgccctgctgccgacgctggcggcgattgatgagctacatctgctgccggaaggcgacgcggcgctgctgcgcgaggcctatctgttcctgcgccggctggaaaacctgctgcaaagcatcaacgatgagcagacccagaccctgccgcaggatgaacttaaccgcgccaggctggcgtgggggatgcataccgaagactgggagacgctgagcgcgcagctggcgagccagatggccaacgtgcggcgagtgtttaatgaactgatcggcgatgatgaggatcagtccccggatgagcaactggccgagtactggcgcgagctgtggcaggatgcgctggaagaagatgacgccagcccggcgctggcgcatttaaacgataccgaccgccgtagcgtgctggcgctgattgccgattttcgtaaagagctggatcggcgcaccatcggcccgcgcggccgccaggtgctggatcagctgatgccgcatctgctgagcgaaatctgctcgcgcgccgatgcgccgctgcctctggcgcggatcacgccgctgttgaccgggatcgtcacccgtaccacctatcttgagctgctgagcgaattccccggcgcgctgaagcacctgatcacgctctgcgcggcgtcgccgatggtcgccagccagctggcgcgccacccgctgctgctggatgagctgctggatcccaacaccctctatcagccgacggcgaccgatgcctatcgcgacgagctgcgccagtacctgctgcgcgtgccggaagaggatgaagagcagcagctggaggcgttgcgccagtttaagcaggcgcagcagctgcatatcgcggcggcggatatcgctggtaccctgccggtgatgaaggtcagcgatcacttaacctggcttgccgaagcgatcctcgacgcggtggtgcagcaggcatgggggcagatggtcgctcgctacggccagccgacccacctgcacgatcgccagggtcgcggcttcgccgtcgtcggctacggtaagcttggcggctgggagctgggctacagctccgatctcgatctggtgttcctccatgactgcccggcggaggtgatgaccgacggcgagcgggagattgacggccgtcagttctacctgcggctggcccagcggatcatgcacctgttcagcacccgcacctcgtccggtattctctacgaagtggacgcccggctgcgtccttctggcgcggcggggatgctggtcaccaccgccgacgcgtttgctgactatcagcagaacgaagcctggacgtgggaacatcaggcgctggtgcgcgcccgcgtggtctatggcgacccggcgctgcaggcgcgctttgacgccattcgtcgcgatatcctgaccaccccgcgggaggggatgaccctgcagaccgaggttcgcgagatgcgcgagaagatgcgcgcccaccttggcaacaaacatcccgatcgttttgatatcaaagccgatgccggcgggatcaccgatattgaatttattactcagtatctggtcctacgctatgccagtgacaagccgaagctgacccgctggtctgacaacgtgcgtattcttgagctgctggcgcagaacgacatcatggacgaggaggaggcgcgcgccttaacgcatgcgtacaccaccttgcgtgatgcgctccatcacctggccctgcaggagcagccgggacacgtggcgccagaggccttcagccgggagcgtcagcaggtcagcgccagctggcagaagtggctgatggcttaa

SEQ ID NO: 22 - DNA, 500 bp, Klebsiella variicola
  gene 1..500: note = PinfC
  source 1..500: mol_type = genomic DNA
agcgtcaggtaccggtcatgattcaccgtgcgattctcggttccctggagcgcttcattggcatcctgaccgaagagttcgctggcttcttcccaacctggattgcaccagtgcaggtagtggtcatgaatattaccgattctcaggctgaatacgttaacgaattgacgcgtaaactacaaaatgcgggcattcgtgtaaaagcagacttgagaaatgagaagattggctttaaaatccgcgagcacactttacgtcgtgtcccgtatatgttggtctgtggcgacaaagaagtcgaagccggcaaagtggccgtgcgcacccgtcgcgggaaagacctcggcagcatggacgtaagtgaagtgattgagaagctgcaacaagagattcgcagccgcagtcttcaacaactggaggaataaggtattaaaggcggaaaacgagttcaaacggcacgtccgaatcgtatcaatggcgagattcgcgccctggaagttcgc

SEQ ID NO: 23 - DNA, 1287 bp, Klebsiella variicola
  gene 1..1287: note = amtB
  source 1..1287: mol_type = genomic DNA
atgaaaatggcaacaatgaaatcgggtctgggggcattagcccttcttccgggactggcaatggccgcgcccgcagtggcggacaaagccgataacgcgtttatgatgatttgcaccgcgctggttctgtttatgaccatcccggggatcgcgctgttttacggcggcctgatccgcggcaaaaacgtcctttccatgctgactcaggtgattgtgacctttggcctggtctgcgtactgtgggtgatttatggctataccctggccttcggcaccggcggcagcttcttcggtagttttgactgggtgatgctgaaaaatattgaactgaaagcgctgatgggcaccttctatcagtacatccacgtggccttccagggctcgttcgcctgtatcaccgtcgggctgatcgtgggggcgctggctgagcgtattcgtttctccgccgtgctgatttttgtggtggtgtggatgacgctctcttatgttccgattgcgcacatggtctggggcggcggtctgctggcgacccacggcgcgctggacttcgcgggcggcaccgttgtacacatcaacgccgcggttgccgggctggtgggtgcgtacatgatgggcaaacgtgtgggcttcggcaaagaagcgttcaaaccgcacaatctgccgatggtgttcaccggaaccgccatcctctacgtgggctggttcggcttcaacgccggctccgccagcgcagcgaacgaaattgccgcattggctttcgtcaacaccgtcgtcgccacagcggctgccatcctggcgtggacctttggcgaatgggccctgcgcggtaaaccttcactgctgggcgcctgctccggggcgattgccggtctggttggcgtcacaccagcctgtgggtatatcggtgtcggtggggcgttgattgtgggtatcgcatctggtctggcgggcatctggggcgtaacggcgctgaaacgctggctgcgggttgatgacccttgcgacgtcttcggcgtccacggcgtctgcggcatcgtcggctgtatcctgaccggtatcttcgcggccacctctctgggcggcgtgggttatgcagaaggcgtcaccatgggccatcagctgctggtgcaactcgagagtatcgcgattaccatcgtctggtcgggcgttgtcgctttcattggctacaaagtggcggacatgaccgtggggctgcgcgtaccagaagagcaggagcgcgaaggactggacgtcaacagccatggcgaaaacgcctacaacgcctga

SEQ ID NO: 24 - DNA, 299 bp, Klebsiella variicola
  gene 1..299: note = Prm8.2
  source 1..299: mol_type = genomic DNA
cgccgtcctcgcagtaccattgcaaccgactttacagcaagaagtgattctggcacgcatggaacaaattcttgccagtcgggctttatccgatgacgaacgcgcacagcttttatatgagcgcggagtgttgtatgatagtctcggtctgagggcattagcgcgaaatgatttttcacaagcgctggcaatccgacccgatatgcctgaagtattcaattacttaggcatttacttaacgcaggcaggcaattttgatgctgcctatgaagcgtttgattctgtacttgagcttgatc

SEQ ID NO: 25 - DNA, 300 bp, Klebsiella variicola
  gene 1..300: note = Prm6.2
  source 1..300: mol_type = genomic DNA
gctaaagttctcggctaatcgctgataacatttgacgcaatgcgcaataaaagggcatcatttgatgccctttttgcacgctttcataccagaacctggctcatcagtgattttttttgtcataatcattgctgagacaggctctgaagagggcgtttatacaccaaaccattcgagcggtagcgcgacggcaagtcagcgttctcctttgcaatagcagggaagaggcgccagaaccgccagcgttgaagcagtttgaacgcgttcagtgtataatccgaaacttaatttcggtttgga

SEQ ID NO: 26 - DNA, 400 bp, Klebsiella variicola
  gene 1..400: note = Prm1.2
  source 1..400: mol_type = genomic DNA
gcccgctgaccgaccagaacttccaccttggactcggctatacccttggcgtgacggcgcgcgataactgggactacatccccattccggtgatcttaccattggcgtcaataggttacggtccggcgactttccagatgacctatattcccggcacctacaataacggtaacgtttacttcgcctgggctcgtatacagttttaattcgctaagtcttagcaataaatgagataagcggtgtgtcttgtggaaaaacaaggactaaagcgttacccactaaaaaagatagcgacttttatcactttttagcaaagttgcactggacaaaaggtaccacaattggtgtactgatactcgacacagcattagtgtcgatttttcatataaaggtaattttg

SEQ ID NO: 27 - DNA, 1536 bp, Klebsiella variicola
  gene 1..1536: note = 16S
  variation 245: note = n is a, c, t, g, unknown or other
  variation 452: note = n is a, c, t, g, unknown or other
  variation 454: note = n is a, c, t, g, unknown or other
  variation 1131..1132: note = n is a, c, t, g, unknown or other
  source 1..1536: mol_type = genomic DNA
ttgaagagtttgatcatggctcagattgaacgctggcggcaggcctaacacatgcaagtcgagcggtagcacagagagcttgctctcgggtgacgagcggcggacgggtgagtaatgtctgggaaactgcctgatggagggggataactactggaaacggtagctaataccgcataacgtcgcaagaccaaagtgggggaccttcgggcctcatgccatcagatgtgcccagatgggattagctngtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttcgggttgtaaagcactttcagcggggaggaaggcgntnaggttaataaccttgtcgattgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcattcgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgatttggaggttgtgcccttgaggcgtggcttccggagctaacgcgttaaatcgaccgcctggggagtacggccgcaaggttaaaactcaaatgaattgacgggggcccgcacaagcggtggagcatgtggtttaattcgatgcaacgcgaagaaccttacctggtcttgacatccacagaactttccagagatggattggtgccttcgggaactgtgagacaggtgctgcatggctgtcgtcagctcgtgttgtgaaatgttgggttaagtcccgcaacgagcgcaacccttatcctttgttgccagcggtnnggccgggaactcaaaggagactgccagtgataaactggaggaaggtggggatgacgtcaagtcatcatggcccttacgaccagggctacacacgtgctacaatggcatatacaaagagaagcgacctcgcgagagcaagcggacctcataaagtatgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 28 - DNA, 882 bp, Klebsiella variicola
  gene 1..882: note = nifH
  source 1..882: mol_type = genomic DNA
atgaccatgcgtcaatgcgctatctacggtaaaggcggtatcggtaaatccaccaccacccagaatctcgtcgcggccctcgccgagatgggtaagaaagtgatgatcgtcggctgcgatccgaaagcggactccacccgtctgatccttcacgctaaagcccagaacaccatcatggagatggcggcggaagtgggctcggtcgaggatctggagctcgaagacgttctgcaaatcggctatggcgatgtccgttgcgccgaatccggcggcccggagccaggcgtcggctgcgccggacgcggggtgatcaccgccatcaacttcctcgaggaagaaggcgcctatgaggaagatttggatttcgtcttctatgacgtcctcggcgacgtagtctgcggcggcttcgccatgccgatccgcgaaaacaaagcccaggagatctacatcgtctgctccggcgagatgatggcgatgtatgccgccaacaatatctccaaggggatcgtgaagtacgcgaaatctggcaaggtgcgcctcggcggcctgatctgtaactcgcgcaaaaccgaccgggaagacgaactgatcatcgccctggcggagaagcttggcacgcagatgatccacttcgttccccgcgacaacattgtgcagcgcgcggagatccgccggatgacggtgatcgagtacgacccgacctgtcagcaggcgaatgaatatcgtcaactggcgcagaagatcgtcaataacaccaaaaaagtggtgccaacgccgtgcaccatggacgagctggaatcgctgctgatggagttcggcatcatggaagaagaagacaccagcatcattggtaaaaccgccgctgaagaaaacgcggcctga

SEQ ID NO: 29 - DNA, 1449 bp, Klebsiella variicola
  gene 1..1449: note = nifD1
  source 1..1449: mol_type = genomic DNA
atgaccaacgcaacaggcgaacgtaaccttgcgctcatccaggaagtcctggaggtgtttcccgaaaccgcgcgcaaagagcgcagaaagcacatgatgatcagcgatccgcagatggagagcgtcggcaagtgcattatctcgaaccgtaaatcgcagcccggggtgatgaccgtgcgtggctgcgcctatgcgggctcgaaaggggtggtgtttgggccaatcaaagacatggcccatatctcgcacggccccatcggctgcggccagtactcgcgcgccggacggcgcaactactataccggcgtcagcggtgtcgacagcttcggcaccctgaacttcacctctgattttcaggagcgcgatattgttttcggcggcgataaaaagctgaccaaactgatcgaagagatggagctgctgttcccgctgaccaaagggatcaccatccagtcggagtgcccggtgggcctgatcggcgatgacatcagcgccgtggccaacgccagcagcaaggcgctggataaaccggtgatcccggtgcgctgcgaaggctttcgcggcgtatcgcaatcgctgggccaccatatcgccaacgacgtggtgcgcgactgggtgctgaacaatcgcgaagggcagccgtttgccagcaccccgtatgatgttgccatcattggcgattacaacatcggcggcgacgcctgggcctcgcgcattctgctggaagagatggggctgcgcgtagtggcgcagtggtccggcgacggcaccctggtggagatggagaacaccccattcgttaagcttaacctcgtccactgctaccgttcgatgaactatatcgcccgccatatggaggagaaacatcagatcccgtggatggaatataacttcttcggcccgaccaaaatcgccgaatcgctgcgcaagatcgccgatcaatttgatgacaccattcgcgccaatgcggaagcggtgatcgccaaatatgaggggcagatggcggccatcatcgccaaatatcgcccgcggctggaggggcgcaaagtgctgctgtacatgggggggctgcggccgcgccacgtcatcggcgcctatgaggatctcgggatggagatcatcgccgccggctacgagtttgcccataacgatgattacgaccgcaccctgccggacctgaaagagggcaccctgctgtttgacgatgccagcagctatgagctggaggccttcgtcaaagcgctgaaacctgacctcatcggctccgggatcaaagagaaatatatcttccagaaaatgggggtgccgttccgccagatgcactcctgggactattccggcccctatcacggctatgacggcttcgccatctttgcccgcgatatggatatgaccctgaacaatccggcgtggaacgaactgactgccccgtggctgaagtctgcgtga

SEQ ID NO: 30 - DNA, 1374 bp, Klebsiella variicola
  gene 1..1374: note = nifD2
  source 1..1374: mol_type = genomic DNA
atgaagggaaaggaaattctggcgctgctggacgaacccgcctgcgagcacaaccagaagcaaaaatccggctgcagcgctcctaagcccggcgcaaccgccggcggctgcgccttcgacggcgcgcagataacgctcctgcccatcgccgacgtcgcgcacctggtgcacggccccatcggctgcgcgggcagctcgtgggataaccgcggcagcgtcagcgccggcccggccctcaaccggctcggctttaccaccgatcttaacgaacaggatgtgattatgggccgcggcgaacgccgcctgttccacgccgtccgtcacatcgtcgaccgctatcatccggcggcggtctttatctacaacacctgcgtaccggcgatggagggggatgacctggaggccgtctgccaggccgcacagaccgccaccggcgtcccggtcatcgccattgacgccgccggtttctacggcagtaaaaatcttggcaaccgaatggcgggcgacgtgatgctcaggcaggtgattggccagcgcgaaccggccccgtggccagacaacacgccctttgccccggcccagcgccacgatatcggcctgattggcgaattcaatatcgccggcgagttctggcaggtccagccgctgctcgacgagctggggatccgcgtcctcggcagcctctccggcgacggccgctttgccgagatccagaccctgcaccgggcgcaggccaatatgctggtgtgctcgcgcgcgctgatcaacgtcgcccgggggctggagctgcgctacggcacgccgtggtttgaaggcagcttctacgggatccgcgccacctccgacgccttgcgccagctggcggcgctgctgggggatgacgacctgtgccgccgcaccgaggcgctgatcgcccgcgaagagcaggcggcggagcaggcgctggcgccgtggcgcgagcagctccgtgggcgcaaagtgttgctctacaccggcggcgtgaaatcctggtcggtggtatcagccctgcaggatctcggcatgaccgtggtggccaccggcacgcggaaatccaccgaggaggacaaacagcggatccgtgagctgatgggcgacgaggcggtgatgcttgaggagggcaatgcccgcaccctgctcgacgtggtgtaccgctatcaggccgacctgatgatcgccggcggacgcaatatgtacaccgcctggaaagcccggctgccgtttctcgatatcaatcaggagcgcgagcacgcctacgccggctatcagggcatcatcaccctcgcccgccagctctgtctgaccctcgccagtcccgtctggccgcaaacgcatacccgcgccccgtggcgctag

SEQ ID NO: 31 - DNA, 1386 bp, Klebsiella variicola
  gene 1..1386: note = nifK1
  source 1..1386: mol_type = genomic DNA
atggcagacattatccgcagtgaaaaaccgctggcggtgagcccgattaaaaccgggcaaccgctcggggcgatcctcgccagcctcgggctggcccaggccatcccgctggtccacggcgcccagggctgcagcgccttcgccaaagttttctttattcagcatttccatgacccggtgccgctgcagtcgacggccatggatccgaccgccacgatcatgggggccgacggcaatatcttcaccgcgctcgacaccctctgccagcgccacagcccgcaggccatcgtgctgctcagcaccggtctggcggaagcgcagggcagcgatatcgcccgggtggtgcgccagtttcgtgaggcgcatccgcgccataacggcgtggcgatcctcaccgtcaataccccggatttttttggctcgatggaaaacggctacagcgcggtgatcgagagcgtgatcgagcagtgggtcgcgccgacgccgcgtccggggcagcggccccggcgggtcaacctgctggtcagccacctctgttcgccaggggatatcgaatggctgggccgctgcgtggaggcctttggcctgcagccggtgatcctgccggacctctcgcagtcaatggatggccacctcggtgaaggggattttacgcccctgacccagggcggcgcctcgctgcgccagattgcccagatgggccagagtctgggcagcttcgccattggcgtgtcgctccagcgggcggcatcgctcctgacccaacgcagccgcggcgacgtgatcgccctgccgcatctgatgaccctcgaccattgcgatacctttatccatcagctggcgaagatgtccggacgccgcgtaccggcctggattgagcgccagcgcggccagctgcaggatgcgatgatcgactgccatatgtggcttcagggccagcgcatggcgatggcggcggagggcgacctgctggcggcgtggtgtgatttcgcccgcagccaggggatgcagcccggcccgctggtcgcccccaccagccaccccagcctgcgccagctgccggtcgatcaggtcgtgccgggggatcttgaggatctgcagcagctgctgagccaccaacccgccgatctgctggtggctaactctcacgcccgcgatctggcggagcagtttgccctgccgctgatccgcgtcggttttcccctcttcgaccggctcggtgagtttcgtcgcgtccgccaggggtacgccggtatgcgagatacgctgtttgagctggccaatctgctgcgcgaccgccatcaccacaccgccctctaccgctcgccgcttcgccagggcgccgaccccctgccggcttcaggagacgcttatgccgcccattaa

SEQ ID NO: 32 - DNA, 183 bp, Klebsiella variicola
  gene 1..183: note = nifK2
  source 1..183: mol_type = genomic DNA
gtgccgctgatccgtctgggctttccgctgttcgaccgccatcatctgcaccgccagaccacctggggctatgaaggcgcaatgaacatcgtcacgacgctggtgaacgccgtgctggaaaaactggaccacgacaccagccagttgggcaaaaccgattacagcttcgacctcgttcgttaa

SEQ ID NO: 33 - DNA, 1485 bp, Klebsiella variicola
  gene 1..1485: note = nifL
  source 1..1485: mol_type = genomic DNA
atgaccctgaatatgatgctcgataacgccgcaccggaggccatcgccggcgcgctgactcaacaacatccggggctgttttttaccatggtggaacaggcctcggtggccatatccctcaccgatgccagcgccaggatcatttacgccaacccagcgttttgccgccagaccggctattcgctggcgcaattgttaaaccagaacccgcgcctgctggccagcagccagacgccgcgcgcgatctatcaggagatgtggcataccctgctccagcgtcagccctggcgcggtcagctgattaatcagcgtcgggacggcggcctgtgcctggtggagattgacatcaccccggtgcttagcccgcaaggggaactggagcattatctggcgatgcagcgggatatcagcgtcagctacaccctcgaacaacggctgcgcaaccatatgaccctgatggaggcggtgctgaataatatccccgccgccgtggtggtggtggacgagcaggatcgggtggtgatggacaacctcgcctacaaaaccttctgcgctgactgcggcggccgggagctgctcaccgagctgcaggtctcccctggccggatgacgcccggcgtggaggcgatcctgccggtagcgctgcgcggggccgcgcgctggctgtcggtaacctgctggccgttgcccggcgtcagtgaagaggccagccgctactttatcgacagcgcgctggcgcggaccctggtggtgatcgccgactgtacccagcagcgtcagcagcaggagcaaggacgccttgaccggctgaagcagcaaatgaccgccggcaagctgctggcggcgatccgcgagtcgctggacgccgcgctgatccagctgaactgcccgattaatatgctggcggcagcccgtcggctgaacggcgagggaagcgggaatgtggcgctggaggccgcctggcgtgaaggggaagaggcgatggcgcggctccagcgctgtcgcccatcgctggaactcgaaaaccccgccgtctggccgctgcagccctttttcgacgatctgtgcgccctctaccgtacccgcttcgatcccgacgggctgcaggtcgacatggcctcaccgcatctgatcggctttggccagcgcaccccgctgctggcgtgcttaagcctgtggctcgaccgcaccctggccctcgccgccgaattgccctccgtgccgctggcgatgcagctctatgccgaggagaacgacggctggctgtcgctgtacctgactgataacgtaccgctgttgcaggtgcgctacgcccactcccccgacgcgctgaactcgccgggtaaaggcatggagctgcggctgatccagaccctggtggcgcaccatcgcggggccattgagctggcttcccgaccgcagggcggcacctgcctgaccctgcgtttcccgctgtttaacaccctgaccggaggtgaagcatga

SEQ ID NO: 34 - DNA, 1575 bp, Klebsiella variicola
  gene 1..1575: note = nifA
  source 1..1575: mol_type = genomic DNA
atgatccctgaatccgacccggacaccaccgtcagacgcttcgacctctctcagcagttcaccgccatgcagcggataagcgtggtgctgagccgggccaccgaggccagcaaaacgctgcaggaggtactcactgtattgcacaacgatgcctttatgcagcacgggatgatctgcctgtacgacagcgagcaggagatcctcagtatcgaagcgctgcagcaaaccggccagcagcccctccccggcagcacgcagatccgctatcgccccggcgagggactggtggggaccgtgctggcccaggggcagtcgctggtgctgccccgggtcgccgacgatcagcgttttctcgaccgcctgagcctctacgattacgatctgccgtttatcgccgtaccgttgatggggcccaacgcccggccaataggggtgctggcggcccagccgatggcgcgccaggaagagcggctgccggcctgcacccgttttctcgaaaccgtcgccaacctcgtcgcccagaccatccggctgatgatccttccggcctcacccgccctgtcgagccgccagccgccgaaggtggaacggccgccggcctgctcgtcgtcgcgcggcgtgggccttgacaatatggtcggcaagagcccggcgatgcgccagatcgtggaggtgatccgtcaggtttcgcgctgggacaccaccgtgctggtgcgcggtgaaagcggcaccgggaaagagctgatcgccaacgccatccatcaccattcgccacgggctggcgccgccttcgtcaaatttaactgcgcggcgctgccggacaccctgctggaaagcgaactgttcggccatgagaaaggcgcctttaccggggcggtgcgtcagcgtaaaggacgttttgagctggcggatggcggcaccctgttcctcgatgagattggtgaaagcagcgcctcgttccaggccaagctgctgcgtatcctccaggagggggagatggagcgggtcggcggcgatgagaccctgcgggtgaatgtccgcatcatcgccgccaccaaccgtcacctggaggaggaggtccggctgggccatttccgcgaggatctctattatcgtctgaacgtgatgcccatcgccctgcccccgctgcgcgagcgtcaggaggacatcgccgagctggcgcacttcctggtgcgcaaaatcggccagcatcaggggcgcacgctgcggatcagcgagggcgcgatccgcctgctgatggagtacagctggccgggtaacgttcgcgaactggagaactgcctcgaacgatcggcggtgatgtcggagagtggcctgatcgatcgcgacgtgatcctcttcactcaccaggatcgtcccgccaaagccctgcctgccagcgggccagcggaagacagctggctggacaacagcctggacgaacgtcagcgactgatcgccgcgctggaaaaagccggctgggtgcaggccaaggcggcacggctgctggggatgacgccgcgccaggtcgcttaccggatccagatcatggatatcaccctgccgcgtctgtag

SEQ ID NO: 35 - DNA, 2838 bp, Klebsiella variicola
  gene 1..2838: note = glnE
  source 1..2838: mol_type = genomic DNA
atgatgccgctttctccgcaattacagcagcactggcagacggtcgctgaccgtctgccagcggattttcccattgcagaactgagcccacaggccaggtcggtcatggcgttcagcgattttgtcgaacagagtgtgatcgcccagccgggctggctgaatgagcttgcggactcctcgccggaggcggaagagtggcggcattacgaggcctggctgcaggatcgcctgcaggccgtcactgacgaagcggggttgatgcgagagctgcgtctcttccgccgccagatgatggtccgcatcgcctgggcgcaggcgctgtcgctggtgagcgaagaagagaccctgcagcagctgagcgccctggcggagaccctgattgtcgccgcccgcgactggctctacgccgcctgctgtaaggagtggggaacgccatgcaatgccgagggccagccgcagccgctgctgatcctcgggatgggaaagctgggcggcggcgagctgaacttctcttccgatatcgatctgatctttgcctggcctgagcatggcgccacccgcggcggccgccgcgagctggataacgcccagttctttacccgtctggggcagcggctgatcaaggcccttgaccagccgacgcaggacggctttgtctatcgggttgacatgcgcctgcggccgtttggcgacagtgggccgctggtactcagctttgcggcactggaagattattaccaggagcagggtcgggactgggaacgctatgcgatggtgaaagcgcggatcatgggcgataacgacggcgtgtacgccagcgagttgcgcgcgatgctccgtcctttcgtcttccgccgttatatcgacttcagcgtgatccagtcgctgcgtaacatgaaaggcatgatcgcccgcgaagtgcggcgtcgcgggctgaaagacaacatcaagctcggcgccggcgggatccgtgaaattgagtttatcgttcaggtctttcagctgatccgcggtggtcgcgaacctgcactgcagcagcgcgccctgctgccgacgctggcggcgattgatgagctacatctgctgccggaaggcgacgcggcgctgctgcgcgaggcctatctgttcctgcgccggctggaaaacctgctgcaaagcatcaacgatgaacagacccagaccctgccgcaggatgaacttaaccgcgccaggctggcgtgggggatgcataccgaagactgggagacgctgagcgcgcagctggcgagccagatggccaacgtgcggcgagtgtttaatgaactgatcggcgatgatgaggatcagtccccggatgagcaactggccgagtactggcgcgagctgtggcaggatgcgctggaagaagatgacgccagcccggcgctggcgcatttaaacgataccgaccgccgtagcgtgctggcgctgattgccgattttcgtaaagagctggatcggcgcaccatcggcccgcgcggccgccaggtgctggatcagctgatgccgcatctgctgagcgaaatctgctcgcgtgccgatgcgccgctgcctctggcgcggatcacgccgctgttgaccgggatcgtcacccgtaccacctatcttgagctgctgagcgaattccccggcgcgctgaagcacctgatcacgctctgcgcggcgtcgccgatggtcgccagccagctggcgcgccacccgctgctgctggatgagctgctggatcccaacaccctctatcagccgacggcgaccgatgcctatcgcgacgagctgcgccagtacctgctgcgcgtgccggaagaggatgaagagcagcagctggaggcgttgcgccagtttaagcaggcgcagcagctgcatatcgcggcggcggatatcgctggtaccctgccggtgatgaaggtcagcgatcacttaacctggcttgccgaagcgatcctcgacgcggtggtgcagcaggcatgggggcagatggtcgctcgctacggtcagccgacccacctgcacgatcgccagggtcgcggcttcgccgttgtcggctacggtaagctcggcggctgggagctgggctacagctccgatctcgatctggtgttcctccatgactgcccggcggaggtgatgaccgacggcgagcgggagattgacggccgtcagttctacctgcggctggcccagcggatcatgcacctgttcagcacccgcacctcgtccggtattctctacgaagtggacgcccggctgcgtccttctggcgcggcggggatgctggtcaccaccgccgacgcgtttgctgactatcagcagaacgaagcctggacgtgggaacatcaggcgctggtgcgcgcccgcgtggtctatggcgacccggcgctgcaggcgcgctttgacgccattcgtcgcgatatcctgaccaccccgcgggaggggacgaccctgcagaccgaggttcgcgagatgcgcgagaagatgcgcgcccaccttggcaacaaacatcccgatcgttttgatatcaaagccgatgccggcgggatcaccgatattgaatttattactcagtatctggtcctacgctatgccagtgacaagccgaagctgacccgctggtctgacaacgtgcgtattcttgagctgctggcgcagaacgacatcatggacgaggaggaggcgcgcgccttaacgcatgcatacaccaccttgcgtgatgcgctccatcacctggccctgcaggagcagccgggacacgtggcgccagaggccttcagccgggagcgtcagcaggtcagcgccagctggcagaagtggctgatggcttaa

SEQ ID NO: 36 - DNA, 1287 bp, Klebsiella variicola
  gene 1..1287: note = amtB
  source 1..1287: mol_type = genomic DNA
atgaaaatggcaacaatgaaatcgggtctgggggcattagcccttcttccgggactggcaatggccgcgcccgcagtggcggacaaagccgataacgcgtttatgatgatttgcaccgcgctggttctgtttatgaccatcccggggatcgcgctgttttacggcggcctgatccgcggcaaaaacgtcctttccatgctgactcaggtgattgtgacctttggcctggtctgcgtactgtgggtgatttatggctataccctggccttcggcaccggcggcagcttcttcggtagctttgactgggtgatgctgaaaaatattgaactgaaagcgctgatgggcaccttctatcagtacatccacgtggccttccagggctcgttcgcctgtatcaccgtcgggctgatcgtgggggcgctggctgagcgtattcgtttctccgccgtgctgattttcgtggtggtgtggatgacgctctcttatgttccgattgcgcacatggtctggggcggcggtctgctggcgacccacggcgcgctggacttcgcgggcggcaccgttgtacacatcaacgccgcggttgccgggctggtgggtgcgtatatgatgggcaaacgtgtgggcttcggcaaagaagcgttcaaaccgcacaatctgccgatggtgttcaccggaaccgccatcctctacgtgggctggttcggcttcaacgccggctccgccagcgcagcgaacgaaattgccgcactggctttcgtcaacaccgtcgtcgccacagcggcagccatcctggcctggacctttggcgaatgggctctgcgcggcaaaccttcactgctgggcgcctgctccggggcgattgccggtctggttggcgtcacaccagcctgtgggtatatcggtgtcggtggggcgttgattgtgggtatcgcatctggtctggcgggcatctggggcgtaacggcgctgaaacgctggctgcgggttgatgacccttgcgacgtcttcggcgtccacggcgtctgcggcatcgtcggctgtatcctgaccggtatcttcgcggccacctctctgggcggcgtgggttatgcagaaggcgtcaccatgggccatcagctgctggtgcaactcgagagtatcgcgattaccatcgtctggtcgggcgttgtcgctttcattggctacaaagtggcggacatgaccgtggggctgcgcgtaccagaagagcaggagcgcgaaggactggacgtcaacagccatggcgaaaacgcctacaacgcctga

SEQ ID NO: 37 - DNA, 1435 bp, Achromobacter spiritinus
  gene 1..1435: note = 16S
  variation 255: note = n is a, c, t, g, unknown or other
  variation 454: note = n is a, c, t, g, unknown or other
  source 1..1435: mol_type = genomic DNA
ctgaagagtttgatcctggctcagattgaacgctagcgggatgccttacacatgcaagtcgaacggcagcacggacttcggtctggtggcgagtggcgaacgggtgagtaatgtatcggaacgtgcctagtagcgggggataactacgcgaaagcgtagctaataccgcatacgccctacgggggaaagcaggggatcgcaagaccttgcactattagagcggccgatatcggattagctagttggtggggtaanggctcaccaaggcgacgatccgtagctggtttgagaggacgaccagccacactgggactgagacacggcccagactcctacgggaggcagcagtggggaattttggacaatgggggaaaccctgatccagccatcccgcgtgtgcgatgaaggccttcgggttgtaaagcacttttggcaggaaagaaacgtcatgggntaataccccgtgaaactgacggtacctgcagaataagcaccggctaactacgtgccagcagccgcggtaatacgtagggtgcaagcgttaatcggaattactgggcgtaaagcgtgcgcaggcggttcggaaagaaagatgtgaaatcccagagcttaactttggaactgcatttttaactaccgggctagagtgtgtcagagggaggtggaattccgcgtgtagcagtgaaatgcgtagatatgcggaggaacaccgatggcgaaggcagcctcctgggataacactgacgctcatgcacgaaagcgtggggagcaaacaggattagataccctggtagtccacgccctaaacgatgtcaactagctgttggggccttcgggccttagtagcgcagctaacgcgtgaagttgaccgcctggggagtacggtcgcaagattaaaactcaaaggaattgacggggacccgcacaagcggtggatgatgtggattaattcgatgcaacgcgaaaaaccttacctacccttgacatgtctggaattctgaagagattcggaagtgctcgcaagagaaccggaacacaggtgctgcatggctgtcgtcagctcgtgtcgtgagatgttgggttaagtcccgcaacgagcgcaacccttgtcattagttgctacgaaagggcactctaatgagactgccggtgacaaaccggaggaaggtggggatgacgtcaagtcctcatggcccttatgggtagggcttcacacgtcatacaatggtcgggacagagggtcgccaacccgcgagggggagccaatcccagaaacccgatcgtagtccggatcgcagtctgcaactcgactgcgtgaagtcggaatcgctagtaatcgcggatcagcatgtcgcggtgaatacgttcccgggtcttgtacacaccgcccgtcacaccatgggagtgggttttaccagaagtagttagcctaacc

SEQ ID NO: 38 - DNA, 1528 bp, Achromobacter marplatensis
  gene 1..1528: note = 16S
  variation 255: note = n is a, c, t, g, unknown or other
  variation 999: note = n is a, c, t, g, unknown or other
  variation 1009: note = n is a, c, t, g, unknown or other
  variation 1437: note = n is a, c, t, g, unknown or other
  source 1..1528: mol_type = genomic DNA
ctgaagagtttgatcctggctcagattgaacgctagcgggatgccttacacatgcaagtcgaacggcagcacggacttcggtctggtggcgagtggcgaacgggtgagtaatgtatcggaacgtgcctagtagcgggggataactacgcgaaagcgtagctaataccgcatacgccctacgggggaaagcaggggatcgcaagaccttgcactattagagcggccgatatcggattagctagttggtggggtaanggctcaccaaggcgacgatccgtagctggtttgagaggacgaccagccacactgggactgagacacggcccagactcctacgggaggcagcagtggggaattttggacaatgggggaaaccctgatccagccatcccgcgtgtgcgatgaaggccttcgggttgtaaagcacttttggcaggaaagaaacgtcatgggttaataccccgtgaaactgacggtacctgcagaataagcaccggctaactacgtgccagcagccgcggtaatacgtagggtgcaagcgttaatcggaattactgggcgtaaagcgtgcgcaggcggttcggaaagaaagatgtgaaatcccagagcttaactttggaactgcatttttaactaccgggctagagtgtgtcagagggaggtggaattccgcgtgtagcagtgaaatgcgtagatatgcggaggaacaccgatggcgaaggcagcctcctgggataacactgacgctcatgcacgaaagcgtggggagcaaacaggattagataccctggtagtccacgccctaaacgatgtcaactagctgttggggccttcgggccttagtagcgcagctaacgcgtgaagttgaccgcctggggagtacggtcgcaagattaaaactcaaaggaattgacggggacccgcacaagcggtggatgatgtggattaattcgatgcaacgcgaaaaaccttacctacccttgacatgtctggaattcngaagagattnggaagtgctcgcaagagaaccggaacacaggtgctgcatggctgtcgtcagctcgtgtcgtgagatgttgggttaagtcccgcaacgagcgcaacccttgtcattagttgctacgaaagggcactctaatgagactgccggtgacaaaccggaggaaggtggggatgacgtcaagtcctcatggcccttatgggtagggcttcacacgtcatacaatggtcgggacagagggtcgccaacccgcgagggggagccaatcccagaaacccgatcgtagtccggatcgcagtctgcaactcgactgcgtgaagtcggaatcgctagtaatcgcggatcagcatgtcgcggtgaatacgttcccgggtcttgtacacaccgcccgtcacaccatgggagtgggttttaccagaagtagttagcctaaccgnaaggggggcgattaccacggtaggattcatgactggggtgaagtcgtaacaaggtagccgtatcggaaggtgcggctggatcacctccttt

SEQ ID NO: 39 - DNA, 1522 bp, Microbacterium murale
  gene 1..1522: note = 16S
  source 1..1522: mol_type = genomic DNA
tacggagagtttgatcctggctcaggatgaacgctggcggcgtgcttaacacatgcaagtcgaacggtgaacacggagcttgctctgtgggatcagtggcgaacgggtgagtaacacgtgagcaacctgcccctgactctgggataagcgctggaaacggcgtctaatactggatatgtgacgtggccgcatggtctgcgtctggaaagaatttcggttggggatgggctcgcggcctatcagcttgttggtgaggtaatggctcaccaaggcgtcgacgggtagccggcctgagagggtgaccggccacactgggactgagacacggcccagactcctacgggaggcagcagtggggaatattgcacaatgggcgcaagcctgatgcagcaacgccgcgtgagggatgacggccttcgggttgtaaacctcttttagcagggaagaagcgaaagtgacggtacctgcagaaaaagcgccggctaactacgtgccagcagccgcggtaatacgtagggcgcaagcgttatccggaattattgggcgtaaagagctcgtaggcggtttgtcgcgtctgctgtgaaatccggaggctcaacctccggcctgcagtgggtacgggcagactagagtgcggtaggggagattggaattcctggtgtagcggtggaatgcgcagatatcaggaggaacaccgatggcgaaggcagatctctgggccgtaactgacgctgaggagcgaaagggtggggagcaaacaggcttagataccctggtagtccaccccgtaaacgttgggaactagttgtggggtccattccacggattccgtgacgcagctaacgcattaagttccccgcctggggagtacggccgcaaggctaaaactcaaaggaattgacggggacccgcacaagcggcggagcatgcggattaattcgatgcaacgcgaagaaccttaccaaggcttgacatatacgagaacgggccagaaatggtcaactctttggacactcgtaaacaggtggtgcatggttgtcgtcagctcgtgtcgtgagatgttgggttaagtcccgcaacgagcgcaaccctcgttctatgttgccagcacgtaatggtgggaactcatgggatactgccggggtcaactcggaggaaggtggggatgacgtcaaatcatcatgccccttatgtcttgggcttcacgcatgctacaatggccggtacaaagggctgcaataccgcgaggtggagcgaatcccaaaaagccggtcccagttcggattgaggtctgcaactcgacctcatgaagtcggagtcgctagtaatcgcagatcagcaacgctgcggtgaatacgttcccgggtcttgtacacaccgcccgtcaagtcatgaaagtcggtaacacctgaagccggtggcctaacccttgtggagggagccgtcgaaggtgggatcggtaattaggactaagtcgtaacaaggtagccgtaccggaaggtgcggctggatcacctccttt

SEQ ID NO: 40 - DNA, 1537 bp, Kluyvera intermedia
  gene 1..1537: note = 16S
  variation 455: note = n is a, c, t, g, unknown or other
  variation 1004: note = n is a, c, t, g, unknown or other
  source 1..1537: mol_type = genomic DNA
attgaagagtttgatcatggctcagattgaacgctggcggcaggcctaacacatgcaagtcgaacggtagcacagagagcttgctctcgggtgacgagtggcggacgggtgagtaatgtctgggaaactgcccgatggagggggataactactggaaacggtagctaataccgcataatgtcgcaagaccaaagagggggaccttcgggcctcttgccatcggatgtgcccagatgggattagcttgttggtgaggtaatggctcaccaaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttcgggttgtaaagcactttcagcggggaggaaggcgatncggttaataaccgtgttgattgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcattcgaaactggcaggcttgagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaattnggcagagatgccttagtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcatatacaaagagaagcgacctcgcgagagcaagcggacctcataaagtatgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 41 - DNA, 882 bp, Kluyvera intermedia
  gene 1..882: note = nifH
  source 1..882: mol_type = genomic DNA
atgaccatgcgtcaatgcgccatttatggcaaaggtgggatcggcaaatccaccaccacgcaaaacctcgtcgccgctctcgcggaaatgggtaaaaaagtgatgatcgtcggctgcgacccgaaagcggactccacccgtctgatcctgcatgcgaaagcacagaacaccattatggagatggccgccgaagtgggttcagtggaagaccttgaactggaagatgtgctgcaaatcggttacggcggcgtgcgttgtgcagaatccggcggcccggagccaggcgtgggttgtgcaggccgcggcgttattaccgccattaacttccttgaagaagaaggcgcctatgtcagcgacctcgactttgtcttctatgacgtcctcggtgacgtggtctgcggcgggttcgccatgccgattcgtgaaaacaaagcgcaagagatctatatcgtctgctccggggaaatgatggcgatgtatgccgctaacaacatctccaaaggcatcgtgaaatacgctaaatccggcaaggtgcgcctgggcgggctgatttgtaactcccgtcagaccgaccgcgaagatgaactgatcatcgcgctggcagaaaaactgggcacccagatgattcactttgtgccacgcgacaacatcgtccagcgcgcggaaattcgccgtatgacggttatcgaatatgacccgaaatgcaaccaggccgacgaataccgcgcgctggcgaacaagatcgtcaacaacaccctgatggtcgtcccgaccccttgcaccatggatgaactggaagagctgctgatggaattcggcattatggatgtggaagacgccagcatcatcggtaaaaccgccgccgaagaaaacgcggcctga

SEQ ID NO: 42 - DNA, 660 bp, Kluyvera intermedia
  misc_feature 1..660: note = Dinitrogenase iron-molybdenum cofactor coding sequence
  source 1..660: mol_type = genomic DNA
atgaacgataacgatgtccttttctggcgcatgctggcgctatttcagtgtctgccggaactgcaacccgcgcagatcctggcctggctgacaggagaacgcgacgacgccttaaccccggcgtacctcgataagcttaacgtccgcgaactggaagcgaccttcccgtctgaaacggcgatgatgtcgcccgcacgctggagccgcgttaacgcgtgccttcacggtacgctgcccgcacacctgcaggtaaaaagcaccactcgtcaggggcaattacgggtagccttttgttcacaggatggattgctgatcaatggtcattttggtcaggggcggctgttttttatctacgcctttgatgaacagggcggatggctacacgcgttacgccgtcttccctcggccccgcaaacccaggagccgaatgaagttcgcgcgcagctcctgagtgattgccacctgctgttttgtgaagccattggcggccctgcggcggcccggctgattcgtcacaatatccacccgatgaaagtgtcgccagggatgtccattgccgcccagtgtgatgccattaccgcactgctgagcggacgtctgccaccgtggctggcaaaacgtcttgagaaagccaacccgctggaagagcgggtgttttaa

SEQ ID NO: 43 - DNA, 1374 bp, Kluyvera intermedia
  gene 1..1374: note = nifD1
  source 1..1374: mol_type = genomic DNA
atgaagggaaatgacattctcgcgctgctggatgaacccgcctgcgaacacaatcacaaacagaaatccggctgtagcgcccctaaacccggtgccacggcgggcggttgcgcgttcgacggcgcgcaaatcaccctgttgccgctgtcggatgtggcgcacctggtccacggaccgattggctgcacgggaagctcctgggataaccggggcagtatgagctccggccccagtctcaaccggctcggctttaccaccgacctgaacgagcaggatgtcattatggggcgcggcgaacggcggcttttccacgcggtgcgtcatatcgtcaaccgttatcaccctgccgccgtgtttatctataacacctgcgttccggcgatggagggtgatgatattgacgccgtctgtcaggcggcggaaaccgccaccggcgtgccagtgattgccgttgatgccgccgggttctatggcagcaaaaaccttggcaaccgtctcgcgggtgaagtgatggttaacaaggtcattggacggcgcccgcccgccccctggccggacgatacccccttcgcgccggaacaccgccacgatatcggcctgattggcgaatttaatatcgccggggagttctggcacgttcagccgctgctcgatgagctgggtattcgcgtgctgggcagcctttccggggatggccgttttagtgaaatccagaccctgcaccacgcgcaggtcaatatgctggtctgctcaagagcgctgatcaatgttgcccgcaccctggaacagcgctatggcaccccctggtttgagggcagtttttacggcgtgcgcgctacctccgatgccctgcgtcaactggcatccctgcttggcgacagcgatctgattgcccgcaccgaagccgttattgcccgcgaagaagccacggcaaatcaggcgctcgccccgtggcgcgaacggctacagggtcgcaaagtgctgctctataccggtggggtgaaatcctggtcggtggtctccgcattgcaggatttagggatgaccgtcgtggcgactggcacccgcaaatctaccgaagaagataagcagcgtattcgcgaattaatgggcgatgacgcgctaatgctggaagaaggcaacgcccgcaccctgctggatgtggtgtaccgctatcaggcggatttgatgatcgctggggggcgtaacatgtataccgcgtacaaagcgcggctgccgtttctggatatcaaccaggagcgtgaacacgcctttgcgggttatcgcggcatcgtcaccctcgcccaacagctttgccagactattgaaagccccgtctggccgcaaacacacgcccgcgcgccgtggcaataa

SEQ ID NO: 44 - DNA, 1449 bp, Kluyvera intermedia
  gene 1..1449: note = nifD2
  source 1..1449: mol_type = genomic DNA
atgagcaatgcaacaggcgaacgtaatctggaaattatccaggaagtgctggagatctttcccgaaaaaacgcgcaaagaacgcagaaagcacatgatggtgaccgacccggagatggaaagcgtcgggaaatgcatcatctctaaccgcaaatcgcagccgggtgtgatgactgtccgcggctgctcctacgccgggtcgaaaggcgtggtttttgggccgattaaagatatggcccacatctcccacggcccgatcggctgtgggcagtactcccgtgccgggcggcgcaactactacaccggggtcagcggcgttgattccttcgggacgctgaactttacctctgattttcaggagcgcgatatcgtcttcggcggcgataaaaagctcaccaaactgattgaggagatggaggaactgttcccgctgaccaaaggcatctccattcagtcggagtgcccggtaggtttaatcggtgacgatatcgaagcggtggcgaatgccagtaaaaaagcgctcaacaagccggtgatcccggtgcgttgcgaaggctttcgcggcgtgtcgcagtcgctcggtcaccatatcgccaacgacgttatccgcgactgggtgctggataaccgcgaagggaagcccttcgaatctaccccctatgacgtggccatcatcggcgattacaacatcgggggggatgcctgggcgtcgcgcattctgcttgaagagatggggttacgcgtggtggcgcagtggtccggtgacggcacgctggtagagatggaaaacaccccgttcgtcaagctgaacctggtgcactgctaccgctctatgaactacatctctcgccatatggaagagaaacacggtatcccgtggatggagtacaacttcttcggcccgaccaaaatcgccgaatcgctgcgtaagatcgccgatcaatttgacgacaccatccgcgccaatgcggaagcggtgatcgccaaatatcaggcgcaaaacgatgcgattatcgccaaataccgcccgcgtctcgaaggccgcaaggtgctgctctatatgggtggcctgcgtcctcgccacgtgattggcgcgtatgaggatttgggcatggagattgtcgccgccgggtatgaatttgcccataacgacgattacgaccgcaccctgccggacctcaaagagggcacgctgttgttcgacgatgccagcagttatgaactggaagccttcgtgaaggcgattaagccggacctcattggctcaggcatcaaggaaaaatacattttccagaaaatgggggtaccgtttcgccagatgcactcctgggattactccggcccgtatcacggctatgacggctttgccatctttgcccgcgatatggacatgacgctcaacaatcccgcctggggcgagttgaccgcaccctggctgaaatcagcctga

SEQ ID NO: 45 - DNA, 1383 bp, Kluyvera intermedia
  gene 1..1383: note = nifK1
  source 1..1383: mol_type = genomic DNA
atggcagatatcatccgtaatcagaaaccgctggcggtaagcccggtaaaaagcggccagccgttaggcgccattctggcgagcctcggctttgagcacagtattccactggtgcacggtgcgcagggatgcagcgcgttcgccaaagtgttttttatccaacattttcatgaccctattccgctgcaatccacggcgatggaccccacctcaacggtcatgggggcggacggcaatatccttgccgcgctcaatacgctgtgccagcgcaacaccccgaaagctatcgtcctgttgagtaccggcctgtctgaggcgcagggcagcgatatcagccgcgtggtacgtcagtttcgtgaggattttccccgccacaaaaatatcgccctcctgacggtcaacaccccggatttttacggcacgctggagaacggctttagtgcggtggtggaaagcgtcatcgaacagtgggtgccggaaaagcctcagcatggcctgcgtaaccggcgggtcaacttgttgttaagtcacctgctgacgcccggtgatgttgagttgctgcgcagctacgtggaggcttttggcctgcaaccggtgatcgtgccggatctttcacagtcgctggatggtcacctggcaagcggtgatttttcgccggtcactcaggggggaacgcccctgcgcattatcgaacagatgggacagagcctgtgcacgtttgctattggcgtgtcgctgtcccgtgcggcatcgctgctggcacagcgtagccgtggcgaggtgatcgtgcttccccatctgatgaccatggaacattgcgaccgttttattcatcaactgaagatcatttccgggcgcgaggttcccgcctggattgagcgccagcgcggacaattgcaggatgcgatgatcgattgtcatatgtggttgcaggatacccggctcgcgctggccgccgagggcgatctgctggcgggctggtgtgatttcgcccgtagccagggcatgctccccggccccgttgtggcgccggtcagccagccgggcctgcaacagcttcccgtggagaaagtggtcattggcgatctggaagatatgcaggatttactctgcgctatgcctgctgacctgctggtcgccaactcccatgccgcagacctggccgaacaattctccatcccgctgatccgcgccgggttccctatcttcgacaggcttggcgaatttcgtcgcgtgcgtcagggataccccggcattcgcgacacgctgtttgagctggcgaacctgatgcgcgaacgtcatcaccacctgcccgtctaccgctcccccctgcgccagcaatttgcccaggacgctgacggaggccgctatgcaacatgttaa

SEQ ID NO: 46 - DNA, 1563 bp, Kluyvera intermedia
  gene 1..1563: note = nifK2
  source 1..1563: mol_type = genomic DNA
atgagccaaactgctgagaaaattgtcacctgtcatccgctgtttgaacaggacgaataccagacgctgtttcgcaataagcgcggtctggaagaggcgcacgacccgcagcgcgtgcaagaggtttttgaatggaccaccacggcggagtatgaagcgctgaactttaagcgtgaagcgttaaccgtcgatccggcaaaggcctgccagcctttaggatcggtactctgctcgctgggttttgccaatacgctgccttatgtgcacggttcccagggctgtgtggcctatttccgcacctattttaaccgtcatttcaaagagccgatcgcttgcgtttccgactctatgacagaggatgcggcggtcttcggcggcaacaacaaccttaacaccgggttgcaaaatgccagcgccctgtacaaaccggaaattgtcgctgtctccactacctgtatggcggaggtcatcggcgatgacctgcaggcctttatcgccaacgccaaaaaggacgggtttattgatgccgccattccggtgccctacgcccatacgccaagttttatcggtagccacatcaccggctgggacaacatgtttgaaggtttcgcccgggcatttaccgccgatcacgtggcgcaaccgggcaaactggcgaagctaaacctggtgaccggttttgaaacctatcttggcaattaccgcgtgctcaaacgcatgatggcccagatggaggtgccctgtagcctgctgtctgacccgtctgaggtgttagatacgccagccgacggccactatcgcatgtatgcgggcggcacaacgcaacaagagatgcgcgacgcccccgatgctatcgacaccctgctgctgcaaccctggcatctggtgaagagtaaaaaagtggtgcaggagtcctggggccagcccgccacagaagtgtccatcccaatgggactgaccgggaccgacgaactgctgatggcagtcagtcagttaaccggcaaaccggtggccgatgaactgacgctggagcgtgggcgcctggtggatatgattctcgattcacacacctggctgcacggtaagaaattcggtctctacggcgatccggattttgtgatggggctgacgcgtttcctgctggaactgggctgcgagccgacggttatcctctgtcataacggtagcaagcgctggcagaaagcgatgaagaaaatgcttgaggcatcgccctacggtcaggagagcgaagtgttcatcaactgcgatctgtggcatttccgctcgctgatgtttacccgcaaaccggactttatgatcggcaactcgtacgccaaattcatccagcgtgacacgctggcgaaaggcgaacagtttgaagttccgctgatccgtcttggcttcccgttgttcgaccgccaccacctgcatcgccagaccacatggggttatgaaggggcgatgaatatcgtcaccaccctggtcaacgccgtgctggaaaaagtcgaccgcgataccatcaaactgggcaaaacggactacagcttcgaccttgtccgctaa

SEQ ID NO: 47 - DNA, 1488 bp, Kluyvera intermedia
  gene 1..1488: note = nifL
  source 1..1488: mol_type = genomic DNA
atgacctttaatatgatgctggagaccagcgcaccgcagcacattgcgggcaacctctcacttcaacatcccggactgttttccacgatggttgaacaggctccgatcgcgatttcgctgaccgacccggacgcgaggattctgtacgctaatccggccttttgtcgccagaccggttatagcctggaagagctgctcaaccagaaccatcgcatactggcaagccaacagacgccgcgcagcatttatcaggaactgtggcaaacgctgctgcaacagatgccctggcgcggtcagctcatcaatcgccgtcgggatggcagcctttatctggctgaggtcgatatcaccccggtcgtcaacaaacagggcgaactggaacactacctcgccatgcaacgtgatatcagcgccagctatgcgctcgaacagcgattgcgcaatcacaccaccatgagcgaggcggtgctgaacaacattcctgccgccgtggtggtggtcaacgagcaggaccaggtagtcatggacaacctcgcctacaaaaccttctgtgccgactgcggtggcaaggagctgctcaccgaactggatttctcccggcgcaaaagcgatctctatgccgggcaaatactgcctgtggtgctgcgcggcgccgtgcgctggctctctgtcacctgctggaccttgccgggggtgagcgaagaagccagccgctactttattgataccgcgctgccccgcaccctggtggtgatcaccgactgcacccagcaacaacaacaggccgaacagggccgtctcgatcgtctcaaacaggagatgaccaccgggaagctgctggccgcgatccgtgaatcgttggatgccgcgctggttcagctaaactgccccatcaatatgctggcggcggcgcgacgtctcaacggtgaagataaccataacgtggcgctggatgccgcgtggcgcgagggggaagaggcgctggcccgcctgcaacgctgccgcccttctctcgatctggaagagagcgcgctgtggcctctgcaaccgctgtttgacgacctgcgcgccctttaccatacccgctataacaatggcgaaaatctgcacgttgaaatggcctctccgcatctggcggggtttggtcagcgcacgcagatccttgcctgtctcagtttgtggctcgaccgtacgctggccctcgccgccgcgctaccggacagaacgctgcatacccagctttacgcccgtgaagaagatggctggctgtccatttggctgacagataatgtgccgctcatccatgtgcgatacgcccactcccccgatgccctgaacgcccccggcaaagggatggagctgcgattgattcaaaccctggttgcccatcatcgcggcgcaatagaactaactacccgccctgaaggcggtacctgcctgaccctgcgattcccgttatttcattcactgaccggaggcccacgatga

SEQ ID NO: 48 - DNA, 1572 bp, Kluyvera intermedia
  gene 1..1572: note = nifA
  source 1..1572: mol_type = genomic DNA
atgacccagcgacccgagtcgggcaccaccgtctggcgttttgatctctcacagcaatttaccgccatgcagcgcatcagcgtggtgttgagtcgcgcaaccgagataagccagacgctgcaggaggtgctgtgtgttctgcataatgacgcatttatgcaacacggcatgctgtgtctgtatgacaaccagcaggaaattctgagtattgaagccttgcaggaggcagaccaacatctgatccccggcagctcgcaaattcgctatcgccctggcgaagggctggtaggagccgtactgtcccagggacaatctcttgtgctgccgcgtgtcgccgacgatcaacgctttctcgacaggcttggcatctatgattacaacctgccgtttatcgccgtccccttaatggggccaggcgcgcagacgattggcgtgctcgccgcgcagccgatggcgcgtctggaggagcggcttccttcctgtacgcgctttctggaaaccgtcgccaatctggtcgcacagacagtccggctgatgaccccgcctgccgccgccacaccgcgcgccgcgattgcccagaccgaacgccagcgcaactgtggcactcctcgccccttcggctttgagaatatggtgggcaaaagcccggccatgcagcagacaatggacattatccgccaggtttcgcgctgggataccacggtactggtgcgcggcgaaagcggcaccggtaaagaacttatcgccaatgctattcatcacaactcccctcgcgccgccgcgccctttgtgaaatttaactgcgcggcgctaccggatacgctactggagagcgaattgttcggccatgaaaaaggggcgttcaccggcgcggttcgccagcgtaaaggacgttttgaactggccgatggcggcacactgtttcttgatgaaattggcgaaagcagcgcctcgttccaggccaaactgctgcgtattttgcaggagggtgaaatggagcgcgttggcggcgacgaaaccctgcgcgtcaatgtgcgtatcatcgccgccaccaaccggaatctggaagaagaggtgcggatgggcaatttccgcgaggatctctattatcgcctcaacgtaatgcccatctccctgcccccgctgcgtgaacgtcaggaggacattgccgagctggcgcactttctggtgcgcaaaatcgcccataaccaggggcgtacgctgcgcatcagtgatggcgccatccgtctgctgatgggttacaactggcccggtaacgtgcgtgagctggaaaattgcctggaacgttcggcagtgatgtcagaaaacggcctgatcgaccgcgatgtggtgctctttaaccaccgtgagaacacgccaaaactcgctatcgccgccgcgccaaaagaggatagctggcttgatcaaacgctggatgaacgtcaacggctgattgccgcgctggaaaaagccgggtgggtgcaggccaaagcggcgcgtctgctgggtatgacgccccgtcaggtcgcctatcggatacaaattatggatatcagcatgcccaggatgtga

SEQ ID NO: 49 - DNA, 2853 bp, Kluyvera intermedia
  gene 1..2853: note = glnE
  source 1..2853: mol_type = genomic DNA
atgatgccgcactctccacagctacagcagcactggcaaactgtactggcccgcttgcctgagtcattcagtgaaacaccgcttagtgaacaagcgcagttagtgcttactttcagtgattttgtgcaggatagccttgccgcgcatcctgactggctggctgagctggaaagcgcaccgccacaggcggacgagtggaagcagtatgcgcaaacccttcgcgaatcgctggaaggtgtgggagatgaggcatcattaatgcgtgcgctgcgcctgttccgtcgccatatgatggtgcgcattgcctgggcgcagtcgctggcgctggtggcagaagatgagacgttgcagcagttgagcgtactggcggagaccctgatcgtcgctgcacgcgactggctttacgatgcctgctgtcgcgagtggggaacgccgtgcaatcagcagggggaaccgcagccgttgctgatcctgggcatgggcaagctgggtggcggggagcttaacttttcgtccgatatcgatctgatttttgcctggccggaaaacggttcaacgcgcggtgggcgacgcgaacttgataacgcccagttttttactcgcttgggacagcgcctgatcaaagtgctcgaccagccgacgcaggatggctttgtctatcgcgtggatatgcggctgcgcccgtttggcgacagcggtccgctggtgctgagttttgccgcgctggaagattattatcaggagcaggggcgcgactgggaacgttatgcgatggtgaaagcccgcattatgggcgataaggacgatgtttacgctggcgaattacgggccatgctgcggccgttcgtcttccgtcgctatatcgatttcagcgttattcagtctctgcgtaacatgaaagggatgattgcccgcgaagtgcgccgccgtggtctgaaagataacattaagctgggcgcgggcggcatccgtgagattgagtttatcgttcaggtgttccagttgatacgcggtgggcgcgagccgtcgttgcagtcccgttcactgttaccgacgctggacgctatcgataagctgggtttgctgccgcctggcgatgcaccggcgttacgccaggcctatttgtatctgcgccgtctggaaaacctgctgcaaagcattaacgacgaacaaacgcagacgctgccgacagatgaactcaatcgcgcgcgtctggcctgggggatgcgggtcgcagactgggaaaccctgaccgctgagcttgaaaagcagatgtctgccgtacgagggatattcaacaccctgattggcgatgacgaagccgaagagcagggggatgcgctctgcgggcaatggagtgagttgtggcaggatgcgtttcaggaagatgacagcacgcctgtgctggcgcacctttctgacgatgatcgccgccgcgtggtcgcgatgattgctgattttcgcaaagagctggataaacgcaccattggcccacgcggccgccaggtgctcgaccatctgatgccgcatctgttgagtgatgtctgctcccgtgaggatgcccctgtaccgttgtctcgcgtgacgccgctgttaacgggaattgtcacgcgtacgacgtatcttgagctgctcagcgagtttcctggtgcgcgtaagcatctgatttcactctgtgccgcctcgccgatggtggccagtaagctggcgcgctatccgttattgctggatgagttgctcgatccgaataccctttatcagcccacggcgatgaatgcctaccgggatgagctacgtcagtatctgctgcgtgtgccggatgacgatgaagagcagcaactggaggcgttacgccagtttaaacaggctcaattgttgcgtgtggcggcagcagatctggcaggcacactccccgtgatgaaagtgagcgatcacttaacatggcttgccgaagccatcattgaagccgtggtacaacaggcgtggagcctgatggtatcgcgttatgggcagccgaaacacttacgcgaccgtgaaggccgtgggtttgcagtggtcggttacggcaaactgggcggttgggagctgggctatagttccgatctggatttgattttccttcatgactgtccggtggacgtgatgactgacggcgagcgggaaatcgatggccgccaattttatctgcgccttgcccagcgcgtgatgcacctgttcagtacgcgcacctcatccgggatcctgtatgaggtagacgcgcgcttgcgcccgtccggtgcggcgggaatgctggtgacctcaaccgaatcctttgccgactaccagcgcaccgaagcctggacctgggaacatcaggcgctggttcgcgcccgcgttgtctatggcgatccacaattaaacgcgcaatttgatgccatccgccgcgatatcaccatgaccgtgcgtaatggtgcaacgttacaaaccgaggtgcgcgagatgcgcgaaaaaatgcgcgcccacttgagcaataagcacaaggatcgctttgatattaaagccgatgagggtggaattaccgatatcgaatttatcacccagtatctggtgctgcgttatgcccatgccaaaccgaaactgacgcgctggtcggacaatgtccgcattctggaagggctggcgcaaaacggcattatggaagagcaggaagcgcaggcacttaccaccgcctatacaacgttgcgtgatgagctgcatcacctggcgctacaggagctgccaggacatgttccggaggcatgttttgtcgctgaacgcgcgatggtgcgagcctgctggaacaagtggttggtggagccgtgcgaggacgcgtaa

SEQ ID NO: 50 - DNA, 1290 bp, Kluyvera intermedia
  gene 1..1290: note = amtB
  source 1..1290: mol_type = genomic DNA
atgaagaaagcactattaaaagcgggtctggcctcgctggcattactgccgtgtctggctatggcagccgatccggttgtcgtcgataaagccgacaatgcctttatgatgatttgcaccgcgctggtgctgtttatgtcaattccgggcatcgccctgttctatggtggtttaatccgcggtaaaaacgtcctttctatgctgacacaggttgcggttacgttcgcactggtgtgcgtgctgtgggtggtttacggctactctctggcctttggcactggcggcagcttcttcggtagcttcgactgggtgatgctgaaaaatattgagctgaaagcgctgatgggcaccatctatcagtacattcacgttgcgttccagggctcgtttgcctgtattaccgtcggcctgattgtcggtgcgctggcagaacgtatccgtttctccgcagtactgattttcgtcgtggtatggctgacgctgtcctacgtgccgatcgcacacatggtctggggcggcggtctgctggcaacccatggcgccatggattttgcgggcggtacagtcgttcacatcaacgcagccgttgcaggcctggtgggtgcttacctgattggcaaacgtgtcggtttcggtaaagaagcgtttaaaccgcacaacctgccgatggtgtttaccggtacggcaatcctctactttggctggttcggattcaacgcgggttctgcaagcgcggcgaacgaaattgcgggtctggcttttgttaacaccgtcgtggcaacagcgggtgcaatcctctcctgggtcttcggtgagtgggcgctgcgcggcaaaccgtctctgttgggtgcctgttctggtgcgattgctggcctcgtgggtatcaccccggcgtgtggttacgttggtgtgggtggcgcgctgatcgtgggcatcgttgcaggcctggcgggtctgtggggcgttaccgcgctgaaacgctggctgcgtgttgacgacccgtgtgatgtcttcggtgttcacggcgtgtgcggtatcgtaggttgtatcatgacaggtatcttcgcagccacttcactgggcggcgtgggttatgccgaaggcgtgaccatgggccatcaggttctggtacaactggaaagtatcgccattactatcgtatggtctggtatcgtcgcctttatcggttacaaactggctgatatgacagtgggtctgcgtgttccggaagatcaggaacgcgaagggctggacgtcaacagccacggcgagaacgcctacaacgcctga

SEQ ID NO: 51 - DNA, 498 bp, Kluyvera intermedia
  gene 1..498: note = PinfC
  source 1..498: mol_type = genomic DNA
ctggggtcactggagcgctttatcggcatcctgaccgaagaatttgccggtttcttcccgacctggctggcccctgttcaggttgtggtgatgaatatcactgattctcaagctgaatatgtcaacgaattgacccgtaaattgcaaaatgcgggcattcgtgtaaaagcggacttgagaaacgagaagattggctttaaaatccgcgagcacactttacgtcgtgtcccttatatgttggtctgtggtgataaagaggtggaagcaggcaaagtggccgttcgcacccgccgcggtaaagacctgggcagcctggacgtaagtgaagtgattgagaagctgcaacaagagattcgcagccgcagtcttcaacaactggaggaataaggtattaaaggcggaaaacgagttcaaacggcacgtccgaatcgtatcaatggcgagattcgcgcccaggaagttcgcttaactggtctggaaggtgagcagctgggtatt

SEQ ID NO: 52 - DNA, 1537 bp, Kosakonia pseudosacchari
  gene 1..1537: note = 16S
  variation 454: note = n is a, c, t, g, unknown or other
  source 1..1537: mol_type = genomic DNA
attgaagagtttgatcatggctcagattgaacgctggcggcaggcctaacacatgcaagtcgaacggtagcacagagagcttgctctcgggtgacgagtggcggacgggtgagtaatgtctgggaaactgcctgatggagggggataactactggaaacggtagctaataccgcataacgtcgcaagaccaaagagggggaccttcgggcctcttgccatcagatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaaggcganacggttaataaccgtgttgattgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcatccgaaactggcaggcttgagtctcgtagagggaggtagaattccaggtgtagcggtgaaatgcgtagagatctggaggaataccggtggcgaaggcggcctcctggacgaagactgacgctcaggtgcgaaagcgtggggagcaaacaggattagataccctggtagtccacgccgtaaacgatgtctatttggaggttgtgcccttgaggcgtggcttccggagctaacgcgttaaatagaccgcctggggagtacggccgcaaggttaaaactcaaatgaattgacgggggcccgcacaagcggtggagcatgtggtttaattcgatgcaacgcgaagaaccttacctggtcttgacatccacagaacttgccagagatggcttggtgccttcgggaactgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 53 - DNA, 882 bp, Kosakonia pseudosacchari
  gene 1..882: note = nifH
  source 1..882: mol_type = genomic DNA
atgaccatgcgtcaatgcgccatttacggcaaaggtgggatcggtaaatcgaccaccacacagaacctggtcgccgcgctggcggagatgggtaagaaagtcatgatcgtcggctgcgatccgaaagccgactccacgcgtttgatcctgcatgcgaaagcgcagaacaccattatggagatggccgccgaagtcggctccgtcgaagacctggaattagaagacgtgctgcaaatcggttacggcggcgtgcgctgcgcggaatccggtggcccggagccaggtgtgggttgtgccggtcgtggcgtgatcaccgcgattaacttcctcgaagaagaaggcgcttacgtgccggatctggattttgttttctacgacgtgctgggcgacgtggtatgcggtggtttcgccatgccgattcgtgaaaacaaagcgcaggagatctacatcgtttgctctggcgaaatgatggcgatgtacgccgccaataacatctccaaaggcatcgtgaaatatgccaaatccggtaaagtgcgcctcggcgggctgatttgtaactcgcgccagaccgaccgcgaagatgaactcatcattgcgctggcggaaaaactcggcacgcaaatgatccactttgttccccgcgacaacattgtgcagcgtgcggaaatccgccgtatgacggttatcgaatatgacccgacctgcaatcaggccaacgaatatcgcagccttgccagcaaaatcgtcaacaacaccaaaatggtggtaccaaccccctgcaccatggatgaactggaagaactgctgatggagttcggcattatggatgtggaagacgccagcatcattggtaaaaccgccgccgaagaaaacgccgtctga

SEQ ID NO: 54 - DNA, 1449 bp, Kosakonia pseudosacchari
  gene 1..1449: note = nifD1
  source 1..1449: mol_type = genomic DNA
atgagcaatgcaacaggcgaacgtaacctggaaatcatcgagcaggtgctggaggttttcccggaaaagacgcgcaaagagcgcagaaaacacatgatggtgacggacccggagcaggagagcgtcggcaagtgcatcatctctaaccgcaaatcgcagccgggcgtgatgaccgtgcgtggctgctcgtatgccggatcaaaaggggtggtatttgggccaatcaaagatatggcgcatatctcccacggcccgatcggctgcgggcagtactcccgcgccgggcggcgtaactactataccggcgtcagcggcgtggacagtttcggcacgctcaacttcacctccgatttccaggagcgcgacatcgtgtttggcggcgacaaaaagctcgccaaactgattgaagagctggaagaactgtttccgctgaccaaaggcatttcgattcagtcggaatgcccggtcggcctgattggcgatgatattgaagccgtggcgaacgccagccgcaaagcgatcaacaaaccggttattccggtgcgttgcgaaggctttcgcggcgtgtcgcaatccctcggtcaccatattgccaacgatgtgatccgcgactgggtactggataaccgcgaaggcaaaccgtttgaatccaccccttacgatgtggcgatcatcggcgattacaacatcggtggcgacgcctgggcctcgcgcattttgctcgaagagatggggttgcgggtggtcgcgcagtggtccggcgacggtacgctggtggagatggaaaacacgccgttcgtcaaactgaacctggtgcactgctaccgctcgatgaactacatctcgcgccatatggaggagaagcacggtattccgtggatggaatacaacttctttggcccgacgaaaatcgcggaatcgctgcgcaaaatcgccgacctgttcgacgacaccattcgcgccaacgccgaagcggtgatcgcccgataccaggcgcagaacgacgccattatcgccaaatatcgcccacgtctggagggtcgcaaagtgttgctctatatgggcgggctgcgtccgcgccatgtgattggcgcctatgaagatctgggaatggagatcatcgccgccggttatgagtttggtcataacgacgattacgaccgcaccctgccggatctgaaagagggcacgctgctgtttgatgacgccagcagctatgagctggaggcgtttgtcaacgcgctgaaaccggatctcatcggttccggcatcaaagagaagtacatctttcagaaaatgggcgtgccgtttcgccagatgcactcctgggattactccggcccgtaccacggctatgacggcttcgccatcttcgcccgcgatatggatatgacgctcaacaaccccgcctggggtcagttgaccgcgccgtggcttaaatccgcctga

SEQ ID NO: 55 - DNA, 1374 bp, Kosakonia pseudosacchari
  gene 1..1374: note = nifD2
  source 1..1374: mol_type = genomic DNA
atgaaggggaacgacatcctggctctgctcgatgaaccagcctgcgagcataaccataaacagaaaaccggctgtagcgcgccaaaacccggcgccaccgccggaggctgcgccttcgacggcgcacagatcaccctgctgccactttccgatgtggcgcatctggtacatggcccgattggctgcgccggcagctcatgggataaccgtggcagcctgagttctggcccgctgattaaccgactcggattcaccactgatttgaacgaacaggatgtcatcatggggcgcggcgagcggcggttgtttcacgcggtgcgccatattgtcgagcgctatcacccggcggcggtatttatttacaacacctgcgttccggctatggaaggcgatgacattgacgcggtctgccaggccgccgcgaccgccaccggtgtgcccgtgattgccgtagatgtggccggtttttacggtagcaaaaacctgggtaaccgcctcgcgggcgaggtgatggtgaaaaaagttatcggcgggcgcgaacccgcgccgtggccggacaatacaccttttgccccggcgcaccgccatgacataggcctgattggcgaatttaacatcgccggcgagttctggcatatccagccgctgcttgatgagctgggtattcgcgtccttggctccctttccggcgacgggcgctttgccgagatccagacgttgcaccgcgcgcaggtcaatatgctggtgtgctccagggcgctgattaatgtcgccagatcgcttgaacaacgttatggcacaccctggtttgaaggcagtttttatggcgttcgcgccacctccgatgccctgcgccagctggcaacactcaccggcgatagcgatttaatggcgcgaaccgaacggctgatcgcacgtgaagagcaagccacagaacaggcgctagcaccgctgcgtgaacggttacacggccggaaagtgctgctctataccggtggcgtgaaatcctggtcggtggtttcggcgctgcaggatctcggcatgacggtcgttgctaccggaacgcgcaaatccaccgaagaggataaacaacgcatccgtgaactgatgggcgatgacgccatcatgctggatgaaggcaatgcccgcgccttgctggatgtggtctatcgctacaaagccgacatgatgatcgcgggcgggcgcaacatgtacaccgcctataaagcgcgtctgccctttctggatatcaaccaggagcgtgaacacgcgtttgccggttatcgcggcatcatcacgcttgccgaacaactttgtcagacgctggaaagcccggtctggccgcaaacacatgcccgcgccccgtggcaataa

SEQ ID NO: 56 - DNA, 1563 bp, Kosakonia pseudosacchari
  gene 1..1563: note = nifK1
  source 1..1563: mol_type = genomic DNA
atgagccagactgctgagaaaatacagaattgccatcccctgtttgaacaggacgcctaccagacactatttgccggtaaacgggcactcgaagaggctcactcgccggagcgggtgcaggaagtgtttcaatggaccaccaccccggaatacgaagcgctgaacttcaaacgcgaagcgctgactatcgacccggcaaaagcctgccagccgctgggggcggtgctctgttcgctggggtttgccaacaccctgccgtatgtgcacggttcacagggttgtgtggcctatttccgtacgtactttaaccgccacttcaaagaaccggtggcctgcgtgtcggattcgatgacggaagacgcggccgtgttcggcgggaataacaacctcaacaccgggttacaaaacgccagcgcactgtataaaccggagattatcgccgtctctaccacctgtatggcggaagtgatcggtgatgatttacaggcgtttatcgccaacgccaaaaaagatggttttctcgatgccgccatccccgtgccctacgcccacacccccagttttatcggtagccatatcaccggctgggacaacatgtttgaaggttttgcccgtacctttaccgcaaaccatcagccacagcccggtaaactttcacgcctgaacctggtgaccgggtttgaaacctatctcggcaatttccgcgtgctgaaacgcatgatggaacaaatggaggtgcaggcgagtgtgctctccgatccgtcggaggtgctggacacccccgccaatggccattaccagatgtacgcgggcggtacgacgcagcaagagatgcgcgaggcaccggatgccatcgacaccctgctgctgcaaccgtggcagctggtgaaaagcaaaaaagtggtgcaggagatgtggaatcagcccgccaccgaggttgccattcccgtcgggctggcaggcacagacgaactgttgatggcgattagccagttaaccggcaaagccattcccgattcgctggcgctggagcgcgggcggctggtcgatatgatgctcgactcccacacctggttacacggtaaaaaattcggtctgtttggcgatccggattttgtcatgggattgacccgcttcctgctggaactgggctgtgaacctgccgtcatcctctgccataacggtaacaaacgctggcaaaaagcgatgaagaaaatgctcgatgcttcaccgtacggccaggagagcgaagtgtttatcaactgcgacttgtggcatttccgctcgctgatgttcacccgccagccggattttatgattggcaactcgtacgccaagtttattcagcgcgacaccttagccaagggcgaacagtttgaagtcccgctgatccgcctcggttttccgctgttcgaccgtcaccatctgcaccgccagaccacctggggctacgagggcgcgatgagcattctcacgacgctggtgaatgcggtactggagaaagtggacaaagagaccatcaagctcggcaaaaccgactacagcttcgatcttatccgttaa

SEQ ID NO: 57 - DNA, 1386 bp, Kosakonia pseudosacchari
  gene 1..1386: note = nifK2
  source 1..1386: mol_type = genomic DNA
atggctgatattgttcgtagtaaaaaaccgctggcggtgagcccgataaaaagcggccagccgctgggggcgatcctggcaagcctgggtttcgaacagtgcataccgctggtacacggcgctcaggggtgcagcgcgttcgcgaaagtgttctttattcaacattttcacgacccgatcccgctgcaatcgacggcgatggacccgacttccaccattatgggcgccgatgaaaacatttttaccgcgctcaatgttctctgccagcgcaacgccgcgaaagccatcgtgctgctcagcaccgggctgtcagaagcccagggcagcgatatttcacgagtggtgcgccagtttcgtgatgactttccgcggcataaaaacgtggcgctgctcaccgtcaacaccccggatttctacggctcgctggaaaacggctacagcgccgtgctggaaagcatgattgaacagtgggtgcccgcgcagcccgccgccagcctgcgcaaccgtcgcgtcaacctgctggtcagccatttactgacgccgggcgatatcgaactgttacgcagttatgtggaagcattcggtctgcaaccggtgattgtgccggatctatcgcagtcgctggacggacatctggccaacggtgatttttcgcccgtcacccaggggggaacaccgctgcgcatgattgaacagatggggcaaaacctggccacttttgtgattggccactcgctggggcgggcggcggcgttactggcgcagcgcagccgtggcgaggtgatcgccctgccgcatctgatgacgcttgatgcgtgcgacacctttatccatcgcctgaaaaccctctccgggcgcgacgtgcccgcgtggattgagcgccagcgcgggcaagtgcaggatgcgatgatcgattgccatatgtggttgcagggcgcggctatcgccatggccgcagaaggcgatcacctggcggcatggtgcgatttcgcccgcagccagggcatgatccccggcccggttgtcgcgccggtcagccagccggggttgcaaaatctgccggttgaaatggtggtcatcggcgatctggaagatatgcaggatcggctttgcgcgacgcccgccgcgttactggtggccaattctcatgccgccgatctcgccacgcagtttgatatgtcgcttatccgcgccgggtttccggtgtatgaccggctgggggaatttcgtcggctgcgccaggggtatagcggcattcgtgacacgctgtttgagctggcgaatgtgatgcgcgaacgccattgcccgcttgcaacctaccgctcgccgctgcgtcagcgcttcggcgacaacgttacgccaggagatcggtatgccgcatgttaa

SEQ ID NO: 58 - DNA, 1488 bp, Kosakonia pseudosacchari
  gene 1..1488: note = nifL
  source 1..1488: mol_type = genomic DNA
atgaccctgaatatgatgatggatgccagcgcgcccgaggccatcgccggtgcgctttcgcaacaacatcctgggctgttttttaccatcgttgaagaagcccccgtcgctatttcactaaccgatgccgaggcacgtattgtctatgccaacccggcattctgccgccagaccggctatgagcttgaggagttgttgcagcaaaatccccgcctgcttgccagtcagcagaccccacgggaaatctaccaggatatgtggcacaccctgttacaacgtcgaccatggcgcgggcaattgatcaaccgccaccgtgacggcagcctttttctggttgagatcgatatcaccccggtgattaacccgtttggcgaactggaacactacctggccatgcagcgcgatatcagcgccggttatgcgctggagcagcggttgcgtaatcacatggcgctgaccgaagcggtgctgaataacattccggcggcggtggtcgtggtcgatgaacgcgatcgtgtggttatggataacctcgcctataaaactttctgtgctgattgcggcggaaaagagctactgagcgaactccatttttcagcccgtaaagcggagctggcaaacggccaggtcttaccggtggtgctgcgcggcgcggtgcgctggttgtcggtcacctgctgggcgctgccaggcgtcagcgaagaagccagtcgctactttattgataataccttgacgcgcacgctggtggtcatcaccgacgacacccagcagcgccagcagcaagagcaaggacggcttgaccgccttaaacagcagatgaccagcggcaaactgctggcggcgatccgcgaagcgcttgacgccgcgctgatccagcttaactgccccatcaatatgctggcggcggcgcggcgtttaaacggcagtgataacagcaacgtagcgctggacgccgcgtggcgcgaaggtgaagaagcgatggcgcggctgaaacggtgccgcccgtcgctggagctggaaagtgccgccgtctggccgctgcaacccttttttgacgacttgcgcgcgctttatcacacccgctacgagcagggtaaaaatttgcaggtcacgctggattcgacgcatctggtgggatttggtcagcgaacccaactgctggcctgcctgagtctgtggctcgatcgcacgctggatattgccgtcgggctgcgtgatttcaccgcccaaacgcagatttacgcccgggaagaagcgggctggctctcgttgtatatcactgacaatgtgccgttgattccgctgcgccatacccattcgccggatgcgcttaacgcaccgggaaaaggtatggagttgcggctgatccagacgctggtagcgcatcacaacggcgcgatagaactcacttcacgccccgaagggggaagctgcctgaccctacgattcccgctatttcattcactgaccggaggttcaaaatga

SEQ ID NO: 59 - DNA, 1575 bp, Kosakonia pseudosacchari
  gene 1..1575: note = nifA
  source 1..1575: mol_type = genomic DNA
atgacccagcgaaccgagtcgggtaataccgtctggcgcttcgatttatcccagcagttcaccgcgatgcagcggataagcgtggttctcagccgggcgaccgaggttgaacagacactccagcaggtgctgtgcgtattgcacaatgacgcctttttgcagcacggcatgatctgtctgtacgacagccagcaggcgattttgactattgaagcgttgcaggaagccgatcagcagttgatccccggcagctcgcaaattcgctaccgtccgggtgaagggctggtcgggacggtgctttcgcaggggcaatcgttagtgctggcgcgtgtggctgacgatcagcgctttcttgaccgcctgggactgtatgattacaacctgccgtttatcgccgtgccgctgatagggccggatgcgcagacttttggcgtgctgacggcgcaaccgatggcgcgttacgaagagcggttacccgcctgcacccgctttctggaaacggtcgcgaatctggtggcgcagaccgtgcgtttgatgacgccgccggctgcacgcccttccccacgcgctgccatcacgccaaccgccagcccgaaatcgtgcagtacttcacgcgcgttcggcttcgaaaatatggtcggcaacagcccggcaatgcgccagaccatggagattatccgtcaggtttcgcgctgggataccaccgttctggtgcgcggcgagagcggcaccggcaaggaactgattgccaacgccatccatcacaattcgccgcgcgccagtgcgccatttgtgaaattcaactgtgcggcgctgccggacacattgcttgaaagcgaattatttggtcatgaaaaaggcgcctttaccggcgcggtacgccagcgtaaaggccgttttgagctggccgatggcggcacgctgtttcttgacgaaattggggaaagcagcgcctcgtttcaggctaagctgctgcgtattttgcaggagggcgaaatggaacgcgtcggtggtgacgagacattgcaagtgaatgtgcgcatcattgccgcgacgaaccgcaaccttgaagatgaagtacgcctgggacattttcgcgaagatctctattaccgcctgaatgtgatgcccatcgccctgccgccgctgcgcgaacgccaggacgacatcgccgaactggcacattttctggtgcgtaaaatcgcccacaaccagaaccgcacgctgcgcattagcgagggcgctatccgcctgctgatgagctacagctggcccggcaatgtgcgcgaactggaaaactgccttgagcgctctgcggtgatgtcggaaaacggtctgatcgatcgggacgtgattttatttaatcatcgcgaccagccagccaaaccgccggttatcagcgtcacgcccgacgataactggctcgataacacccttgacgagcgccagcggctgattgccgcgctggaaaaagcgggatgggtacaagccaaagccgcccgcttgctggggatgacgccgcgccaggtcgcttatcgtattcagaccatggatatcaccctgccaaggctataa

SEQ ID NO: 60 - DNA, 2850 bp, Kosakonia pseudosacchari
  gene 1..2850: note = glnE
  source 1..2850: mol_type = genomic DNA
atgccgcaccacgcaggattgtcgcagcactggcaaacggttttttctcgtctgccggaagcgctcaccgcgcaaccattgagcgcgcaggcgcagtcagtgctcacttttagtgattttgttcaggacagcatcatcgtgcatcctgagtggctggcagagcttgaaagcgcaccgccgccagcgaacgagtggcaacactacgcgcaatggctgcaagcggcgctggagggcgtcaccgatgaaacctcgctgatgcgcacgctgcggctgtttcgccgtcgcattatggtgcgcatcgcctggagtcaggcgctacagttggtggcggaagaggatatcctgcaacagctcagcgtgctggcggaaactctgatcgtcgccgcgcgcgactggctctatgacgcctgctgccgtgagtggggaacgccgtgcaatccgcaaggcgtcgcgcagccgatgctggtgctcggcatgggcaaacttggcggcggcgaactcaatttctcatccgatatcgatttgatttttgcctggccggaaaatggcaccacgcgcggcggacgccgtgaactggataacgcgcagttttttacccgccttggtcaacggctaattaaagtcctcgaccagcccacgcaggatggctttgtctaccgcgtcgatatgcgcttgcgtccctttggcgacagcggcccgctggtgctgagttttgccgcgctggaagattactaccaggagcaggggcgcgactgggaacgatacgcgatggtgaaagcgcgcattatgggggacaacgacggcgaccatgcgcgagagttgcgcgccatgctgcgcccgttcgttttccgccgctatatcgacttcagcgtgatccagtctctgcgcaacatgaaaggcatgattgcccgcgaagtgcggcgtcgcggcctgaaggacaacataaaactcggcgcgggcggtattcgcgaaatagagtttatcgtgcaggttttccagttgattcgcggcggtcgcgagcctgcgctgcaatcgcgttcgctgttgccgacgcttgctgccattgatcaactacatctgctgccggatggtgatgcaccccggctgcgcgaggcgtatttgtggctgcgacggctggaaaacttgctgcaaagcattaatgacgaacagacacagacgctgccggccgatgatttgaatcgcgcgcgcctcgcctggggaatgggcaaagagagctgggaagcgctctgcgaaacgctggaagcgcatatgtcggcggtgcggcagattttcaacgatctgattggcgatgatgaaacggattcgccggaagatgcgctttctgagggctggcgcgaattgtggcaggatgcgttgcaggaagaggactctacgcccgtgctggcgcatctttccgaggacgatcgccgccgcgtggtggcgctgattgctgattttcgcaaagagctggataaacgcaccattggcccgcgcgggcgacaggtactcgatcacttaatgccgcatctgctcagcgatgtatgctcgcgtgacgatgcgccagtgccgctgtcgcgtctgacgccgctgctcaccggtattattacgcgcaccacttaccttgagctgctgagtgaattccccggtgcgctgaaacacctcatttccctgtgcgccgcgtcgccgatggtggccagccaactggcgcgctacccgatcctgctcgatgaactgctcgacccgaacacgctctatcaaccgacggcgatgaacgcctatcgcgatgaactgcgacaatacctgttgcgcgtgccggaagaggatgaagagcagcaactggaggcgctacggcagtttaagcaggcgcagttgttgcgcgtagcggcggcggatatcgccggtacgttacccgtcatgaaagtgagcgatcacttaacctggctggcggaagcgattatcgatgcggtggtgcagcaagcctggaaccagatggtggcgcgttacggccagccgacgcatctgcacgatcgcgaagggcgcggtttcgccgtggtcggttacggcaaacttggcggctgggaattaggttacagctccgatctggatctggtgttcctgcacgactgccccatggatgtgatgaccgatggcgagcgtgaaatcgatggccgccagttctatttgcgcctcgcgcagcgcgtgatgcacctgttcagcacgcgcacgtcgtccggcattctttatgaagtcgatgcgcgtttgcgcccgtccggcgcggccggaatgctggtgaccactgcggaagcgttcgccgattatcaaaaaaatgaagcctggacatgggagcatcaggcgctggcgcgtgcgcgcgtggtgtacggcgatccgcaactgaccgccgaatttgacgccattcgccgcgatatcctgatgacctcccgcgatgccgctaccctgcaaaccgaagtgcgggaaatgcgtgagaaaatgcgcgcccatcttggtaacaagcacaaagaccgtttcgatctgaaagccgatgaaggcggtatcaccgatattgagtttatcgctcagtatctggtgctgcgctttgcccatgagaagccgaaactgacgcgctggtcggataatgtgcgcatcctcgaagggctggcgcaaaacggcatcatggatgagcaggaagcgcaggcattgacgctggcgtacaccacgttgcgtgatgagctgcaccacctggcgctgcaagagctgccaggacatgtggcgctctcctgttttgtcgccgagcgtgcgcttatcaaaaccagctgggacaagtggctggtggaaccgtgcgccccggcgtaa

SEQ ID NO: 61 - DNA, 1287 bp, Kosakonia pseudosacchari
  gene 1..1287: note = amtB
  source 1..1287: mol_type = genomic DNA
atgaaaaacacaacattaaaaacggctcttgcttcgctggcgttgctgccaggcctggcgatggcggctcccgctgtggcggataaagccgacaacggctttatgatgatttgcaccgcgctggtgctgtttatgaccattccgggcattgcgctgttctacggcggtttgatccgcggtaaaaacgtgctgtcgatgctgacgcaggttgccgtcaccttcgctctggtgtgcatcctgtgggtggtttacggctactctctggcatttggcgagggcaacagcttcttcggcagtttcaactgggcgatgttgaaaaacatcgaattgaaagccgtgatgggcagcatttatcagtacatccacgtggcgttccagggctcctttgcttgtatcaccgttggcctgattgtcggtgcgctggctgagcgtattcgcttctctgcggtgctgatttttgtggtggtatggctgacgctttcttatgtgccgattgcgcacatggtctggggtggcggtctgctggcaacccacggcgcgctggatttcgcgggcggtacggttgttcacatcaacgccgcgatcgcaggtctggtgggggcttacctgattggcaaacgcgtgggctttggcaaagaagcgttcaaaccgcataacctgccgatggtcttcaccggcaccgcgatcctctatgttggctggtttggcttcaacgccggctctgcaagctcggcgaacgaaatcgctgcgctggctttcgtgaacacggttgttgccactgcggccgctattctggcgtgggtatttggcgagtgggcaatgcgcggtaagccgtctctgctcggtgcctgttctggtgccatcgcgggtctggttggtatcaccccggcgtgcggttatgtgggtgtcggcggcgcgctgattgtgggtctgattgccggtctggcagggctgtggggcgttactgcactgaaacgtatgttgcgtgttgatgacccatgcgatgtcttcggtgtgcacggcgtgtgcggcatcgtgggttgtatcctgaccggtatcttcgcgtctacgtcgctgggcggtgtcggtttcgctgaaggggtgaccatgggccatcaggtactggtacagctggaaagcgttgccatcactatcgtgtggtctggcgtggtggcctttatcggttacaaactggcggatatgacggtaggcctgcgcgtaccggaagagcaagagcgtgaagggctggatgtgaacagccacggcgaaaatgcgtataacgcctga

SEQ ID NO: 62 - DNA, 500 bp, Kosakonia pseudosacchari
  gene 1..500: note = PinfC
  source 1..500: mol_type = genomic DNA
ttcttggttctctggagcgctttatcggcatcctgactgaagaatttgcaggcttcttcccaacctggcttgcacccgtgcaggtagttgtgatgaacatcactgattcgcaggctgaatacgttaacgaattgacccgtaaactgcaaaatgcgggcattcgtgtaaaagcagacttgagaaacgagaagattggctttaaaatccgcgagcacactttacgtcgtgtcccttatatgctggtttgtggtgacaaagaggtcgaagccggcaaagttgctgtgcgtacccgtcgcggtaaagacctgggtagcctggacgtaaatgatgttatcgagaagctgcaacaagagattcgcagccgcagtcttcaacaactggaggaataaggtattaaaggcggaaaacgagttcaaacggcgcgtcccaatcgtattaatggcgagattcgcgccacggaagttcgcttaacaggtctggaaggcgagcagcttggtatt

SEQ ID NO: 63 - DNA, 348 bp, Kosakonia pseudosacchari
  gene 1..348: note = Prm1
  source 1..348: mol_type = genomic DNA
cgttctgtaataataaccggacaattcggactgattaaaaaagcgccctcgcggcgctttttttatattctcgactccatttaaaataaaaaatccaatcggatttcactatttaaactggccattatctaagatgaatccgatggaagctcgctgttttaacacgcgttttttaaccttttattgaaagtcggtgcttctttgagcgaacgatcaaatttaagtggattcccatcaaaaaaatattctcaacctaaaaaagtttgtgtaatacttgtaacgctacatggagattaactcaatctagagggtattaataatgaatcgtactaaactggtactgggcgc

SEQ ID NO: 64 - DNA, 339 bp, Kosakonia pseudosacchari
  gene 1..339: note = Prm7
  source 1..339: mol_type = genomic DNA
cgcgtcaggttgaacgtaaaaaagtcggtctgcgcaaagcacgtcgtcgtccgcagttctccaaacgttaattggtttctgcttcggcagaacgattggcgaaaaaacccggtgcgaaccgggtttttttatggataaagatcgtgttatccacagcaatccattgattatctcttctttttcagcatttccagaatcccctcaccacaaagcccgcaaaatctggtaaactatcatccaattttctgcccaaatggctgggattgttcattttttgtttgccttacaacgagagtgacagtacgcgcgggtagttaactcaacatctgaccggtcgat

SEQ ID NO: 65 - DNA, 1538 bp, Enterobacter sp.
  gene 1..1538: note = 16S
  variation 62: note = n is a, c, t, g, unknown or other
  variation 265: note = n is a, c, t, g, unknown or other
  variation 457: note = n is a, c, t, g, unknown or other
  variation 469: note = n is a, c, t, g, unknown or other
  variation 539: note = n is a, c, t, g, unknown or other
  variation 551..552: note = n is a, c, t, g, unknown or other
  variation 559: note = n is a, c, t, g, unknown or other
  variation 561: note = n is a, c, t, g, unknown or other
  variation 576: note = n is a, c, t, g, unknown or other
  variation 587: note = n is a, c, t, g, unknown or other
  variation 591: note = n is a, c, t, g, unknown or other
  variation 655..657: note = n is a, c, t, g, unknown or other
  variation 665: note = n is a, c, t, g, unknown or other
  variation 678: note = n is a, c, t, g, unknown or other
  variation 706: note = n is a, c, t, g, unknown or other
  variation 710: note = n is a, c, t, g, unknown or other
  misc_feature 714: note = n is a, c, t, g, unknown or other
  variation 718: note = n is a, c, t, g, unknown or other
  variation 735: note = n is a, c, t, g, unknown or other
  variation 755: note = n is a, c, t, g, unknown or other
  variation 759: note = n is a, c, t, g, unknown or other
  variation 796: note = n is a, c, t, g, unknown or other
  variation 1006..1007: note = n is a, c, t, g, unknown or other
  variation 1016..1017: note = n is a, c, t, g, unknown or other
  variation 1133: note = n is a, c, t, g, unknown or other
  source 1..1538: mol_type = genomic DNA
ttgaagagtttgatcatggctcagattgaacgctggcggcaggcctaacacatgcaagtcgngcggaagcacaggagagcttgctctctgggtgacgagcggcggacgggtgagtaatgtctgggaaactgcctgatggagggggataactactggaaacggtagctaataccgcataacgtcgcaagaccaaagagggggaccttcgggcctcttgccatcagatgtgcccagatgggattagctagtaggtggggtaacggcncacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaaggtgttgnggttaataaccncagcaattgacgttacccgcagaagaagcaccggctaactccgtgccagcagccgcggtaatacggaggntgcaagcgttanncggaatnantgggcgtaaagcgtncgcaggcggtntgtnaagtcggatgtgaaatccccgggctcaacctgggaactgcattcgaaactggcaggctagagtnnngtagaggngggtagaattccnggtgtagcggtgaaatgcgtagagatcnggangaanaccngtggcgaaggcggcccnctggacaaagactgacgctnaggngcgaaagcgtggggagcaaacaggattagataccctngtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaacttnncagagatgnnttggtgccttcgggaactctgagacaggtgctgcatggctgtcgtcagctcgtgttgtgaaatgttgggttaagtcccgcaacgagcgcaacccttatcctttgttgccagcggtncggccgggaactcaaaggagactgccagtgataaactggaggaaggtggggatgacgtcaagtcatcatggcccttacgagtagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 66 - DNA, 882 bp, Enterobacter sp.
  gene 1..882: note = nifH
  source 1..882: mol_type = genomic DNA
atgaccatgcgtcaatgtgccatttacggcaaaggtggtatcggtaaatccactaccacgcaaaacctggtcgccgcgctggcggagatgggcaagaaagtaatgatcgtcggctgcgacccgaaagcagactccactcgtctgatcctgcatgcgaaagcgcagaacaccattatggagatggcggctgaagtcggctccgtggaagaccttgaactggaagatgtgctgcaaatcggttacggcgacgtacgctgcgcagaatccggcggcccggaaccaggcgttggctgtgctggtcgcggggtaattaccgccatcaacttcctggaagaagaaggcgcctatgttcccgacctcgatttcgtcttttacgacgtgttgggcgacgtggtgtgcggggggttcgccatgccgattcgcgaaaacaaagcgcaggagatctacatcgtctgctccggcgaaatgatggcgatgtacgccgccaacaacatctctaaaggcatcgtgaaatacgccaaatccggcaaagtgcgccttggcgggctgatctgtaactcccgtcagaccgaccgcgaagatgagctgatcatagcgctggcggaaaaactcggcacccagatgatccacttcgtgccgcgcgacaacatcgtgcaacgcgctgaaatccgccgtatgacggtgattgagtacgatccgaaatgcaaccaggccaatgaataccgcacgctggcgaacaagatcgtcaacaacaccaaaatggtcgtgccaacgcccatcaccatggacgaactggaagagctgttgatggaattcggcattatggatgtggaagacaccagcattatcggtaaaaccgccgcagaagaaaacgcggtttga

SEQ ID NO: 67 - DNA, 1449 bp, Enterobacter sp.
  gene 1..1449: note = nifD1
  source 1..1449: mol_type = genomic DNA
atgagcaatgcaacaggcgaacgtaatctggagatcatccaggaagtgctggagatctttccggaaaaaacgcgcaaagaacgcagaaagcacatgatggtgagcgacccggagatggaaagcgtcgggaaatgcatcatctccaaccgtaagtcgcagcccggcgtaatgaccgtgcgcggttgctcttacgccggttctaaaggggtggtattcgggccgatcaaagatatggcccatatttcccacggcccggtcggctgcggtcagtactcccgcgccgggcggcgtaactactacaccggcgtcagcggtgtggatagcttcggtacgctcaactttacctccgattttcaggagcgcgatatcgtgtttggcggcgataaaaagctgaccaaactgattgaagagatggagacgctgttcccgctgaccaaagggatctccattcagtccgaatgcccggtcggcctgattggcgacgacattgaagccgttgccaacgccagccgcaaagccatcaataaaccggtcattccggtgcgctgcgaaggttttcgcggcgtttcccagtcactcggtcaccacattgccaacgacgtgatccgcgactgggtactggataaccgcgaaggcaagccgtttgaggccggtccttatgacgtggcgatcatcggcgattacaacatcggcggcgatgcctgggcgtcgcgcattttgctcgaagagatgggcctgcgcgtggtggcgcagtggtccggcgacggcacgctggttgagatggagaacacgccgttcgtcaaactcaaccttgtgcactgctaccgctcaatgaactatatctcccgccatatggaggagaaacacggtattccgtggatggagtacaacttcttcggtccgaccaaagtcgccgaatcgttgcgcaaaatcgccgatatgtttgatgacaccattcgcgccaacgccgaagcggtgatcgccaaatatcaggcgcagaacgacgccatcatcgccaaataccgtccgcgtctggaaggccgcaaagtgctgctgtatatgggcggtttacgtcctcgccatgtgattggcgcttatgaagatctggggatggaaattatcgctgcgggttatgaattcgcccacaacgatgactacgaccgcaccctgccggatctgaaagaaggcaccttgctgttcgacgatgccagcagttatgaactggaagcctttgtcaaagcgctgaagccggatctgatcggctccggcattaaagagaagtacatcttccagaaaatgggcgtgccgtttcgccagatgcactcctgggattactccggcccctatcacggttatgacggctttgccatcttcgcccgcgatatggatatgacgatcaacaaccccgcgtggggccagttgaccgcgccgtggctgaaatccgcctga

SEQ ID NO: 68 - DNA, 1092 bp, Enterobacter sp.
  gene 1..1092: note = nifD2
  variation 709: note = n is a, c, t, g, unknown or other
  source 1..1092: mol_type = genomic DNA
atgggacgcggcgagcgccgcctgttccatgccgtgcgccacatcgtcaaccgctaccacccggccgccgtctttatctataacacctgcgttcccgcgatggagggcgacgatatcgaagccgtctgccaggcggcagaaaccgccatcggcgtaccggtgattgccgttgatgtcgccgggttttacggcagcaaaaatctcggcaaccggttggccggtgaagtgatggtgaaaaaggtgattggcgggcgtgaacccgcgccgtggccggaagataccccttttgccccggcgcaccgccacgatatcgggctgattggcgaattcaatattgccggagagttctggcatattcagccgctgctcgatgagctgggtattcgcgtgctcggcagcctctccggcgacgggcgcttcagtgaaatccagacgctgcaccgggcgcaggtcaatatgctggtctgctccagggcgctgatcaacgtcgcccgctcgctggagcagcgctacggcacgccgtggtttgaaggcagtttttatggtgttcgcgccacctctgacgccctgcgccaactggcggcgctgaccggagaccgcgatctgatgcagcgcaccgaacagctcattgcccgcgaagagcagcaaacagagcaggcgctggccccgctgcgcgagcgcctgcgcgggcgcaaagcgctgctctatnccggcggcgtgaaatcctggtcggtggtttcggcgcttcaggatctgggcatggaagtggtggcgaccggcacgcgcaaatccaccgaagaggataaacagcgcatccgcgaactgatgggcgccgacgcgctgatgcttgatgaaggtaacgcccgctcgctgctggacgtggtttaccgctacaaggcggacatgatgatcgccgggggacgcaatatgtacaccgcctacaaagcgcggctgccgttcctcgatatcaatcaggagcgcgagcacgcctttgccggctaccgcggcattgtcaccctggccgaacagctctgcctgaccatggaaagcccggtctggccgcaaacccattcccgcgcaccgtggcaataa

SEQ ID NO: 69 - DNA, 846 bp, Enterobacter sp.
  gene 1..846: note = nifK
  source 1..846: mol_type = genomic DNA
atgatggagcaaatggacgtgccgtgcagcctgctttccgatccctccgaagtgctggataccccggctgacgggcattaccacatgtatgcgggcggtacgacccagcaggagatgcgcgaagcgcctgacgctatcgacaccctgctgctgcaaccctggcaactggtgaaaaccaaaaaagtggtgcaggaaagctggaaccagcccgctaccgaggtgcaaatcccaatggggctggccggaaccgacgagctgctgatgacggtaagccagttaaccggcaaagccattccggatagcttagcgctggaacgcggtcggctggtggatatgatgctcgactcccacacctggctgcacggcaagaaattcggcctgttcggtgacccggattttgtcatggggctgacccgcttcctgctggaactgggctgcgaaccgacggtgattctgtgccataacggcagcaagcgctggcagaaagcgatgaagaaaatgcttgaagcctcgccgtacgggaaagagagcgaagtctttatcaactgcgatttgtggcatttccgctcgctgatgtttacccgtcagccggactttatgatcggcaactcctacgccaagtttatccagcgcgatacgctggcgaagggtgagcagtttgaagtgccgctgatccgcctggggttcccgctgttcgatcgccaccatctgcaccgccagaccacctggggttacgaaggggccatgagtatcctcaccacgctggttaatgcggtgctggagaaagtcgacagagagaccatcaagctcggcaaaaccgactacagcttcgatcttatccgttaa

SEQ ID NO: 70 - DNA, 1095 bp, Enterobacter sp.
  gene 1..1095: note = nifL
  variation 942: note = n is a, c, t, g, unknown or other
  source 1..1095: mol_type = genomic DNA
atgcagcgcgacatcagcaccagctacgcgctggaacaacggctgcgcaatcatatgacgctgaccgaagccgtcttgaataacattccggcggcggttgtagtggtggatgaacgcgatcgggtggtgatggataacctcgcctacaaaaccttttgcgccgattgcggcggtaaagaactactcaccgaaatcaacttttccgcccataaggcggagctggcgcagggcctggtactgccggtagtgctgcgcggcaccgtgcgctggttgtccgttacctgttgggcgctgccgggcgtcagcgaagaagcaggccgctactttattgatagcgccgtgccgcgcacgctggtggtgatcaccgataatactcagcagcagcaacaacaggagcaggggcgtcttgatcgtctgaagcagcagataaccagcggtaaattgctggcggcgatccgcgaatcgctggacgccgcgctggtacaactcaattgcccaattaatatgctggccgccgcacgccgcttaaatggcgacgagcatagcaatctggcgctggatgccgcatggcgtgaaggcgaagaagcgatggcgcggttgcagcgctgccgcccgtcgctggaactggaaagcccggcagtctggccgctccagccgttccttgacgatctgcgtgccctgtatcacacccgatataaccagggcgaaaacctgcaaattgagctggaatcccccgacctggtgggctttggccagcgaacacaactgcttgcctgcctgagcctgtggctcgacagaaccctggatattgccgcggagctacgtgatttcacggtacagactcaactttacgcccgcgaagagagcggctggctgtcgttctatttaaacgacaatgtgccgctgattcaggtgcgctacacccattcacccgatgcactnaatgcgcccggtaaaggcatggagctgcggctgatccagacgctggtcgcccaccatcgaggcgcaatagaactgacctcacgccctcagggaggcacctgtctgatcctgcgtttcccattattttactcgctgacaggaggctcactatga

SEQ ID NO: 71 - DNA, 219 bp, Enterobacter sp.
  gene 1..219: note = nifA partial gene
  source 1..219: mol_type = genomic DNA
atgactcagcgaaccgagtcgggtacaaccgtctggcgctttgacctctcccaacagtttacagccatgcagcgtatcagtgtggtgttaagccgcgcgacggagatcgggcagacgctacaggaagtgctgtgcgtgctgcacaacgatgcctttatgcagcacgggatgatctgtccgtacgcgcgggtgcgcgtcttcgcgagcgtatggctttga

SEQ ID NO: 72 - DNA, 1635 bp, Enterobacter sp.
  gene 1..1635: note = glnE
  variation 234: note = n is a, c, t, g, unknown or other
  variation 274: note = n is a, c, t, g, unknown or other
  variation 1535: note = n is a, c, t, g, unknown or other
  source 1..1635: mol_type = genomic DNA
atgcgcgtggaagactggtcaacgctgaccgaacggctcgatgcccatatggcaggcgtgcgccgaatctttaacgaactgatcggtgatgacgaaagtgagtcgcaggacgatgcgctctccgagcactggcgcgagctgtggcaggacgcgcttcaggaagatgacaccacgccggtgctgacgcacttaaccgacgacgcgcgccatcgcgtggtggcgctgatcgctganttccgtcttgagctgaacaaacgcgccatcggcccgcgtngtcgccaggtgctggatcacctgatgccgcacctgctgagcgaagtctgctcgcgtgccgatgcgccggtgccgctgtcgcggatgatgcccctgctgagcgggattatcacccgtactacctaccttgaactcctgagcgagttccctggcgcgcttaagcacctgatttcactctgcgccgcgtcgccgatggtggccaacaagctggcgcgttacccgctgctgctggatgagctgctcgatccgaataccctttatcaaccgacggcgaccgacgcctaccgggacgaactgcgtcagtatctgctgcgcgtgccggaagaagacgaagagcaacagctggaggcgctgcgtcagtttaagcaggcccagatgctgcgcgtggcggccgcagatattgccggaacgctgccggtgatgaaagtgagcgatcacttaacctggcttgcggaagcgattatcgacgcggtggtgcatcaggcctgggtgcagatggtggcgcgctatggccagccgaaacatctggctgaccgtgatggtcgcggcttcgcggtggtgggttacggtaagctcggcggttgggagctgggctatagctccgatctggatttaatcttcctccacgactgcccggttgatgtgatgaccgacggcgagcgcgagattgacgggcgtcagttctacctgcgcctggcgcagcgcatcatgcacctgttcagcacccgcacctcgtcgggcattttgtatgaagtggatgcccgtctgcgcccgtccggcgcggcgggcatgctggtcacctcgacggagtccttcgctgattaccagaagaatgaagcctggacgtgggagcatcaggcgctggtgcgcgcccgtgtggtgtatggcgatccgctgctgaaaacgcagtttgacgtgattcgtaaggaagtcatgaccaccgtgcgcgatggcagcacgctgcaaacggaagtgcgcgaaatgcgcgagaaaatgcgcgcgcacttaggcaataaacatcgcgatcgctttgatattaaagccgatgagggcggtattaccgatattgagtttattacccagtatctggtgttgctgcacgcgcatgacaagccgaagctgacgcgctggtcggataacgtgcgcattctggaactgctggcgcaaaacgacattatggacgagcaggaggcgcaggccttaacccgtgcctatacaacgcttcgcgatgagctccatcatctggcgttgcaggagcagcngggacacgtggcgctggactgtttcaccgctgaacgcgctcaggtaacggccagctggcagaagtggctggtggaaccgtgcgtaacaaatcaagtgtga

SEQ ID NO: 73 - DNA, 1316 bp, Enterobacter sp.
  gene 1..1316: note = 16S
  variation 43: note = n is a, c, t, g, unknown or other
  variation 247: note = n is a, c, t, g, unknown or other
  variation 784..785: note = n is a, c, t, g, unknown or other
  variation 794..795: note = n is a, c, t, g, unknown or other
  source 1..1316: mol_type = genomic DNA
agatgtgcccagatgggattagctagtaggtggggtaacggcncacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaaggtgttgtggttaataaccncagcaattgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcattcgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaacttnncagagatgnnttggtgccttcgggaactctgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgagtagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 74 - DNA, 882 bp, Enterobacter sp.; identical to SEQ ID NO: 66
  gene 1..882: note = nifH
  source 1..882: mol_type = genomic DNA

SEQ ID NO: 75 - DNA, 1449 bp, Enterobacter sp.; identical to SEQ ID NO: 67
  gene 1..1449: note = nifD1
  source 1..1449: mol_type = genomic DNA

SEQ ID NO: 76 - DNA, 1374 bp, Enterobacter sp.
  gene 1..1374: note = nifD2
  source 1..1374: mol_type = genomic DNA
atgaaggggaacgagatcctggctttgctcgatgaacctgcctgcgagcacaaccataaacagaaatccggctgcagcgcgccgaaacccggcgcgacagcgggcggctgcgcctttgacggtgcgcagatcaccctgctgccactctccgatgttgcccacctggtacacggccccattggttgtaccggtagctcatgggataaccgtggcagcttcagttccggcccgacgatcaaccggctgggttttaccaccgatctgagcgaacaggatgtgatcatgggacgcggcgagcgccgcctgttccatgccgtgcgccacatcgtcaaccgctaccacccggccgccgtctttatctataacacctgcgttcccgcgatggagggcgacgatatcgaagccgtctgccaggcggcagaaaccgccatcggcgtaccggtgattgccgttgatgtcgccgggttttacggcagcaaaaatctcggcaaccggttggccggtgaagtgatggtgaaaaaggtgattggcgggcgtgaacccgcgccgtggccggaagataccccttttgccccggcgcaccgccacgatatcgggctgattggcgaattcaatattgccggagagttctggcatattcagccgctgctcgatgagctgggtattcgcgtgctcggcagcctctccggcgacgggcgcttcagtgaaatccagacgctgcaccgggcgcaggtcaatatgctggtctgctccagggcgctgatcaacgtcgcccgctcgctggagcagcgctacggcacgccgtggtttgaaggcagtttttatggtgttcgcgccacctctgacgccctgcgccaactggcggcgctgaccggagaccgcgatctgatgcagcgcaccgaacagctcattgcccgcgaagagcagcaaacagagcaggcgctggccccgctgcgcgagcgcctgcgcgggcgcaaagcgctgctctataccggcggcgtgaaatcctggtcggtggtttcggcgcttcaggatctgggcatggaagtggtggcgaccggcacgcgcaaatccaccgaagaggataaacagcgcatccgcgaactgatgggcgccgacgcgctgatgcttgatgaaggtaacgcccgctcgctgctggacgtggtttaccgctacaaggcggacatgatgatcgccgggggacgcaatatgtacaccgcctacaaagcgcggctgccgttcctcgatatcaatcaggagcgcgagcacgcctttgccggctaccgcggcattgtcaccctggccgaacagctctgcctgaccatggaaagcccggtctggccgcaaacccattcccgcgcaccgtggcaataa

SEQ ID NO: 77 - DNA, 1563 bp, Enterobacter sp.
  gene 1..1563: note = nifK1
  source 1..1563: mol_type = genomic DNA
atgagccaaagtgctgagaaaattcaaaactgtcatccgctgtttgaacaggatgcgtaccagatgctgtttaaagataaacggcaactggaagaggcccacgatccggcgcgcgtgcaggaggtctttcaatggaccaccaccgccgagtatgaagcgcttaactttcaacgcgaagcgctgactatcgatccggccaaagcctgccagccgctgggtgcggtactgtgctcgctgggctttgccaataccctgccctatgttcacggctcccaggggtgcgtggcctatttccgcacctattttaaccgtcactttaaagagccgattgcctgtgtttctgactcgatgacggaagatgcggcagtattcggcggcaacaacaacctgaacaccgggttgcagaacgccagcgccctctacaagccggaaatcattgccgtctccaccacctgtatggcggaggtcatcggcgacgacctgcaggcgtttattgctaacgccaaaaaagacggctttatcgacgcggcgatcccggtgccttacgcgcacacgccaagctttatcggcagccatatcaccggctgggacaatatgtttgagggcttcgcccgtacctttaccgccgattacagcggacaaccgggcaaattaccgcgtatcaatctggtcagcggatttgaaacctatctcggtaatttccgcgtgctgaaacgcatgatggagcaaatggacgtgccgtgcagcctgctttccgatccctccgaagtgctggataccccggctgacgggcattaccacatgtatgcgggcggtacgacccagcaggagatgcgcgaagcgcctgacgctatcgacaccctgctgctgcaaccctggcaactggtgaaaaccaaaaaagtggtgcaggaaagctggaaccagcccgctaccgaggtgcaaatcccaatggggctggccggaaccgacgagctgctgatgacggtaagccagttaaccggcaaagccattccggatagcttagcgctggaacgcggtcggctggtggatatgatgctcgactcccacacctggctgcacggcaagaaattcggcctgttcggtgacccggattttgtcatggggctgacccgcttcctgctggaactgggctgcgaaccgacggtgattctgtgccataacggcagcaagcgctggcagaaagcgatgaagaaaatgcttgaagcctcgccgtacgggaaagagagcgaagtctttatcaactgcgatttgtggcatttccgctcgctgatgtttacccgtcagccggactttatgatcggcaactcctacgccaagtttatccagcgcgatacgctggcgaagggtgagcagtttgaagtgccgctgatccgcctggggttcccgctgttcgatcgccaccatctgcaccgccagaccacctggggttacgaaggggccatgagtatcctcaccacgctggttaatgcggtgctggagaaagtcgacagagagaccatcaagctcggcaaaaccgactacagcttcgatcttatccgttaa

SEQ ID NO: 78 - DNA, 1386 bp, Enterobacter sp.
  gene 1..1386: note = nifK2
  source 1..1386: mol_type = genomic DNA
atggcagaaattatccgtagtaaaaagccgctggccgtcagcccggtaaaaagtggccagccgctgggcgcgattctggcgagcatgggctttgaacagagcattccgctggttcatggcgctcaggggtgcagcgccttcgcgaaggtcttttttatccagcattttcacgatccgatcccgctgcaatcgacggcaatggacccgacatcgaccattatgggtgccgatgagaacatctttaccgcgctgaatgtgctgtgttcacgcaacaacccgaaagcgattgttctgctgagcactggcctttccgaggcgcagggcagcgatatttcgcgcgtggtgcgccagttccgcgatgaatatccgcgccataaaggggtggcgctgctgaccgtcaacacgccggatttttacggcagcctggaaaacggctacagcgcggtgctggagagcatggttgaacagtgggtgccggaaaaaccgcagccgggcgtgcgcaatcgccgcgtgaacctgctgctcagccatttgcttacgccgggcgacattgagctgctgcgaagttatgtcgaggcatttggcctgcagccggtgatggtgccggatctttcccagtcgctggatggccatctcgccagcggggatttctcgccaattacccagggcggcagcagcctgcggctgattgaacagatgggacagagtcttggcacgttcgccattggcgtatccctctcccgcgccgcgcaattgctggcgcagcgcagccatgcggaagtggtcaccctgccgcatctgatgaccatgagccagtgcgatacgtttattcatcaactgaagcgcctctccgggcgcgatgttccggcgtggatcgaacgccagcgcgggcaactgcaggatgcgatgatcgattgtcatatgtggttgcagggcgcgcctgtcgcgctggccgccgagggcgatctgctcgccgcctggtgcgatttcgcctgcgatatgggcatggtgcccggcccggtggtggcgccggtgagccagaaagggttgcaggatctgccggtcgaaaaagtcattatcggcgatctggaggatatgcaggatctgttgtgtgaaacgcctgcatcgctgctcgtctctaattctcacgccgctgatttggccgggcagttcgacattccgctggtgcgcgccggtttccccctgttcgaccgtctgggcgagtttcgccgcgtgcgccagggttacgccgggatgcgcgacaccttgtttgagctggcgaatgcgctgcgcgatcgccatcatcatcttgccgcttatcactcgccgctgcgccagcgtttttacgaacccgcatcttcgggaggtgactatgcaacatgttaa

SEQ ID NO: 79 - DNA, 1488 bp, Enterobacter sp.
  gene 1..1488: note = nifL
  source 1..1488: mol_type = genomic DNA
atgaccctgaatatgatgatggacgccaccgcgcccgccgagatcgccggagcgctctcacaacagcatcccgggttgtttttcaccatggttgaacaggcgcccgtcgcgatttcactgaccgatgccgatgcccacattctctacgccaaccccgcgttttgtcgccagtcggggtatgaactggaagagttgttgcagcaaaacccgcgcctgcttgccagtaagcagacgccgcgtgaaatctaccaggaaatgtggcacaccctgctgcaacaccgtccgtggcgcggacaactgatcaaccgtcgccgcgacggcagcctgtttctggtggaaatcgacatcaccccactgtttgatgcgttcggcaaactcgaacattacctggccatgcagcgcgacatcagcaccagctacgcgctggaacaacggctgcgcaatcatatgacgctgaccgaagccgtcttgaataacattccggcggcggttgtagtggtggatgaacgcgatcgggtggtgatggataacctcgcctacaaaaccttttgcgccgattgcggcggtaaagaactactcaccgaaatcaacttttccgcccataaggcggagctggcgcagggcctggtactgccggtagtgctgcgcggcaccgtgcgctggttgtccgttacctgttgggcgctgccgggcgtcagcgaagaagcaggccgctactttattgatagcgccgtgccgcgcacgctggtggtgatcaccgataatactcagcagcagcaacaacaggagcaggggcgtcttgatcgtctgaagcagcagataaccagcggtaaattgctggcggcgatccgcgaatcgctggacgccgcgctggtacaactcaattgcccaattaatatgctggccgccgcacgccgcttaaatggcgacgagcatagcaatctggcgctggatgccgcatggcgtgaaggcgaagaagcgatggcgcggttgcagcgctgccgcccgtcgctggaactggaaagcccggcagtctggccgctccagccgttccttgacgatctgcgtgccctgtatcacacccgatataaccagggcgaaaacctgcaaattgagctggaatcccccgacctggtgggctttggccagcgaacacaactgcttgcctgcctgagcctgtggctcgacagaaccctggatattgccgcggagctacgtgatttcacggtacagactcaactttacgcccgcgaagagagcggctggctgtcgttctatttaaacgacaatgtgccgctgattcaggtgcgctacacccattcacccgatgcactcaatgcgcccggtaaaggcatggagctgcggctgatccagacgctggtcgcccaccatcgaggcgcaatagaactgacctcacgccctcagggaggcacctgtctgatcctgcgtttcccattattttactcgctgacaggaggctcactatga

SEQ ID NO: 80 - DNA, 1575 bp, Enterobacter sp.
  gene 1..1575: note = nifA
  source 1..1575: mol_type = genomic DNA
atgactcagcgaaccgagtcgggtacaaccgtctggcgctttgacctctcccaacagtttacagccatgcagcgtatcagtgtggtgttaagccgcgcgacggagatcgggcagacgctacaggaagtgctgtgcgtgctgcacaacgatgcctttatgcagcacgggatgatctgtctgtacgacagtaagcaagcgatcctttccattgaagccttgcatgaggccgatcagcagttaattcccggcagttcacagattcgctaccgtccgggcgaagggctggtaggcacggtgctttcacagggacagtcgctggtactgccctgtgtctccgacgatcggcgttttctcgatcgcctgggattgtatgattacagcttgccgtttatcgccgtgccgctgatggggccaaactcgcagcctatcggcgtgctggccgcccagcctatggcgcgttacgaggagcggctgcccgcctgcacgcgttttcttgaaaccgtcgccaatctggtggcgcaaaccgttcgcctgatgacaccgcccagcgtcgcgtctccaccccgtgctgctgccgcgcagattgccagccagcgcgggtgcgcgtcttcgcgagcgtatggctttgaaaacatggtcggtaaaagcgcggctatgcgtcagacgctggaaattattcgccaggtatcacgctgggacaccaccgtgctggtgcgtggcgaaagcggaaccggtaaagagttgatagccaacgctatccaccacaattcaccgcgcgccgccgcgccgtttgtcaaattcaactgcgcggcgctgcccgatacgctgctggagagtgaactcttcggtcatgaaaaaggcgcgtttaccggcgcggtgcgccagcgcaaaggccgtttcgaactggcggatggcggtacgctgtttcttgatgagatcggcgaaagtagcgcctcgtttcaggcgaaattgctgcgtatcttgcaggaaggcgaaatggaacgcgtcggcggcgacgaaacgctgcgggtgaatgtacggatcattgccgccaccaaccgcaatctggaagaggaagtgcggctgggtaattttcgcgaagatctctactatcgccttaatgtgatgccgatctccctgcccccgctccgcgagcgtcaggaggacatcgtcgagctggcgcattttctggtgcgcaaaatcgcgcaaaaccagaaccgcacgctgcgcatcagcgatggcgcgatccgtttgttgatgagctatagctggcctggaaacgtgcgtgagctggaaaactgccttgagcgatcggcggtgatgtcggaaaacgggctgatcgatcgcgacgtgattttgtttcaccacagggaaaatctgccaaaaacgccacagaccagtgcgccgcgcgaagagagctggctcgatcagaacctcgatgagcgacaaagattgatcgccgcgctggagaaagccggttgggtacaggcaaaagccgcgcgcctgctgggaatgaccccgcgccaggtggcctatcgtattcagacgatggacattgccatgccgagattgtag

SEQ ID NO: 81 - DNA, 2856 bp, Enterobacter sp.
  gene 1..2856: note = glnE
  source 1..2856: mol_type = genomic DNA
atgccgctttcttcgcagttacagcagcagtggcagaccgtttgcgaacgtctgcctgagtcattaccggcgtcatcgttaagcgagcaggcaaagagcgtgctcgtcttcagtgattttgtgcaggaaagtatcaccgccaacccgaactggctggcggaacttgagaacgcaccaccgcaggcagaagagtggcggcactatgctggctggctgcaaactgtactcgaagacgttacggatgaggccacgctgatgcgcgtcctgcgccagttccgtcgtcgggtgatggtgcgcattgcctgggctcaggcgctggaactggtgagcgaagagagtacgctgcagcagttaagcgagctggcgcaaacgttgattgtcgccgcgcgagactggctctatgccgcctgctgtaaagagtggggcacgccgtgcagcgaggaaggggttcctcagccgctgttgattctggggatgggaaagctgggcggctgcgagctgaacttctcctctgatatcgacctgatttttgcctggccggagaacggctccacgcgcggaggccgccgcgagctggacaacgcgcagttctttacccgtctcggccagcgcctgattaaagcgctggatcagcccacgcaggacggttttgtttaccgcgtggacatgcgcctgcgtccgtttggcgacagcgggccgctggtgctgagctttgcggcgctggaagattattaccaggagcaaggtcgcgactgggagcgttacgcgatggtcaaagcgcggatcatgggcgacagcgacgacgcttatgccaacgagctgcgcgccatgctgcgtccgttcgtgttccgtcgctatatcgacttcagcgtcatccagtccctgcgaaatatgaaagggatgattgcccgcgaggtgcgccgccgtgggctgaaagacaatatcaagctcggtgcgggcggcatccgcgaaatcgaatttatcgtccaggtcttccagcttattcgcggcggacgcgagccgtcgctgcagtcccgttccttattaccgacgctgagcgccattgcgcagctgcatctcctgccggacggcgacgcgcaaaccctgcgcgaggcctatcttttcctgcgtcgtctggaaaacctgctgcaaagcattaatgacgaacagacccaaaccctgccgggcgacgaccttaaccgggcgcgtctggcctggggaatgcgcgtggaagactggtcaacgctgaccgaacggctcgatgcccatatggcaggcgtgcgccgaatctttaacgaactgatcggtgatgacgaaagtgagtcgcaggacgatgcgctctccgagcactggcgcgagctgtggcaggacgcgcttcaggaagatgacaccacgccggtgctgacgcacttaaccgacgacgcgcgccatcgcgtggtggcgctgatcgctgatttccgtcttgagctgaacaaacgcgccatcggcccgcgtggtcgccaggtgctggatcacctgatgccgcacctgctgagcgaagtctgctcgcgtgccgatgcgccggtgccgctgtcgcggatgatgcccctgctgagcgggattatcacccgtactacctaccttgaactcctgagcgagttccctggcgcgcttaagcacctgatttcactctgcgccgcgtcgccgatggtggccaacaagctggcgcgttacccgctgctgctggatgagctgctcgatccgaataccctttatcaaccgacggcgaccgacgcctaccgggacgaactgcgtcagtatctgctgcgcgtgccggaagaagacgaagagcaacagctggaggcgctgcgtcagtttaagcaggcccagatgctgcgcgtggcggccgcagatattgccggaacgctgccggtgatgaaagtgagcgatcacttaacctggcttgcggaagcgattatcgacgcggtggtgcatcaggcctgggtgcagatggtggcgcgctatggccagccgaaacatctggctgaccgtgatggtcgcggcttcgcggtggtgggttacggtaagctcggcggttgggagctgggctatagctccgatctggatttaatcttcctccacgactgcccggttgatgtgatgaccgacggcgagcgcgagattgacgggcgtcagttctacctgcgcctggcgcagcgcatcatgcacctgttcagcacccgcacctcgtcgggcattttgtatgaagtggatgcccgtctgcgcccgtccggcgcggcgggcatgctggtcacctcgacggagtccttcgctgattaccagaagaatgaagcctggacgtgggagcatcaggcgctggtgcgcgcccgtgtggtgtatggcgatccgctgctgaaaacgcagtttgacgtgattcgtaaggaagtcatgaccaccgtgcgcgatggcagcacgctgcaaacggaagtgcgcgaaatgcgcgagaaaatgcgcgcgcacttaggcaataaacatcgcgatcgctttgatattaaagccgatgagggcggtattaccgatattgagtttattacccagtatctggtgttgctgcacgcgcatgacaagccgaagctgacgcgctggtcggataacgtgcgcattctggaactgctggcgcaaaacgacattatggacgagcaggaggcgcaggccttaacccgtgcctatacaacgcttcgcgatgagctccatcatctggcgttgcaggagcagccgggacacgtggcgctggactgtttcaccgctgaacgcgctcaggtaacggccagctggcagaagtggctggtggaaccgtgcgtaacaaatcaagtgtga

SEQ ID NO: 82 - DNA, 1290 bp, Enterobacter sp.
  gene 1..1290: note = amtB
  source 1..1290: mol_type = genomic DNA
atgaagatagcaacacttaaaacgggtctgggttcgctggcactgctgccgggcctggcgctggctgctgcacctgcggtggcagacaaagccgataacgcctttatgatgatcagcaccgcgctggtgctgttcatgtccattccgggcattgcgctgttctatggcggcctgatccgtggcaaaaacgttctctccatgctgacgcaggttgccgtaacgttcgcgctggtctgcgtactgtgggtggtttacggttactcgctggctttcggcacgggcaacgcgttctttggtaacttcgactgggtgatgctgaaaaatattgaactgaccgcgctgatgggcagtttctaccagtatattcacgttgctttccagggctcgttcgcctgcattaccgtcgggctgattgtaggcgcgcttgccgagcgtattcgtttctctgcggtcctgatcttcgtggtggtctggctgacgctctcctatgtgccgattgcgcacatggtctggggtggcggtctgctggcgacgcatggcgcgctggacttcgcgggcggtaccgttgtgcacattaacgccgcggtagcgggtctggttggcgcatacctgattggcaaacgcgtgggcttcggtaaagaagcgttcaaaccgcacaacctgccgatggtcttcaccggtaccgcgatcctctactttggctggtttggtttcaacgccggctcagcaagtgccgcgaacgaaatcgccgcgctggccttcgtgaataccgttgtggccacggcaggtgcaatcctctcctgggtctttggcgagtgggctgtgcgcggtaaaccttctctgctgggtgcctgttcgggggcgattgctggtctggtcggtatcaccccagcatgtggttatgtcggtgtgggtggcgcgctgctggtcggcctggtgtcaggtctggcgggtctgtggggcgtgacggcgctgaaacgtattctgcgcgttgatgacccttgcgatgtgtttggcgtgcacggcgtgtgcggcatcgtcggctgtatcatgaccggtatctttgcagcgaaatcgctgggtggcgtgggctacgcagaaggcgtcaccatggcccatcaggtgctggtgcagctggaaagtattgctgtcaccgtggtgtggtctgccgttgtcgctttcattggctacaaactggcggacatgacggttggtctgcgcgtgccggaagagcaggaacgcgaaggtctggacgtcaacagccacggcgagaatgcgtataacgcatga

SEQ ID NO: 83 - DNA, 1299 bp, Azospirillum lipoferum
  gene 1..1299: note = 16S
  variation 294: note = n is a, c, t, g, unknown or other
  variation 299: note = n is a, c, t, g, unknown or other
  variation 302: note = n is a, c, t, g, unknown or other
  variation 309..310: note = n is a, c, t, g, unknown or other
  variation 332: note = n is a, c, t, g, unknown or other
  variation 346: note = n is a, c, t, g, unknown or other
  variation 351: note = n is a, c, t, g, unknown or other
  variation 438: note = n is a, c, t, g, unknown or other
  variation 445: note = n is a, c, t, g, unknown or other
  variation 454: note = n is a, c, t, g, unknown or other
  variation 469: note = n is a, c, t, g, unknown or other
  variation 477: note = n is a, c, t, g, unknown or other
  variation 492: note = n is a, c, t, g, unknown or other
  variation 504: note = n is a, c, t, g, unknown or other
  variation 555: note = n is a, c, t, g, unknown or other
  variation 775: note = n is a, c, t, g, unknown or other
  source 1..1299: mol_type = genomic DNA
gccgagagaggggcccgcgtcggattaggtagttggtgaggtaatggctcaccaagccttcgatccgtagctggtctgagaggatgatcagccacactgggactgagacacggcccagactcctacgggaggcagcagtggggaatattggacaatgggcgcaagcctgatccagcaatgccgcgtgagtgatgaaggccttagggttgtaaagctctttcgcacgcgacgatgatgacggtagcgtgagaagaagccccggctaacttcgtgccagcagccgcggtaatacgnagggngcnagcgttnntcggaattactgggcgtaaagngcgcgtaggcggcntgttnagtcagaagtgaaagccccgggctcaacctgggaatagcttttgatactggcaggcttgagttccggagaggatggtggaattcccngtgtagnggtgaaatncgtagatattgggangaacaccngtggcgaaggcggcnatctggacgganactgacgctgaggcgcgaaagcgtggggagcaaacaggattagataccctngtagtccacgccgtaaacgatgaatgctagacgtcggggtgcatgcacttcggtgtcgccgctaacgcattaagcattccgcctggggagtacggccgcaaggttaaaactcaaaggaattgacgggggcccgcacaagcggtggagcatgtggtttaattcgaagcaacgcgcagaaccttaccaacccttgacatgtccactttgggctcgagagatngggtccttcagttcggctgggtggaacacaggtgctgcatggctgtcgtcagctcgtgtcgtgagatgttgggttaagtcccgcaacgagcgcaacccctaccgtcagttgccatcattcagttgggcactctggtggaaccgccggtgacaagccggaggaaggcggggatgacgtcaagtcctcatggcccttatgggttgggctacacacgtgctacaatggcggtgacagtgggaagcgaagtcgcgagatggagcaaatccccaaaagccgtctcagttcggatcgcactctgcaactcgagtgcgtgaagttggaatcgctagtaatcgcggatcagcacgccgcggtgaatacgttcccgggccttgtacacaccgcccgtcacaccatgggagttggttttacccgaaggtggtgcgctaaccgcaaggaggcagccaaccacggtaaggtcagcgactggggtgaagtcgtaacaaggtagccgtaggggaacctgcggctggatcacctccttt

SEQ ID NO: 84 - DNA, 897 bp, Azospirillum lipoferum
  gene 1..897: note = nifH
  source 1..897: mol_type = genomic DNA
atggccaaagcgcctctgcgtcagatcgccttttacggcaagggcggtatcggcaagtccaccacctctcagaacacgctggccgcgctggtcgagctggatcagaggatcctgatcgtcggctgcgacccgaaggccgactcgacccgcctgatcctgcacgcaaaggcccaggacaccgtcctgcatctggccgccgaggccggctcggtcgaggatctggagctcgaggacgttctcaagatcggctacaagaacatcaagtgcgtcgagtccggcggtccggagccgggggtcggctgcgccggccgcggcgtcatcacctcgatcaacttcctggaagagaacggcgcctacgacgacgtggactatgtgtcctacgacgtgctgggcgacgtggtctgcggcggcttcgccatgccgatccgcgagaacaaggcccaggaaatctacatcgtcatgtccggcgagatgatggcgctgtacgccgccaacaacatcgccaagggcatcctgaagtacgcgcacagcggcggcgtccgtctcggcggcctgatctgcaacgagcgccagaccgacaaggaatgggatctggccgacgcgctggccaagcgcctgggctccaagctgatccacttcgtgccgcgcgacaacatcgtccagcacgccgagctgcgccgcatgacggtcatcgagtacgccccggacagcaagcaggccggcgaataccgcgcgctcgccaacaagatccatgcgaactccggccagggttgcatcccgaccccgatcaccatggaagagctggaagagatgctgatggacttcggcatcatgaagaccgaggagcagcagctcgccgagctcgccgccaaggaagcggcgaaggccggcgcctga

SEQ ID NO: 85 - DNA, 1440 bp, Azospirillum lipoferum
  gene 1..1440: note = nifD1
  source 1..1440: mol_type = genomic DNA
atgagcctgtccgagaacaccacggtcgacgtcaagaacctcgtcaacgaagtcctcgaagcctatcccgaaaaatcccgcaagcgccgcgccaagcacctgaacgtgctggaggccgaggccaaggactgcggcgtcaagtcgaacgtcaagtccatccccggcgtcatgaccatccgcggctgcgcctatgccggctccaagggcgtggtgtggggtccgatcaaggacatgatccacatctcccacggtccggtcggctgcggctactactcctggtccggccgccgcaactactacatcggcgacaccggtgtggacagctggggcacgatgcacttcacctccgacttccaggagaaggacatcgtcttcggcggcgacaagaagctgcacaaggtcatcgaggaaatcaacgagctgttcccgctggtgaacggcatctcgatccagtcggaatgcccgatcggcctgatcggcgacgacatcgaggctgtcgcccgcgccaagtcggcggaaatcggcaagccggtcatccccgtgcgctgcgaaggcttccgcggcgtgtcccagtcgctgggccaccacatcgccaacgacgccatccgagactgggtgttcgagaagacggaacccaaggccggcttcgtctccaccccctatgacgtcaccatcatcggcgactacaacatcggcggcgacgcctggtcgtcccgcatcctgctggaggagatcggcctgcgcgtgatcgcccagtggtcgggcgacggcacgctcgccgaactggagaacacgccgaaggccaaggtcaacctgatccactgctaccgctcgatgaactacatcgcgcgccacatggaagagaagttcaacattccttggatggaatacaacttcttcggcccgagccagatcgccgaatccctgcgcaagatcgccgctctcttcgacgacaagatcaaggagaacgccgagaaggtcatcgcccgctaccagccgatggtcgatgcggtcatcgccaagtacaagccgcggctcgaaggcaagaaggtcatgatctacgtcggcggcctgcgtccccgccacgtcgtcgatgcctaccatgacctcggcatggagatcaccggcaccggctacgagttcgcccacaacgacgactatcagcgcacgcagcactacgtgaaggaaggcacgctgatctacgacgacgtcaccgcgttcgaactggagaagttcgtcgaggcgatgcgtcccgacctcgtcgcgtcgggcatcaaggaaaagtacgtgttccagaagatgggcctgccgttccgccagatgcacagctgggactactccggcccgtaccacggctatgacggcttcgcgatcttcgcccgcgacatggacctggccatcaacaaccccgtctggggcgtgatgaaggccccgttctga

SEQ ID NO: 86 - DNA, 1419 bp, Azospirillum lipoferum
  gene 1..1419: note = nifD2
  source 1..1419: mol_type = genomic DNA
atgctccaggacaagatccaggatgtcttcaacgaaccgggctgcgcgaccaaccaagccaaatcggccaaggagaagaagaagggctgcaccaagtcgctgaaaccgggggcggcagccggcggctgcgcctatgacggggcgatgatcgtgctccagccgatcgccgacgccgcccatctggtccatggccccatcgcctgcctcggaaacagttgggacaaccgcggctccaaatcctccggctcgcagctctaccgcaccggcttcaccaccgatctgtcggaactggacgtcatcggcggcggcgagaagaagctctaccgcgccatcaaggagatcgttcagcaatacgacccgccggccgtcttcgtctatcagacctgcgtgcccgccatgaccggcgacgacatcgccgcggtctgcaagttcgccacgcagaagctgggcaagccggtgatcccggtggactcgccgggcttcgtcgggtcgaagaatctcggcaacaagctggccggcgaagccctgctggagcatgtcatcggcacggtcgaaccggactacaccaccccgaccgacgtctgcatcatcggcgaatacaaccttgccggcgagctgtggctggtcaagccgctgctggacgagatcggcatccgcctcctgtcctgcatttccggcgacggccgctaccgggaggtggcgcaggcccaccgcgcccgcgtcaccatgatggtgtgcagccaggcgctggtgaatgtcgggcgcaagatggaggagcgctacggcatcccctatttcgaggggtccttctacggcgtgtccgacatgtcggacaccctgcgcaccatgacccgcatgctggtggagcgcggcgccgacaagggcctgatcgaccgggcggagggcgtgatcgcgcgggaggaaagccgggtctggcgccggctggaaccctacaagccgcgcttcgacggcaagcgcgtccttctcttcaccggcggcgtcaagagctggtcgatggtcagcgcgctggagggtgcggggctgaccatcctcggcacctccaccaagaaatcgaccagggaggacaaggagcgcatcaagaagatgaagggcgaagagttccaccagtgggacgatttgaagccgcgcgacatctacaggatgctggccgacgatcaggccgacatcatgatgtccggcggccgctcgcagttcatctcgctgaaggccaaggttccctggctcgacatcaaccaggagcgccaccacgcctatgccggctatgacggcatcgtcaatctctgcgaggagatcgacaaaacgctgtcgaatccgatctggcgtcaggtgcgtcagccggcaccgtgggagtccggcgcgtcctccacccttctggcttcctcgatggcggcggagtga

SEQ ID NO: 87 - DNA, 1383 bp, Azospirillum lipoferum
  gene 1..1383: note = nifK
  source 1..1383: mol_type = genomic DNA
atgtcccacatccagcgcttcccctccgccgccaaggccgcctccaccaacccgctgaagatgagccagccgctgggtgcggctctggcctatctcggcgtcgaccgctgcctgccgctgttccatggctcgcagggctgcaccgccttcgggctggtcctgctggtgcgccatttccgcgaggcgatcccgctccagaccacggcgatggatcaggtcgccaccatcctcggcggctacgacaatctggagcaggcgatccgcaccatcgtcgagcgcaaccagcccgccatgatcggcgtcgccaccaccggcgtcaccgagaccaagggcgaggatatggccggacagtacacgctgttccgccagcgcaaccccgacttggccgacacggccctggtcttcgccaacacccccgacttcgccggcggcttcgaggacggcttcgccgccgcggtcaccgcgatggtcgagcggttggtcgaaccgtcgccggtgcgcatcccgacccaggtcaacgtgctggccggctgccatctgtcccccggcgacgtggaggaactgcgcgacatcatcgaaggcttcggcctgtcgccgatcttcctgcccgacctgtcgctgtcgatggcgggccgccagccggccgacttcaccgccacctcgctgggcggcgtgaccgtcgatcagatccgcgccatgggcgcttcggccctcaccatcgtggtcggtgagcatatgcgggtggccggtaacgcgctggagctgaagaccgacgtgcccagccatttcttcaaccgcctgaccgggctggaggcgacggacaagctggtccggctgctgatggagttgtcgggcaagccggcgcccgcccggctgcggcgccagcgcgaaagcctggtcgatgccatgctcgacgggcatttcttctacagccgcaagcgcatcgccgtcgcgctggagcccgacctgctctatgccgtcaccggcttcctcgccgacatgggggccgaggtgatcgccgcggtgtccccgacgcagagcccggtgctggagcggttgaaggccgccaccatcatggtcggcgatcattccgacgtggagacgctggcccgcgacgccgacctgatcgtctccaactcgcacgggcggcagggagccgcgcggatcggcgtggctctgcaccgcatgggcctgccgctgttcgaccggctgggggccggcctgcgcgtccaggtcggctaccgcggcacgcgggaactgctgtgcgacatcggcaacctgttcctcgcccgcgagatggaccacgagcacgggcacgagagccacgaccacggggaatcccacggctgcggaggcggatcatgcggatgcaacgccgtctga

SEQ ID NO: 88 - DNA, 1560 bp, Azospirillum lipoferum
  gene 1..1560: note = nifK1
  source 1..1560: mol_type = genomic DNA
atgaccgacaagctttcgcagagcgccgacaaggtcctcgaccactacaccctcttccggcagcccgaatacgcggcgatgttcgagaagaagaagaccgagttcgagtacggccattcggacgaggaagtcgcccgcgtgtccgaatggaccaagtccgaggactacaaggcgaagaacttcgcccgtgaagcggtcgtcatcaacccgaccaaggcctgccagccgatcggcgcaatgttcgccgcccagggcttcgaaggcaccctgcccttcgtccacggctcccagggctgcgtcgcctattaccgcacccacctgacccgtcacttcaaggagccgaacagcgcggtctcctcgtcgatgacggaggacgcggcggtgttcggcggcctgaacaacatgatcgacggcctggcgaacgcctatgcgctctacaagccgaagatgatcgcggtgatgaccacctgcatggccgaagtcatcggcgacgatttgcagggcttcatcgccaatgcgaagaccaaggacagcgtcccggccgacttcccggtcccctacgcccacaccccggccttcgtcggcagccacatcgtcggctacgacaacatgatcaaggggatcctgaccaacttctggggtacgtcggagaatttcgacacacccaagaccgagcagatcaacctgatcccgggcttcgacggcttcgccgtcggcaacaaccgcgaactgaagcgcatcgccggcgaattcggcgtgaagctgcaaatcctgtccgacgtgtccgacaatttcgacacgccgatggatggcgagtaccgcatgtatgacggcggcaccaccatcgaggagaccaaggaggccctgcacgccaaggccaccatctccatgcaggagtacaacacgacccagaccctgcaattctgcaaggagaagggtcaggaagtcgccaagttcaactacccgatgggcgtcaccggcaccgacgagctgctgctgaagctcgccgaactgtcgggcaagccggtcccggccagcctgaagctggagcgcggccgtctggtcgacgccatcgccgacagccacacccacatgcacggcaagcgcttcgccgtctatggcgacccggacttctgcctgggcatgtccaagttcctgctggagctgggtgcggagccggtgcacatcctgtcgacgtcgggctccaagaagtgggagaagcaggtccagaaggtgctggacggctcgcccttcggcgcctcgggcaaggcccatggcggcaaggatctgtggcacctgcgttcgctgatcttcaccgacaaggtggactacatcatcggcaacagctacggcaagtatctggagcgcgacaccaaggttccgctgatccgcctgacctacccgatcttcgaccgccaccaccaccaccgctacccgacctggggctaccagggcgcgctgaacgtgctggtacggatcctggaccggatcttcgaggacatcgacgccaacaccaacatcgtcggccagaccgactactcgttcgacctgatccgctga

SEQ ID NO: 89 - DNA, 1530 bp, Azospirillum lipoferum
  gene 1..1530: note = nifA
  source 1..1530: mol_type = genomic DNA
atgttgacctctgatattgttggcaaattgcgctgcatcgcagcagaccccaaagcgggcatcgcaaggggcctcgacaccgggacgacgaagatcggtcccgtttgggagggtgacgtgggcgacaccgtggatttcgaagcgctgcgccagcgggcggtccactccctgttcgaacatctggaatccatgtgcgtcggcgccgtcgccgtcgaccacaccggccgcatcgcctggatggacgagaagtacaaggctctgctgggcgttcccgacgacccgcgcggccggcaggtggaggacgtcatccccaacagccagctgcgccgggtgatcgacagcggccagccgcagccgctggacatcatggagttcgacgaccggtccttcgtggtgacgcggatgccgctgttcggcaccgacggttcgatcatcggcgccatcggcttcgtgctgttcgaccgcgccgaatatctccgcccgctggtccgcaaatacgagaagatgcaggaggagctggcccgcacccagcaggagctggcgcatgagcgccgcgccaaatactccttctcgcagttcctgggcgccagcgaatcgatccgcgagatcaagcggctggggcgccgcgccgcccagatggattcgaccgtcctgctgctgggcgaaaccgggaccggcaaggagctgctggcccaggccatccattccgccagcccgcgggcgtccaagcccttcgtcggcgtcaatgtcgccgccattccggaaaccctgctggaggcggagttcttcggcgtcgcccccggcgccttcaccggcgccgaccgccgccaccgcgacggcaagttccagctcgccaacggcggcaccctgttcctcgacgagatcggcgacatgccgctgccggtgcaggccaagcttctgcgcgtgctgcaggagcgggagatcgagccgctcggctccaacaaggtggtgcgggtcgatgtccgcatcatcgccgccaccagccgtgacctgcacgccctggtgcgtgagaagcagttccgcgccgacctctattaccggctgaatgtggtgccgatcaccctgccgccgctgcgcgaccggccggaggacatcgagagcatcgccgaccgcatcctggaacagctggcgatccagcagggcacgccgccgcgcgagctgctggaatcggcggtgcaggtgctgcgcgactatgactggcccggcaatgtgcgcgagctttacaacacgctggaacgggtggtggcgctgaccgatgcgccgatcctgaccgcgccgcacatccgcagcgtgctgcccggccagcatccggccggcgcgtcggccctgccgctggcggccggcgcgcggccgttgcaggaggtgctgcacgccgccgagcgccacgccatcgccgcggcgcttgaggaggcgaacggcgtcaaggcgcgggcggcgaagctgctgggcatttcgcgcgcgtcgctgtacgaacgcatggtgacgctggggttgggggcgacgcagtag

SEQ ID NO: 90 - DNA, 3030 bp, Azospirillum lipoferum
  gene 1..3030: note = glnE
  source 1..3030: mol_type = genomic DNA
atgccgagtcccatcgcgttctcaagccccttgccgaagcctttcgacagcgcgcaggcggcgctggggatggagcgctggcgccagcaggccgccgcggcggagccggagacccgcgcctgggcggaagccttcgccgattcggagaccggccgggcgctgatcggggcggtgtgcggcaacagcccgtatctcggccacagcctgacgcgggagttgcccttcgtcgcccgtacagtgcaggacggcttcgacgacaccttcgccgcgctgatcgccgctctccatgccgagcatggcgaggagaaatcgatggaccggctgatggccggcctgcgggtggcgaagcggcgggcggcgctgctgatcgcgctggccgacatcgccggcgcgtggccgctgttccgcgtcaccggcgccctgtcggagctggcggagacgggggtgcagctggccgcgaatttcctgctgcgccgcgccagggaggcggggacgctgacgctgccggatccgcagcgaccgtgggtcggttcgggcctgatcgttttgggcatgggtaagcttggcgggcgcgaactcaactattccagcgacatcgacctgatcgtcctgtatgacgacgctgttgtgcagacgccccagccggacaacctcgcgcgaaccttcatcaggctcgcacgcgatcttgtccgcattatggatgaacggaccaaggacggctacgtcttccgcaccgaccttcggcttaggcccgatcccggcgccacgccgctggcggtttccgtctccgcagccgaaatttattacggcagcgtcggtcagaactgggaacgcgcggcgatgatcaaggcccgtcccatcgccggcgatctggaggcgggcgcctcctttgtccgcttcctggagcccttcgtctggcgccgcaacctggatttcgccgccatccaggacatccattcgatcaaacgccagatcaacgcccacaagggccaccgcgaggtgacggtcaacggccacgacatcaaggtcggccgcggcggcatccgcgagatcgagttcttcgcccagacccagcagctgatcttcggcgggcgcgacccgcgcgtgcgaatcgctccgaccctgatggcgaacgaggcgctgcgcgacgtcggccgcgtgccgccgcagacggtggaagagcttgccggggcctatcatttcctgcgccgtgtcgaacatcgcatccagatgatcgacgaccagcagacccatcgtattcccgccgacgatgccggggtggcgcatttggccaccttcctcggctatgacgaccccgccgccttccgggcggaactgctggcgacgctggggcaggtggaggaccgctatgccgagctgttcgaggaggcgccgtcgctttccggccccggcaatctggtcttcaccggcaccgaccccgatccgggcacgatggagacgctgaagggcatgggcttcgccgatccggcccgcgtcatcagcgtggtgtcggcctggcatcgcggccgctaccgcgccacccggtcgggccgggcgcgggagctgctgacggagctgacgccggccctgctgagtgcgctgaccaagaccccggcccccgattcggcgctgatgaacttcgacgatttcctcggcaagctgccggccggcgtcggtctgttctcgctgttcgtcgccaatccctggctcctggagctggtggcggagatcatgggcatcgcgccgcagatggcgcagacgctgtcgcgcaacccgtcgctgctcgacgccgtgctgtcgccggacttcttcgacccgctgccgggcaaggaggacgggctggccgacgaccacgcccgcgtgatggcgccggcccgcgatttcgaggatgcgctgaccctgtcgcggcgctggaccaacgaccagcgcttccgcgccggggtgcatatcctgcgcggcatcaccgatggcgaccgctgcggcgccttcctggccgatctggccgacatcgtcgtccccgaccttgcccgccgggtggaggaggagttcgcccagcgccacggccatattcccggcggcgcctgggtggtggtggcgatgggcaagctcggcagccggcagctgaccatcacgtccgacatcgacctgatcgtcatctacgatgtggcgccgggccaagggggcgggggcggtccccgcttgtcggatggtgccaagccgctgtcgcccaacgagtattacatcaagctgactcagcgtctgaccaacgccattaccgcgccgatgggcgacggccggctctacgaggtcgacatgcggctgcgcccgtcgggcaacgccgggccgctcgccacctcgctggacgctttcctgaaatatcaggcgaccgatgcctggacctgggagcatatggccctgacccgcgcccgggtgatcggcggtgatgcggagctggccgggcgggtgtcggcagcgatccgctcggtgctgacggcgccgcgcgatgccgaccggctgctgtgggacgtggccgacatgcggcggcggatcgagaaggagttcgggacgaccaatgtctggaacgtcaaatacgcccgcggcggcctgatcgacatcgagttcatcgcccagtacctgcaactgcgccatggtcacgagcggccggacatcctgcacatcggcaccgccaaggcgctgggctgcgccgcccggacgggcgcgctggcgccggaggtggcggaggatctggagacgacgctgcggctgtggcggcgggtgcagggctttctgcggttgaccaccgccggggtgctcgatcccaatcaggtgtcgcccagcctgctggccgggctggtccgcgccgcctttcctgctgactttcagggcgagcgtgagcctggcactgttgacttccccgaactggaccacaaaatccgtgccgtcgccgcccgcgcccatggtcatttcaagaccctggtcgaggaaccggcgggccgtctggccccacccgccaccacgcctccagcctga

SEQ ID NO: 91 - DNA, 1440 bp, Azospirillum lipoferum
  gene 1..1440: note = amtB1
  source 1..1440: mol_type = genomic DNA
atgaaccgtctgttccttatggccgcaccgatgatggcggttgctctgggcgcggtcggcatgccggccgcagcccttgcccaggatccggcggctgccgccgctgccgcggctgcggctgcggctgccgccgctgctgccgcaccggcggctccggcgctgaatggcggcgacaccgcctggatgctcatctccaccgcgctggtgctgatgatgaccatccccggcctggcgctgttctacggcggcatggtccgcaagatgaacgtgctgtcgacggtgatgcagagcttcgccatcacctgcctgatcagcgtcctgtggtacgtcatcggctacagcctggccttcaccggcaccggtgcctatgtcggcggtctcgaccggctgttcctcaacgggctcgacttcacgaaggccttcgtgctgggcgaggcgaccgggtcgggcgtcccgacgaccatccccgagccggtcttcatgatgttccagatgacctttgcgatcatcaccccggccctgatcaccggcgccttcgccgaccgcatgaagttctcctccctgctggtcttcaccgcgctgtggtcgatcgtggtctatgcgccgatcgcccactgggtctggtacccgtcgggcttcctgttcggcctgggcgtgctggacttcgccggcggcacggtcgtgcacatcaacgccggcgtcgccggcctggtcgccgcgctggtgatcggcaagcgcaagggctacccgaaggaagccttcatgccgcacaacctggtgctgtcgctgatcggcgcctcgctgctgtgggtcggctggttcggcttcaacgccggttcggccctgaccgccggtccgcgtgccggcatggcgctggccgccacgcacatcgccaccgccggtgccgccatgggctggctgttcgcggagtggatcgtcaagggcaagccgtcgatcctcggcatcatctccggcgccgtcgccggcctggtcgcggtgaccccggccgccggcttcgtcgacccgacgggcgccatcgtcatcggcatcgtcgccggcgtggtctgcttctggtcggccaccagcctcaagcacatgctgggctatgacgacagcctggacgccttcggcgtgcacggcgtcggcggcctgatcggcgccatcctgaccggcgtcttcgccaagatgtcggtgtccaacagcgaaggcggcttcgcctccgtcctgcaggccgacccgaaggccacgctgggcctgctggaaggcaacgccgccgccgtctggatccaggtccagggcgtcctctacaccatggtctggtgcgccatcgccaccttcgtcctgctgaagatcgtcgatgtggtcatgggcctgcgcgtcgaagaggatgtggagcgcgacggtctcgacctcgccctgcatggcgagagcatccactaa

SEQ ID NO: 92 - DNA, 1227 bp, Azospirillum lipoferum
  gene 1..1227: note = amtB2
  source 1..1227: mol_type = genomic DNA
atggatgcggcaaagacgggtggcgacgtccttttcgtgctgatgggcgcggtgatggtgctggcgatgcattgcggcttcgccctgctggaggtcgggacggtccggcgcaagaatcaggtcaacgcgctggtgaagatcctgtcggacttcgccatgtcgaccatcgcctattttttcgtcggttatgccgtggcctacggcatcgacttcttcgccgacgcccacacgctggtcggcaagggaagcggcgggttcgcggcctatggctacgatctggtgaagttcttcttcctggcgaccttcgccgccgcggtgccggccatcgtctcgggcggcatcgccgagcgtgctaggttctggccgcaggccgccgccacgctggcgctgatcgcgctgttctatccattgctggaaggcacggtctggggcacccgcttcggcctgcaaagctggatggccgcgaccttcggccagcctttccacgacttcgccggatctgtggtggtgcatgccttcggcggctgggtggcgctgggtgccgtgctgaacctcggcaaccgccgcggccgctaccgtccgaacggctcgctgatcgccattccgccgtcgaacatccccttcctggcgctgggcgcctgggtgctgtgcgtggggtggttcggcttcaacgtgatgagcgcccaggtgctggatggcgtgacgggtctggtggcgctgaactcgctgatggcgatggtcggcggcatcgtcacctcgctggtgatcagccgcaccgatcccggcttcgtccacaacggcgcgctggccggtctggtggcggtctgcgccgggtccgacgtgatgcacccgctgggcgcgctggtcaccggcggcatcgccgggctgctgttcgtctgggccttcaacaaatgccagatcgactggaagatcgacgacgtgctgggcgtctggccgctgcacggtctgtgcggcctgaccggcggcctgctggccggcgtcttcgggcaggaggcactgggcggccttggcggcgtgtcgatcctcagccagatcgtcggcacggcaagcggcgccagcttcggattcgtctcgggtctggcggtctacggcctgctgcgcgtcaccgtcggcatccgcctcgatcccgagcaggagtacaagggcgccgacttgtcgttgcaccatatcaccgcgtacccggaagaggacgcgccgaccctgtaa

SEQ ID NO: 93 - DNA, 613 bp, synthetic construct
  misc_feature 1..613: note = Synthetic polynucleotide
  misc_feature 1..613: note = delta-nifL::PinfC
  source 1..613: mol_type = other DNA
atgaccctgaatatgatgatggattcttggttctctggagcgctttatcggcatcctgactgaagaatttgcaggcttcttcccaacctggcttgcacccgtgcaggtagttgtgatgaacatcactgattcgcaggctgaatacgttaacgaattgacccgtaaactgcaaaatgcgggcattcgtgtaaaagcagacttgagaaacgagaagattggctttaaaatccgcgagcacactttacgtcgtgtcccttatatgctggtttgtggtgacaaagaggtcgaagccggcaaagttgctgtgcgtacccgtcgcggtaaagacctgggtagcctggacgtaaatgatgttatcgagaagctgcaacaagagattcgcagccgcagtcttcaacaactggaggaataaggtattaaaggcggaaaacgagttcaaacggcgcgtcccaatcgtattaatggcgagattcgcgccacggaagttcgcttaacaggtctggaaggcgagcagcttggtattgcgatagaactcacttcacgccccgaagggggaagctgcctgaccctacgattcccgctatttcattcactgaccggaggttcaaaatga

SEQ ID NO: 94 - DNA, 1613 bp, synthetic construct
  misc_feature 1..1613: note = Synthetic polynucleotide
  misc_feature 1..1613: note = delta-nifL::PinfC with 500bp flank
  source 1..1613: mol_type = other DNA
accggataagagagaaaagtgtcgacgtcggtccggttgatattgaccggcgcatccgccagctcgcccagtttttggtggatctgtttggcgattttgcgggtcttgccggtgtcggtgccgaaaaaaataccaatatttgccataacacacgctcctgttgaaaaagagatcccgcggggaaaatgcggtgaacatgtcagctattgcgaagagtgtgccagttttgctcacgggcaaaagctgcaccagaatgggtattaatgcaccagcctggcgctttttttcgcggcacgtcccctcgctaatgcccgtctggcgcggctttgacgctgataaggcgctgaataccgatctggatcaaggttttgtcgggttatcgtccaaaaggtgcactctttgcatggttataagtgcctgacatggtgtccgggcgaacgtcgccaggtggcacaaattgtcagaactacgacacgactaaccgaccgcaggagtgtgcgatgaccctgaatatgatgatggattcttggttctctggagcgctttatcggcatcctgactgaagaatttgcaggcttcttcccaacctggcttgcacccgtgcaggtagttgtgatgaacatcactgattcgcaggctgaatacgttaacgaattgacccgtaaactgcaaaatgcgggcattcgtgtaaaagcagacttgagaaacgagaagattggctttaaaatccgcgagcacactttacgtcgtgtcccttatatgctggtttgtggtgacaaagaggtcgaagccggcaaagttgctgtgcgtacccgtcgcggtaaagacctgggtagcctggacgtaaatgatgttatcgagaagctgcaacaagagattcgcagccgcagtcttcaacaactggaggaataaggtattaaaggcggaaaacgagttcaaacggcgcgtcccaatcgtattaatggcgagattcgcgccacggaagttcgcttaacaggtctggaaggcgagcagcttggtattgcgatagaactcacttcacgccccgaagggggaagctgcctgaccctacgattcccgctatttcattcactgaccggaggttcaaaatgacccagcgaaccgagtcgggtaataccgtctggcgcttcgatttatcccagcagttcaccgcgatgcagcggataagcgtggttctcagccgggcgaccgaggttgaacagacactccagcaggtgctgtgcgtattgcacaatgacgcctttttgcagcacggcatgatctgtctgtacgacagccagcaggcgattttgactattgaagcgttgcaggaagccgatcagcagttgatccccggcagctcgcaaattcgctaccgtccgggtgaagggctggtcgggacggtgctttcgcaggggcaatcgttagtgctggcgcgtgtggctgacgatcagcgctttcttgaccgcctgggactgtatgattacaacctgccgtttatcgccgtgccgctgatagggccggatgcgcagacttttggcgtgctgacggcgcaaccgatggcgcgttacgaagagcggttacccgcctgcacccgctttctggaaacggtc

SEQ ID NO: 95 - DNA, 1185 bp, synthetic construct
  misc_feature 1..1185: note = Synthetic polynucleotide
  misc_feature 1..1185: note = glnE-delta-AR-2
  source 1..1185: mol_type = other DNA
tccctgtgcgccgcgtcgccgatggtggccagccaactggcgcgctacccgatcctgctcgatgaactgctcgacccgaacacgctctatcaaccgacggcgatgaacgcctatcgcgatgaactgcgacaatacctgttgcgcgtgccggaagaggatgaagagcagcaactggaggcgctacggcagtttaagcaggcgcagttgttgcgcgtagcggcggcggatatcgccggtacgttacccgtcatgaaagtgagcgatcacttaacctggctggcggaagcgattatcgatgcggtggtgcagcaagcctggaaccagatggtggcgcgttacggccagccgacgcatctgcacgatcgcgaagggcgcggtttcgccgtggtcggttacggcaaacttggcggctgggaattaggttacagctccgatctggatctggtgttcctgcacgactgccccatggatgtgatgaccgatggcgagcgtgaaatcgatggccgccagttctatttgcgcctcgcgcagcgcgtgatgcacctgttcagcacgcgcacgtcgtccggcattctttatgaagtcgatgcgcgtttgcgcccgtccggcgcggccggaatgctggtgaccactgcggaagcgttcgccgattatcaaaaaaatgaagcctggacatgggagcatcaggcgctggcgcgtgcgcgcgtggtgtacggcgatccgcaactgaccgccgaatttgacgccattcgccgcgatatcctgatgacctcccgcgatgccgctaccctgcaaaccgaagtgcgggaaatgcgtgagaaaatgcgcgcccatcttggtaacaagcacaaagaccgtttcgatctgaaagccgatgaaggcggtatcaccgatattgagtttatcgctcagtatctggtgctgcgctttgcccatgagaagccgaaactgacgcgctggtcggataatgtgcgcatcctcgaagggctggcgcaaaacggcatcatggatgagcaggaagcgcaggcattgacgctggcgtacaccacgttgcgtgatgagctgcaccacctggcgctgcaagagctgccaggacatgtggcgctctcctgttttgtcgccgagcgtgcgcttatcaaaaccagctgggacaagtggctggtggaaccgtgcgccccggcgtaa

SEQ ID NO: 96 - DNA, 2085 bp, synthetic construct
  misc_feature 1..2085: note = Synthetic polynucleotide
  misc_feature 1..2085: note = glnE-delta-AR-2 with 500bp flank
  source 1..2085: mol_type = other DNA
taaagcgagcgctcacttacgtgatctgttgacgcagtccgaagcgaccattacttcagccgtttcagcagatacggcggtgtggagtgcgcaatcagccctggcgaaactggtgctcaccgagtggttagtgacgcagggctggcgaaccttccttgatgaaaaagcgcaggctaagtttgccgactcctttaaacgctttgctgacgttcatctgtcacgcagcgccgccgagctgaaaaaagcctttgcccagccgctgggcgacagctatcgcgaccagttaccgcggctggcgcgtgatatcgacagcgcgttattgctggccggacattacgatcgcgcgcgcgccgtggagtggctggaaaactggcaggggcttcagcacgctattgaaacgcgccagagagttgaaatcgaacatttccgtaataccgccattacccaggagccgttctggttgcacagcggaaaacgttaacgaaaggatatttcgcatgtccctgtgcgccgcgtcgccgatggtggccagccaactggcgcgctacccgatcctgctcgatgaactgctcgacccgaacacgctctatcaaccgacggcgatgaacgcctatcgcgatgaactgcgacaatacctgttgcgcgtgccggaagaggatgaagagcagcaactggaggcgctacggcagtttaagcaggcgcagttgttgcgcgtagcggcggcggatatcgccggtacgttacccgtcatgaaagtgagcgatcacttaacctggctggcggaagcgattatcgatgcggtggtgcagcaagcctggaaccagatggtggcgcgttacggccagccgacgcatctgcacgatcgcgaagggcgcggtttcgccgtggtcggttacggcaaacttggcggctgggaattaggttacagctccgatctggatctggtgttcctgcacgactgccccatggatgtgatgaccgatggcgagcgtgaaatcgatggccgccagttctatttgcgcctcgcgcagcgcgtgatgcacctgttcagcacgcgcacgtcgtccggcattctttatgaagtcgatgcgcgtttgcgcccgtccggcgcggccggaatgctggtgaccactgcggaagcgttcgccgattatcaaaaaaatgaagcctggacatgggagcatcaggcgctggcgcgtgcgcgcgtggtgtacggcgatccgcaactgaccgccgaatttgacgccattcgccgcgatatcctgatgacctcccgcgatgccgctaccctgcaaaccgaagtgcgggaaatgcgtgagaaaatgcgcgcccatcttggtaacaagcacaaagaccgtttcgatctgaaagccgatgaaggcggtatcaccgatattgagtttatcgctcagtatctggtgctgcgctttgcccatgagaagccgaaactgacgcgctggtcggataatgtgcgcatcctcgaagggctggcgcaaaacggcatcatggatgagcaggaagcgcaggcattgacgctggcgtacaccacgttgcgtgatgagctgcaccacctggcgctgcaagagctgccaggacatgtggcgctctcctgttttgtcgccgagcgtgcgcttatcaaaaccagctgggacaagtggctggtggaaccgtgcgccccggcgtaagtgtggtatcatcgcgcgcaaattttgtatctctcaggagacaggaatgaaagttacgctgccagagttcaatcaagccggtgtcatggtggtgggtgatgtgatgctggatcgctactggtacggcccaaccagccgcatttctccggaagcgccagttccggttgttaaagtcgatactattgaagagcgaccgggcggtgcggcaaacgtggcgatgaacattgcctcgctgggcgcaacggcgcgtctggttggcctgactggcattgatgatgcggcgcgcgcgctgagcaaagcgctggcggatgttaatgttaaatgtgacttcgtttctgttccgactcaccccaccatcactaagctgcgcgtgctgtcgcgtaaccagcaactgattcgc

SEQ ID NO: 97 - DNA, 461 bp, synthetic construct
  misc_feature 1..461: note = Synthetic polynucleotide
  misc_feature 1..461: note = delta-nifL::Prm1
  source 1..461: mol_type = other DNA
atgaccctgaatatgatgatggatgccagccgttctgtaataataaccggacaattcggactgattaaaaaagcgccctcgcggcgctttttttatattctcgactccatttaaaataaaaaatccaatcggatttcactatttaaactggccattatctaagatgaatccgatggaagctcgctgttttaacacgcgttttttaaccttttattgaaagtcggtgcttctttgagcgaacgatcaaatttaagtggattcccatcaaaaaaatattctcaacctaaaaaagtttgtgtaatacttgtaacgctacatggagattaactcaatctagagggtattaataatgaatcgtactaaactggtactgggcgcaactcacttcacgccccgaagggggaagctgcctgaccctacgattcccgctatttcattcactgaccggaggttcaaaatga

SEQ ID NO: 98 - DNA, 1461 bp, synthetic construct
  misc_feature 1..1461: note = Synthetic polynucleotide
  misc_feature 1..1461: note = delta-nifL::Prm1 with 500bp flank
  source 1..1461: mol_type = other DNA
accggataagagagaaaagtgtcgacgtcggtccggttgatattgaccggcgcatccgccagctcgcccagtttttggtggatctgtttggcgattttgcgggtcttgccggtgtcggtgccgaaaaaaataccaatatttgccataacacacgctcctgttgaaaaagagatcccgcggggaaaatgcggtgaacatgtcagctattgcgaagagtgtgccagttttgctcacgggcaaaagctgcaccagaatgggtattaatgcaccagcctggcgctttttttcgcggcacgtcccctcgctaatgcccgtctggcgcggctttgacgctgataaggcgctgaataccgatctggatcaaggttttgtcgggttatcgtccaaaaggtgcactctttgcatggttataagtgcctgacatggtgtccgggcgaacgtcgccaggtggcacaaattgtcagaactacgacacgactaaccgaccgcaggagtgtgcgatgaccctgaatatgatgatggatgccagccgttctgtaataataaccggacaattcggactgattaaaaaagcgccctcgcggcgctttttttatattctcgactccatttaaaataaaaaatccaatcggatttcactatttaaactggccattatctaagatgaatccgatggaagctcgctgttttaacacgcgttttttaaccttttattgaaagtcggtgcttctttgagcgaacgatcaaatttaagtggattcccatcaaaaaaatattctcaacctaaaaaagtttgtgtaatacttgtaacgctacatggagattaactcaatctagagggtattaataatgaatcgtactaaactggtactgggcgcaactcacttcacgccccgaagggggaagctgcctgaccctacgattcccgctatttcattcactgaccggaggttcaaaatgacccagcgaaccgagtcgggtaataccgtctggcgcttcgatttatcccagcagttcaccgcgatgcagcggataagcgtggttctcagccgggcgaccgaggttgaacagacactccagcaggtgctgtgcgtattgcacaatgacgcctttttgcagcacggcatgatctgtctgtacgacagccagcaggcgattttgactattgaagcgttgcaggaagccgatcagcagttgatccccggcagctcgcaaattcgctaccgtccgggtgaagggctggtcgggacggtgctttcgcaggggcaatcgttagtgctggcgcgtgtggctgacgatcagcgctttcttgaccgcctgggactgtatgattacaacctgccgtttatcgccgtgccgctgatagggccggatgcgcagacttttggcgtgctgacggcgcaaccgatggcgcgttacgaagagcggttacccgcctgcacccgctttctggaaacggtc

SEQ ID NO: 99 - DNA, 1185 bp, synthetic construct; identical to SEQ ID NO: 95
  misc_feature 1..1185: note = Synthetic polynucleotide
  misc_feature 1..1185: note = glnE-delta-AR-2
  source 1..1185: mol_type = other DNA

SEQ ID NO: 100 - DNA, 2085 bp, synthetic construct; identical to SEQ ID NO: 96
  misc_feature 1..2085: note = Synthetic polynucleotide
  misc_feature 1..2085: note = glnE-delta-AR-2 with 500bp flank
  source 1..2085: mol_type = other DNA

SEQ ID NO: 101 - DNA, 461 bp, synthetic construct; identical to SEQ ID NO: 97
  misc_feature 1..461: note = Synthetic polynucleotide
  misc_feature 1..461: note = delta-nifL::Prm1
  source 1..461: mol_type = other DNA

SEQ ID NO: 102 - DNA, 1461 bp, synthetic construct; identical to SEQ ID NO: 98
  misc_feature 1..1461: note = Synthetic polynucleotide
  misc_feature 1..1461: note = delta-nifL::Prm1 with 500bp flank
  source 1..1461: mol_type = other DNA

SEQ ID NO: 103 - DNA, 1185 bp, synthetic construct; identical to SEQ ID NO: 95
  misc_feature 1..1185: note = Synthetic polynucleotide
  misc_feature 1..1185: note = glnE-delta-AR-2
  source 1..1185: mol_type = other DNA

SEQ ID NO: 104 - DNA, 2085 bp, synthetic construct; identical to SEQ ID NO: 96
  misc_feature 1..2085: note = Synthetic polynucleotide
  misc_feature 1..2085: note = glnE-delta-AR-2 with 500bp flank
  source 1..2085: mol_type = other DNA

SEQ ID NO: 105 - DNA, 452 bp, synthetic construct
  misc_feature 1..452: note = Synthetic polynucleotide
  misc_feature 1..452: note = delta-nifL::Prm7
  source 1..452: mol_type = other DNA
atgaccctgaatatgatgatggatgccagccgcgtcaggttgaacgtaaaaaagtcggtctgcgcaaagcacgtcgtcgtccgcagttctccaaacgttaattggtttctgcttcggcagaacgattggcgaaaaaacccggtgcgaaccgggtttttttatggataaagatcgtgttatccacagcaatccattgattatctcttctttttcagcatttccagaatcccctcaccacaaagcccgcaaaatctggtaaactatcatccaattttctgcccaaatggctgggattgttcattttttgtttgccttacaacgagagtgacagtacgcgcgggtagttaactcaacatctgaccggtcgataactcacttcacgccccgaagggggaagctgcctgaccctacgattcccgctatttcattcactgaccggaggttcaaaatga

SEQ ID NO: 106 - DNA, 1451 bp, synthetic construct
  misc_feature 1..1451: note = Synthetic polynucleotide
  misc_feature 1..1451: note = delta-nifL::Prm7 with 500bp flank
  source 1..1451: mol_type = other DNA
accggataagagagaaaagtgtcgacgtcggtccggttgatattgaccggcgcatccgccagctcgcccagtttttggtggatctgtttggcgattttgcgggtcttgccggtgtcggtgccgaaaaaaataccaatatttgccataacacacgctcctgttgaaaaagagatcccgcggggaaaatgcggtgaacatgtcagctattgcgaagagtgtgccagttttgctcacgggcaaaagctgcaccagaatgggtattaatgcaccagcctggcgctttttttcgcggcacgtcccctcgctaatgcccgtctggcgcggctttgacgctgataaggcgctgaataccgatctggatcaaggttttgtcgggttatcgtccaaaaggtgcactctttgcatggttataagtgcctgacatggtgtccgggcgaacgtcgccaggtggcacaaattgtcagaactacgacacgactaaccgaccgcaggagtgtgcgatgaccctgaatatgatgatggatgccagccgcgtcaggttgaacgtaaaaaagtcggtctgcgcaaagcacgtcgtcgtccgcagttctccaaacgttaattggtttctgcttcggcagaacgattggcgaaaaaacccggtgcgaaccgggtttttttatggataaagatcgtgttatccacagcaatccattgattatctcttctttttcagcatttccagaatcccctcaccacaaagcccgcaaaatctggtaaactatcatccaattttctgcccaaatggctgggattgttcattttttgtttgccttacaacgagagtgacagtacgcgcgggtagttaactcaacatctgaccggtcgataactcacttcacgccccgaagggggaagctgcctgaccctacgattcccgctatttcattcactgaccggaggttcaaaatgacccagcgaaccgagtcgggtaataccgtctggcgcttcgatttatcccagcagttcaccgcgatgcagcggataagcgtggttctcagccgggcgaccgaggttgaacagacactccagcaggtgctgtgcgtattgcacaatgacgcctttttgcagcacggcatgatctgtctgtacgacagccagcaggcgattttgactattgaagcgttgcaggaagccgatcagcagttgatccccggcagctcgcaaattcgctaccgtccgggtgaagggctggtcgggacggtgctttcgcaggggcaatcgttagtgctggcgcgtgtggctgacgatcagcgctttcttgaccgcctgggactgtatgattacaacctgccgtttatcgccgtgccgctgatagggccggatgcgcagacttttggcgtgctgacggcgcaaccgatggcgcgttacgaagagcggttacccgcctgcacccgctttctggaaacggt

SEQ ID NO: 107 - DNA, 1191 bp, synthetic construct
  misc_feature 1..1191: note = Synthetic polynucleotide
  misc_feature 1..1191: note = glnE-delta-AR-2
  source 1..1191: mol_type = other DNA
atggcgctgaagcacctgatcacgctctgcgcggcgtcgccgatggtcgccagccagctggcgcgccacccgctgctgctggatgagctgctggatcccaacaccctctatcagccgacggcgaccgatgcctatcgcgacgagctgcgccagtacctgctgcgcgtgccggaagaggatgaagagcagcagctggaggcgttgcgccagtttaagcaggcgcagcagctgcatatcgcggcggcggatatcgctggtaccctgccggtgatgaaggtcagcgatcacttaacctggcttgccgaagcgatcctcgacgcggtggtgcagcaggcatgggggcagatggtcgctcgctacggccagccgacccacctgcacgatcgccagggtcgcggcttcgccgtcgtcggctacggtaagcttggcggctgggagctgggctacagctccgatctcgatctggtgttcctccatgactgcccggcggaggtgatgaccgacggcgagcgggagattgacggccgtcagttctacctgcggctggcccagcggatcatgcacctgttcagcacccgcacctcgtccggtattctctacgaagtggacgcccggctgcgtccttctggcgcggcggggatgctggtcaccaccgccgacgcgtttgctgactatcagcagaacgaagcctggacgtgggaacatcaggcgctggtgcgcgcccgcgtggtctatggcgacccggcgctgcaggcgcgctttgacgccattcgtcgcgatatcctgaccaccccgcgggaggggatgaccctgcagaccgaggttcgcgagatgcgcgagaagatgcgcgcccaccttggcaacaaacatcccgatcgttttgatatcaaagccgatgccggcgggatcaccgatattgaatttattactcagtatctggtcctacgctatgccagtgacaagccgaagctgacccgctggtctgacaacgtgcgtattcttgagctgctggcgcagaacgacatcatggacgaggaggaggcgcgcgccttaacgcatgcgtacaccaccttgcgtgatgcgctccatcacctggccctgcaggagcagccgggacacgtggcgccagaggccttcagccgggagcgtcagcaggtcagcgccagctggcagaagtggctgatggcttaa

SEQ ID NO: 108 - DNA, 2191 bp, synthetic construct
  misc_feature 1..2191: note = Synthetic polynucleotide
  misc_feature 1..2191: note = glnE-delta-AR-2 with 500bp flank
  source 1..2191: mol_type = other DNA
cgtaaggcgaccacccagctccgcgcgttgctgaacgacgctgaagccgttctgctggccgcggacaccgccgacgaggcgttattccgcaccgaggtcgtcggcgccaaactggccctgactgaatggctggtccagcgcggctggcgtccgttcctcaacgaggcaggagagaaaaaaatagccggatcgttcaaacggtttgccgatattaacctctcgcgggtggcggccgagctgcgcagcgccgtgcagcatctggcggttgaagatgccgccgaccagttgccgaagctgtcccgcgacatcgacagcgtccagctgctggcgggcgcctatggcgacgccgtcgcgccgtggctggagaactggcaggagcttcaccgtgcaatagcacatgacgatcgcagcgtctttgaatatttccgtcgccaggcgctggctgccgagccgttctggctgcatagtggaaaacgataatttcaggccagggagcccttatggcgctgaagcacctgatcacgctctgcgcggcgtcgccgatggtcgccagccagctggcgcgccacccgctgctgctggatgagctgctggatcccaacaccctctatcagccgacggcgaccgatgcctatcgcgacgagctgcgccagtacctgctgcgcgtgccggaagaggatgaagagcagcagctggaggcgttgcgccagtttaagcaggcgcagcagctgcatatcgcggcggcggatatcgctggtaccctgccggtgatgaaggtcagcgatcacttaacctggcttgccgaagcgatcctcgacgcggtggtgcagcaggcatgggggcagatggtcgctcgctacggccagccgacccacctgcacgatcgccagggtcgcggcttcgccgtcgtcggctacggtaagcttggcggctgggagctgggctacagctccgatctcgatctggtgttcctccatgactgcccggcggaggtgatgaccgacggcgagcgggagattgacggccgtcagttctacctgcggctggcccagcggatcatgcacctgttcagcacccgcacctcgtccggtattctctacgaagtggacgcccggctgcgtccttctggcgcggcggggatgctggtcaccaccgccgacgcgtttgctgactatcagcagaacgaagcctggacgtgggaacatcaggcgctggtgcgcgcccgcgtggtctatggcgacccggcgctgcaggcgcgctttgacgccattcgtcgcgatatcctgaccaccccgcgggaggggatgaccctgcagaccgaggttcgcgagatgcgcgagaagatgcgcgcccaccttggcaacaaacatcccgatcgttttgatatcaaagccgatgccggcgggatcaccgatattgaatttattactcagtatctggtcctacgctatgccagtgacaagccgaagctgacccgctggtctgacaacgtgcgtattcttgagctgctggcgcagaacgacatcatggacgaggaggaggcgcgcgccttaacgcatgcgtacaccaccttgcgtgatgcgctccatcacctggccctgcaggagcagccgggacacgtggcgccagaggccttcagccgggagcgtcagcaggtcagcgccagctggcagaagtggctgatggcttaactataaaatcgggtgtgctattatcgcgcgcaaagtttgcgtctcgcaggagagagtcatgaaagtaacgctgccggagtttgaacgtgcaggagtgttggtggtgggtgatgtgatgctggaccgctactggtacggccccaccagtcgtatttccccggaagccccggtgccggtggtgaaggtggaaaatatcgaagaacgtcctggcggcgcggcaaacgtagcgatgaacatcgcctccctgggggcaacgtcgcgcctggtgggattgaccgggattgatgacgctgcccgcgcgctgagccaggcgctggccaatgtgaatgtgaagtgcgacttcgtctccgtcccgactcacccgaccatcaccaagctgcgggtgctgtcgcgcaatcagcagctgatccgcctcgactttgaagagggcttctccggcgtggatccgcagccgatgcatgagcgcattcagcaggcgctgggagccattggcgcactgg

SEQ ID NO: 109 - DNA, 613 bp, synthetic construct
  misc_feature 1..613: note = Synthetic polynucleotide
  misc_feature 1..613: note = delta-nifL::PinfC
  source 1..613: mol_type = other DNA
atgaccctgaatatgatgctagaagcgtcaggtaccggtcatgattcaccgtgcgattctcggttccctggagcgcttcattggcatcctgaccgaagagttcgctggcttcttcccaacctggattgcaccagtgcaggtagtggtcatgaatattaccgattctcaggctgaatacgttaacgaattgacgcgtaaactacaaaatgcgggcattcgtgtaaaagcagacttgagaaatgagaagattggctttaaaatccgcgagcacactttacgtcgtgtcccgtatatgttggtctgtggcgacaaagaagtcgaagccggcaaagtggccgtgcgcacccgtcgcgggaaagacctcggcagcatggacgtaagtgaagtgattgagaagctgcaacaagagattcgcagccgcagtcttcaacaactggaggaataaggtattaaaggcggaaaacgagttcaaacggcacgtccgaatcgtatcaatggcgagattcgcgccctggaagttcgcgccattgagctggcttcccgaccgcagggcggcacctgcctgaccctgcgtttcccgctgtttaacaccctgaccggaggtgaagcatga

SEQ ID NO: 110 - DNA, 1613 bp, synthetic construct
  misc_feature 1..1613: note = Synthetic polynucleotide
  misc_feature 1..1613: note = delta-nifL::PinfC with 500bp flank
  source 1..1613: mol_type = other DNA
ggccgtcgcccagcgtcggcgtccccaacagcagggccgggtaggccagcaggtccgccagcgtggcgcggttaatattgaccggggcggcggcggcctcccccagctgcttgtggatcattttcgcgatcttgcgggttttaccggtatcggtaccaaagaaaatgccaatgttcgccatagtacgctcctgtcggaatggtgttgaaaaaaggaatgacgacagaggtattgcgaaggctgtgccaggttgccctgcaccgcgacggcccatccctgccccatcaggatcgcttcgcatcacgatgccgcgcgccaaaggcgcacccggcggggcgaaaggtaaaaatccgtgaattttccccctgtcggatcaatgtttcgcgtggtcgttccgataagggcgcacactttgcatggttatccgggttcggcttaccccgccgcgttttgcgcacggtgtcggacaatttgtcataactgcgacacaggagtttgcgatgaccctgaatatgatgctagaagcgtcaggtaccggtcatgattcaccgtgcgattctcggttccctggagcgcttcattggcatcctgaccgaagagttcgctggcttcttcccaacctggattgcaccagtgcaggtagtggtcatgaatattaccgattctcaggctgaatacgttaacgaattgacgcgtaaactacaaaatgcgggcattcgtgtaaaagcagacttgagaaatgagaagattggctttaaaatccgcgagcacactttacgtcgtgtcccgtatatgttggtctgtggcgacaaagaagtcgaagccggcaaagtggccgtgcgcacccgtcgcgggaaagacctcggcagcatggacgtaagtgaagtgattgagaagctgcaacaagagattcgcagccgcagtcttcaacaactggaggaataaggtattaaaggcggaaaacgagttcaaacggcacgtccgaatcgtatcaatggcgagattcgcgccctggaagttcgcgccattgagctggcttcccgaccgcagggcggcacctgcctgaccctgcgtttcccgctgtttaacaccctgaccggaggtgaagcatgatccctgaatccgacccggacaccaccgtcagacgcttcgacctctctcagcagttcaccgccatgcagcggataagcgtggtgctgagccgggccaccgaggccagcaaaacgctgcaggaggtgctcagcgtattacacaacgatgcctttatgcagcacgggatgatctgcctgtacgacagcgagcaggagatcctcagtatcgaagcgctgcagcaaaccggccagcagcccctccccggcagcacgcagatccgctatcgccccggcgagggactggtggggaccgtgctggcccaggggcagtcgctggtgctgccccgggtcgccgacgatcagcgttttctcgaccgcctgagcctctacgattacgatctgccgtttatcgccgtaccgttgatggggcccaacgcccggccaataggggtgctggcggcccagccgatggcgcgccaggaagagcggctgccggcctgcacccgttttctcgaaaccgtc

SEQ ID NO: 111 - DNA, 1155 bp, synthetic construct
  misc_feature 1..1155: note = Synthetic polynucleotide
  misc_feature 1..1155: note = glnE-delta-AR-2 36bp deletion
  source 1..1155: mol_type = other DNA
atggcgctgaagcacctgatcacgctctgcgcggcgtcgccgatggtcgccagccagctggcgcgccacccgctgctgctggatgagctgctggatcccaacaccctctatcagccgacggcgaccgatgcctatcgcgacgagctgcgccagtacctgctgcgcgtgccggaagaggatgaagagcagcagctgcatatcgcggcggcggatatcgctggtaccctgccggtgatgaaggtcagcgatcacttaacctggcttgccgaagcgatcctcgacgcggtggtgcagcaggcatgggggcagatggtcgctcgctacggccagccgacccacctgcacgatcgccagggtcgcggcttcgccgtcgtcggctacggtaagcttggcggctgggagctgggctacagctccgatctcgatctggtgttcctccatgactgcccggcggaggtgatgaccgacggcgagcgggagattgacggccgtcagttctacctgcggctggcccagcggatcatgcacctgttcagcacccgcacctcgtccggtattctctacgaagtggacgcccggctgcgtccttctggcgcggcggggatgctggtcaccaccgccgacgcgtttgctgactatcagcagaacgaagcctggacgtgggaacatcaggcgctggtgcgcgcccgcgtggtctatggcgacccggcgctgcaggcgcgctttgacgccattcgtcgcgatatcctgaccaccccgcgggaggggatgaccctgcagaccgaggttcgcgagatgcgcgagaagatgcgcgcccaccttggcaacaaacatcccgatcgttttgatatcaaagccgatgccggcgggatcaccgatattgaatttattactcagtatctggtcctacgctatgccagtgacaagccgaagctgacccgctggtctgacaacgtgcgtattcttgagctgctggcgcagaacgacatcatggacgaggaggaggcgcgcgccttaacgcatgcgtacaccaccttgcgtgatgcgctccatcacctggccctgcaggagcagccgggacacgtggcgccagaggccttcagccgggagcgtcagcaggtcagcgccagctggcagaagtggctgatggcttaa

SEQ ID NO: 112 - DNA, 2155 bp, synthetic construct
  misc_feature 1..2155: note = Synthetic polynucleotide
  misc_feature 1..2155: note = glnE-delta-AR-2 36bp deletion
  source 1..2155: mol_type = other DNA
cgtaaggcgaccacccagctccgcgcgttgctgaacgacgctgaagccgttctgctggccgcggacaccgccgacgaggcgttattccgcaccgaggtcgtcggcgccaaactggccctgactgaatggctggtccagcgcggctggcgtccgttcctcaacgaggcaggagagaaaaaaatagccggatcgttcaaacggtttgccgatattaacctctcgcgggtggcggccgagctgcgcagcgccgtgcagcatctggcggttgaagatgccgccgaccagttgccgaagctgtcccgcgacatcgacagcgtccagctgctggcgggcgcctatggcgacgccgtcgcgccgtggctggagaactggcaggagcttcaccgtgcaatagcacatgacgatcgcagcgtctttgaatatttccgtcgccaggcgctggctgccgagccgttctggctgcatagtggaaaacgataatttcaggccagggagcccttatggcgctgaagcacctgatcacgctctgcgcggcgtcgccgatggtcgccagccagctggcgcgccacccgctgctgctggatgagctgctggatcccaacaccctctatcagccgacggcgaccgatgcctatcgcgacgagctgcgccagtacctgctgcgcgtgccggaagaggatgaagagcagcagctgcatatcgcggcggcggatatcgctggtaccctgccggtgatgaaggtcagcgatcacttaacctggcttgccgaagcgatcctcgacgcggtggtgcagcaggcatgggggcagatggtcgctcgctacggccagccgacccacctgcacgatcgccagggtcgcggcttcgccgtcgtcggctacggtaagcttggcggctgggagctgggctacagctccgatctcgatctggtgttcctccatgactgcccggcggaggtgatgaccgacggcgagcgggagattgacggccgtcagttctacctgcggctggcccagcggatcatgcacctgttcagcacccgcacctcgtccggtattctctacgaagtggacgcccggctgcgtccttctggcgcggcggggatgctggtcaccaccgccgacgcgtttgctgactatcagcagaacgaagcctggacgtgggaacatcaggcgctggtgcgcgcccgcgtggtctatggcgacccggcgctgcaggcgcgctttgacgccattcgtcgcgatatcctgaccaccccgcgggaggggatgaccctgcagaccgaggttcgcgagatgcgcgagaagatgcgcgcccaccttggcaacaaacatcccgatcgttttgatatcaaagccgatgccggcgggatcaccgatattgaatttattactcagtatctggtcctacgctatgccagtgacaagccgaagctgacccgctggtctgacaacgtgcgtattcttgagctgctggcgcagaacgacatcatggacgaggaggaggcgcgcgccttaacgcatgcgtacaccaccttgcgtgatgcgctccatcacctggccctgcaggagcagccgggacacgtggcgccagaggccttcagccgggagcgtcagcaggtcagcgccagctggcagaagtggctgatggcttaactataaaatcgggtgtgctattatcgcgcgcaaagtttgcgtctcgcaggagagagtcatgaaagtaacgctgccggagtttgaacgtgcaggagtgttggtggtgggtgatgtgatgctggaccgctactggtacggccccaccagtcgtatttccccggaagccccggtgccggtggtgaaggtggaaaatatcgaagaacgtcctggcggcgcggcaaacgtagcgatgaacatcgcctccctgggggcaacgtcgcgcctggtgggattgaccgggattgatgacgctgcccgcgcgctgagccaggcgctggccaatgtgaatgtgaagtgcgacttcgtctccgtcccgactcacccgaccatcaccaagctgcgggtgctgtcgcgcaatcagcagctgatccgcctcgactttgaagagggcttctccggcgtggatccgcagccgatgcatgagcgcattcagcaggcgctgggagccattggcgcactgg

SEQ ID NO: 113 - DNA, 412 bp, synthetic construct
  misc_feature 1..412: note = Synthetic polynucleotide
  misc_feature 1..412: note = delta-nifL::Prm8.2
  source 1..412: mol_type = other DNA
atgaccctgaatatgatgctcgacgccgtcctcgcagtaccattgcaaccgactttacagcaagaagtgattctggcacgcatggaacaaattcttgccagtcgggctttatccgatgacgaacgcgcacagcttttatatgagcgcggagtgttgtatgatagtctcggtctgagggcattagcgcgaaatgatttttcacaagcgctggcaatccgacccgatatgcctgaagtattcaattacttaggcatttacttaacgcaggcaggcaattttgatgctgcctatgaagcgtttgattctgtacttgagcttgatcgccattgagctggcttcccgaccgcagggcggcacctgcctgaccctgcgtttcccgctgtttaacaccctgaccggaggtgaagcatga

SEQ ID NO: 114 - DNA, 1389 bp, synthetic construct
  misc_feature 1..1389: note = Synthetic polynucleotide
  misc_feature 1..1389: note = delta-nifL::Prm8.2 with 500bp flank
  source 1..1389: mol_type = other DNA
cccaacagcagggccgggtaggccagcaggtccgccagcgtggcgcggttaatattgaccggggcggcggcggcctcccccagctgcttgtggatcattttcgcgatcttgcgggttttaccggtatcggtaccaaagaaaatgccaatgttcgccatagtacgctcctgtcggaatggtgttgaaaaaaggaatgacgacagaggtattgcgaaggctgtgccaggttgccctgcaccgcgacggcccatccctgccccatcaggatcgcttcgcatcacgatgccgcgcgccaaaggcgcacccggcggggcgaaaggtaaaaatccgtgaattttccccctgtcggatcaatgtttcgcgtggtcgttccgataagggcgcacactttgcatggttatccgggttcggcttaccccgccgcgttttgcgcacggtgtcggacaatttgtcataactgcgacacaggagtttgcgatgaccctgaatatgatgctcgacgccgtcctcgcagtaccattgcaaccgactttacagcaagaagtgattctggcacgcatggaacaaattcttgccagtcgggctttatccgatgacgaacgcgcacagcttttatatgagcgcggagtgttgtatgatagtctcggtctgagggcattagcgcgaaatgatttttcacaagcgctggcaatccgacccgatatgcctgaagtattcaattacttaggcatttacttaacgcaggcaggcaattttgatgctgcctatgaagcgtttgattctgtacttgagcttgatcgccattgagctggcttcccgaccgcagggcggcacctgcctgaccctgcgtttcccgctgtttaacaccctgaccggaggtgaagcatgatccctgaatccgacccggacaccaccgtcagacgcttcgacctctctcagcagttcaccgccatgcagcggataagcgtggtgctgagccgggccaccgaggccagcaaaacgctgcaggaggtgctcagcgtattacacaacgatgcctttatgcagcacgggatgatctgcctgtacgacagcgagcaggagatcctcagtatcgaagcgctgcagcaaaccggccagcagcccctccccggcagcacgcagatccgctatcgccccggcgagggactggtggggaccgtgctggcccaggggcagtcgctggtgctgccccgggtcgccgacgatcagcgttttctcgaccgcctgagcctctacgattacgatctgccgtttatcgccgtaccgttgatggggcccaacgcccggccaataggggtgctggcggcccagccgatggcgcgccaggaagagcggctgccggcctgcacccgttttctcgaaaccgtc

SEQ ID NO: 115 - DNA, 1155 bp, synthetic construct; identical to SEQ ID NO: 111
  misc_feature 1..1155: note = Synthetic polynucleotide
  misc_feature 1..1155: note = glnE-delta-AR-2 36bp deletion
  source 1..1155: mol_type = other DNA

SEQ ID NO: 116 - DNA, 2155 bp, synthetic construct; identical to SEQ ID NO: 112
  misc_feature 1..2155: note = Synthetic polynucleotide
  misc_feature 1..2155: note = glnE-delta-AR-2 36bp deletion
  source 1..2155: mol_type = other DNA

SEQ ID NO: 117 - DNA, 413 bp, synthetic construct
  misc_feature 1..413: note = Synthetic polynucleotide
  misc_feature 1..413: note = delta-nifL::Prm6.2
  source 1..413: mol_type = other DNA
atgaccctgaatatgatgctcgagctaaagttctcggctaatcgctgataacatttgacgcaatgcgcaataaaagggcatcatttgatgccctttttgcacgctttcataccagaacctggctcatcagtgattttttttgtcataatcattgctgagacaggctctgaagagggcgtttatacaccaaaccattcgagcggtagcgcgacggcaagtcagcgttctcctttgcaatagcagggaagaggcgccagaaccgccagcgttgaagcagtttgaacgcgttcagtgtataatccgaaacttaatttcggtttggagccattgagctggcttcccgaccgcagggcggcacctgcctgaccctgcgtttcccgctgtttaacaccctgaccggaggtgaagcatga

SEQ ID NO: 118 - DNA, 1413 bp, synthetic construct
  misc_feature 1..1413: note = Synthetic polynucleotide
  misc_feature 1..1413: note = delta-nifL::Prm6.2 with 500bp flank
  source 1..1413: mol_type = other DNA
ggccgtcgcccagcgtcggcgtccccaacagcagggccgggtaggccagcaggtccgccagcgtggcgcggttaatattgaccggggcggcggcggcctcccccagctgcttgtggatcattttcgcgatcttgcgggttttaccggtatcggtaccaaagaaaatgccaatgttcgccatagtacgctcctgtcggaatggtgttgaaaaaaggaatgacgacagaggtattgcgaaggctgtgccaggttgccctgcaccgcgacggcccatccctgccccatcaggatcgcttcgcatcacgatgccgcgcgccaaaggcgcacccggcggggcgaaaggtaaaaatccgtgaattttccccctgtcggatcaatgtttcgcgtggtcgttccgataagggcgcacactttgcatggttatccgggttcggcttaccccgccgcgttttgcgcacggtgtcggacaatttgtcataactgcgacacaggagtttgcgatgaccctgaatatgatgctcgagctaaagttctcggctaatcgctgataacatttgacgcaatgcgcaataaaagggcatcatttgatgccctttttgcacgctttcataccagaacctggctcatcagtgattttttttgtcataatcattgctgagacaggctctgaagagggcgtttatacaccaaaccattcgagcggtagcgcgacggcaagtcagcgttctcctttgcaatagcagggaagaggcgccagaaccgccagcgttgaagcagtttgaacgcgttcagtgtataatccgaaacttaatttcggtttggagccattgagctggcttcccgaccgcagggcggcacctgcctgaccctgcgtttcccgctgtttaacaccctgaccggaggtgaagcatgatccctgaatccgacccggacaccaccgtcagacgcttcgacctctctcagcagttcaccgccatgcagcggataagcgtggtgctgagccgggccaccgaggccagcaaaacgctgcaggaggtgctcagcgtattacacaacgatgcctttatgcagcacgggatgatctgcctgtacgacagcgagcaggagatcctcagtatcgaagcgctgcagcaaaccggccagcagcccctccccggcagcacgcagatccgctatcgccccggcgagggactggtggggaccgtgctggcccaggggcagtcgctggtgctgccccgggtcgccgacgatcagcgttttctcgaccgcctgagcctctacgattacgatctgccgtttatcgccgtaccgttgatggggcccaacgcccggccaataggggtgctggcggcccagccgatggcgcgccaggaagagcggctgccggcctgcacccgttttctcgaaaccgtc

SEQ ID NO: 119 - DNA, 513 bp, synthetic construct
  misc_feature 1..513: note = Synthetic polynucleotide
  misc_feature 1..513: note = delta-nifL::Prm1.2
  source 1..513: mol_type = other DNA
atgaccctgaatatgatgctcgagcccgctgaccgaccagaacttccaccttggactcggctatacccttggcgtgacggcgcgcgataactgggactacatccccattccggtgatcttaccattggcgtcaataggttacggtccggcgactttccagatgacctatattcccggcacctacaataacggtaacgtttacttcgcctgggctcgtatacagttttaattcgctaagtcttagcaataaatgagataagcggtgtgtcttgtggaaaaacaaggactaaagcgttacccactaaaaaagatagcgacttttatcactttttagcaaagttgcactggacaaaaggtaccacaattggtgtactgatactcgacacagcattagtgtcgatttttcatataaaggtaattttggccattgagctggcttcccgaccgcagggcggcacctgcctgaccctgcgtttcccgctgtttaacaccctgaccggaggtgaagcatga

SEQ ID NO: 120 - DNA, 1513 bp, synthetic construct
  misc_feature 1..1513: note = Synthetic polynucleotide
  misc_feature 1..1513: note = delta-nifL::Prm1.2 with 500bp flank
  source 1..1513: mol_type = other DNA
ggccgtcgcccagcgtcggcgtccccaacagcagggccgggtaggccagcaggtccgccagcgtggcgcggttaatattgaccggggcggcggcggcctcccccagctgcttgtggatcattttcgcgatcttgcgggttttaccggtatcggtaccaaagaaaatgccaatgttcgccatagtacgctcctgtcggaatggtgttgaaaaaaggaatgacgacagaggtattgcgaaggctgtgccaggttgccctgcaccgcgacggcccatccctgccccatcaggatcgcttcgcatcacgatgccgcgcgccaaaggcgcacccggcggggcgaaaggtaaaaatccgtgaattttccccctgtcggatcaatgtttcgcgtggtcgttccgataagggcgcacactttgcatggttatccgggttcggcttaccccgccgcgttttgcgcacggtgtcggacaatttgtcataactgcgacacaggagtttgcgatgaccctgaatatgatgctcgagcccgctgaccgaccagaacttccaccttggactcggctatacccttggcgtgacggcgcgcgataactgggactacatccccattccggtgatcttaccattggcgtcaataggttacggtccggcgactttccagatgacctatattcccggcacctacaataacggtaacgtttacttcgcctgggctcgtatacagttttaattcgctaagtcttagcaataaatgagataagcggtgtgtcttgtggaaaaacaaggactaaagcgttacccactaaaaaagatagcgacttttatcactttttagcaaagttgcactggacaaaaggtaccacaattggtgtactgatactcgacacagcattagtgtcgatttttcatataaaggtaattttggccattgagctggcttcccgaccgcagggcggcacctgcctgaccctgcgtttcccgctgtttaacaccctgaccggaggtgaagcatgatccctgaatccgacccggacaccaccgtcagacgcttcgacctctctcagcagttcaccgccatgcagcggataagcgtggtgctgagccgggccaccgaggccagcaaaacgctgcaggaggtgctcagcgtattacacaacgatgcctttatgcagcacgggatgatctgcctgtacgacagcgagcaggagatcctcagtatcgaagcgctgcagcaaaccggccagcagcccctccccggcagcacgcagatccgctatcgccccggcgagggactggtggggaccgtgctggcccaggggcagtcgctggtgctgccccgggtcgccgacgatcagcgttttctcgaccgcctgagcctctacgattacgatctgccgtttatcgccgtaccgttgatggggcccaacgcccggccaataggggtgctggcggcccagccgatggcgcgccaggaagagcggctgccggcctgcacccgttttctcgaaaccgtc

SEQ ID NO: 121 - DNA, 1155 bp, synthetic construct; identical to SEQ ID NO: 111
  misc_feature 1..1155: note = Synthetic polynucleotide
  misc_feature 1..1155: note = glnE-delta-AR-2 36bp deletion
  source 1..1155: mol_type = other DNA

SEQ ID NO: 122 - DNA, 2155 bp, synthetic construct; identical to SEQ ID NO: 112
  misc_feature 1..2155: note = Synthetic polynucleotide
  misc_feature 1..2155: note = glnE-delta-AR-2 36bp deletion
  source 1..2155: mol_type = other DNA

SEQ ID NO: 123 - DNA, 613 bp, synthetic construct; identical to SEQ ID NO: 109
  misc_feature 1..613: note = Synthetic polynucleotide
  misc_feature 1..613: note = delta-nifL::PinfC
  source 1..613: mol_type = other DNA

SEQ ID NO: 124 - DNA, 1613 bp, synthetic construct; identical to SEQ ID NO: 110
  misc_feature 1..1613: note = Synthetic polynucleotide
  misc_feature 1..1613: note = delta-nifL::PinfC with 500bp flank
  source 1..1613: mol_type = other DNA

SEQ ID NO: 125 - DNA, 1191 bp, synthetic construct; identical to SEQ ID NO: 107
  misc_feature 1..1191: note = Synthetic polynucleotide
  misc_feature 1..1191: note = glnE-delta-AR-2
  source 1..1191: mol_type = other DNA

SEQ ID NO: 126 - DNA, 2191 bp, synthetic construct; identical to SEQ ID NO: 108
  misc_feature 1..2191: note = Synthetic polynucleotide
  misc_feature 1..2191: note = glnE-delta-AR-2 with 500bp flank
  source 1..2191: mol_type = other DNA

SEQ ID NO: 127 - DNA, 1188 bp, synthetic construct
  misc_feature 1..1188: note = Synthetic polynucleotide
  misc_feature 1..1188: note = glnE-delta-AR-2
  source 1..1188: mol_type = other DNA
atggcgctcaaacagttaatccgtctgtgtgccgcctcgccgatggtcgcgacacaacttgcacgtcatcctttattgctcgatgaactgctcgacccgcgcacgctttaccagccgattgagccgggcgcttaccgcgacgaactgcgtcagtatctgatgcgggtgccaacagaagacgaagaacagcagcttgaagccgtgcgccagttcaaacaggcccagcatttgcgtatcgcagccggggatatttccggggcattgccggtgatgaaagtcagtgaccatttaacctaccttgccgaggccattctcgatgtcgtggtgcagcatgcgtgggaacaaatggtcgtaaaatacgggcagcccgcgcatcttcagcaccgtgaggggcgcggttttgccgtggtcggttacgggaaactcggtggctgggagctgggttatagctcagatctggatctggtcttcctgctcgattgcgcgccggaggtgatgacggacggcgaacgcagcatcgacggacgtcagttttatcttcggctggcgcagcgcattatgcacttattcagcacccggacatcgtcaggcattctttacgaggttgatccgcgtctgcgaccttccggcgcatccggcatgctggtcagtaccattgaagcgtttgcagattatcaggccaatgaagcctggacgtgggagcatcaggcgctggttcgcgcgcgcgtggtttacggggatccgcaactgacacagcaatttaacgccacgcgtcgcgacattctttgccgccagcgcgatggcgacggcctgcgtaaggaggtccgtgaaatgcgcgagaaaatgtatgcccatctggggagtaaaaaagcccacgagtttgatctgaaagccgatccgggtggcatcacggatattgaattcattgcacaatacctggttctgcgtttcgcgcatgatgagccgaagctgacgcgctggtctgataacgtgcggatttttgaactgatggcacgatatgacatcatgccggaagaggaagcgcgccatctgacgcaggcttatgtgacgctgcgcgatgaaattcatcatctggcgttgcaggaacacagcgggaaagtggccgcggacagctttgctactgagcgcgcgcagatccgtgccagctgggcaaagtggctcggctga

SEQ ID NO: 128 - DNA, 2188 bp, synthetic construct
  misc_feature 1..2188: note = Synthetic polynucleotide
  misc_feature 1..2188: note = glnE-delta-AR-2 with 500bp flank
  source 1..2188: mol_type = other DNA
cggtactggaacagaaatcggcggatgcgcaggaaatttgttatgacacggcctgtctgaagtgcaagttagtgcttacttcctggctggcaacctcaggctggacgccgtttattgatgataaatctgcgaagaaactggacgcttccttcaaacgttttgctgacatcatgctcggtcgtaccgcagcggatctgaaagaagcctttgcgcagccactgacggaagaaggttatcgcgatcagctggcgcgcctgaaacgccagatcattaccttccatttgcttgccggtgcttaccctgaaaaagacgtcgatgcgtatattgccggctgggtggacctgcaacaggccatcgttcagcagcaacacgcctgggaggattcggcccgttctcacgcggtgatgatggatgctttctggttaaacgggcaacctcgttaactgactgactagcctgggcaaactgcccgggcttttttttgcaaggaatctgatttcatggcgctcaaacagttaatccgtctgtgtgccgcctcgccgatggtcgcgacacaacttgcacgtcatcctttattgctcgatgaactgctcgacccgcgcacgctttaccagccgattgagccgggcgcttaccgcgacgaactgcgtcagtatctgatgcgggtgccaacagaagacgaagaacagcagcttgaagccgtgcgccagttcaaacaggcccagcatttgcgtatcgcagccggggatatttccggggcattgccggtgatgaaagtcagtgaccatttaacctaccttgccgaggccattctcgatgtcgtggtgcagcatgcgtgggaacaaatggtcgtaaaatacgggcagcccgcgcatcttcagcaccgtgaggggcgcggttttgccgtggtcggttacgggaaactcggtggctgggagctgggttatagctcagatctggatctggtcttcctgctcgattgcgcgccggaggtgatgacggacggcgaacgcagcatcgacggacgtcagttttatcttcggctggcgcagcgcattatgcacttattcagcacccggacatcgtcaggcattctttacgaggttgatccgcgtctgcgaccttccggcgcatccggcatgctggtcagtaccattgaagcgtttgcagattatcaggccaatgaagcctggacgtgggagcatcaggcgctggttcgcgcgcgcgtggtttacggggatccgcaactgacacagcaatttaacgccacgcgtcgcgacattctttgccgccagcgcgatggcgacggcctgcgtaaggaggtccgtgaaatgcgcgagaaaatgtatgcccatctggggagtaaaaaagcccacgagtttgatctgaaagccgatccgggtggcatcacggatattgaattcattgcacaatacctggttctgcgtttcgcgcatgatgagccgaagctgacgcgctggtctgataacgtgcggatttttgaactgatggcacgatatgacatcatgccggaagaggaagcgcgccatctgacgcaggcttatgtgacgctgcgcgatgaaattcatcatctggcgttgcaggaacacagcgggaaagtggccgcggacagctttgctactgagcgcgcgcagatccgtgccagctgggcaaagtggctcggctgagggtttttattcggctaacaggcgcttgtgatattatccggcgcattgtatttacccgatttgatttatctgttttggagtcttgggatgaaagtgactttgcctgattttcaccgcgcaggtgtgctggttgtcggtgacgtaatgttagaccgttactggtatggcccgaccaatcgtatttctccggaagctccggtgccggtggtgaaggtcagtaccattgaagagcggcctggcggtgcagctaacgtggcgatgaacatttcatctctgggcgcctcttcctgtctgatcggcctgaccggcgtagacgacgctgcgcgtgccctcagtgagcgtctggcagaagtgaaagttaactgcgatttcgtcgcactatccacacatcctaccatcaccaaactgcgaattttgtcccgtaaccagcaactgatccgcctcgactttgaggaaggttttgaaggcgttgatctcgagccgatgctgaccaaaataga

SEQ ID NO: 129 - DNA, 635 bp, synthetic construct
  misc_feature 1..635: note = Synthetic polynucleotide
  misc_feature 1..635: note = delta-nifL::Prm6.1
  source 1..635: mol_type = other DNA
atgagcatcacggcgttatcagcatcatttcctgaggggaatatcgccagccgcttgtcgctgcaacatccttcactgttttataccgtggttgaacaatcttcggtggcgatttcgctgaccgatccgcaggcgcgcatttgttatgccaatccggcattctgccgccagacgggttttgcacttgagacacttttgggcgagaaccaccgtctgctggaattttttttcacaaagcgtagcgttattgaatcgcacattttaaactgttggccgctgtggaagcgaatattggtgaaaggtgcggttttaaggcctttttctttgactctctgtcgttacaaagttaatatgcgcgccctccgtctctgaagctctcggtgaacattgttgcgaggcaggatgcgagctggttgtgttttgacattaccgataatgtgccgcgtgaacgggtgcgttatgcccgcccggaagcggcgttttcccgtccggggaatggcatggagctgcgccttatccagacgctgatcgcccatcatcgcggttctttagatctctcggtccgccctgatggcggcaccttgctgacgttacgcctgccggtacagcaggttatcaccggaggcttaaaatga

SEQ ID NO: 130 - DNA, 1635 bp, synthetic construct
  misc_feature 1..1635: note = Synthetic polynucleotide
  misc_feature 1..1635: note = delta-nifL::Prm6.1 with 500bp flank
  source 1..1635: mol_type = other DNA
tgtttcgtctcgaggccgggcaactgagcggccccgttgaaaccgacctgggctggcatctgttgttgtgcgaacaaattcgcctgccgcaacccttgccgaaagccgaagccttaacgcgggtgcgtcagcaactgattgcccggcaacagaaacattatcagcgccagtggctgcaacaactgatcaacgcctgagcctgttctccttcttgttgatgcagacgggttaatgcccgttttgcacgaaaaatgcacataaattgcctgcgttgccttataacagcgcagggaaatcctgcctccggccttgtgccacaccgcgctttgcctggtttgtggtaaaaactggcccgctttgcatcctgatgcttaaaacaccccgttcagatcaacctttgggcagataagcccgcgaaaggcctgcaaattgcacggttattccgggtgagtatatgtgtgatttgggttccggcattgcgcaataaaggggagaaagacatgagcatcacggcgttatcagcatcatttcctgaggggaatatcgccagccgcttgtcgctgcaacatccttcactgttttataccgtggttgaacaatcttcggtggcgatttcgctgaccgatccgcaggcgcgcatttgttatgccaatccggcattctgccgccagacgggttttgcacttgagacacttttgggcgagaaccaccgtctgctggaattttttttcacaaagcgtagcgttattgaatcgcacattttaaactgttggccgctgtggaagcgaatattggtgaaaggtgcggttttaaggcctttttctttgactctctgtcgttacaaagttaatatgcgcgccctccgtctctgaagctctcggtgaacattgttgcgaggcaggatgcgagctggttgtgttttgacattaccgataatgtgccgcgtgaacgggtgcgttatgcccgcccggaagcggcgttttcccgtccggggaatggcatggagctgcgccttatccagacgctgatcgcccatcatcgcggttctttagatctctcggtccgccctgatggcggcaccttgctgacgttacgcctgccggtacagcaggttatcaccggaggcttaaaatgacccagttacctaccgcgggcccggttatccggcgctttgatatgtctgcccagtttacggcgctttatcgcatcagcgtggcgctgagtcaggaaagcaacaccgggcgcgcactggcggcgatcctcgaagtgcttcacgatcatgcatttatgcaatacggcatggtgtgtctgtttgataaagaacgcaatgcactctttgtggaatccctgcatggcatcgacggcgaaaggaaaaaagagacccgccatgtccgttaccgcatgggggaaggcgtgatcggcgcggtgatgagccagcgtcaggcgctggtgttaccgcgcatttcagacgatcagcgttttctcgaccgcctgaatatttacgattacagcctgccgttgattggcgtgccgatccccggtgcggataatcagccatcgggcgtgctggtggcacagccgatggcgttgcacgaagaccggctgactgccagtacgcggtttttagaaatggtc

SEQ ID NO: 131 - DNA, 635 bp, synthetic construct; identical to SEQ ID NO: 129
  misc_feature 1..635: note = Synthetic polynucleotide
  misc_feature 1..635: note = delta-nifL::Prm6.1
  source 1..635: mol_type = other DNA

SEQ ID NO: 132 - DNA, 1635 bp, synthetic construct; identical to SEQ ID NO: 130
  misc_feature 1..1635: note = Synthetic polynucleotide
  misc_feature 1..1635: note = delta-nifL::Prm6.1 with 500bp flank
  source 1..1635: mol_type = other DNA

SEQ ID NO: 133 - DNA, 786 bp, synthetic construct
  misc_feature 1..786: note = Synthetic polynucleotide
  misc_feature 1..786: note = delta-nifL::Prm7.1
  source 1..786: mol_type = other DNA
atgagcatcacggcgttatcagcatcatttcctgaggggaatatcgccagccgcttgtcgctgcaacatccttcactgttttataccgtggttgaacaatcttcggtggcgatttcgctgaccgatccgcaggcgcgcatttgttatgccaatccggcattctgccgccagacgggttttgcacttgagacacttttgggcgagaaccaccgtctgctggttaaaaacgtgaccacgagcattaataaacgccacgaaatgtggcgtttatttattcaaaaagtatcttctttcataaaaagtgctaaatgcagtagcagcaaaattgggataagtcccatggaatacggctgttttcgctgcaatttttaactttttcgtaaaaaaagatgtttctttgagcgaacgatcaaaatatagcgttaaccggcaaaaaattattctcattagaaaatagtttgtgtaatacttgtaacgctacatggagattaacttaatctagagggttttataccgtctctgaagctctcggtgaacattgttgcgaggcaggatgcgagctggttgtgttttgacattaccgataatgtgccgcgtgaacgggtgcgttatgcccgcccggaagcggcgttttcccgtccggggaatggcatggagctgcgccttatccagacgctgatcgcccatcatcgcggttctttagatctctcggtccgccctgatggcggcaccttgctgacgttacgcctgccggtacagcaggttatcaccggaggcttaaaatga

SEQ ID NO: 134 - DNA, 1786 bp, synthetic construct
  misc_feature 1..1786: note = Synthetic polynucleotide
  misc_feature 1..1786: note = delta-nifL::Prm7.1 with 500bp flank
  source 1..1786: mol_type = other DNA
gtttcgtctcgaggccgggcaactgagcggccccgttgaaaccgacctgggctggcatctgttgttgtgcgaacaaattcgcctgccgcaacccttgccgaaagccgaagccttaacgcgggtgcgtcagcaactgattgcccggcaacagaaacattatcagcgccagtggctgcaacaactgatcaacgcctgagcctgttctccttcttgttgatgcagacgggttaatgcccgttttgcacgaaaaatgcacataaattgcctgcgttgccttataacagcgcagggaaatcctgcctccggccttgtgccacaccgcgctttgcctggtttgtggtaaaaactggcccgctttgcatcctgatgcttaaaacaccccgttcagatcaacctttgggcagataagcccgcgaaaggcctgcaaattgcacggttattccgggtgagtatatgtgtgatttgggttccggcattgcgcaataaaggggagaaagacatgagcatcacggcgttatcagcatcatttcctgaggggaatatcgccagccgcttgtcgctgcaacatccttcactgttttataccgtggttgaacaatcttcggtggcgatttcgctgaccgatccgcaggcgcgcatttgttatgccaatccggcattctgccgccagacgggttttgcacttgagacacttttgggcgagaaccaccgtctgctggttaaaaacgtgaccacgagcattaataaacgccacgaaatgtggcgtttatttattcaaaaagtatcttctttcataaaaagtgctaaatgcagtagcagcaaaattgggataagtcccatggaatacggctgttttcgctgcaatttttaactttttcgtaaaaaaagatgtttctttgagcgaacgatcaaaatatagcgttaaccggcaaaaaattattctcattagaaaatagtttgtgtaatacttgtaacgctacatggagattaacttaatctagagggttttataccgtctctgaagctctcggtgaacattgttgcgaggcaggatgcgagctggttgtgttttgacattaccgataatgtgccgcgtgaacgggtgcgttatgcccgcccggaagcggcgttttcccgtccggggaatggcatggagctgcgccttatccagacgctgatcgcccatcatcgcggttctttagatctctcggtccgccctgatggcggcaccttgctgacgttacgcctgccggtacagcaggttatcaccggaggcttaaaatgacccagttacctaccgcgggcccggttatccggcgctttgatatgtctgcccagtttacggcgctttatcgcatcagcgtggcgctgagtcaggaaagcaacaccgggcgcgcactggcggcgatcctcgaagtgcttcacgatcatgcatttatgcaatacggcatggtgtgtctgtttgataaagaacgcaatgcactctttgtggaatccctgcatggcatcgacggcgaaaggaaaaaagagacccgccatgtccgttaccgcatgggggaaggcgtgatcggcgcggtgatgagccagcgtcaggcgctggtgttaccgcgcatttcagacgatcagcgttttctcgaccgcctgaatatttacgattacagcctgccgttgattggcgtgccgatccccggtgcggataatcagccatcgggcgtgctggtggcacagccgatggcgttgcacgaagaccggctgactgccagtacgcggtttttagaaatggtcg

SEQ ID NO: 135 - DNA, 993 bp, synthetic construct
  misc_feature 1..993: note = Synthetic polynucleotide
  misc_feature 1..993: note = delta-nifL::Prm1.2
  source 1..993: mol_type = other DNA
atgagcatcacggcgttatcagcatcatttcctgaggggaatatcgccagccgcttgtcgctgcaacatccttcactgttttataccgtggttgaacaatcttcggtggcgatttcgctgaccgatccgcaggcgcgcatttgttatgccaatccggcattctgccgccagacgggttttgcacttgagacacttttgggcgagaaccaccgtctgctggtgaacatcactgatgcacaagctacctatgtcgaagaattaactaaaaaactgcaagatgcaggcattcgcgttaaagccgacttgagaaatgagaagattggctttaaaattcgcgaacacacgctacgccgtgttccttatatgttagtttgtggcgataaagaggtcgaagcaggcaaagttgctgttcgtacccgccgcggcaaagacttaggaagcatggatgttagcgaagtcgttgacaaactgctggcggaaatccgcagcagaagtcttcatcaactggaggaataaagtattaaaggcggaaaacgagttcaaccggcgcgtcctaatcgcattaacaaagagattcgcgcgcaagaagttcgcctcacaggcgtcgatggcgagcagattggtattgtcagtctgaatgaagctcttgaaaaagctgaggaagcgggcgtcgatttagtagaaatcagtccgaatgccgagccgccagtttgtcgaatcccgtctctgaagctctcggtgaacattgttgcgaggcaggatgcgagctggttgtgttttgacattaccgataatgtgccgcgtgaacgggtgcgttatgcccgcccggaagcggcgttttcccgtccggggaatggcatggagctgcgccttatccagacgctgatcgcccatcatcgcggttctttagatctctcggtccgccctgatggcggcaccttgctgacgttacgcctgccggtacagcaggttatcaccggaggcttaaaatga

SEQ ID NO: 136 - DNA, 1993 bp, synthetic construct
  misc_feature 1..1993: note = Synthetic polynucleotide
  misc_feature 1..1993: note = delta-nifL::Prm1.2 with 500bp flank
  source 1..1993: mol_type = other DNA
tgtttcgtctcgaggccgggcaactgagcggccccgttgaaaccgacctgggctggcatctgttgttgtgcgaacaaattcgcctgccgcaacccttgccgaaagccgaagccttaacgcgggtgcgtcagcaactgattgcccggcaacagaaacattatcagcgccagtggctgcaacaactgatcaacgcctgagcctgttctccttcttgttgatgcagacgggttaatgcccgttttgcacgaaaaatgcacataaattgcctgcgttgccttataacagcgcagggaaatcctgcctccggccttgtgccacaccgcgctttgcctggtttgtggtaaaaactggcccgctttgcatcctgatgcttaaaacaccccgttcagatcaacctttgggcagataagcccgcgaaaggcctgcaaattgcacggttattccgggtgagtatatgtgtgatttgggttccggcattgcgcaataaaggggagaaagacatgagcatcacggcgttatcagcatcatttcctgaggggaatatcgccagccgcttgtcgctgcaacatccttcactgttttataccgtggttgaacaatcttcggtggcgatttcgctgaccgatccgcaggcgcgcatttgttatgccaatccggcattctgccgccagacgggttttgcacttgagacacttttgggcgagaaccaccgtctgctggtgaacatcactgatgcacaagctacctatgtcgaagaattaactaaaaaactgcaagatgcaggcattcgcgttaaagccgacttgagaaatgagaagattggctttaaaattcgcgaacacacgctacgccgtgttccttatatgttagtttgtggcgataaagaggtcgaagcaggcaaagttgctgttcgtacccgccgcggcaaagacttaggaagcatggatgttagcgaagtcgttgacaaactgctggcggaaatccgcagcagaagtcttcatcaactggaggaataaagtattaaaggcggaaaacgagttcaaccggcgcgtcctaatcgcattaacaaagagattcgcgcgcaagaagttcgcctcacaggcgtcgatggcgagcagattggtattgtcagtctgaatgaagctcttgaaaaagctgaggaagcgggcgtcgatttagtagaaatcagtccgaatgccgagccgccagtttgtcgaatcccgtctctgaagctctcggtgaacattgttgcgaggcaggatgcgagctggttgtgttttgacattaccgataatgtgccgcgtgaacgggtgcgttatgcccgcccggaagcggcgttttcccgtccggggaatggcatggagctgcgccttatccagacgctgatcgcccatcatcgcggttctttagatctctcggtccgccctgatggcggcaccttgctgacgttacgcctgccggtacagcaggttatcaccggaggcttaaaatgacccagttacctaccgcgggcccggttatccggcgctttgatatgtctgcccagtttacggcgctttatcgcatcagcgtggcgctgagtcaggaaagcaacaccgggcgcgcactggcggcgatcctcgaagtgcttcacgatcatgcatttatgcaatacggcatggtgtgtctgtttgataaagaacgcaatgcactctttgtggaatccctgcatggcatcgacggcgaaaggaaaaaagagacccgccatgtccgttaccgcatgggggaaggcgtgatcggcgcggtgatgagccagcgtcaggcgctggtgttaccgcgcatttcagacgatcagcgttttctcgaccgcctgaatatttacgattacagcctgccgttgattggcgtgccgatccccggtgcggataatcagccatcgggcgtgctggtggcacagccgatggcgttgcacgaagaccggctgactgccagtacgcggtttttagaaatggtc

SEQ ID NO: 137 - DNA, 993 bp, synthetic construct; identical to SEQ ID NO: 135
  misc_feature 1..993: note = Synthetic polynucleotide
  misc_feature 1..993: note = delta-nifL::Prm1.2
  source 1..993: mol_type = other DNA

SEQ ID NO: 138 - DNA, 1993 bp, synthetic construct; identical to SEQ ID NO: 136
  misc_feature 1..1993: note = Synthetic polynucleotide
  misc_feature 1..1993: note = delta-nifL::Prm1.2 with 500bp flank
  source 1..1993: mol_type = other DNA

SEQ ID NO: 139 - DNA, 1188 bp, synthetic construct; identical to SEQ ID NO: 127
  misc_feature 1..1188: note = Synthetic polynucleotide
  misc_feature 1..1188: note = glnE-delta-AR-2
  source 1..1188: mol_type = other DNA

SEQ ID NO: 140 - DNA, 2188 bp, synthetic construct; identical to SEQ ID NO: 128
  misc_feature 1..2188: note = Synthetic polynucleotide
  misc_feature 1..2188: note = glnE-delta-AR-2 with 500bp flank
  source 1..2188: mol_type = other DNA

SEQ ID NO: 141 - DNA, 663 bp, synthetic construct
  misc_feature 1..663: note = Synthetic polynucleotide
  misc_feature 1..663: note = delta-nifL::Prm3.1
  source 1..663: mol_type = other DNA
atgagcatcacggcgttatcagcatcatttcctgaggggaatatcgccagccgcttgtcgctgcaacatccttcactgttttataccgtggttgaacaatcttcggtggcgatttcgctgaccgatccgcaggcgcgcatttgttatgccaatccggcattctgccgccagacgggttttgcacttgagacacttttgggcgagaaccaccgtctgctggtacagtagcgcctctcaaaaatagataaacggctcatgtacgtgggccgtttattttttctacccataatcgggaaccggtgttataatgccgcgccctcatattgtggggatttcttaatgacctatcctgggtcctaaagttgtagttgacattagcggagcactaacccgtctctgaagctctcggtgaacattgttgcgaggcaggatgcgagctggttgtgttttgacattaccgataatgtgccgcgtgaacgggtgcgttatgcccgcccggaagcggcgttttcccgtccggggaatggcatggagctgcgccttatccagacgctgatcgcccatcatcgcggttctttagatctctcggtccgccctgatggcggcaccttgctgacgttacgcctgccggtacagcaggttatcaccggaggcttaaaatga

SEQ ID NO: 142 - DNA, 1663 bp, synthetic construct
  misc_feature 1..1663: note = Synthetic polynucleotide
  misc_feature 1..1663: note = delta-nifL::Prm3.1 with 500bp flank
  source 1..1663: mol_type = other DNA
tgtttcgtctcgaggccgggcaactgagcggccccgttgaaaccgacctgggctggcatctgttgttgtgcgaacaaattcgcctgccgcaacccttgccgaaagccgaagccttaacgcgggtgcgtcagcaactgattgcccggcaacagaaacattatcagcgccagtggctgcaacaactgatcaacgcctgagcctgttctccttcttgttgatgcagacgggttaatgcccgttttgcacgaaaaatgcacataaattgcctgcgttgccttataacagcgcagggaaatcctgcctccggccttgtgccacaccgcgctttgcctggtttgtggtaaaaactggcccgctttgcatcctgatgcttaaaacaccccgttcagatcaacctttgggcagataagcccgcgaaaggcctgcaaattgcacggttattccgggtgagtatatgtgtgatttgggttccggcattgcgcaataaaggggagaaagacatgagcatcacggcgttatcagcatcatttcctgaggggaatatcgccagccgcttgtcgctgcaacatccttcactgttttataccgtggttgaacaatcttcggtggcgatttcgctgaccgatccgcaggcgcgcatttgttatgccaatccggcattctgccgccagacgggttttgcacttgagacacttttgggcgagaaccaccgtctgctggtacagtagcgcctctcaaaaatagataaacggctcatgtacgtgggccgtttattttttctacccataatcgggaaccggtgttataatgccgcgccctcatattgtggggatttcttaatgacctatcctgggtcctaaagttgtagttgacattagcggagcactaacccgtctctgaagctctcggtgaacattgttgcgaggcaggatgcgagctggttgtgttttgacattaccgataatgtgccgcgtgaacgggtgcgttatgcccgcccggaagcggcgttttcccgtccggggaatggcatggagctgcgccttatccagacgctgatcgcccatcatcgcggttctttagatctctcggtccgccctgatggcggcaccttgctgacgttacgcctgccggtacagcaggttatcaccggaggcttaaaatgacccagttacctaccgcgggcccggttatccggcgctttgatatgtctgcccagtttacggcgctttatcgcatcagcgtggcgctgagtcaggaaagcaacaccgggcgcgcactggcggcgatcctcgaagtgcttcacgatcatgcatttatgcaatacggcatggtgtgtctgtttgataaagaacgcaatgcactctttgtggaatccctgcatggcatcgacggcgaaaggaaaaaagagacccgccatgtccgttaccgcatgggggaaggcgtgatcggcgcggtgatgagccagcgtcaggcgctggtgttaccgcgcatttcagacgatcagcgttttctcgaccgcctgaatatttacgattacagcctgccgttgattggcgtgccgatccccggtgcggataatcagccatcgggcgtgctggtggcacagccgatggcgttgcacgaagaccggctgactgccagtacgcggtttttagaaatggtc

SEQ ID NO: 143 - DNA, 1188 bp, synthetic construct; identical to SEQ ID NO: 127
  misc_feature 1..1188: note = Synthetic polynucleotide
  misc_feature 1..1188: note = glnE-delta-AR-2
  source 1..1188: mol_type = other DNA

SEQ ID NO: 144 - DNA, 2188 bp, synthetic construct; identical to SEQ ID NO: 128
  misc_feature 1..2188: note = Synthetic polynucleotide
  misc_feature 1..2188: note = glnE-delta-AR-2 with 500bp flank
  source 1..2188: mol_type = other DNA

SEQ ID NO: 145 - DNA, 613 bp, synthetic construct
  misc_feature 1..613: note = Synthetic polynucleotide
  misc_feature 1..613: note = delta-nifL::PinfC
  source 1..613: mol_type = other DNA
atgagcatcacggcgttatcagctgaatatcactgactcacaagctacctatgtcgaagaattaactaaaaaactgcaagatgcaggcattcgcgttaaagccgacttgagaaatgagaagattggctttaaaattcgcgaacacacgctacgccgtgttccttatatgttagtttgtggcgataaagaggtcgaagcaggcaaagttgctgttcgtactcgtcgcggcaaagacttaggaagcatggatgttagcgaagtcgttgacaaactgctggcggaaatccgcagcagaagtcatcatcaactggaggaataaagtattaaaggcggaaaacgagttcaaccggcgcgtcctaatcgcattaacaaagagattcgcgcgcaagaagttcgcctcaccggcgtcgatggcgagcagattggtattgtcagtctgaatgaagctcttgaaaaagctgaggaagcgggcgtcgatttagtagaaatcagtccgaatgccgagccgccagtttgtcgaatctctttagatctctcggtccgccctgatggcggcaccttgctgacgttacgcctgccggtacagcaggttatcaccggaggcttaaaatga

SEQ ID NO: 146 - DNA, 1613 bp, synthetic construct
  misc_feature 1..1613: note = Synthetic polynucleotide
  misc_feature 1..1613: note = delta-nifL::PinfC with 500bp flank
  source 1..1613: mol_type = other DNA
tgtttcgtctcgaagccgggcaactgagcagccccgttgaaaccgaactgggctggcatctgttgttgtgcgaacaaattcgcctgccgcaacccttgccgaaagccgaggccttaacgcgggtgcgtcagcaactgattgcccggcaacagaatcattatcagcgccagtggctgcaacaactgatcaacgcctgagcctgttctccttcttgttggtgcagacgggttaatgcccgttttgcacgaaaaatgcacataaactgccttcgctgccttataacagcgcatggaaatcctgcctcctgccttgtgccacgccgcgctttgcctggtttgtggtaaaaactggcccgctttgcatcctgatgtttaaaacaccccgttcagatcaacctttgggcagataagcccgcgaaaggcctgcaaattgcacggttattccgggtgagtatatgtgtgatttgggttccggcattgcgcaataaaggggagaaagacatgagcatcacggcgttatcagctgaatatcactgactcacaagctacctatgtcgaagaattaactaaaaaactgcaagatgcaggcattcgcgttaaagccgacttgagaaatgagaagattggctttaaaattcgcgaacacacgctacgccgtgttccttatatgttagtttgtggcgataaagaggtcgaagcaggcaaagttgctgttcgtactcgtcgcggcaaagacttaggaagcatggatgttagcgaagtcgttgacaaactgctggcggaaatccgcagcagaagtcatcatcaactggaggaataaagtattaaaggcggaaaacgagttcaaccggcgcgtcctaatcgcattaacaaagagattcgcgcgcaagaagttcgcctcaccggcgtcgatggcgagcagattggtattgtcagtctgaatgaagctcttgaaaaagctgaggaagcgggcgtcgatttagtagaaatcagtccgaatgccgagccgccagtttgtcgaatctctttagatctctcggtccgccctgatggcggcaccttgctgacgttacgcctgccggtacagcaggttatcaccggaggcttaaaatgacccagttacctaccgcgggcccggttatccggcgctttgatatgtctgcccagtttacggcgctttatcgcatcagcgtggcgctgagtcaggaaagcaataccgcgcgcgcactggcggcgatcctcgaagtgcttcacgatcatgcatttatgcaatacggcatggtgtgtctgttcgataaagaacgcaatgcactgtttgtggaatccctgcatggcatcgacggcgaaaggaaaaaagaaacccgccatgtccgttaccgcatgggggaaggcgtgatcggcgcggtgatgagccagcgtcaggcgctggtgttaccgcgcatttcagacgatcagcgttttctcgaccgcctgaatatttacgattacagcctgccgctgattggtgtgccgatccccggtgcggataatcagcctgcgggtgtgctggtggcacagccgatggcgttgcacgaagaccggctggctgccagtacgcggtttttagaaatggtc

SEQ ID NO: 147 - DNA, 426 bp, synthetic construct
  misc_feature 1..426: note = Synthetic polynucleotide
  misc_feature 1..426: note = delta-nifL::Prm5
  source 1..426: mol_type = other DNA
atgaccctgaatatgatgatggatgccggcggacatcatcgcgacaaacaatattaataccggcaaccacaccggcaatttacgagactgcgcaggcatcctttctcccgtcaatttctgtcaaataaagtaaaagaggcagtctacttgaattacccccggctggttgagcgtttgttgaaaaaaagtaactgaaaaatccgtagaatagcgccactctgatggttaattaacctattcaattaagaattatctggatgaatgtgccattaaatgcgcagcataatggtgcgttgtgcgggaaaactgcttttttttgaaagggttggtcagtagcggaaacaactcacttcacaccccgaagggggaagttgcctgaccctacgattcccgctatttcattcactgaccggaggttcaaaatga

SEQ ID NO: 148 - DNA, 1426 bp, synthetic construct
  misc_feature 1..1426: note = Synthetic polynucleotide
  misc_feature 1..1426: note = delta-nifL::Prm5 with 500bp flank
  source 1..1426: mol_type = other DNA
accggatacgagagaaaagtgtctacatcggttcggttgatattgaccggcgcatccgccagcccgcccagtttctggtggatctgtttggcgattttgcgggtcttgccggtgtcggtgccgaaaaaaataccaatatttgccataacacacgctcctgttgaaaaagagatcccgccgggaaatgcggtgaacgtgtctgatattgcgaagagtgtgccagttttggtcgcgggcaaaacctgcaccagtttggttattaatgcaccagtctggcgctttttttcgccgagtttctcctcgctaatgcccgccaggcgcggctttggcgctgatagcgcgctgaataccgatctggatcaaggttttgtcgggttatcagccaaaaggtgcactctttgcatggttatacgtgcctgacatgttgtccgggcgacaaacggcctggtggcacaaattgtcagaactacgacacgactaactgaccgcaggagtgtgcgatgaccctgaatatgatgatggatgccggcggacatcatcgcgacaaacaatattaataccggcaaccacaccggcaatttacgagactgcgcaggcatcctttctcccgtcaatttctgtcaaataaagtaaaagaggcagtctacttgaattacccccggctggttgagcgtttgttgaaaaaaagtaactgaaaaatccgtagaatagcgccactctgatggttaattaacctattcaattaagaattatctggatgaatgtgccattaaatgcgcagcataatggtgcgttgtgcgggaaaactgcttttttttgaaagggttggtcagtagcggaaacaactcacttcacaccccgaagggggaagttgcctgaccctacgattcccgctatttcattcactgaccggaggttcaaaatgacccagcgaaccgagtcgggtaataccgtctggcgcttcgatttgtcccagcagttcactgcgatgcagcgcataagcgtggtactcagccgggcgaccgaggtcgatcagacgctccagcaagtgctgtgcgtattgcacaatgacgcctttttgcagcacggcatgatctgtctgtacgacagccagcaggcgattttgaatattgaagcgttgcaggaagccgatcagcagttaatccccggcagctcgcaaatccgctatcgtccgggcgaagggctggtcgggacggtgctttcgcagggccaatcattagtgctggcgcgcgttgctgacgatcagcgctttcttgaccggctcgggttgtatgattacaacctgccgtttatcgccgtgccgctgatagggccagatgcgcagactttcggtgtgctgacggcacaacccatggcgcgttacgaagagcgattacccgcctgcacccgctttctggaaacggtc

SEQ ID NO: 149 - DNA, 461 bp, synthetic construct
  misc_feature 1..461: note = Synthetic polynucleotide
  misc_feature 1..461: note = delta-nifL::Prm1
  source 1..461: mol_type = other DNA
atgaccctgaatatgatgatggatgccggccgtcctgtaataataaccggacaattcggactgattaaaaaagcgcccttgtggcgctttttttatattcccgcctccatttaaaataaaaaatccaatcggatttcactatttaaactggccattatctaagatgaatccgatggaagctcgctgttttaacacgcgttttttaaccttttattgaaagtcggtgcttctttgagcgaacgatcaaatttaagtggattcccatcaaaaaaatattctcaacctaaaaaagtttgtgtaatacttgtaacgctacatggagattaactcaatctagagggtattaataatgaatcgtactaaactggtactgggcgcaactcacttcacaccccgaagggggaagttgcctgaccctacgattcccgctatttcattcactgaccggaggttcaaaatga

SEQ ID NO: 150 - DNA, 1461 bp, synthetic construct
  misc_feature 1..1461: note = Synthetic polynucleotide
  misc_feature 1..1461: note = delta-nifL::Prm1 with 500bp flank
  source 1..1461: mol_type = other DNA
accggatacgagagaaaagtgtctacatcggttcggttgatattgaccggcgcatccgccagcccgcccagtttctggtggatctgtttggcgattttgcgggtcttgccggtgtcggtgccgaaaaaaataccaatatttgccataacacacgctcctgttgaaaaagagatcccgccgggaaatgcggtgaacgtgtctgatattgcgaagagtgtgccagttttggtcgcgggcaaaacctgcaccagtttggttattaatgcaccagtctggcgctttttttcgccgagtttctcctcgctaatgcccgccaggcgcggctttggcgctgatagcgcgctgaataccgatctggatcaaggttttgtcgggttatcagccaaaaggtgcactctttgcatggttatacgtgcctgacatgttgtccgggcgacaaacggcctggtggcacaaattgtcagaactacgacacgactaactgaccgcaggagtgtgcgatgaccctgaatatgatgatggatgccggccgtcctgtaataataaccggacaattcggactgattaaaaaagcgcccttgtggcgctttttttatattcccgcctccatttaaaataaaaaatccaatcggatttcactatttaaactggccattatctaagatgaatccgatggaagctcgctgttttaacacgcgttttttaaccttttattgaaagtcggtgcttctttgagcgaacgatcaaatttaagtggattcccatcaaaaaaatattctcaacctaaaaaagtttgtgtaatacttgtaacgctacatggagattaactcaatctagagggtattaataatgaatcgtactaaactggtactgggcgcaactcacttcacaccccgaagggggaagttgcctgaccctacgattcccgctatttcattcactgaccggaggttcaaaatgacccagcgaaccgagtcgggtaataccgtctggcgcttcgatttgtcccagcagttcactgcgatgcagcgcataagcgtggtactcagccgggcgaccgaggtcgatcagacgctccagcaagtgctgtgcgtattgcacaatgacgcctttttgcagcacggcatgatctgtctgtacgacagccagcaggcgattttgaatattgaagcgttgcaggaagccgatcagcagttaatccccggcagctcgcaaatccgctatcgtccgggcgaagggctggtcgggacggtgctttcgcagggccaatcattagtgctggcgcgcgttgctgacgatcagcgctttcttgaccggctcgggttgtatgattacaacctgccgtttatcgccgtgccgctgatagggccagatgcgcagactttcggtgtgctgacggcacaacccatggcgcgttacgaagagcgattacccgcctgcacccgctttctggaaacggtc

SEQ ID NO: 151 - DNA, 461 bp, synthetic construct; identical to SEQ ID NO: 149
  misc_feature 1..461: note = Synthetic polynucleotide
  misc_feature 1..461: note = delta-nifL::Prm1
  source 1..461: mol_type = other DNA

SEQ ID NO: 152 - DNA, 1461 bp, synthetic construct; identical to SEQ ID NO: 150
  misc_feature 1..1461: note = Synthetic polynucleotide
  misc_feature 1..1461: note = delta-nifL::Prm1 with 500bp flank
  source 1..1461: mol_type = other DNA

SEQ ID NO: 153 - DNA, 1188 bp, synthetic construct
  misc_feature 1..1188: note = Synthetic polynucleotide
  misc_feature 1..1188: note = glnE-delta-AR-2
  source 1..1188: mol_type = other DNA
atggcactgaaacacctcatttccctgtgtgccgcgtcgccgatggttgccagtcagctggcgcgctacccgatcctgcttgatgaattgctcgacccgaatacgctctatcaaccgacggcgatgaatgcctatcgcgatgagctgcgccaatacctgctgcgcgtgccggaagatgatgaagagcaacagcttgaggcgctgcggcagtttaagcaggcgcagttgctgcgcgtggcggcggcggatattgccggtacgttgccagtaatgaaagtgagcgatcacttaacctggctggcggaagcgattattgatgcggtggtgcagcaagcctgggggcagatggtggcgcgttatggccagccaacgcatctgcacgatcgcgaagggcgcggttttgcggtggtcggttatggcaagctgggcggctgggagctgggttacagctccgatctggatctggtattcctgcacgactgcccgatggatgtgatgaccgatggcgagcgtgaaatcgatggtcgccagttctatttgcgtctcgcgcagcgcgtgatgcacctgtttagcacgcgcacgtcgtccggcatcctttatgaagttgatgcgcgtctgcgtccatctggcgctgcggggatgctggtcactactacggaatcgttcgccgattaccagcaaaacgaagcctggacgtgggaacatcaggcgctggcccgtgcgcgcgtggtgtacggcgatccgcaactgaccgccgaatttgacgccattcgccgcgatattctgatgacgcctcgcgacggcgcaacgctgcaaaccgacgtgcgagaaatgcgcgagaaaatgcgtgcccatcttggcaacaagcataaagaccgcttcgatctgaaagccgatgaaggcggtatcaccgacatcgagtttatcgcccaatatctggtgctgcgctttgcccatgacaagccgaaactgacgcgctggtcggataatgtgcgcattctcgaagggctggcgcaaaacggcatcatggaggagcaggaagcgcaggcattgacgctggcgtacaccacattgcgtgatgagctgcaccacctggcgctgcaagagttgccgggacatgtggcgctctcctgttttgtcgccgagcgtgcgcttattaaaaccagctgggacaagtggctggtggaa

SEQ ID NO: 154 - DNA, 2206 bp, synthetic construct
  misc_feature 1..2206: note = Synthetic polynucleotide
  misc_feature 1..2206: note = glnE-delta-AR-2 with 500bp flank
  source 1..2206: mol_type = other DNA
gcgcaaagcgagtgctcacttacgtgatctgttgacacaatctgaagcgaccataacttctgccgtttcagcgaatacggcggtgtggagcgcacaatcagccctggcgaagctggtgctcaccgagtggctagtgacgcagggctggcgaaccttccttgatgaaaaagcgcaggccaaattcgccgactcctttaaacgctttgctgacatccatctgtcacgcagcgccgccgagctgaaaaaagcctttgcccaaccgctgggcgacagctatcgcgaccagttgccgcgcctggcgcgtgatatcgactgcgcgttactgctggccgggcattacgatcgcgcgcgcgccgtggaatggctggaaaactggcaggggcttcagcacgccattgaaacgcgccagagagtcgaaatcgaacatttccgtaataccgcgattacccaggagccgttctggttgcacagcggaaaacgttaacgaaaggatatttcgcatggcactgaaacacctcatttccctgtgtgccgcgtcgccgatggttgccagtcagctggcgcgctacccgatcctgcttgatgaattgctcgacccgaatacgctctatcaaccgacggcgatgaatgcctatcgcgatgagctgcgccaatacctgctgcgcgtgccggaagatgatgaagagcaacagcttgaggcgctgcggcagtttaagcaggcgcagttgctgcgcgtggcggcggcggatattgccggtacgttgccagtaatgaaagtgagcgatcacttaacctggctggcggaagcgattattgatgcggtggtgcagcaagcctgggggcagatggtggcgcgttatggccagccaacgcatctgcacgatcgcgaagggcgcggttttgcggtggtcggttatggcaagctgggcggctgggagctgggttacagctccgatctggatctggtattcctgcacgactgcccgatggatgtgatgaccgatggcgagcgtgaaatcgatggtcgccagttctatttgcgtctcgcgcagcgcgtgatgcacctgtttagcacgcgcacgtcgtccggcatcctttatgaagttgatgcgcgtctgcgtccatctggcgctgcggggatgctggtcactactacggaatcgttcgccgattaccagcaaaacgaagcctggacgtgggaacatcaggcgctggcccgtgcgcgcgtggtgtacggcgatccgcaactgaccgccgaatttgacgccattcgccgcgatattctgatgacgcctcgcgacggcgcaacgctgcaaaccgacgtgcgagaaatgcgcgagaaaatgcgtgcccatcttggcaacaagcataaagaccgcttcgatctgaaagccgatgaaggcggtatcaccgacatcgagtttatcgcccaatatctggtgctgcgctttgcccatgacaagccgaaactgacgcgctggtcggataatgtgcgcattctcgaagggctggcgcaaaacggcatcatggaggagcaggaagcgcaggcattgacgctggcgtacaccacattgcgtgatgagctgcaccacctggcgctgcaagagttgccgggacatgtggcgctctcctgttttgtcgccgagcgtgcgcttattaaaaccagctgggacaagtggctggtggaaccgtgcgccccggcgtaagtgtggtatcatcgcgcgcaaattttgtatctctcaggagacaggaatgaaagtgacgctgccagagtttaagcaagccggtgtaatggtggtgggtgatgtgatgctggatcgttactggtatggcccaaccagccgtatctctccggaagcgccagtcccggttgttaaagtcgataccattgaagagcgtcctggcggcgcggcaaacgtggcgatgaatatcgcctcactgggcgccacggcgcgtctggttggcctgactggcattgacgatgcggcgcgcgcgctgagcaaagcgctggccgatgttaacgttaaatgtgacttcgtttctgttccgacgcatcccaccatcactaagctgcgcgtgctgtcgcgtaaccagcagctgattcgcctggactttgaagagggttttgaaggagtcgatccgcaaccgatgcatgaacgcatcagccaggcgcttggtaatattggcgcgctggtgctgtcggatt

SEQ ID NO: 155 - DNA, 1563 bp, synthetic construct
  misc_feature 1..1563: note = Synthetic polynucleotide
  misc_feature 1..1563: note = glnE-delta-AR-1
  source 1..1563: mol_type = other DNA
atgtttaacgatctgattggcgatgatgaaacggattcgccggaagatgcgctttctgagagctggcgcgaattgtggcaggatgcgttgcaggaggaggattccacgcccgtgctggcgcatctctcagaggacgatcgccgccgcgtggtggcgctgattgccgattttcgcaaagagttggataaacgcaccattggcccgcgagggcggcaggtactcgatcacttaatgccgcatctgctcagcgatgtatgctcgcgcgacgatgcgccagtaccgctgtcacgcctgacgccgctgctcaccggaattattacccgcaccacttaccttgagctgctaagtgaatttcccggcgcactgaaacacctcatttccctgtgtgccgcgtcgccgatggttgccagtcagctggcgcgctacccgatcctgcttgatgaattgctcgacccgaatacgctctatcaaccgacggcgatgaatgcctatcgcgatgagctgcgccaatacctgctgcgcgtgccggaagatgatgaagagcaacagcttgaggcgctgcggcagtttaagcaggcgcagttgctgcgcgtggcggcggcggatattgccggtacgttgccagtaatgaaagtgagcgatcacttaacctggctggcggaagcgattattgatgcggtggtgcagcaagcctgggggcagatggtggcgcgttatggccagccaacgcatctgcacgatcgcgaagggcgcggttttgcggtggtcggttatggcaagctgggcggctgggagctgggttacagctccgatctggatctggtattcctgcacgactgcccgatggatgtgatgaccgatggcgagcgtgaaatcgatggtcgccagttctatttgcgtctcgcgcagcgcgtgatgcacctgtttagcacgcgcacgtcgtccggcatcctttatgaagttgatgcgcgtctgcgtccatctggcgctgcggggatgctggtcactactacggaatcgttcgccgattaccagcaaaacgaagcctggacgtgggaacatcaggcgctggcccgtgcgcgcgtggtgtacggcgatccgcaactgaccgccgaatttgacgccattcgccgcgatattctgatgacgcctcgcgacggcgcaacgctgcaaaccgacgtgcgagaaatgcgcgagaaaatgcgtgcccatcttggcaacaagcataaagaccgcttcgatctgaaagccgatgaaggcggtatcaccgacatcgagtttatcgcccaatatctggtgctgcgctttgcccatgacaagccgaaactgacgcgctggtcggataatgtgcgcattctcgaagggctggcgcaaaacggcatcatggaggagcaggaagcgcaggcattgacgctggcgtacaccacattgcgtgatgagctgcaccacctggcgctgcaagagttgccgggacatgtggcgctctcctgttttgtcgccgagcgtgcgcttattaaaaccagctgggacaagtggctggtggaaccgtgcgccccggcgtaa

SEQ ID NO: 156 - DNA, 461 bp, synthetic construct; identical to SEQ ID NO: 149
  misc_feature 1..461: note = Synthetic polynucleotide
  misc_feature 1..461: note = delta-nifL::Prm1
  source 1..461: mol_type = other DNA

SEQ ID NO: 157 - DNA, 1461 bp, synthetic construct; identical to SEQ ID NO: 150
  misc_feature 1..1461: note = Synthetic polynucleotide
  misc_feature 1..1461: note = delta-nifL::Prm1 with 500bp flank
  source 1..1461: mol_type = other DNA

SEQ ID NO: 158 - DNA, 2563 bp, synthetic construct
  misc_feature 1..2563: note = Synthetic polynucleotide
  misc_feature 1..2563: note = glnE-delta-AR-1 with 500bp flank
  source 1..2563: mol_type = other DNA
gcgcaaagcgagtgctcacttacgtgatctgttgacacaatctgaagcgaccataacttctgccgtttcagcgaatacggcggtgtggagcgcacaatcagccctggcgaagctggtgctcaccgagtggctagtgacgcagggctggcgaaccttccttgatgaaaaagcgcaggccaaattcgccgactcctttaaacgctttgctgacatccatctgtcacgcagcgccgccgagctgaaaaaagcctttgcccaaccgctgggcgacagctatcgcgaccagttgccgcgcctggcgcgtgatatcgactgcgcgttactgctggccgggcattacgatcgcgcgcgcgccgtggaatggctggaaaactggcaggggcttcagcacgccattgaaacgcgccagagagtcgaaatcgaacatttccgtaataccgcgattacccaggagccgttctggttgcacagcggaaaacgttaacgaaaggatatttcgcatgtttaacgatctgattggcgatgatgaaacggattcgccggaagatgcgctttctgagagctggcgcgaattgtggcaggatgcgttgcaggaggaggattccacgcccgtgctggcgcatctctcagaggacgatcgccgccgcgtggtggcgctgattgccgattttcgcaaagagttggataaacgcaccattggcccgcgagggcggcaggtactcgatcacttaatgccgcatctgctcagcgatgtatgctcgcgcgacgatgcgccagtaccgctgtcacgcctgacgccgctgctcaccggaattattacccgcaccacttaccttgagctgctaagtgaatttcccggcgcactgaaacacctcatttccctgtgtgccgcgtcgccgatggttgccagtcagctggcgcgctacccgatcctgcttgatgaattgctcgacccgaatacgctctatcaaccgacggcgatgaatgcctatcgcgatgagctgcgccaatacctgctgcgcgtgccggaagatgatgaagagcaacagcttgaggcgctgcggcagtttaagcaggcgcagttgctgcgcgtggcggcggcggatattgccggtacgttgccagtaatgaaagtgagcgatcacttaacctggctggcggaagcgattattgatgcggtggtgcagcaagcctgggggcagatggtggcgcgttatggccagccaacgcatctgcacgatcgcgaagggcgcggttttgcggtggtcggttatggcaagctgggcggctgggagctgggttacagctccgatctggatctggtattcctgcacgactgcccgatggatgtgatgaccgatggcgagcgtgaaatcgatggtcgccagttctatttgcgtctcgcgcagcgcgtgatgcacctgtttagcacgcgcacgtcgtccggcatcctttatgaagttgatgcgcgtctgcgtccatctggcgctgcggggatgctggtcactactacggaatcgttcgccgattaccagcaaaacgaagcctggacgtgggaacatcaggcgctggcccgtgcgcgcgtggtgtacggcgatccgcaactgaccgccgaatttgacgccattcgccgcgatattctgatgacgcctcgcgacggcgcaacgctgcaaaccgacgtgcgagaaatgcgcgagaaaatgcgtgcccatcttggcaacaagcataaagaccgcttcgatctgaaagccgatgaaggcggtatcaccgacatcgagtttatcgcccaatatctggtgctgcgctttgcccatgacaagccgaaactgacgcgctggtcggataatgtgcgcattctcgaagggctggcgcaaaacggcatcatggaggagcaggaagcgcaggcattgacgctggcgtacaccacattgcgtgatgagctgcaccacctggcgctgcaagagttgccgggacatgtggcgctctcctgttttgtcgccgagcgtgcgcttattaaaaccagctgggacaagtggctggtggaaccgtgcgccccggcgtaagtgtggtatcatcgcgcgcaaattttgtatctctcaggagacaggaatgaaagtgacgctgccagagtttaagcaagccggtgtaatggtggtgggtgatgtgatgctggatcgttactggtatggcccaaccagccgtatctctccggaagcgccagtcccggttgttaaagtcgataccattgaagagcgtcctggcggcgcggcaaacgtggcgatgaatatcgcctcactgggcgccacggcgcgtctggttggcctgactggcattgacgatgcggcgcgcgcgctgagcaaagcgctggccgatgttaacgttaaatgtgacttcgtttctgttccgacgcatcccaccatcactaagctgcgcgtgctgtcgcgtaaccagcagctgattcgcctggactttgaagagggttttgaaggagtcgatccgcaaccgatgcatgaacgcatcagccaggcgcttggtaatattggcgcgctggtgctgtcggatt

SEQ ID NO: 159 - DNA, 1563 bp, synthetic construct; identical to SEQ ID NO: 155
  misc_feature 1..1563: note = Synthetic polynucleotide
  misc_feature 1..1563: note = glnE-delta-AR-1
  source 1..1563: mol_type = other DNA

SEQ ID NO: 160 - DNA, 2563 bp, synthetic construct; identical to SEQ ID NO: 158
  misc_feature 1..2563: note = Synthetic polynucleotide
  misc_feature 1..2563: note = glnE-delta-AR-1 with 500bp flank
  source 1..2563: mol_type = other DNA

SEQ ID NO: 161 - DNA, 426 bp, synthetic construct; identical to SEQ ID NO: 147
  misc_feature 1..426: note = Synthetic polynucleotide
  misc_feature 1..426: note = delta-nifL::Prm5
  source 1..426: mol_type = other DNA

SEQ ID NO: 162 - DNA, 1426 bp, synthetic construct; identical to SEQ ID NO: 148
  misc_feature 1..1426: note = Synthetic polynucleotide
  misc_feature 1..1426: note = delta-nifL::Prm5 with 500bp flank
  source 1..1426: mol_type = other DNA

SEQ ID NO: 163 - DNA, 461 bp, synthetic construct; identical to SEQ ID NO: 149
  misc_feature 1..461: note = Synthetic polynucleotide
  misc_feature 1..461: note = delta-nifL::Prm1
  source 1..461: mol_type = other DNA

SEQ ID NO: 164 - DNA, 1461 bp, synthetic construct; identical to SEQ ID NO: 150
  misc_feature 1..1461: note = Synthetic polynucleotide
  misc_feature 1..1461: note = delta-nifL::Prm1 with 500bp flank
  source 1..1461: mol_type = other DNA

SEQ ID NO: 165 - DNA, 1188 bp, synthetic construct; identical to SEQ ID NO: 153
  misc_feature 1..1188: note = Synthetic polynucleotide
  misc_feature 1..1188: note = glnE-delta-AR-2
  source 1..1188: mol_type = other DNA

SEQ ID NO: 166 - DNA, 2206 bp, synthetic construct; identical to SEQ ID NO: 154
  misc_feature 1..2206: note = Synthetic polynucleotide
  misc_feature 1..2206: note = glnE-delta-AR-2 with 500bp flank
  source 1..2206: mol_type = other DNA

SEQ ID NO: 167 - DNA, 199 bp, synthetic construct
  misc_feature 1..199: note = Synthetic polynucleotide
  misc_feature 1..199: note = delta-amtB
  source 1..199: mol_type = other DNA
tttcgctgaaggtgtgaccatgggccatcaggtgctggtgcagctggaaagtgttgccatcactatcgtgtggtctggcgtggtggcctttattggttacaaactggcggacatgacggtaggcctgcgcgtaccggaagaacaagaacgtgaagggctggatgtaaacagccacggcgaaaacgcctataacgcctga

SEQ ID NO: 168 - DNA, 1199 bp, synthetic construct
  misc_feature 1..1199: note = Synthetic polynucleotide
  misc_feature 1..1199: note = delta-amtB with 500bp flank
  source 1..1199: mol_type = other DNA
tttcctttctgactctgcccgtccgggcgcactaacggcctgaaatactccctcttttcattcctggcacaacgattgcaatgtctgttgcgtgttagctgcggccattatcgaattcgactggagggggatctatgaagctggttaccgtggtgattaagccattcaaacttgaagacgtgcgtgaagcgctttcttctattggtattcaagggttgaccgtaactgaagtgaaaggctttggccgtcagaagggtcacgctgagctgtaccgcggtgcggaatatagcgttaatttcctgccgaaagtgaaaattgatgtggcgatcgctgacgatcaactcgatgaagtaatcgatgtgatcagcaaagcggcctacaccggaaaaattggcgacggcaaaattttcgttgctgagctgcaacgcgtcattcgtattcgtaccggcgaagccgacgaagcggcactgtaatacaagacacacagtgatggggatcggtttcgctgaaggtgtgaccatgggccatcaggtgctggtgcagctggaaagtgttgccatcactatcgtgtggtctggcgtggtggcctttattggttacaaactggcggacatgacggtaggcctgcgcgtaccggaagaacaagaacgtgaagggctggatgtaaacagccacggcgaaaacgcctataacgcctgattgcgttgagttatctcctgagcataaaaaagcctccattcggaggcttttctttttttaagtttaaagcgcggttagttgcgattgcgcatgacgccttcctgcacgctggacgcgaccagcacaccctcttgcgtatagaactcgccgcgcacaaaaccgcgagcgctggaggctgacgtgctttccacactgtagagcagccattcgttcatattaaacgggcgatggaaccacatggagtggtcaatggtggcaacctgcataccgcgctcaaggaagcccacgccgtgcggctgaagtgcaaccggcaggaagttaaagtctgaggcatatccaagcagatattgatgtacgcgaaaatcgtccggcaccgtgccgtttgcgcggatccatacctggcgggtgggatcggcaacgtggcctttcagcgggttatgaaactcaaccgggcggatctccagtggtttatcactaagaaacttctctttggcctgcgg

SEQ ID NO: 169 - DNA, 1563 bp, synthetic construct; identical to SEQ ID NO: 155
  misc_feature 1..1563: note = Synthetic polynucleotide
  misc_feature 1..1563: note = glnE-delta-AR-1
  source 1..1563: mol_type = other DNA

SEQ ID NO: 170 - DNA, 2563 bp, synthetic construct; identical to SEQ ID NO: 158
  misc_feature 1..2563: note = Synthetic polynucleotide
  misc_feature 1..2563: note = glnE-delta-AR-1 with 500bp flank
  source 1..2563: mol_type = other DNA

SEQ ID NO: 171 - DNA, 461 bp, synthetic construct; identical to SEQ ID NO: 149
  misc_feature 1..461: note = Synthetic polynucleotide
  misc_feature 1..461: note = delta-nifL::Prm1
  source 1..461: mol_type = other DNA

SEQ ID NO: 172 - DNA, 1461 bp, synthetic construct; identical to SEQ ID NO: 150
  misc_feature 1..1461: note = Synthetic polynucleotide
  misc_feature 1..1461: note = delta-nifL::Prm1 with 500bp flank
  source 1..1461: mol_type = other DNA

SEQ ID NO: 173 - DNA, 199 bp, synthetic construct; identical to SEQ ID NO: 167
  misc_feature 1..199: note = Synthetic polynucleotide
  misc_feature 1..199: note = delta-amtB
  source 1..199: mol_type = other DNA

SEQ ID NO: 174 - DNA, 1199 bp, synthetic construct; identical to SEQ ID NO: 168
  misc_feature 1..1199: note = Synthetic polynucleotide
  misc_feature 1..258: note = delta-amtB with 500bp flank
  source 1..1199: mol_type = other DNA

SEQ ID NO: 175 - DNA, 607 bp, synthetic construct
  misc_feature 1..607: note = Synthetic polynucleotide
  misc_feature 1..607: note = delta-nifL::PinfC
  source 1..607: mol_type = other DNA
atgacctttaatatgatgcctggggtcactggagcgctttatcggcatcctgaccgaagaatttgccggtttcttcccgacctggctggcccctgttcaggttgtggtgatgaatatcactgattctcaagctgaatatgtcaacgaattgacccgtaaattgcaaaatgcgggcattcgtgtaaaagcggacttgagaaacgagaagattggctttaaaatccgcgagcacactttacgtcgtgtcccttatatgttggtctgtggtgataaagaggtggaagcaggcaaagtggccgttcgcacccgccgcggtaaagacctgggcagcctggacgtaagtgaagtgattgagaagctgcaacaagagattcgcagccgcagtcttcaacaactggaggaataaggtattaaaggcggaaaacgagttcaaacggcacgtccgaatcgtatcaatggcgagattcgcgcccaggaagttcgcttaactggtctggaaggtgagcagctgggtattgcaatagaactaactacccgccctgaaggcggtacctgcctgaccctgcgattcccgttatttcattcactgaccggaggcccacgatga

SEQ ID NO: 176 - DNA, 1607 bp, synthetic construct
  misc_feature 1..1607: note = Synthetic polynucleotide
  misc_feature 1..1607: note = delta-nifL::PinfC with 500bp flank
  source 1..1607: mol_type = other DNA
ggtacgacaaaaacgtctccagcgacgtgcggttaatattgactggcgcatccgccacatcccccagtttttgctggatcagtttggcgattttgcgggtttttcccgtgtcactgccaaaaaaaataccaatgttagccatgtcgcgctcctgttgagaaagaataaggccgcctgcaaacggcggatatcccttctcctgttgcgaaggctgtgccaggtttttttaaggccttctgtgcactgaaatgggtgaaaaaatgactcttttttgtgcaggcaccgtcctctctccgctatccagacctgctttgaaggcctctgagggccaaatcagggccaaaacacgaatcaggatcaatgtttcggcgcgttacctgttcgaaaggtgcactctttgcatggttaatcacacccaatcagggctgcggatgtcgggcgtttcacaacacaaaatgttgtaaatgcgacacagccgggcctgaaaccaggagcgtgtgatgacctttaatatgatgcctggggtcactggagcgctttatcggcatcctgaccgaagaatttgccggtttcttcccgacctggctggcccctgttcaggttgtggtgatgaatatcactgattctcaagctgaatatgtcaacgaattgacccgtaaattgcaaaatgcgggcattcgtgtaaaagcggacttgagaaacgagaagattggctttaaaatccgcgagcacactttacgtcgtgtcccttatatgttggtctgtggtgataaagaggtggaagcaggcaaagtggccgttcgcacccgccgcggtaaagacctgggcagcctggacgtaagtgaagtgattgagaagctgcaacaagagattcgcagccgcagtcttcaacaactggaggaataaggtattaaaggcggaaaacgagttcaaacggcacgtccgaatcgtatcaatggcgagattcgcgcccaggaagttcgcttaactggtctggaaggtgagcagctgggtattgcaatagaactaactacccgccctgaaggcggtacctgcctgaccctgcgattcccgttatttcattcactgaccggaggcccacgatgacccagcgacccgagtcgggcaccaccgtctggcgttttgatctctcacagcaatttaccgccatgcagcgcatcagcgtggtgttgagtcgcgcaaccgagataagccagacgctgcaggaggtgctgtgtgttctgcataatgacgcatttatgcaacacggcatgctgtgtctgtatgacaaccagcaggaaattctgagtattgaagccttgcaggaggcagaccaacatctgatccccggcagctcgcaaattcgctatcgccctggcgaagggctggtaggagccgtactgtcccagggacaatctcttgtgctgccgcgtgtcgccgacgatcaacgctttctcgacaggcttggcatctatgattacaacctgccgtttatcgccgtccccttaatggggccaggcgcgcagacgattggcgtgctcgccgcgcagccgatggcgcgtctggaggagcggcttccttcctgtacgcgctttctggaaaccgtc

SEQ ID NO: 177 - DNA, 1536 bp, Kosakonia sacchari
  gene 1..1536: note = 16S-1
  source 1..1536: mol_type = genomic DNA
ttgaagagtttgatcatggctcagattgaacgctggcggcaggcctaacacatgcaagtcgaacggtagcacagagagcttgctctcgggtgacgagtggcggacgggtgagtaatgtctgggaaactgcctgatggagggggataactactggaaacggtagctaataccgcataacgtcgcaagaccaaagagggggaccttcgggcctcttgccatcagatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttcgggttgtaaagcactttcagcggggaggaagggagtaaggttaataaccttattcattgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcatccgaaactggcaggcttgagtctcgtagagggaggtagaattccaggtgtagcggtgaaatgcgtagagatctggaggaataccggtggcgaaggcggcctcctggacgaagactgacgctcaggtgcgaaagcgtggggagcaaacaggattagataccctggtagtccacgccgtaaacgatgtctatttggaggttgtgcccttgaggcgtggcttccggagctaacgcgttaaatagaccgcctggggagtacggccgcaaggttaaaactcaaatgaattgacgggggcccgcacaagcggtggagcatgtggtttaattcgatgcaacgcgaagaaccttacctggtcttgacatccacagaactttccagagatggattggtgccttcgggaactgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagtaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 178 - DNA, 1537 bp, Kosakonia sacchari
  gene 1..1537: note = 16S-2
  variation 450: note = n is a, c, t, g, unknown or other
  variation 452: note = n is a, c, t, g, unknown or other
  variation 455: note = n is a, c, t, g, unknown or other
  variation 473: note = n is a, c, t, g, unknown or other
  source 1..1537: mol_type = genomic DNA
ttgaagagtttgatcatggctcagattgaacgctggcggcaggcctaacacatgcaagtcgaacggtagcacagagagcttgctctcgggtgacgagtggcggacgggtgagtaatgtctgggaaactgcctgatggagggggataactactggaaacggtagctaataccgcataacgtcgcaagaccaaagagggggaccttcgggcctcttgccatcagatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttcgggttgtaaagcactttcagcggggaggaaggnantanggttaataacctgtgttnattgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcatccgaaactggcaggcttgagtctcgtagagggaggtagaattccaggtgtagcggtgaaatgcgtagagatctggaggaataccggtggcgaaggcggcctcctggacgaagactgacgctcaggtgcgaaagcgtggggagcaaacaggattagataccctggtagtccacgccgtaaacgatgtctatttggaggttgtgcccttgaggcgtggcttccggagctaacgcgttaaatagaccgcctggggagtacggccgcaaggttaaaactcaaatgaattgacgggggcccgcacaagcggtggagcatgtggtttaattcgatgcaacgcgaagaaccttacctggtcttgacatccacagaacttagcagagatgctttggtgccttcgggaactgtgagacaggtgctgcatggctgtcgtcagctcgtgttgtgaaatgttgggttaagtcccgcaacgagcgcaacccttatcctttgttgccagcggttaggccgggaactcaaaggagactgccagtgataaactggaggaaggtggggatgacgtcaagtcatcatggcccttacgaccagggctacacacgtgctacaatggcgcatacaaagagaagcgacctcgcgagagtaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 179 - DNA, 882 bp, Kosakonia sacchari
  gene 1..882: note = nifH
  source 1..882: mol_type = genomic DNA
atgaccatgcgtcaatgcgccatttacggcaaaggtgggatcggcaaatcgaccaccacacagaacctggtcgccgcgctggcggagatgggtaaaaaagtcatgattgtcggctgtgacccgaaagccgattccacgcgtttgatcctgcatgcgaaagcgcagaacaccattatggagatggctgctgaagtcggctccgtggaagacctggagttagaagacgtgctgcaaatcggttacggcggcgtgcgctgcgcagagtccggcggcccggagccaggcgtgggctgtgccggtcgcggggtgatcaccgcgattaacttcctcgaagaagaaggcgcttacgtgccggatctcgattttgttttctacgacgtgctgggcgacgtggtatgcggtggtttcgccatgccgattcgtgaaaacaaagcgcaggagatctacatcgtttgctctggcgaaatgatggcgatgtacgccgccaacaacatctccaaaggcatcgtgaaatacgccaaatccggtaaagtgcgcctcggcgggctgatttgtaactcgcgccagaccgaccgtgaagatgaactgatcattgcgctggcagaaaaactcggcacgcagatgatccactttgttccccgcgacaacattgtgcagcgtgcggaaatccgccgtatgacggttatcgaatatgacccgacctgcaatcaggcgaacgaatatcgcagccttgccagcaaaatcgtcaacaacaccaaaatggtggtgcccaccccctgcaccatggatgaactggaagaactgctgatggagttcggcattatggatgtggaagacaccagcatcattggtaaaaccgccgccgaagaaaacgccgtctga

SEQ ID NO: 180 - DNA, 1449 bp, Kosakonia sacchari
  gene 1..1449: note = nifD2
  source 1..1449: mol_type = genomic DNA
atgagcaatgcaacaggcgaacgcaacctggagataatcgagcaggtgctcgaggttttcccggagaagacgcgcaaagaacgcagaaaacacatgatggtgacggacccggagcaggaaagcgtcggtaagtgcatcatctctaaccgcaaatcgcagccaggcgtgatgaccgtgcgcggctgctcgtatgccggttcgaaaggggtggtatttgggccaatcaaggatatggcgcatatctcgcatggcccaatcggctgcggccaatactcccgcgccgggcggcggaactactacaccggcgtcagcggcgtggacagcttcggcacgctcaacttcacctccgattttcaggagcgcgacatcgtgtttggcggcgataaaaagctcgccaaactgattgaagagctggaagagctgttcccgctgaccaaaggcatttcgattcagtcggaatgcccggtcggcctgattggcgatgacattgaggccgtcgcgaacgccagccgcaaagccatcaacaaaccggttattccggtgcgttgcgaaggctttcgcggcgtgtcgcaatccctcggtcaccatattgccaacgatgtgatccgcgactgggtgctggataaccgcgaaggcaaaccgttcgaatccaccccttacgatgtggcgatcatcggcgattacaacatcggcggcgatgcctgggcttcgcgcattttgctcgaagagatgggcttgcgggtggtggcacagtggtctggcgacggtacgctggtggagatggaaaacacgccgttcgtcaaactgaacctggtgcattgttaccgctcaatgaactacatctcgcgccatatggaggagaagcacggtattccgtggatggaatacaacttctttggtccgacgaaaatcgcggaatcgctgcgcaaaatcgccgaccagtttgacgacaccattcgcgccaacgccgaagcggtgatcgccagataccaggcgcaaaacgacgccattatcgccaaatatcgcccgcgtctggaggggcgcaaagtgctgctttatatgggcgggctgcgtccgcgccatgtgattggcgcctatgaagacctgggaatggagatcatcgctgccggttatgagttcggtcataacgatgattacgaccgcaccttgccggatctgaaagagggcacgctgctgtttgatgatgccagcagttatgagctggaggcgttcgtcaacgcgctgaaaccggatctcatcggttccggcatcaaagagaagtacatctttcagaaaatgggcgtgccgtttcgccagatgcactcctgggattactccggcccgtaccacggctatgacggcttcgccatcttcgcccgcgatatggatatgacgctcaacaaccccgcgtggggccagttgaccgcgccgtggctgaaatccgcctga

SEQ ID NO: 181 - DNA, 1563 bp, Kosakonia sacchari
  gene 1..1563: note = nifK2
  source 1..1563: mol_type = genomic DNA
atgagccagactgctgagaaaatacagaattgccatcccctgtttgaacaggatgcttaccagacgctgtttgccggtaaacgggcactcgaagaggcgcactcgccggagcgggtgcaggaagtgtttcaatggaccactaccccggaatatgaagcgctgaactttaaacgcgaagcgctgactatcgacccggcaaaagcctgccagccgctgggcgcggtgctctgttcgctggggtttgccaataccctaccgtatgtgcacggttcacagggttgcgtggcctatttccgcacgtactttaaccgccactttaaagaaccggtggcctgcgtgtcggattcaatgacggaagacgcggcggtgttcggcgggaataacaacctcaacaccggcttacaaaacgccagcgcgctgtataaaccggagattatcgccgtctctaccacctgtatggcggaagtgatcggtgatgatttgcaggcctttatcgccaacgccaaaaaagatggttttctcgatgccgccatccccgtgccctacgcgcacacccccagttttatcggcagccatatcaccggctgggataacatgtttgaaggttttgcccggacctttacggcagaccatgaagctcagcccggcaaactttcacgcatcaacctggtgaccgggtttgaaacctatctcggcaatttccgcgtgctgaaacgcatgatggaacaaatggaggtgccggcgagtgtgctctccgatccgtcggaagtgctggatactcccgccaacgggcattaccagatgtacgcgggcgggacgacgcagcaagagatgcgcgaggcgccggatgctatcgacaccctgttgctgcagccctggcaactggtgaaaagcaaaaaagtggtgcaggagatgtggaatcagcccgccaccgaggtttctgttcccgttgggctggcaggaacagacgaactgttgatggcgattagccagttaaccggcaaggccattcccgattcactggcgctggagcgcgggcggctggtcgatatgatgctcgattcccacacctggttgcacggtaaaaaattcggcctgtttggcgatccggattttgtcatgggattgacccgtttcctgctggagctgggctgcgaaccgaccgttatcctctgccacaacggtaacaaacgctggcagaaagcaatgaagaaaatgcttgacgcctcgccgtacggccaggagagcgaagtgtttatcaactgcgatttgtggcatttccgctcgctgatgtttacccgccagccggattttatgattggcaactcgtacggcaagttcattcagcgcgacaccttagccaaaggcgagcagtttgaagttccgctgatccgcctcggttttcccctgttcgaccgccaccatctgcaccgccagaccacctggggctacgagggcgccatgagcattctcactacccttgtgaatgcggtactggagaaagtggacaaagagaccatcaagctcggcaaaaccgactacagcttcgatcttatccgttaa

SEQ ID NO: 182 - DNA, 1488 bp, Kosakonia sacchari
  gene 1..1488: note = nifL
  source 1..1488: mol_type = genomic DNA
atgaccctgaatatgatgatggatgccggcgcgcccgaggcaatcgccggtgcgctttcgcgacaccatcctgggctgttttttaccatcgttgaagaagcgcccgtcgccatttcgctgactgatgccgacgcacgcattgtctatgccaacccggctttctgccgccagaccggctatgaactagaagcgttgttgcagcaaaatccccgcctgcttgcaagtcgccaaaccccacgggaaatctatcaggatatgtggcacaccttgttacaacgccgaccgtggcgcgggcaattgattaaccgccaccgcgacggcagcctgtatctggtcgagatcgatatcaccccggtgattaacccgtttggcgaactggaacactacctggcaatgcagcgcgatatcagcgccagttatgcgctggagcagcggttgcgcaatcacatgacgctgaccgaagcggtgctgaataacattccggcggcggtggttgtagtggatgaacgcgatcatgtggttatggataaccttgcctacaaaacgttctgtgccgactgcggcggaaaagagctcctgagcgaactcaatttttcagcccgaaaagcggagctggcaaacggccaggtcttaccggtggtgctgcgcggtgaggtgcgctggttgtcggtgacctgctgggcgctgccgggcgtcagcgaagaagccagtcgctactttattgataacaggctgacgcgcacgctggtggtgatcaccgacgacacccaacaacgccagcagcaggaacagggccgacttgaccgccttaaacagcagatgaccaacggcaaactactggcagcgatccgcgaagcgcttgacgccgcgctgatccagcttaactgccccatcaatatgctggcggcggcgcgacgtttaaacggcagtgataacaacaatgtggcgctcgacgccgcgtggcgcgaaggtgaagaggcgatggcgcggctgaaacgttgccgcccgtcgctggaactggaaagtgcggccgtctggccgctgcaacccttttttgacgatctgcgcgcgctttatcacacccgctacgagcaggggaaaaatttgcaggtcacgctggattcccatcatctggtgggatttggtcagcgtacgcaactgttagcctgcctgagtctgtggctcgatcgcacgctggatattgccgccgggctgggtgatttcaccgcgcaaacgcagatttacgcccgcgaagaagagggctggctctctttgtatatcactgacaatgtgccgctgatcccgctgcgccacacccactcgccggatgcgcttaacgctccgggaaaaggcatggagctgcgcctgatccagacgctggtggcacaccaccacggcgcaatagaactcacttcacaccccgaagggggaagttgcctgaccctacgattcccgctatttcattcactgaccggaggttcaaaatga

SEQ ID NO: 183 - DNA, 1575 bp, Kosakonia sacchari
  gene 1..1575: note = nifA
  source 1..1575: mol_type = genomic DNA
atgacccagcgaaccgagtcgggtaataccgtctggcgcttcgatttgtcccagcagttcactgcgatgcagcgcataagcgtggtactcagccgggcgaccgaggtcgatcagacgctccagcaagtgctgtgcgtattgcacaatgacgcctttttgcagcacggcatgatctgtctgtacgacagccagcaggcgattttgaatattgaagcgttgcaggaagccgatcagcagttaatccccggcagctcgcaaatccgctatcgtccgggcgaagggctggtcgggacggtgctttcgcagggccaatcattagtgctggcgcgcgttgctgacgatcagcgctttcttgaccggctcgggttgtatgattacaacctgccgtttatcgccgtgccgctgatagggccagatgcgcagactttcggtgtgctgacggcacaacccatggcgcgttacgaagagcgattacccgcctgcacccgctttctggaaacggtcgctaacctggtcgcgcaaaccgtgcgtttgatggcaccaccggcagtgcgcccttccccgcgcgccgccataacacaggccgccagcccgaaatcctgcacggcctcacgcgcatttggttttgaaaatatggtcggtaacagtccggcgatgcgccagaccatggagattatccgtcaggtttcgcgctgggacaccaccgttctggtacgcggcgagagtggcaccggcaaggagctgattgccaacgccatccaccaccattcgccgcgtgccggtgcgccatttgtgaaattcaactgtgcggcgctgccggacacactgctggaaagcgaattgttcggtcacgagaaaggggcatttaccggcgcggtacgccagcgtaaaggccgttttgagctggccgatggcggcacgctgtttcttgacgagatcggcgagagtagcgcctcgtttcaggctaagctgctgcgcattttgcaggaaggcgaaatggaacgcgtcggcggcgacgagacattgcaagtgaatgtgcgcattattgccgcgacgaaccgcaatcttgaagatgaagtccggctggggcactttcgcgaagatctctattatcgcctgaatgtgatgcccatcgccctgccgccactacgcgaacgccaggaggacattgccgagctggcgcactttctggtgcgtaaaatcgcccataaccagagccgtacgctgcgcattagcgagggcgctatccgcctgctgatgagctacaactggcccggtaatgtgcgcgaactggaaaactgccttgagcgctcagcggtgatgtcggagaacggtctgatcgatcgggatgtgattttgtttaatcatcgcgaccagccagccaaaccgccagttatcagcgtctcgcatgatgataactggctcgataacaaccttgacgagcgccagcggctgattgcggcgctggaaaaagcgggatgggtacaagccaaagccgcgcgcttgctggggatgacgccgcgccaggtcgcctatcgtattcagacgatggatataaccctgccaaggctataa

SEQ ID NO: 184 - DNA, 2850 bp, Kosakonia sacchari
  gene 1..2850: note = glnE
  source 1..2850: mol_type = genomic DNA
atgccgcaccacgcaggattgtcgcagcactggcaaacggtattttctcgtctgccggaatcgctcaccgcgcagccattgagcgcgcaggcgcagtcagtgctcacttttagtgattttgttcaggacagcatcatcgcgcatcctgagtggctggcagagcttgaaagcgcgccgccgcctgcgaacgaatggcaacactatgcgcaatggctgcaagcggcgctggatggcgtcaccgatgaagcctcgctgatgcgcgcgctgcggctgtttcgccgtcgcatcatggtgcgcatcgcctggagccaggcgttacagttggtggcggaagaagatatcctgcaacagcttagcgtgctggcggaaaccctgatcgtcgccgcgcgcgactggctttatgaggcctgctgccgtgagtggggaacgccgagcaatccacaaggcgtggcgcagccgatgctggtactcggcatgggcaaactgggtggcggcgaactcaatttctcatccgatatcgatttgattttcgcctggccggaaaatggcgcaacgcgcggtggacgccgtgagctggataacgcgcaatttttcactcgccttggtcaacggctgattaaagtcctcgaccagccaacgcaggatggctttgtctaccgcgtcgatatgcgcttgcgcccgtttggcgacagcggcccgctggtgctgagctttgccgcgctggaagattactaccaggagcaggggcgcgattgggaacgctacgcgatggtgaaagcgcgcattatgggcgataacgacggcgaccatgcgcgggagttgcgcgcaatgctgcgcccgtttgttttccgccgttatatcgacttcagcgtgattcagtccctgcgtaacatgaaaggcatgattgcccgcgaagtgcgtcgccgtggcctgaaggacaacattaagctcggcgcgggcgggatccgcgaaatagaatttatcgtccaggttttccagctgattcgcggcggtcgcgagcctgcactgcaatcgcgttcactgttgccgacgcttgctgccatagatcaactgcatctgctgccggatggcgacgcaacccggctgcgcgaggcgtatttgtggctgcgacggctggagaacctgctgcaaagcatcaatgacgaacagacacagacgctgccgggcgatgaactgaatcgcgcgcgcctcgcctggggaatgggcaaagatagctgggaagcgctctgcgaaacgctggaagcgcatatgtcggcggtgcgtcagatatttaacgatctgattggcgatgatgaaacggattcgccggaagatgcgctttctgagagctggcgcgaattgtggcaggatgcgttgcaggaggaggattccacgcccgtgctggcgcatctctcagaggacgatcgccgccgcgtggtggcgctgattgccgattttcgcaaagagttggataaacgcaccattggcccgcgagggcggcaggtactcgatcacttaatgccgcatctgctcagcgatgtatgctcgcgcgacgatgcgccagtaccgctgtcacgcctgacgccgctgctcaccggaattattacccgcaccacttaccttgagctgctaagtgaatttcccggcgcactgaaacacctcatttccctgtgtgccgcgtcgccgatggttgccagtcagctggcgcgctacccgatcctgcttgatgaattgctcgacccgaatacgctctatcaaccgacggcgatgaatgcctatcgcgatgagctgcgccaatacctgctgcgcgtgccggaagatgatgaagagcaacagcttgaggcgctgcggcagtttaagcaggcgcagttgctgcgcgtggcggcggcggatattgccggtacgttgccagtaatgaaagtgagcgatcacttaacctggctggcggaagcgattattgatgcggtggtgcagcaagcctgggggcagatggtggcgcgttatggccagccaacgcatctgcacgatcgcgaagggcgcggttttgcggtggtcggttatggcaagctgggcggctgggagctgggttacagctccgatctggatctggtattcctgcacgactgcccgatggatgtgatgaccgatggcgagcgtgaaatcgatggtcgccagttctatttgcgtctcgcgcagcgcgtgatgcacctgtttagcacgcgcacgtcgtccggcatcctttatgaagttgatgcgcgtctgcgtccatctggcgctgcggggatgctggtcactactacggaatcgttcgccgattaccagcaaaacgaagcctggacgtgggaacatcaggcgctggcccgtgcgcgcgtggtgtacggcgatccgcaactgaccgccgaatttgacgccattcgccgcgatattctgatgacgcctcgcgacggcgcaacgctgcaaaccgacgtgcgagaaatgcgcgagaaaatgcgtgcccatcttggcaacaagcataaagaccgcttcgatctgaaagccgatgaaggcggtatcaccgacatcgagtttatcgcccaatatctggtgctgcgctttgcccatgacaagccgaaactgacgcgctggtcggataatgtgcgcattctcgaagggctggcgcaaaacggcatcatggaggagcaggaagcgcaggcattgacgctggcgtacaccacattgcgtgatgagctgcaccacctggcgctgcaagagttgccgggacatgtggcgctctcctgttttgtcgccgagcgtgcgcttattaaaaccagctgggacaagtggctggtggaaccgtgcgccccggcgtaa

SEQ ID NO: 185 - DNA, 1536 bp, Kosakonia sacchari
  gene 1..1536: note = 16S-3
  variation 766..776: note = n is a, c, t, g, unknown or other
  variation 905: note = n is a, c, t, g, unknown or other
  source 1..1536: mol_type = genomic DNA
attgaagagtttgatcatggctcagattgaacgctggcggcaggcctaacacatgcaagtcgaacggtagcacagagagcttgctctcgggtgacgagtggcggacgggtgagtaatgtctgggaaactgcctgatggagggggataactactggaaacggtagctaataccgcataacgtcgcaagaccaaagagggggaccttcgggcctcttgccatcagatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttcgggttgtaaagcactttcagcggggaggaagggagtaaggttaataaccttgctcattgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcatccgaaactggcaggcttgagtctcgtagagggaggtagaattccaggtgtagcggtgaaatgcgtagagatctggaggaataccggtggcgaaggcggcctcctggacgaagactgacgctcaggtgcgaaagnnnnnnnnnnnaacaggattagataccctggtagtccatgccgtaaacgatgtctactagccgttggggcctttgaggctttagtggcgcagctaacgcgataagtagaccgcctggggagtacggtcgcaagactaaanctcaaatgaattgacgggggcccgcacaagcggtggagcatgtggtttaattcgatgcaacgcgaagaaccttacctggccttgacatagtaagaattttccagagatggattggtgccttcgggaacttacatacaggtgctgcatggctgtcgtcagctcgtgtcgtgagatgttgggttaagtcccgcaacgagcgcaacccttgtcattagttgctacatttagttgggcactctaatgagactgccggtgacaaaccggaggaaggtggggatgacgtcaagtcctcatggcccttataggtggggctacacacgtcatacaatggctggtacaaagggttgccaacccgcgagggggagctaatcccataaaaccagtcgtagtccggatcgcagtctgcaactcgactgcgtgaagtcggaatcgctagtaatcgtggatcagaatgtcacggtgaatacgttcccgggtcttgtacacaccgcccgtcacaccatgggagcgggttctgccagaagtagttagcttaaccgcaaggagggcgattaccacggcagggttcgtgactggggtgaagtcgtaacaaggtagccgtatcggaaggtgcggctggatcacctccttt

SEQ ID NO: 186 - DNA, 882 bp, Kosakonia sacchari; identical to SEQ ID NO: 179
  gene 1..882: note = nifD1
  source 1..882: mol_type = genomic DNA

SEQ ID NO: 187 - DNA, 1386 bp, Kosakonia sacchari
  gene 1..1386: note = nifK1
  source 1..1386: mol_type = genomic DNA
atggccgaaattctgcgcagtaaaaaaccgctggcggtcagcccgataaaaagcggccagccgctgggggcgatcctcgcaagcctgggtgtcgaacagtgcataccgctggtacacggcgcacagggatgtagcgcgttcgcgaaggtgttctttattcaacattttcacgatccgatcccgctgcaatcgacggcgatggatccgacttccaccattatgggcgccgatgaaaacatttttaccgcgctcaatgtgctctgccagcgcaacgccgcgaaagccattgtgctgctcagcaccgggctttcagaagcccagggcagcgacatttcgcgggtggtgcgccagtttcgtgatgattttccccggcataaaggcgttgcgctgctcaccgtcaacacacccgatttctacggctcgctggaaaacggctacagcgccgtgctggaaagcatgattgaacagtgggtacccgcacagcccgccgccagcctgcgcaaccgccgtgtcaacctgctggtcagccatttactgacaccaggcgatatcgaactgttgcgcagttatgttgaagccttcggcctgcaaccggtgattgtgccggatctgtcgctgtcgctggacgggcatctggcagacggtgatttttcgcctgttacccaagggggaacatcgctgcgcatgattgaacagatggggcaaaacctggccacctttgtgattggcgcctcgctgggccgtgcggcggcgttactggcgcagcgcagccgtggcgaggtgatcgccctgccgcatctgatgacgcttgcagcctgcgacacgtttattcatcgactgaaaaccctctccgggcgcgatgtccccgcgtggattgagcgccagcgcggccaagttcaggatgcgatgatcgattgccatatgtggctgcagggtgcggctatcgccatggcagcagaaggcgatcacctggcggcatggtgcgatttcgcccgcagccagggcatgatccccggcccgattgtcgcaccggtcagccagccggggttgcaaaatctgccggttgaaaccgtggttatcggcgatctggaagatatgcaggatcggctttgcgcgacgcccgccgcgttactggtggccaattctcatgccgccgatctcgccacgcagtttgatttgtcacttatccgcgccgggttcccggtgtatgaccggctgggggaatttcgtcgcctgcgccaggggtacagcggcattcgtgacacgctgtttgagctggcgaatgtgatgcgcgagcgccatcacccgcttgcaacctaccgctcgccgctgcgccagcacgccgacgacaacgttacgcctggagatctgtatgccgcatgttaa

SEQ ID NO: 188 - DNA, 1287 bp, Kosakonia sacchari
  gene 1..1287: note = amtB
  source 1..1287: mol_type = genomic DNA
atgaaaaacacaacattaaaaacagcgcttgcttcgctggcgttactgcctggcctggcgatggcggctcccgctgtggcggataaagccgacaacggctttatgatgatttgcaccgcgctggtgctgtttatgaccattccgggcattgcgctgttctacggcggtttgatccgcggtaaaaacgtgctgtcgatgctgacgcaggttgccgtcaccttcgcactggtgtgcattctgtgggtggtgtatggctactcgctggcatttggcgagggcaacagcttcttcgggagttttaactgggcgatgttgaaaaacatcgaactgaaagccgtgatgggcagcatttatcagtatatccacgtggcgttccagggttccttcgcctgtatcaccgttggcctgattgtcggtgcactggctgagcgtattcgcttctctgcggtgctgatttttgtggtggtatggctgacgctttcttacgtgccgattgcacacatggtgtggggcggcggtctgctggcaacccacggtgcgctggatttcgcaggcggtacggttgttcacatcaacgctgcgattgcaggtctggtgggggcttacctgattggcaaacgcgtgggctttggcaaagaagcattcaaaccgcataacctgccgatggtcttcactggcaccgctatcctgtatgttggctggtttggtttcaacgccggctccgcaagctcggcgaacgaaattgctgcgctggccttcgtgaacactgtcgttgccactgctgccgctattctggcgtgggtatttggcgaatgggcaatgcgcggcaagccgtctctgctcggtgcctgttctggtgccatcgcgggtctggttggtatcacccccgcctgtggttatgtgggtgtcggcggtgcgctgattgtgggtctgattgccggtctggctgggctgtggggcgttactgcgctgaaacgtatgttgcgtgtcgatgacccgtgtgacgtattcggtgtgcacggcgtgtgcggcatcgtgggctgtatcctgacgggtatcttcgcctctacgtcgctgggtggtgtcggtttcgctgaaggtgtgaccatgggccatcaggtgctggtgcagctggaaagtgttgccatcactatcgtgtggtctggcgtggtggcctttattggttacaaactggcggacatgacggtaggcctgcgcgtaccggaagaacaagaacgtgaagggctggatgtaaacagccacggcgaaaacgcctataacgcctga

SEQ ID NO: 189 - DNA, 348 bp, Kosakonia sacchari
  gene 1..348: note = Prm1
  source 1..348: mol_type = genomic DNA
cgtcctgtaataataaccggacaattcggactgattaaaaaagcgcccttgtggcgctttttttatattcccgcctccatttaaaataaaaaatccaatcggatttcactatttaaactggccattatctaagatgaatccgatggaagctcgctgttttaacacgcgttttttaaccttttattgaaagtcggtgcttctttgagcgaacgatcaaatttaagtggattcccatcaaaaaaatattctcaacctaaaaaagtttgtgtaatacttgtaacgctacatggagattaactcaatctagagggtattaataatgaatcgtactaaactggtactgggcgc

SEQ ID NO: 190 - DNA, 313 bp, Kosakonia sacchari
  gene 1..313: note = Prm5
  source 1..313: mol_type = genomic DNA
ggacatcatcgcgacaaacaatattaataccggcaaccacaccggcaatttacgagactgcgcaggcatcctttctcccgtcaatttctgtcaaataaagtaaaagaggcagtctacttgaattacccccggctggttgagcgtttgttgaaaaaaagtaactgaaaaatccgtagaatagcgccactctgatggttaattaacctattcaattaagaattatctggatgaatgtgccattaaatgcgcagcataatggtgcgttgtgcgggaaaactgcttttttttgaaagggttggtcagtagcggaaac

SEQ ID NO: 191 - DNA, 1485 bp, Rahnella aquatilis; identical to SEQ ID NO: 19
  gene 1..1485: note = nifL
  source 1..1485: mol_type = genomic DNA

SEQ ID NO: 192 - DNA, 1575 bp, Rahnella aquatilis; identical to SEQ ID NO: 20
  gene 1..1575: note = nifA
  source 1..1575: mol_type = genomic DNA

SEQ ID NO: 193 - DNA, 1540 bp, Rahnella aquatilis
  gene 1..1540: note = 16S-1
  source 1..1540: mol_type = genomic DNA
attgaagagtttgatcatggctcagattgaacgctggcggcaggcctaacacatgcaagtcgagcggcagcgggaagtagcttgctactttgccggcgagcggcggacgggtgagtaatgtctgggaaactgcctgatggagggggataactactggaaacggtagctaataccgcatgacctcgaaagagcaaagtgggggatcttcggacctcacgccatcggatgtgcccagatgggattagctagtaggtgaggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttagggttgtaaagcactttcagcgaggaggaaggcatcatacttaatacgtgtggtgattgacgttactcgcagaagaagcaccggctaactccgtgccagcagccgcggtaatacggagggtgcaagcgttaatcggaattactgggcgtaaagcgcacgcaggcggtttgttaagtcagatgtgaaatccccgagcttaacttgggaactgcatttgaaactggcaagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaatttgccagagatggcgaagtgccttcgggaactctgagacaggtgctgcatggctgtcgtcagctcgtgttgtgaaatgttgggttaagtcccgcaacgagcgcaacccttatcctttgttgccagcacgtaatggtgggaactcaaaggagactgccggtgataaaccggaggaaggtggggatgacgtcaagtcatcatggcccttacgagtagggctacacacgtgctacaatggcatatacaaagagaagcgaactcgcgagagcaagcggacctcataaagtatgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 194 - DNA, 1540 bp, Rahnella aquatilis
  gene 1..1540: note = 16S-2
  variation 267: note = n is a, c, t, g, unknown or other
  source 1..1540: mol_type = genomic DNA
attgaagagtttgatcatggctcagattgaacgctggcggcaggcctaacacatgcaagtcgagcggcatcgggaagtagcttgctactttgccggcgagcggcggacgggtgagtaatgtctgggaaactgcctgatggagggggataactactggaaacggtagctaataccgcatgacctcgaaagagcaaagtgggggatcttcggacctcacgccatcggatgtgcccagatgggattagctagtaggtgaggtaatggctnacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttagggttgtaaagcactttcagcgaggaggaaggcatcatacttaatacgtgtggtgattgacgttactcgcagaagaagcaccggctaactccgtgccagcagccgcggtaatacggagggtgcaagcgttaatcggaattactgggcgtaaagcgcacgcaggcggtttgttaagtcagatgtgaaatccccgagcttaacttgggaactgcatttgaaactggcaagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaatttgccagagatggcgaagtgccttcgggaactctgagacaggtgctgcatggctgtcgtcagctcgtgttgtgaaatgttgggttaagtcccgcaacgagcgcaacccttatcctttgttgccagcacgtgatggtgggaactcaaaggagactgccggtgataaaccggaggaaggtggggatgacgtcaagtcatcatggcccttacgagtagggctacacacgtgctacaatggcatatacaaagagaagcgaactcgcgagagcaagcggacctcataaagtatgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 195 - DNA, 1540 bp, Rahnella aquatilis
  gene 1..1540: note = 16S-3
  variation 455: note = n is a, c, t, g, unknown or other
  source 1..1540: mol_type = genomic DNA
attgaagagtttgatcatggctcagattgaacgctggcggcaggcctaacacatgcaagtcgagcggcagcgggaagtagcttgctactttgccggcgagcggcggacgggtgagtaatgtctgggaaactgcctgatggagggggataactactggaaacggtagctaataccgcatgacctcgaaagagcaaagtgggggatcttcggacctcacgccatcggatgtgcccagatgggattagctagtaggtgaggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttagggttgtaaagcactttcagcgaggaggaaggcancatacttaatacgtgtggtgattgacgttactcgcagaagaagcaccggctaactccgtgccagcagccgcggtaatacggagggtgcaagcgttaatcggaattactgggcgtaaagcgcacgcaggcggtttgttaagtcagatgtgaaatccccgagcttaacttgggaactgcatttgaaactggcaagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaatttgccagagatggcgaagtgccttcgggaactctgagacaggtgctgcatggctgtcgtcagctcgtgttgtgaaatgttgggttaagtcccgcaacgagcgcaacccttatcctttgttgccagcacgtgatggtgggaactcaaaggagactgccggtgataaaccggaggaaggtggggatgacgtcaagtcatcatggcccttacgagtagggctacacacgtgctacaatggcatatacaaagagaagcgaactcgcgagagcaagcggacctcataaagtatgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 196 - DNA, 1540 bp, Rahnella aquatilis
  gene 1..1540: note = 16S-4
  variation 70: note = n is a, c, t, g, unknown or other
  source 1..1540: mol_type = genomic DNA
attgaagagtttgatcatggctcagattgaacgctggcggcaggcctaacacatgcaagtcgagcggcancgggaagtagcttgctactttgccggcgagcggcggacgggtgagtaatgtctgggaaactgcctgatggagggggataactactggaaacggtagctaataccgcatgacctcgaaagagcaaagtgggggatcttcggacctcacgccatcggatgtgcccagatgggattagctagtaggtgaggtaatggcttacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttagggttgtaaagcactttcagcgaggaggaaggcatcacacttaatacgtgtggtgattgacgttactcgcagaagaagcaccggctaactccgtgccagcagccgcggtaatacggagggtgcaagcgttaatcggaattactgggcgtaaagcgcacgcaggcggtttgttaagtcagatgtgaaatccccgagcttaacttgggaactgcatttgaaactggcaagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaatttgccagagatggcgaagtgccttcgggaactctgagacaggtgctgcatggctgtcgtcagctcgtgttgtgaaatgttgggttaagtcccgcaacgagcgcaacccttatcctttgttgccagcacgtgatggtgggaactcaaaggagactgccggtgataaaccggaggaaggtggggatgacgtcaagtcatcatggcccttacgagtagggctacacacgtgctacaatggcatatacaaagagaagcgaactcgcgagagcaagcggacctcataaagtatgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 197 - DNA, 1540 bp, Rahnella aquatilis
  gene 1..1540: note = 16S-5
  source 1..1540: mol_type = genomic DNA
attgaagagtttgatcatggctcagattgaacgctggcggcaggcctaacacatgcaagtcgagcggcagcgggaagtagcttgctactttgccggcgagcggcggacgggtgagtaatgtctgggaaactgcctgatggagggggataactactggaaacggtagctaataccgcatgacctcgaaagagcaaagtgggggatcttcggacctcacgccatcggatgtgcccagatgggattagctagtaggtgaggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttagggttgtaaagcactttcagcgaggaggaaggcatcacacttaatacgtgtgttgattgacgttactcgcagaagaagcaccggctaactccgtgccagcagccgcggtaatacggagggtgcaagcgttaatcggaattactgggcgtaaagcgcacgcaggcggtttgttaagtcagatgtgaaatccccgagcttaacttgggaactgcatttgaaactggcaagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaatttgccagagatggcgaagtgccttcgggaactctgagacaggtgctgcatggctgtcgtcagctcgtgttgtgaaatgttgggttaagtcccgcaacgagcgcaacccttatcctttgttgccagcacgtgatggtgggaactcaaaggagactgccggtgataaaccggaggaaggtggggatgacgtcaagtcatcatggcccttacgagtagggctacacacgtgctacaatggcatatacaaagagaagcgaactcgcgagagcaagcggacctcataaagtatgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 198 - DNA, 1009 bp, Rahnella aquatilis
  gene 1..1009: note = 16S-6
  variation 296: note = n is a, c, t, g, unknown or other
  variation 311: note = n is a, c, t, g, unknown or other
  variation 973: note = n is a, c, t, g, unknown or other
  source 1..1009: mol_type = genomic DNA
gtagctaataccgcatgacctcgaaagagcaaagtgggggatcttcggacctcacgccatcggatgtgcccagatgggattagctagtaggtgaggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttagggttgtaaagcactttcagcgaggaggaaggcatcanacttaatacgtgtgntgattgacgttactcgcagaagaagcaccggctaactccgtgccagcagccgcggtaatacggagggtgcaagcgttaatcggaattactgggcgtaaagcgcacgcaggcggtttgttaagtcagatgtgaaatccccgagcttaacttgggaactgcatttgaaactggcaagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaatttgccagagatggcgaagtgccttcgggaactctgagacaggtgctgcatggctgtcgtcagctcgtgttgtgaaatgttgggttaagtcccgcaacgagcgcaacccttatcctttgttgccagcacgtnatggtgggaactcaaaggagactgccggtgataaac

SEQ ID NO: 199 - DNA, 1519 bp, Rahnella aquatilis
  gene 1..1519: note = 16S-7
  source 1..1519: mol_type = genomic DNA
attgaagagtttgatcatggctcagattgaacgctggcggcaggcctaacacatgcaagtcgagcggcagcgggaagtagcttgctactttgccggcgagcggcggacgggtgagtaatgtcctgatggaggggataactactggaacggtagctaataccgcacctcgaaagagcaaagtgggggatcttcggacctcacgccatcggatgtgcccagatgggattagctagtaggtgaggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttagggttgtaaagcactttcagcgaggaggaaggcatcatacttaatacgtgtggtgattgacgttactcgcagaagaagcaccggctaactccgtgccagcagccgcggtaatacggagggtgcaagcttaatcggaattactgggcgtaaagcgcacgcaggcggttgttaagtcagatgtgaaatccccgagcttaacttgggaactgcatttgaaactggcaagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattaataccctggtagtccacgctgtaacgatgtcgacttggaggttgtgccctgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaatttgccagagatggcgaagtgccttcgggaactctgagacaggtgctgcatggctgtcgtcagctcgtgttgtgaaatgttgggttaagtcccgcaacgagcgcaacccttatcctttgttgccagcacgtaatggtgggaactcaaaggagactgccggtgataaaccggaggaaggtggggatgacgtcaagtcatcatggcccttacgagtagggctacacacgtgctacaatggcatatacaaagagaagcgaactcgcgagagcaagcggacctcataaagtatgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 200 - DNA, 882 bp, Rahnella aquatilis; identical to SEQ ID NO: 13
  gene 1..882: note = nifH1
  source 1..882: mol_type = genomic DNA

SEQ ID NO: 201 - DNA, 1113 bp, Rahnella aquatilis; identical to SEQ ID NO: 14
  gene 1..1113: note = nifH2
  source 1..1113: mol_type = genomic DNA

SEQ ID NO: 202 - DNA, 1374 bp, Rahnella aquatilis; identical to SEQ ID NO: 15
  gene 1..1374: note = nifD1
  source 1..1374: mol_type = genomic DNA

SEQ ID NO: 203 - DNA, 1449 bp, Rahnella aquatilis; identical to SEQ ID NO: 16
  gene 1..1449: note = nifD2
  source 1..1449: mol_type = genomic DNA

SEQ ID NO: 204 - DNA, 1386 bp, Rahnella aquatilis; identical to SEQ ID NO: 17
  gene 1..1386: note = nifK1
  source 1..1386: mol_type = genomic DNA

SEQ ID NO: 205 - DNA, 1563 bp, Rahnella aquatilis; identical to SEQ ID NO: 18
  gene 1..1563: note = nifK2
  source 1..1563: mol_type = genomic DNA

SEQ ID NO: 206 - DNA, 2838 bp, Rahnella aquatilis; identical to SEQ ID NO: 21
  gene 1..2838: note = glnE
  source 1..2838: mol_type = genomic DNA

SEQ ID NO: 207 - DNA, 449 bp, Rahnella aquatilis
  gene 1..449: note = Prm4
  source 1..449: mol_type = genomic DNA
agtctgaactcatcctgcggcagtcggtgagacgtatttttgaccaaagagtgatctacatcacggaattttgtggttgttgctgcttaaaagggcaaatctacccttagaatcaactgttatatcagggggattcagagagatattaggaatttgcacaagcgcacaatttaaccacatcatgataacgccatgtaaaacaaagataaaaaaacaaaatgcagtgacttacatcgcaagcaaggcattttcttatccaattgctcaaagtttggcctttcatatcgcaacgaaaatgcgtaatatacgcgcccttgcggacatcagtatggtcattcctagttcatgcgcatcggacaccaccagcttacaaattgcctgattgcggccccgatggccggtatcactgaccgaccatttcgtgccttatgtcatgcgatgggggctgg

SEQ ID NO: 208 - DNA, 500 bp, Rahnella aquatilis
  gene 1..500: note = Prm1.2
  source 1..500: mol_type = genomic DNA
tgaacatcactgatgcacaagctacctatgtcgaagaattaactaaaaaactgcaagatgcaggcattcgcgttaaagccgacttgagaaatgagaagattggctttaaaattcgcgaacacacgctacgccgtgttccttatatgttagtttgtggcgataaagaggtcgaagcaggcaaagttgctgttcgtacccgccgcggcaaagacttaggaagcatggatgttagcgaagtcgttgacaaactgctggcggaaatccgcagcagaagtcttcatcaactggaggaataaagtattaaaggcggaaaacgagttcaaccggcgcgtcctaatcgcattaacaaagagattcgcgcgcaagaagttcgcctcacaggcgtcgatggcgagcagattggtattgtcagtctgaatgaagctcttgaaaaagctgaggaagcgggcgtcgatttagtagaaatcagtccgaatgccgagccgccagtttgtcgaatc

SEQ ID NO: 209 - DNA, 170 bp, Rahnella aquatilis
  gene 1..170: note = Prm3.1
  source 1..170: mol_type = genomic DNA
tacagtagcgcctctcaaaaatagataaacggctcatgtacgtgggccgtttattttttctacccataatcgggaaccggtgttataatgccgcgccctcatattgtggggatttcttaatgacctatcctgggtcctaaagttgtagttgacattagcggagcactaac

SEQ ID NO: 210 - DNA, 142 bp, Rahnella aquatilis
  gene 1..142: note = Prm6.1
  source 1..142: mol_type = genomic DNA
aattttttttcacaaagcgtagcgttattgaatcgcacattttaaactgttggccgctgtggaagcgaatattggtgaaaggtgcggttttaaggcctttttctttgactctctgtcgttacaaagttaatatgcgcgccct

SEQ ID NO: 211 - DNA, 293 bp, Rahnella aquatilis
  gene 1..293: note = Prm7.1
  source 1..293: mol_type = genomic DNA
ttaaaaacgtgaccacgagcattaataaacgccacgaaatgtggcgtttatttattcaaaaagtatcttctttcataaaaagtgctaaatgcagtagcagcaaaattgggataagtcccatggaatacggctgttttcgctgcaatttttaactttttcgtaaaaaaagatgtttctttgagcgaacgatcaaaatatagcgttaaccggcaaaaaattattctcattagaaaatagtttgtgtaatacttgtaacgctacatggagattaacttaatctagagggttttata

SEQ ID NO: 212 - DNA, 1188 bp, synthetic construct; identical to SEQ ID NO: 127
  misc_feature 1..1188: note = Synthetic polynucleotide
  misc_feature 1..1188: note = glnE-delta-AR-2
  source 1..1188: mol_type = other DNA

SEQ ID NO: 213 - DNA, 2188 bp, synthetic construct; identical to SEQ ID NO: 128
  misc_feature 1..2188: note = Synthetic polynucleotide
  misc_feature 1..2188: note = glnE-delta-AR-2 with 500bp flank
  source 1..2188: mol_type = other DNA

SEQ ID NO: 214 - DNA, 524 bp, synthetic construct
  misc_feature 1..524: note = Synthetic polynucleotide
  misc_feature 1..524: note = delta-nifL::null-v1
  source 1..524: mol_type = other DNA
atgagcatcacggcgttatcagcatcatttcctgaggggaatatcgccagccgcttgtcgctgcaacatccttcactgttttataccgtggttgaacaatcttcggtggcgatttcgctgaccgatccgcaggcgcgcatttgttatgccaatccggcattctgccgccagacgggttttgcacttgagacacttttgggcgagaaccaccgtctgctggagtctgaactcatcctgcgatgggggctgggccgtctctgaagctctcggtgaacattgttgcgaggcaggatgcgagctggttgtgttttgacattaccgataatgtgccgcgtgaacgggtgcgttatgcccgcccggaagcggcgttttcccgtccggggaatggcatggagctgcgccttatccagacgctgatcgcccatcatcgcggttctttagatctctcggtccgccctgatggcggcaccttgctgacgttacgcctgccggtacagcaggttatcaccggaggcttaaaatga

SEQ ID NO: 215 - DNA, 1524 bp, synthetic construct
  misc_feature 1..1524: note = Synthetic polynucleotide
  misc_feature 1..1524: note = delta-nifL::null-v1 with 500bp flank
  source 1..1524: mol_type = other DNA
tgtttcgtctcgaggccgggcaactgagcggccccgttgaaaccgacctgggctggcatctgttgttgtgcgaacaaattcgcctgccgcaacccttgccgaaagccgaagccttaacgcgggtgcgtcagcaactgattgcccggcaacagaaacattatcagcgccagtggctgcaacaactgatcaacgcctgagcctgttctccttcttgttgatgcagacgggttaatgcccgttttgcacgaaaaatgcacataaattgcctgcgttgccttataacagcgcagggaaatcctgcctccggccttgtgccacaccgcgctttgcctggtttgtggtaaaaactggcccgctttgcatcctgatgcttaaaacaccccgttcagatcaacctttgggcagataagcccgcgaaaggcctgcaaattgcacggttattccgggtgagtatatgtgtgatttgggttccggcattgcgcaataaaggggagaaagacatgagcatcacggcgttatcagcatcatttcctgaggggaatatcgccagccgcttgtcgctgcaacatccttcactgttttataccgtggttgaacaatcttcggtggcgatttcgctgaccgatccgcaggcgcgcatttgttatgccaatccggcattctgccgccagacgggttttgcacttgagacacttttgggcgagaaccaccgtctgctggagtctgaactcatcctgcgatgggggctgggccgtctctgaagctctcggtgaacattgttgcgaggcaggatgcgagctggttgtgttttgacattaccgataatgtgccgcgtgaacgggtgcgttatgcccgcccggaagcggcgttttcccgtccggggaatggcatggagctgcgccttatccagacgctgatcgcccatcatcgcggttctttagatctctcggtccgccctgatggcggcaccttgctgacgttacgcctgccggtacagcaggttatcaccggaggcttaaaatgacccagttacctaccgcgggcccggttatccggcgctttgatatgtctgcccagtttacggcgctttatcgcatcagcgtggcgctgagtcaggaaagcaacaccgggcgcgcactggcggcgatcctcgaagtgcttcacgatcatgcatttatgcaatacggcatggtgtgtctgtttgataaagaacgcaatgcactctttgtggaatccctgcatggcatcgacggcgaaaggaaaaaagagacccgccatgtccgttaccgcatgggggaaggcgtgatcggcgcggtgatgagccagcgtcaggcgctggtgttaccgcgcatttcagacgatcagcgttttctcgaccgcctgaatatttacgattacagcctgccgttgattggcgtgccgatccccggtgcggataatcagccatcgggcgtgctggtggcacagccgatggcgttgcacgaagaccggctgactgccagtacgcggtttttagaaatggtc

SEQ ID NO: 216 - DNA, 266 bp, synthetic construct
  misc_feature 1..266: note = Synthetic polynucleotide
  misc_feature 1..266: note = delta-nifL::null-v2
  source 1..266: mol_type = other DNA
atgagcatcacggcgttatcagcatcatttcctgaggggaatatcgccagccgcttgtcgctgcaacatccttcactgttttataccgtggttgaacaatcttcggtggcgatttcgctgaccgatccgcaggcgcgcatttgttatgccaatccggcattctgccgccagacgggttttgcacttgagacacttttgggcgagaaccaccgtctgctggttaaagcctgccggtacagcaggttatcaccggaggcttaaaatga

SEQ ID NO: 217 - DNA, 1266 bp, synthetic construct
  misc_feature 1..1266: note = Synthetic polynucleotide
  misc_feature 1..1266: note = delta-nifL::null-v2 with 500bp flank
  source 1..1266: mol_type = other DNA
tgtttcgtctcgaggccgggcaactgagcggccccgttgaaaccgacctgggctggcatctgttgttgtgcgaacaaattcgcctgccgcaacccttgccgaaagccgaagccttaacgcgggtgcgtcagcaactgattgcccggcaacagaaacattatcagcgccagtggctgcaacaactgatcaacgcctgagcctgttctccttcttgttgatgcagacgggttaatgcccgttttgcacgaaaaatgcacataaattgcctgcgttgccttataacagcgcagggaaatcctgcctccggccttgtgccacaccgcgctttgcctggtttgtggtaaaaactggcccgctttgcatcctgatgcttaaaacaccccgttcagatcaacctttgggcagataagcccgcgaaaggcctgcaaattgcacggttattccgggtgagtatatgtgtgatttgggttccggcattgcgcaataaaggggagaaagacatgagcatcacggcgttatcagcatcatttcctgaggggaatatcgccagccgcttgtcgctgcaacatccttcactgttttataccgtggttgaacaatcttcggtggcgatttcgctgaccgatccgcaggcgcgcatttgttatgccaatccggcattctgccgccagacgggttttgcacttgagacacttttgggcgagaaccaccgtctgctggttaaagcctgccggtacagcaggttatcaccggaggcttaaaatgacccagttacctaccgcgggcccggttatccggcgctttgatatgtctgcccagtttacggcgctttatcgcatcagcgtggcgctgagtcaggaaagcaacaccgggcgcgcactggcggcgatcctcgaagtgcttcacgatcatgcatttatgcaatacggcatggtgtgtctgtttgataaagaacgcaatgcactctttgtggaatccctgcatggcatcgacggcgaaaggaaaaaagagacccgccatgtccgttaccgcatgggggaaggcgtgatcggcgcggtgatgagccagcgtcaggcgctggtgttaccgcgcatttcagacgatcagcgttttctcgaccgcctgaatatttacgattacagcctgccgttgattggcgtgccgatccccggtgcggataatcagccatcgggcgtgctggtggcacagccgatggcgttgcacgaagaccggctgactgccagtacgcggtttttagaaatggtc

SEQ ID NO: 218 - DNA, 943 bp, synthetic construct
  misc_feature 1..943: note = Synthetic polynucleotide
  misc_feature 1..943: note = delta-nifL::Prm4
  source 1..943: mol_type = other DNA
atgagcatcacggcgttatcagcatcatttcctgaggggaatatcgccagccgcttgtcgctgcaacatccttcactgttttataccgtggttgaacaatcttcggtggcgatttcgctgaccgatccgcaggcgcgcatttgttatgccaatccggcattctgccgccagacgggttttgcacttgagacacttttgggcgagaaccaccgtctgctggagtctgaactcatcctgcggcagtcggtgagacgtatttttgaccaaagagtgatctacatcacggaattttgtggttgttgctgcttaaaagggcaaatctacccttagaatcaactgttatatcagggggattcagagagatattaggaatttgcacaagcgcacaatttaaccacatcatgataacgccatgtaaaacaaagataaaaaaacaaaatgcagtgacttacatcgcaagcaaggcattttcttatccaattgctcaaagtttggcctttcatatcgcaacgaaaatgcgtaatatacgcgcccttgcggacatcagtatggtcattcctagttcatgcgcatcggacaccaccagcttacaaattgcctgattgcggccccgatggccggtatcactgaccgaccatttcgtgccttatgtcatgcgatgggggctgggccgtctctgaagctctcggtgaacattgttgcgaggcaggatgcgagctggttgtgttttgacattaccgataatgtgccgcgtgaacgggtgcgttatgcccgcccggaagcggcgttttcccgtccggggaatggcatggagctgcgccttatccagacgctgatcgcccatcatcgcggttctttagatctctcggtccgccctgatggcggcaccttgctgacgttacgcctgccggtacagcaggttatcaccggaggcttaaaatga

SEQ ID NO: 219 - DNA, 1943 bp, synthetic construct
  misc_feature 1..1943: note = Synthetic polynucleotide
  misc_feature 1..1943: note = delta-nifL::Prm4 with 500bp flank
  source 1..1943: mol_type = other DNA
tgtttcgtctcgaggccgggcaactgagcggccccgttgaaaccgacctgggctggcatctgttgttgtgcgaacaaattcgcctgccgcaacccttgccgaaagccgaagccttaacgcgggtgcgtcagcaactgattgcccggcaacagaaacattatcagcgccagtggctgcaacaactgatcaacgcctgagcctgttctccttcttgttgatgcagacgggttaatgcccgttttgcacgaaaaatgcacataaattgcctgcgttgccttataacagcgcagggaaatcctgcctccggccttgtgccacaccgcgctttgcctggtttgtggtaaaaactggcccgctttgcatcctgatgcttaaaacaccccgttcagatcaacctttgggcagataagcccgcgaaaggcctgcaaattgcacggttattccgggtgagtatatgtgtgatttgggttccggcattgcgcaataaaggggagaaagacatgagcatcacggcgttatcagcatcatttcctgaggggaatatcgccagccgcttgtcgctgcaacatccttcactgttttataccgtggttgaacaatcttcggtggcgatttcgctgaccgatccgcaggcgcgcatttgttatgccaatccggcattctgccgccagacgggttttgcacttgagacacttttgggcgagaaccaccgtctgctggagtctgaactcatcctgcggcagtcggtgagacgtatttttgaccaaagagtgatctacatcacggaattttgtggttgttgctgcttaaaagggcaaatctacccttagaatcaactgttatatcagggggattcagagagatattaggaatttgcacaagcgcacaatttaaccacatcatgataacgccatgtaaaacaaagataaaaaaacaaaatgcagtgacttacatcgcaagcaaggcattttcttatccaattgctcaaagtttggcctttcatatcgcaacgaaaatgcgtaatatacgcgcccttgcggacatcagtatggtcattcctagttcatgcgcatcggacaccaccagcttacaaattgcctgattgcggccccgatggccggtatcactgaccgaccatttcgtgccttatgtcatgcgatgggggctgggccgtctctgaagctctcggtgaacattgttgcgaggcaggatgcgagctggttgtgttttgacattaccgataatgtgccgcgtgaacgggtgcgttatgcccgcccggaagcggcgttttcccgtccggggaatggcatggagctgcgccttatccagacgctgatcgcccatcatcgcggttctttagatctctcggtccgccctgatggcggcaccttgctgacgttacgcctgccggtacagcaggttatcaccggaggcttaaaatgacccagttacctaccgcgggcccggttatccggcgctttgatatgtctgcccagtttacggcgctttatcgcatcagcgtggcgctgagtcaggaaagcaacaccgggcgcgcactggcggcgatcctcgaagtgcttcacgatcatgcatttatgcaatacggcatggtgtgtctgtttgataaagaacgcaatgcactctttgtggaatccctgcatggcatcgacggcgaaaggaaaaaagagacccgccatgtccgttaccgcatgggggaaggcgtgatcggcgcggtgatgagccagcgtcaggcgctggtgttaccgcgcatttcagacgatcagcgttttctcgaccgcctgaatatttacgattacagcctgccgttgattggcgtgccgatccccggtgcggataatcagccatcgggcgtgctggtggcacagccgatggcgttgcacgaagaccggctgactgccagtacgcggtttttagaaatggtc

SEQ ID NO: 220 - DNA, 2864 bp, Kosakonia sacchari
  gene 1..2864: note = glnD with flanking sequences
  source 1..2864: mol_type = genomic DNA
ttctgacattagcaaacgcgataaacggtccgctctgcgggccgttttttttaccgctcttccctgcagcgtcatacatataaacgacttttcatttttcattatctcttttaagttgttttgctatgcatatgcactccagacgggtggcccataatgagcaacatcacttcagaacaatttgtcagcgcggtacaacccacccttcccggccagcctgaatatccaggggtttggtcacaagacgaattccagtgcgccaatatcaaagcgcatattgaggctttccagcagtggctcgggcacgtctttgacgccggaatttcagccgagcaattaattgaagcgcgcaccgaatttattgatcaacttctgcaacgcttatggctggagtacggttttggcgacgtcagtgataccgcgctggttgcggtaggcggttacggtcgcggcgaactgcatccgctatcggatattgatttactgatcctcagccgcagcaaactttccgacgaacaagcgcagaagattggcgaactgctcacgctgctgtgggatgtcaaactcgaagtgggacacagcgttcgtacgctggatgagtgcttacaggaagggttatcggatctcactgtcgcgaccaacttaattgaatcacgtctgctgattggcgacgtcgcgctgtttctggaactgcaaaagcatatttttagcgacagcttctggccatcggaaacttttttcccggcaaaagtggcggaacaaaacgagcgccaccagcgttatcacggcaccagttataacctggaacccgacataaaaagtagccctggtggtctgcgcgatattcacaccctgcaatgggttgctcgccgtcactttggtgccacatcgctggatgaaatggtgggctttggctttctgaccgaagccgagcgcaatgagctgaacgagtgtctgcacctgctttggcgtatccgcttcgcgctgcatctggaagtgagccgctacgacaaccgcctgctgttcgaccgccaactgagcgtagcgcagcgtctgaattatgtcggtgaaggcaaccagcctgtcgaacatatgatgaaggatttcttccgcgttacccgccgcgtcgcggagctgaaccagatgctgttacagctctttgatgaagcgattctggcgctgaccaccgacgagaaaccgcgcccgattgacgatgaattccaactgcgcggttcgcttatcgatctgcgtgacgaaacgctgtttatccgtgagccgcaggccatcctgcgcatgttctatatcatggtgcgcaacagcaccattaccggcatctactccaccacgctgcgccacttgcgccacgcccgccgccatctgacgcagccgctgtgttacatcgcggaagcacgctctattttcctcagcatgttgcgccatccaggcgccgtcagccgcggtttgctgccgatgcaccgccacagcgtgttgtgggcttatatgccacagtggtcgcacatcgtcggtcagatgcagtttgacctgttccacgcctataccgtggatgagcacaccattcgcgtattgctgaagctggaaagttttgccaaagaagagacgcgcaataagcaccctctatgcgtggaactgtggccgcgtttgacccacccggagctgatcctgatcgcggcgttgttccatgatatcgccaaaggacgcggcggcgaccactcggtgctgggtgcgcaggatgtgctgaagttcgctgaattacacgggcttaactcacgcgaaacccagctggttgcctggctggtgcgccatcatctgttgatgtcggtcaccgcccagcgccgtgatattcaggatccggaagtcatcaagcagtttgccgaagaggtgcaaacggaaaaccgtctgcgcttcctcgtctgtctgaccgtggcggatatctgtgccaccaacgaaaccctgtggaacagttggaagcagagcttgctgcgcgagctctatttcgcgactgaaaaacagctgcgtcgcggcatgcaaaacacgccggatatgcgcgaacgcgtgcgccatcaccagcttcaggcgctggcgttgctgcgtatggataatattgatgaagagcagttgcatcagatttgggcgcgctgtcgggcgaactattttgttcgccatagcccaaaccagcttgcctggcatgcgcgccacctgctgcaacatgatctgagcaagccgatgattttgctcagcccacaggctacgcgcggcggcaccgagatctttatctggagcccggatcgcccgtatctgtttgcagcggtgtgcgccgagcttgatcgtcgcaacctgagcgttcacgacgcgcagatttttaccacccgcgacggcatggcaatggatacctttattgtgctggaaccggacggaagcccgctctccgccgatcgccacgacgcgatttgtttcgggctggagcaggccattacgcaacacacctggcagccgccgcaaccgcgccgtcagcctgccaaattgcgccactttaccgtcgataccgaagtgaatttcctgccaacccataccgacagaaaatcgtttcttgaattgattgcccttgaccagccagggctgcttgcccgcgtcgggcaggtgtttgcggatctgggaatttcgctgcacggggcgagaattacaaccattggtgagcgagtagaagatttatttataatcgcgactgccgaccggcgtggccttaataatgcgctgcaaaaggaggtacaacaacggttgacagaggccctcaatccaaacgataaagggtgattatttttttacacagatggaaagagtaaaca

SEQ ID NO: 221 - DNA, 426 bp, synthetic construct; identical to SEQ ID NO: 147
  misc_feature 1..426: note = delta-nifL::Prm5
  source 1..426: mol_type = other DNA

SEQ ID NO: 222 - DNA, 1563 bp, synthetic construct; identical to SEQ ID NO: 155
  misc_feature 1..1563: note = glnE-delta-AR
  source 1..1563: mol_type = other DNA

SEQ ID NO: 223 - DNA, 188 bp, synthetic construct
  misc_feature 1..188: note = delta-glnD
  source 1..188: mol_type = other DNA
ttctgacattagcaaacgcgataaacggtccgctctgcgggccgttttttttaccgctcttccctgcagcgtcatacatataaacgacttttcatttttcattatctcttttaagttgttttgctatgcatatgcactccagacgggtggcccatattatttttttacacagatggaaagagtaaaca